(19) United States
(12) Patent Application Publication
(10) Pub. No.: US 2007/0253978 A1
(43) Pub. Date: Nov. 1, 2007

(54) Copy choice recombination and uses thereof

(76) Inventor: Henry L. Niman, Pittsburgh, PA (US)

Correspondence Address: LAHIVE & COCKFIELD, LLP, ONE POST OFFICE SQUARE, BOSTON, MA, 02109-2127, US

(21) Appl. No.: 11/648,859

(22) Filed: Dec. 29, 2006

Related U.S. Application Data
(63) Continuation of application No. PCT/US05/22873, filed on Jun. 23, 2005.
(60) Provisional application No. 60/585,306, filed on Jul. 2, 2004.
(60) Provisional application No. 60/587,580, filed on Jul. 13, 2004.
(60) Provisional application No. 60/590,162, filed on Jul. 21, 2004.
(60) Provisional application No. 60/692,060, filed on Jun. 16, 2005.

(51) Int. Cl. A61K 39/00; A01K 67/027; C12P 1/04; C12Q 1/68

(52) U.S. Cl. 424/206.1; 424/204.1; 435/41; 435/5; 800/3; 800/4

(57) ABSTRACT

The instant invention provides methods for determining, predicting and characterizing the genetic variability of a range of organisms, including, e.g., viruses, microbes, cells and multicellular organisms. Accordingly, the invention provides methods for identifying virulent pathogens, genetic mutations within pathogens that are relevant to animal health, and methods and compositions for prophylactic or therapeutic intervention against such pathogens.

### RELATED APPLICATIONS

This application is related to U.S. Provisional Application No. 60/585,306 filed on Jul. 2, 2004, U.S. Provisional Application No. 60/587,580, filed on Jul. 13, 2004, U.S. Provisional Application No. 60/590,162 filed on Jul. 21, 2004, and U.S. Provisional Application No. 60/______ filed on Jun. 16, 2005, the entire contents of which are incorporated herein by reference.

The contents of all references, patents and published patent applications cited throughout this application are hereby incorporated herein by reference in their entirety.

### BACKGROUND

Despite remarkable achievements in the development of molecular genetics for understanding human and animal disease as well as determining the genetic nature of pathogens, rules for prediction or prognosis of future disease and pathogen virulence remain elusive. Typically, genetic alterations in cell genomes resulting in disease (or disease susceptibility) or genetic sequence of virulent pathogens is collected a posteriori, cataloged, and then used to make a diagnosis, and determine an appropriate therapeutic. Accordingly, responding therapeutically to the sequelae of genome instability in a subject suffering from a genetic disease (or disease susceptibility) and/or pathogen is typically reactive rather than proactive. This is especially true in treating, for example, human and animal response to pathogens such as viruses.

Development of viral vaccines presents unique challenges to modern medicine (see, for example, Ault, A. (2004) Science, 303:1280). Due to the constant evolution of many pathogens, and in particular, viruses, development of effective vaccines is often a difficult, and imperfect, process. Reliable prediction of the future molecular evolution of viral genomes would be expected to advance mankind's ability to combat such pathogens both prophylactically and therapeutically.

Viruses are the smallest of parasites, and are completely dependent upon the cells they infect for their reproduction. Of the viruses that infect humans, many infect their hosts without producing overt symptoms, while others (e.g., influenza) produce a well-characterized set of symptoms. Importantly, although symptoms can vary with the virulence of the infecting strain, identical viral strains can have drastically different effects depending upon the health and immune response of the host.

A better understanding of the molecular events that lead to genome instability not only for understanding human and animal disease but also the evolution of pathogens such as viruses, is needed. Indeed, the ability to predict the molecular evolution of pathogenic genomes would be broadly expected to enhance the design of anti-pathogenic agents.

### SUMMARY OF THE INVENTION

The instant invention is based at least in part on the discovery that genome instability across a wide array of organisms, including eukaryotic cells, prokaryotic cells, and viruses occurs as a function of a newly-identified mechanism termed copy-choice recombination. Heretofore, random mutations, gene translocations, and/or gene reassortment were thought to be the predominant mechanisms of viral gene evolution. Indeed, until recently, it was believed that viral evolution has been primarily due to the accumulation of small mutations in the viral genome. However, this mechanism explains only a small part of the evolution of viruses.

The newly-identified mechanism described herein can account for the acquisition of gene mutations between two or more gene sequences in a cellular or organismal context. Accordingly, the mechanism disclosed herein is predictive for mutations that can occur in multicellular organisms, eukaryotic cells, prokaryotic cells, in pathogens and microbes, and, in particular, viruses.

Without being bound by theory, the invention provides a mechanism of genetic evolution based upon recombination or acquisition of a previously existing sequence(s) by gene copy recombination, i.e., referred to herein as copy choice recombination rather than through the introduction of de novo genetic mutation(s) based on, e.g., polymerase proof-reading errors, spontaneous point mutations, and the like.

This mechanism of genetic change can be readily exploited to provide predictive rules by which genetic changes in the genomes of eukaryotic cells, prokaryotic cells, pathogens, microbes, viruses, and the like can be forecast. Accordingly, the likelihood of a genetic alteration appearing in a given genome allows for a priori intervention, e.g., the prediction or prognosis of genetic disease or disorder, or emergence or appearance of a strain of pathogen, e.g., a virulent strain, such that therapy can be rationally designed.

The predictive rules of the invention, i.e., of copy-choice recombination include, e.g., 1) that the prediction that genetic alterations are acquired in tracts that resemble the haplotypes documented in higher eukaryotic genomic sequence, 2) that the prediction that genetic alterations typically comprise a high frequency of nucleic acid base transitions, and/or 3) that the prediction that genetic alterations are acquired from an existing gene sequence(s) from a parental nucleic acid sequence.

In one embodiment, the predicative rules of the invention can be used to improve human or animal health by forecasting the likelihood of a disease or disorder or the pharmacogenomic responsiveness of a subject.

In another embodiment, the predicative rules of the invention can be used to improve human or animal health by forecasting the likelihood of the appearance or emergence of a pathogen, for example, a virulent strain of virus, thereby allowing for therapeutic intervention, for example, administering of an anti-pathogenic agent, for example, an antiviral and/or vaccine (e.g. passive or active vaccine).

In another embodiment, the invention provides for a comparison of parental viral strains with their mutant progeny viral strains which can be used to define and elucidate selective pressures on rapid evolution. The identification of recombinants can be used to identify genetic instability, which is currently evident in many viruses throughout the world, for example, influenza. The parental viruses can also be used to create recombinants prior to detection in field isolates and such recombinants can be used to make protective vaccines against future recombinants, which cause significant disruptions in animal husbandry and human health.

In still another embodiment, the invention provides rules that can be applied, e.g., to predict the genetic composition and, optionally, associated phenotypic traits (e.g., drug resistance) of viruses or bacteriae that arise from the mixing within a single host organism of distinct “parental” viruses or bacteriae (e.g., ebola, flu and/or HIV; foot and mouth and Newcastle disease; SARS, HIV and/or astroviruses; HIV and coronavirus; distinct drug-resistant bacterial strains, etc.).

In yet another embodiment, the invention provides methods of generating libraries of diverse viral sequences to be used, for example, in the manufacture of viral vaccines, or for testing of antiviral compounds. The invention further provides methods of identifying parental viral strains.

The instant invention also provides methods for monitoring the efficacy of viral vaccines and for monitoring the diversity of a viral population.

Accordingly, the invention has several advantages, which include, but are not limited to, the following:
providing rules for determining the nature of genetic alteration occurring or likely to occur in eukaryotes, prokaryotes, and pathogens and predicting future genetic alterations;
providing methods for prognosis of genetic alterations in a human or animal and/or pharmacogenomic responsiveness of a human or animal;
providing methods for determining gene sequences in a human or animal suitable for modulating thereby preventing or treating a disease or disorder;
methods for identifying rational candidate pathogens for therapeutic intervention;
methods and compositions relating to vaccines and vaccine development against pathogen targets, for example, viral pathogens; and
methods and compositions relating to vaccines and vaccine development against influenza, for example, virulent strains of influenza such as H5N2, H5N1, H7N2, H7N3, H7N7, H1N1, H9N2, WSN/33, H6N1, H1N2, H2N2, H3N2, H3N8 and H2N9.

Other features and advantages of the invention will be apparent from the following detailed description and claims.

### BRIEF DESCRIPTION OF THE DRAWINGS

FIG. 1 depicts a schematic representation of recombination between two copies of DNA. The left panel depicts a single crossover event, while the right panel depicts a double crossover event.

FIGS. 2A-H depict the location of polymorphisms in the PB2, PB1, PA, HA, NP, NA, MP, and NS genes, respectively, from influenza viruses isolated from the Hong Kong pandemic of 2001.

FIGS. 3A-H depict the location of polymorphisms in the PB2, PB1, PA, HA, NP, NA, MP, and NS genes, respectively, from influenza viruses isolated in Hong Kong during 2002-2003.

FIGS. 4A-J depict examples of viral recombination. FIGS. 4A-D depict polymorphisms in two isolates (31.2 and 31.4) from a chicken in Hong Kong. FIG. 4E depicts recombination in the NA gene of a H9N2 isolate from Korea. FIG. 4F depicts polymorphisms in the PA gene of a Korean avian isolate. FIG. 4G depicts polymorphisms in PB2 isolated from a Korean swine indicative of recombination between human lab strain WSN/33 and Korean swine isolates from 2004. FIG. 4H depicts recombination in human HA genes isolated in 2002.

FIG. 5 depicts the Ebola and influenza strain sequences for a conserved 18 nucleotide tract.

FIGS. 6A-C depict additional examples of viral recombination among influenza strains.

FIGS. 7A and 7B depict additional examples of viral recombination among influenza strains.

FIGS. 8A and 8B depict additional examples of viral recombination among influenza strains.

### DETAILED DESCRIPTION

In order to provide a clear understanding of the specification and claims, the following definitions are conveniently provided below.

Definitions

The term, “parental viral strains” is intended to mean the two, or more, viral strains in a population that supply the genetic material to the mutant progeny viral strains in the population through a copy choice recombination mechanism. The parental viral strains are two or more strains of virus that are present in a recently (e.g., within one, two, three, six, twelve, or more months) isolated population of viruses. In one aspect the parental viral strains are the most prevalent sequences in a population. In another aspect, the parental viral strains are the most diverse sequences in a population.

The term, “mutant progeny viral strains” as used herein is intended to mean the viral progeny derived from the parental viral strains. In one embodiment, the mutant progeny viral strains are created by a copy-choice recombination mechanism using the genetic material provided by the parental strains. In one embodiment, the mutant progeny viral strains are isolated from a population of viruses based on one or more desired criteria, e.g., nucleotide sequence, polypeptide sequence, virulence, host range, or tropism.

Similarly, the term “parental bacteria strains” is intended to mean the two, or more, bacteria strains in a population that supply the genetic material to the mutant progeny bacteria strains in the population through a copy choice recombination mechanism. The parental bacteria strains are two or more strains of bacteria that are present in a recently (e.g., within one, two, three, six, twelve, or more months) isolated population of bacteria. In one aspect the parental bacteria strains are the most prevalent sequences in a population. In another aspect, the parental bacteria strains are the most diverse sequences in a population.

Likewise, the term “mutant progeny bacteria strains” as used herein is intended to mean the bacteria progeny derived from the parental bacteria strains. In one embodiment, the mutant progeny bacteria strains are created by a copy-choice recombination mechanism using the genetic material provided by the parental strains. In one embodiment, the mutant progeny bacteria strains are isolated from a population of bacteria based on one or more desired criteria, e.g., nucleotide sequence, polypeptide sequence, drug resistance, pathogenicity, infectivity, etc.

The term “copy choice recombination” as used herein is intended to mean the mechanism of viral or bacterial recombination in which a progeny viral or bacterial strain is made in a cell or organism that has been infected by two or more parent viral strains and the genetic material of the progeny is a mix of the genetic material of the parent strains. Without being bound by mechanism, copy choice mechanism results from the DNA or RNA replication machinery starting on DNA or RNA from one parent and switching to the DNA or RNA from a second parental strain during duplication of a piece of DNA or RNA. This process can happen one or more times thereby resulting in progeny virus or bacteria that has a DNA or RNA sequence that is a mix of the two parental strains.

Sequences produced by copy-choice recombination, e.g., progeny sequences, can contain any number of nucleotide changes, including one or more nucleotide changes as compared with parental sequences, e.g., 2-5, 5-10, 10-20, 20-50, 50-100, 100-500, 500 or greater changes, typically by recombination, e.g. copy-choice recombination, occurring within a given length of nucleic acid, between two or more strands of nucleic acid, e.g., within two nucleotides or more, e.g., 3-5, 5-10, 10-100, 100-1 kb, 1 kb-10 kb, 10 kb or more, or any range or interval thereof.

The term “transition/transversion ratio” as used herein is intended to denote a ratio between the number of times a given sequence has a transition, e.g., the substitution of a purine for a purine, or a pyrimidine for a pyrimidine, versus the number of times the sequence has a transversion, e.g., a purina for a pyrimidine or a pyrimidine for a purine. One would expect the ratio to be 0.5 if it were a random process. However, looking at multiple data sets, it has been determined that the ration is often 2 or higher, indicative that the process is not random and that transitions are favored over transversions (see the Exemplification).

The present invention, at least in part, is based on the surprising observation that recombination, rather than de novo mutation, is a driving force of viral evolution. The present observation that progeny strains of influenza are effectively derived as haplotypes from divergent, “parental” strains of influenza reveals that dual infections of a single cell or organism with two or more distinct strains of virus (or distinct types of virus, e.g., influenza and HIV, or distinct strains of bacteria) can accelerate viral evolution. The present invention therefore provides rules for predicting the outcome of such real-world or controlled mixing experiments. In certain aspects of the invention, these rules can be applied to predict progeny influenza strains that represent optimal vaccine targets, based upon knowledge (optionally real-time knowledge) of the genetic makeup of the prevalent influenza strains in a population.

In an additional aspect, the rules of the invention may be applied to enable prediction of the genomic composition and/or phenotypic traits e.g., drug resistance, of progeny bacterial strains derived from at least two parental strains of bacteria. Such bacteria can then be used, e.g., in subsequent drug screening steps.

In one aspect, the instant invention provides a method for identifying parental viral strains in a population of viruses, wherein the population comprises parental viral strains and mutant progeny viral strains, comprising the steps of: obtaining the nucleic acid or polypeptide sequence of one or more viral genes from a number of isolated viral strains from the population, the number sufficient to allow for identification of the viral strains most prevalent in the population, the viral strains having the greatest sequence divergence in the population, or both; identifying the viral strains most prevalent in the population, or viral strains with the greatest sequence divergence in the population, or both; wherein the most prevalent viral sequences, or the viral sequences with the greatest divergence are the parental viral strains.

In one embodiment, the parental viral strains are the two most prevalent sequences in the population. In another embodiment, the parental strains are the two strains with greatest sequence divergence.

In one embodiment, the viruses used in the methods of the invention are from a period of time sufficient to allow for the determination of the parental and mutant progeny viral strains. For example, the period of time in which isolated viruses can be used in the methods of the invention can be 1 month, 2 months, 3 months, 4 months, 6 months, 1 year, 2 years, 3 years, 4 years, 5 years, or more. In one exemplary embodiment, the viruses used in the methods of the invention are from one outbreak season, e.g., one influenza season.

In another embodiment, the methods of the invention use viruses from a defined geographic area, e.g., one in which infected hosts have reasonable chance of interacting. For example, defined geographic areas are southeast Asia, or the continental United States.

In a related embodiment, the most prevalent viral sequences, or the viral sequences with the greatest sequence divergence are determined by aligning multiple nucleic acid or polypeptide sequences. In another related embodiment, the mutant progeny viral strains are formed by recombination according to a copy-choice mechanism.

Sequence alignments can be done using, for example, a mathematical algorithm. In one embodiment, the percent identity between two amino acid sequences is determined using the Needleman and Wunsch (J. Mol. Biol. 48:444-453 (1970)) algorithm which has been incorporated into the GAP program in the GCG software package (available at http://www.gcg.com), using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

Further, algorithms based upon sequence alignment programs can be developed that automatically compare viral sequences deposited in a database to determine the sequence identity in a population. Bioinformatic approaches can be used to monitor the amount of sequence diversity as a function of time, and location, thereby alerting medical professionals as to when their intervention, i.e., immunization, efforts should be increased. A Bioinformatics approach would be particularly useful for viral populations where there are large databases that would be difficult to align and/or sort, e.g., by date or location, manually, e.g., HIV or influenza. Bioinformatics can be used to determine the parental viral strains in a population of viruses and/or determine the mutant viral progeny viruses in a population of viruses by sorting the nucleic acid or polypeptide sequences by, for example, the number and/or location of non-identical nucleotides or amino acids, respectively.

In another aspect, bioinformatics can be used to evaluate databases of viral sequences to identify historically significant sequence variations in a viral gene sequence. The emergence of a previously identified sequence polymorphism is indicative of copy-choice recombination. Without being bound by mechanism, the emergence of a sequence polymorphism in a population of viruses that has not been observed for some time is a sign that there has been copy-choice recombination between two viruses. This approach will allow one of skill in the art to identify, in silico, mutant progeny viral strains that may be problematic, e.g., have high infectivity. Further, analysis of viral sequences in a database for the presence of a known sequence polymorphism that is normally not found in a given geographic area can indicate that copy-choice recombination has occurred.

In one aspect, the methods of the invention may use a computer based program to identify multiple cross-over points in mutant progeny viral strains. Due to the high number of cross-over points in some genes formed by copy choice recombination (often 10-100 cross-over points per gene) computer algorithms will be useful tools to determine the precise location of cross-over points. These computer algorithms may compare a large database of viral sequences to determine the location of cross-overs in a parental viral strain that gave rise to mutant progeny viral strains. The precise mapping of these locations in combination with analysis of the various polymorphisms will allow one of skill in the art to classify viruses based on genotype rather than the serotype classification currently used.

In one embodiment, mutant progeny viral strains can be produced by a copy-choice recombination mechanism in combination with reassortment. In another embodiment, the mutant viral progeny viruses are produced by copy-choice recombination in the absence of reassortment.

Further, in vitro or in vivo techniques can be used to selectively recombine individual genes from different viruses in the population to produce mutant viral progeny viruses. For example, a number of genes from a population of viruses can be analyzed using, for example, sequence alignments. One of skill in the art can isolate genes with desired sequences from the population and use those genes to infect a host cell, egg, or animal to produce a desired set of recombinants. In this situation the genes used to infect the host can come from multiple different viruses (e.g., 3, 4, 5, 6, 7, 8, 9, 10, 11, 12, 13, 14, 15 or more different viruses).

In one aspect of the invention, the methods of the invention can be used with any viruses that infect a subject. The term “subject” is intended to include organisms which are capable of having a viral infection. Examples of subjects include mammals, e.g., humans, dogs, cows, horses, pigs, sheep, goats, cats, mice, rabbits, rats, and transgenic non-human animals, or birds, e.g., ducks, chicken, geese, and swans. In certain embodiments, the subject is a human. Similarly, the term “host” is intended to include organisms, e.g., mammals, e.g., humans, dogs, cows, horses, pigs, sheep, goats, cats, mice, rabbits, rats, or birds, e.g., ducks, chicken, geese, and swans, and transgenic non-human animals, that harbor a viral strain, nucleotide sequences that recombine via copy-choice recombination, etc.

In one embodiment, the viruses are RNA viruses. In one embodiment, the RNA viruses are single-stranded RNA viruses. In one embodiment, the single-stranded RNA viruses are positive-sense RNA viruses. In another embodiment, the single-stranded RNA viruses are negative-sense RNA viruses. In a related embodiment, the RNA viruses are double-stranded RNA viruses. In one related embodiment, the double-stranded RNA viruses are positive-strand RNA viruses. In another embodiment, the double-stranded RNA viruses are negative-strand RNA viruses.

In another embodiment, the viruses are DNA viruses. In one embodiment, the DNA viruses are single-stranded DNA viruses. In another embodiment, the DNA viruses are double-stranded DNA viruses.

In one embodiment, the viruses are influenza viruses. In another embodiment, the viruses are coronavirus viruses, e.g., SARS CoV.

In one embodiment, the protein or nucleic acid sequences are from influenza viruses.

In one embodiment, the influenza nucleic acid or polypeptide sequences are selected from the group consisting of: HA, NA, NP, PA, PB1, PB2, MP, and NS, or combinations thereof.

In one embodiment, the nucleic acid or polypeptide sequences are obtained by sequencing the isolated viral strains. In another embodiment, the sequences are obtained by sequencing nucleic acid molecules isolated from a subject (e.g., a human or animal) or a tissue sample. In another embodiment, the nucleic acid or polypeptide sequences are obtained from a publicly available database. In certain embodiments, the sufficient number is 5, 10, 20, 30, 40, 50 or more viral sequences.

In one embodiment, the one or more viral genes is at least two, three, four or five or more genes.

In another aspect, the invention provides a method of producing a viral vaccine, comprising: infecting a host animal, host animal cell, cell line, egg cell, bacterial cell, or cell extract which supports viral replication with the parental viral strains identified according to the methods described above; and isolating mutant progeny viral strains from the host animal cell line, egg cell, bacterial cell, or cell extract which supports viral replication.

Viral vaccines of the present invention can be, for example, live vaccines, killed vaccines, attenuated vaccines or subunit vaccines (see, for example, Fields Virology, (1996) Third Edition, Lippencott-Raven Publishers, Philadelphia, pp. 467-469.) Further examples of vaccine production are, for example, Meadors et al. (1986) Vaccine: 179-184, Poland et al. (1990) J. Infect. Disease 878-882, Fenner et al. The Biology of Animal Viruses; New York, Academic Press, 1974:543-586, Saban et al. (1973) J. Biol. Stand. 115-118, and Lowrie et al., DNA Vaccines: Methods and Protocols, Humana Press, New Jersey, 1999.

An attenuated whole organism vaccine uses a non-pathogenic form of the desired virus. Non-pathogenicity may be induced by growing the virus in abnormal conditions. Those mutants that are selected by the abnormal medium are usually limited in their ability to grow in the host and be pathogenic. The advantage of the attenuated vaccine is that the attenuated pathogen simulates an infection without conferring the disease. Since the virus is still living, it provides continual antigenic stimulation giving sufficient time for memory cell production. Also, in the case of viruses where cell-mediated immunity is usually desired, attenuated pathogens are capable of replicating within host cells. Genetic engineering techniques are being used to bypass these disadvantages by removing one or more of the genes that cause virulence.

An inactivated whole organism vaccine uses viruses which are killed and are no longer capable of replicating within the host. The viruses are inactivated by heat or chemical means while assuring that the surface antigens are intact. Inactivated vaccines are generally safe, but are not entirely risk free. Multiple boosters are usually necessary in order to generate continual antigen exposure, as the dead organism is incapable of sustaining itself in the host, and is quickly cleared by the immune system.

One or more polypeptides, or fragments thereof, that are presented by a virus can be formulated into a vaccine that elicits an immune response in a host. These so called “subunit” vaccines often alleviate the safety concerns associates with whole virus vaccines.

In a related embodiment, the method further comprises attenuating the mutant progeny viral strains to make an attenuated viral vaccine. In another embodiment, the method further comprises killing the mutant progeny viral strains to make a killed viral vaccine. In another embodiment, the method further comprises isolating viral antigens, or portions thereof, from the mutant progeny viral strains to make a subunit viral vaccine.

In another embodiment, the invention provides a method of immunizing a subject against a virus comprising: administering to the subject the attenuated virus vaccine in an amount sufficient to immunize the subject. In one embodiment, the subject is a mammal, e.g., a human, in another embodiment the subject is a bird.

In one embodiment, the method of immunizing a subject (e.g., a human or animal) against a virus comprising: administering to the subject a killed, or attenuated, virus vaccine in an amount sufficient to immunize the subject. In another embodiment, the invention provides a method of immunizing a subject against a virus comprising administering to the subject the subunit virus vaccine in an amount sufficient to immunize the subject. In one embodiment, the parental strains are influenza viral strains. In another embodiment, the parental strains are coronavirus viral strains.

In one aspect, the invention provides a method of immunizing a subject (e.g., a human or animal) against a virus comprising: administering to the subject a first virus representing the first parental viral strain and a second virus representing a second parental viral strain, the first and second parental viral strains identified according to the methods described herein, in an amount sufficient to immunize the subject.

In one embodiment, the parental viral strains are attenuated prior to administering to the subject. In another embodiment, the parental viral strains are killed prior to administering to the subject. In another embodiment, the method comprises isolating viral antigens, or portions thereof, from the parental viral strains to make a subunit viral vaccine prior to administering to the subject.

In one embodiment, the parental viral strains are influenza viral strains. In another embodiment, the parental viral strains are coronavirus viral strains.

In another aspect, the invention provides a viral vaccine composition comprising the parental viral strains identified according to the methods described herein, or antigens, or portions of antigens, therefrom.

In one embodiment, the viral vaccine further comprises mutant progeny viral strains derived from the parental viral strains, or antigens, or portions of antigens, therefrom. In another embodiment, the vaccine comprises two viral strains, or antigens, or portions of antigens from two viral strains.

In another embodiment, the vaccine composition, comprising mutant progeny viral strains, or antigens or portions of antigens therefrom, is made by recombination according to a copy-choice mechanism of two viral strains whose genomes are made up of non-identical nucleic acid sequences. In one embodiment, the two viral strains are parental viral strains identified according to the methods described herein.

In one embodiment, the mutant progeny viral strains are produced by recombination according to a copy-choice mechanism in a host animal. In another embodiment, the mutant progeny viral strains are produced by recombination according to a copy-choice mechanism in cell culture.

In one embodiment, subjects who should be given a viral vaccine can be determined based on the genotype of the current viral strains in a population. Similarly, the type of vaccine a given subject should receive can be determined based on the genotype of the current viral stains in a population. For example, current viral isolated can be classified by the number of polymorphisms that they have. In one embodiment, the polymorphisms are ones that have been identified in isolates from pervious outbreaks. The identification of sequence polymorphisms in a population of viral isolates can be used to form an exposure timeline. This time line can be used to determine the age group susceptibility to a viral infection. For example, a new isolate with a number of polymorphisms identified in 1970 may be less of a concern to those people born prior to 1970, whereas this same isolate may produce more severe infection in those subjects born after 1970. Based on this timeline, medical professionals can determine which subjects should be administered a vaccine, or what vaccine a given subject should receive.

In another aspect, the invention provides a method of identifying the stability of a genome in a population of viruses, comprising: obtaining the nucleic acid or polypeptide sequence of one or more viral genes from a sufficient number of isolated viruses from the population; comparing the number of recombinant viral sequences in the isolated viruses; wherein the greater the number of distinct viral sequences, the greater the instability of the viral genome.

In another aspect, the invention provides a method of identifying the stability of a genome in a population of viruses, comprising: comparing the nucleic acid or polypeptide sequence of one or more viral genes from a sufficient number of isolated viruses from the population; comparing the diversity between parental viral sequences in the isolated viruses; wherein the greater the diversity of distinct viral sequences, the greater the instability of the viral genome.

Genetic stability can be used to measure environmental or experimental effects on genetic stability. This measurement can be determined actively or passively. Thus animals can be immunized and then co-infected with two parental strains and the progeny can be monitored to see the amount of recombination that occurs. This approach can be used to measure the ability of a vaccine to reduce or eliminate recombinants. Similarly, assaying a natural population at different time points can be used to measure environmental effects on recombination. The amount of genetic stability (or instability) can be used to identify times when aggressive intervention is necessary, even in the absence of overt disease.

In another aspect, the invention provides a method of immunizing a subject (e.g., a human or animal) against a virus comprising: administering to the subject mutant progeny viral strains, or antigens or portions of antigens therefrom, made by recombination according to a copy-choice mechanism of two viral strains whose genomes are made up of non-identical nucleic acid sequences.

In another aspect, the invention provides a method of immunizing a subject (e.g., a human or animal) against a virus comprising: determining the parental viral strains in a population of viruses; allowing the parental viral strains to recombine according to a copy-choice mechanism to produce mutant progeny viral strains; administering the parental viral strains, or mutant progeny viral strains, or antigens or portions of antigens therefrom, in an amount sufficient to immunize the subject.

In another aspect, the invention provides a method for identifying parental influenza strains in a population of influenza viruses, wherein the population comprises parental influenza strains and mutant progeny influenza strains, comprising the steps of: obtaining the nucleic acid or polypeptide sequence of one or more influenza genes from a number of isolated influenza strains from the population, the number sufficient to allow for identification of the influenza strains most prevalent in the population, the influenza strains having the greatest sequence divergence in the population, or both; identifying the influenza strains most prevalent in the population, or influenza strains with the greatest sequence divergence in the population, or both; wherein the most prevalent influenza sequences, or the influenza sequences with the greatest divergence are the parental influenza strains.

In a related embodiment, the invention provides a method of producing an influenza vaccine, comprising: infecting a host animal with the parental influenza strains identified; and isolating mutant progeny influenza strains from the host animal.

In a related embodiment, the invention provides a method of immunizing a subject against an influenza virus comprising: administering to the subject a first influenza virus representing the first parental influenza strain and a second influenza virus representing a second parental influenza strain, the first and second parental influenza strains identified according to the methods described herein, in an amount sufficient to immunize the subject.

In one aspect, the invention provides a method of producing a library of recombinant viral strains comprising: infecting a host cell or animal with two or more viral strains; allowing for recombination of the viruses by a copy choice mechanism of the two or more viral strains, thereby creating a library of viral strains. In one embodiment, the library of recombination viral strains can be isolate for vaccine production.

In a related embodiment, the viral strains may be different species of viruses. For example, the first virus could be influenza and the second virus could be a coronavirus, e.g., SARS. In a related embodiment, the identification of a DNA sequence from one species' genome that originated in the genome of a distinct species is indicative that this segment of DNA confers an advantageous property to the virus, i.e., increased infectivity or virulence. Targeting these regions of DNA would provide for effective anti-viral therapy.

In a related embodiment, the library of viral strains can be created in a host cell or animal that has been given an antiviral compound. In a related embodiment, the viral strains that are created in the presence of an antiviral compound are indicative of the antiviral resistant strains that will occur in a population of subjects treated with the antiviral compound.

In another aspect, the invention provides a vaccine composition, comprising mutant progeny influenza strains, or antigens or portions of antigens therefrom, made by recombination according to a copy-choice mechanism of two influenza strains whose genomes are made up of non-identical nucleic acid sequences.

In other embodiments, art-recognized methods of gene therapy, e.g., RNAi, may be employed to target viral strains, optionally in a strain and/or otherwise sequence-specific manner, e.g., via use of miRNA, siRNA, shRNA, or other such agents.

In another aspect, the invention provides a method for identifying parental coronavirus strains in a population of coronavirus viruses, wherein the population comprises parental coronavirus strains and mutant progeny coronavirus strains, comprising the steps of: obtaining the nucleic acid or polypeptide sequence of one or more coronavirus genes from a number of isolated coronavirus strains from the population, the number sufficient to allow for identification of the coronavirus strains most prevalent in the population, the coronavirus strains having the greatest sequence divergence in the population, or both; identifying the coronavirus strains most prevalent in the population, or coronavirus strains with the greatest sequence divergence in the population, or both; wherein the most prevalent coronavirus sequences, or the coronavirus sequences with the greatest divergence are the parental coronavirus strains.

In a related embodiment, the invention provides a method of producing a coronavirus vaccine, comprising: infecting a host animal with the parental coronavirus strains identified; and isolating mutant progeny coronavirus strains from the host animal.

In another aspect, the invention provides a method of immunizing a subject against an coronavirus virus comprising: administering to the subject a first coronavirus virus representing the first parental coronavirus strain and a second coronavirus virus representing a second parental coronavirus strain, the first and second parental coronavirus strains identified according to the methods described herein in an amount sufficient to immunize the subject.

In one aspect, the invention provides a vaccine composition, comprising mutant progeny coronavirus strains, or antigens or portions of antigens therefrom, made by recombination according to a copy-choice mechanism of two coronavirus strains whose genomes are made up of non-identical nucleic acid sequences.

In another aspect, the invention provides a method of producing mutant progeny viral strains for the manufacture of a viral vaccine comprising; infecting a cell or animal with two non-identical viral strains; allowing for recombination of the non-identical viral strains according to a copy-choice mechanism; thereby producing mutant progeny viral strains. In one embodiment, the method further comprises isolating the mutant progeny viral strains from the host cell or animal.

In one aspect, the invention provides a method of determining the efficacy of a vaccine comprising: obtaining the nucleic acid or polypeptide sequence of one or more viral genes from a number of isolated viral strains from a population that has been treated with a viral vaccine, the number sufficient to allow for number of mutant progeny viral strains in the population; wherein, the lower the number of different mutant progeny viral strain sequences, the greater the efficacy of the vaccine.

In another embodiment, the invention provides a method of predicting the sequence of one or more genes in a mutant progeny viral strain comprising obtaining the sequence of one of more of the genes from a parental viral strain, determining the location of possible recombination events, thereby predicting the sequence of one or more genes in a mutant progeny viral strain. In a related embodiment, the viral strain is selected from the group consisting of an influenza viral strain, a corona viral strain, and an HIV viral strain. In another related embodiment, the method further comprises using the predicted sequence of the mutant progeny viral strain to develop a vaccine against said virus.

In another aspect, the invention provides a method of producing mutant progeny viral strains comprising infecting a cell or animal with two non-identical viral strains, allowing for recombination of the non-identical viral strains according to a copy-choice mechanism, thereby producing mutant progeny viral strains. In a related embodiment, the method further comprises isolating said mutant progeny viral strains.

In a related aspect, the invention provides a method of producing mutant progeny virus(es) comprising infecting a cell or animal with two or more non-identical viruses (e.g., ebola and influenza), allowing for recombination of the non-identical viruses according to a copy-choice recombinant mechanism, thereby producing mutant progeny virus(es). In related embodiments, the method further comprises isolating and/or raising vaccine(s) to said mutant virus(es).

In a further aspect, the invention provides a method of producing mutant progeny bacterial strains comprising infecting a cell or animal with two or more non-identical bacterial strains, allowing for recombination of the non-identical bacterial strains according to a copy-choice recombinant mechanism, thereby producing mutant progeny bacterial strains. In a related embodiment, the method further comprises isolating said mutant progeny viral strains. In another embodiment, the method further comprises assessing a phenotypic trait of a mutant progeny bacteria (e.g., drug resistance, assessed, e.g., via compound screening assays).

In a related aspect, copy-choice recombination is responsible for the occurrence of non-mendelian inheritance in certain plants, e.g., Arabidopsis. Thus, the invention provides a method for predicting and/or performing non-mendelian inheritance via copy-choice recombination in plants (e.g., Arabidopsis), provided two or more non-identical parental plants.

In a related aspect, the invention provides a method of predicting a phenotypic trait (e.g., virulence, drug resistance, etc.) of a mutant progeny virus, bacteria or plant through assessment of the range of mutant progeny possible via copy-choice recombination from two or more parental viruses, bacteriae or plants.

In another aspect, the invention provides a method of producing a population of recombinant genes comprising introducing into a cell two or more non-identical copies of a gene, allowing for recombination of the genes, thereby producing a population of recombinant genes. In a related embodiment, the recombination occurs via a copy-choice mechanism. In a related embodiment, the method further comprises isolating one or more members of the population of recombinant genes. In one embodiment, the genes are viral genes. In another embodiment, the genes are from non-viral species, e.g., plants or animals.

### EXEMPLIFICATION

Throughout the examples, the following materials and methods were used unless otherwise stated.

Materials and Methods

In general, the practice of the present invention employs, unless otherwise indicated, conventional techniques of chemistry, molecular biology, recombinant DNA technology, immunology (especially, e.g., antibody technology), and standard techniques in electrophoresis. See, e.g., Sambrook, Fritsch and Maniatis, Molecular Cloning: Cold Spring Harbor Laboratory Press (1989); Antibody Engineering Protocols (Methods in Molecular Biology), 510, Paul, S., Humana Pr (1996); Antibody Engineering: A Practical Approach (Practical Approach Series, 169), McCafferty, Ed., Irl Pr (1996); Antibodies: A Laboratory Manual, Harlow et al., C.S.H.L. Press, Pub. (1999); and Current Protocols in Molecular Biology, eds. Ausubel et al., John Wiley & Sons (1992).

### Example 1

### Influenza A Emergence and Evolution Via Recombination

Influenza A is thought to evolve gradually via point mutations and abruptly via reshuffling of its eight segmented genes. Here, Influenza A evolution has been shown to be driven by recombination in hosts infected with two distinct viruses. Most polymorphisms of closely related viruses are bimorphisms, involving third base codon changes, which are silent at the protein level. The recombination generates both versions of the nascent genes and both viruses are viable. The recombination redistributes existing polymorphisms, allowing prediction of the genetic composition of new viruses, before they emerge. This recombination mechanism is common. It generates pandemic H5N1 influenza, as well as most or all, rapidly evolving genomes.

The looming H5N1 flu pandemic has attracted considerable attention (Peiris et al 2004; Fouchier et al 2005; Osterholm 2005). To determine the molecular mechanisms behind the evolution and emergence of H5N1, recent sequences of isolates from Hong Kong were compared to determine the genetic basis for the evolution of a pandemic strain that has spread through eastern Asia and caused human fatalities in Vietnam, Thailand, and Cambodia.

Influenza has a segmented genome and the reassortment of the eight genes has been used to classify the H5N1 isolates (Guan et al 2002, Alexandr et al 2003). Changes in influenza genetic composition have been described as drifts and shifts (Webster et al 1992). The drifts have been characterized as gradual changes due to replication errors by an RNA polymerase lacking a proof-reading function. Shifts are thought to involve more dramatic changes in genetic composition due to reassortment of the eight sub-genomic RNAs.

Analysis of the changes of H5N1 isolates indicated that both drifts and shifts were caused by homologous recombination, which generated complementary genomes. Dual infections lead to the recombinants, which resemble point mutations when two closely related viruses recombine. The same mechanism between more distantly related virus can produce much more dramatic changes and create two viable genomes which are complementary. Most of the polymorphisms are transitions, so the positions are largely binary and most of the RNA changes are synonymous. Thus, the polymorphisms in the genetic population are largely bimorphisms represented by either a purine or pyrimidine. The redundancy in the genetic code produces synonymous changes for almost all third base transitions.

Although many changes are found throughout the database indicating a large number of alterations would produce viable progeny, the changes are limited largely by the resident gene pool and the composition of the recombinants is related to the prevalence of the polymorphisms in co-circulating genotypes. The newly formed recombinants can be predicted by the prevailing gene pools.

These complementary polymorphisms are used in virtually all biological systems. They are common in rapidly evolving viruses, but similar mechanisms drive repair of polymorphisms at the cellular level. Moreover, non-homologous recombination is used to link together viral genomes that have similar pathologies such as Ebola and pandemic H1N1 and H5N1 influenza.

Evolution of pandemic H5N1 can be traced to 2001H5N1 Hong Kong isolates. The live market isolates formed five groupings based on reasserted genes (Guan et al. 2002). Representative isolates generated a neurotropic version isolated from mouse brain (Alexander et al 2003). The isolates in Hong Kong had major polymorphisms that were present in at least 20% of the isolates.

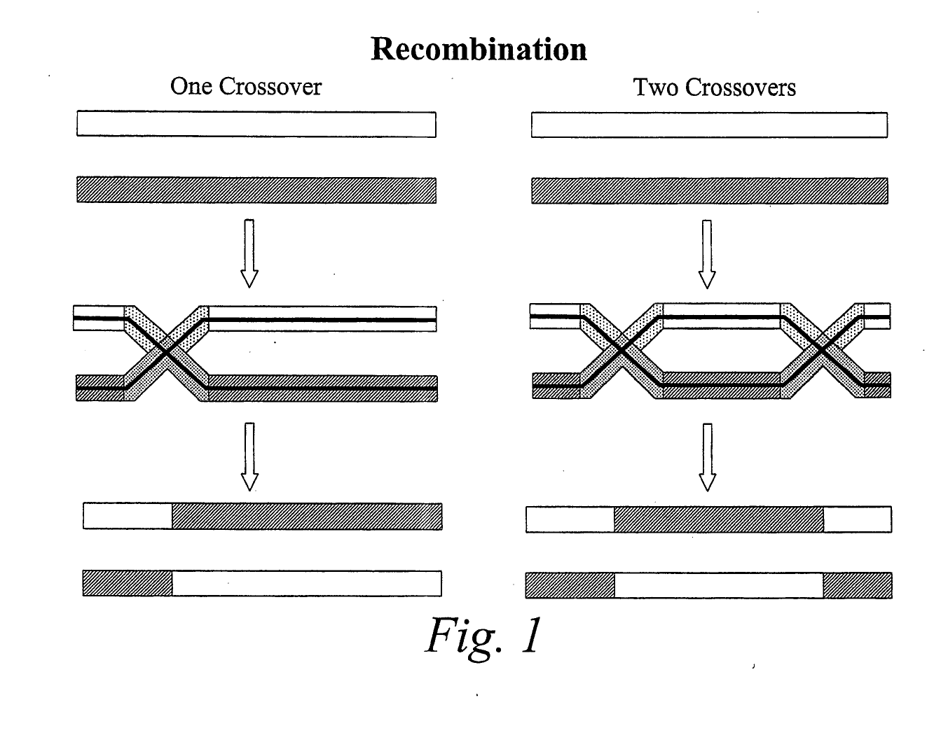

The polymorphisms for all eight genes are listed in FIG. 1 (FIGS. 1A-1H). For each gene, the isolates segregated out into two major genotypes designated Allele 1 and Allele 2. Allele 1 was composed of Group A and for some genes, Group B. Allele 2 was generally Groups C-E. These groupings were present across all eight genes and the polymorphisms were coded with regard to the emerging pandemic strain found in Vietnam and Thailand. The two alleles complement each other and for most positions the polymorphisms were bimorphisms incorporating a purine or pyrimidine at third base positions, thereby producing synonymous changes.

The complementary nature of the bimorphisms suggested the two alleles were generated via homologous recombination. The use of only a purine or pyrimidine at a third base position generates two RNA versions of the same protein. The number of bimorphisms for each gene suggested some of these genes had already recombined to generate one version that was highly homologous to the pandemic strain and another version that contained the alternate purine or pyrimidine. Thus, for PB2, HA, NA, and NS the number of polymorphisms was small (11-39) and the bimorphisms that matched the pandemic strain were evenly divided between two alleles. In contrast, PB1 had already recombined to place most of the matching bimorphisms in allele 1 and there were 111 polymorphisms. Similarly, PA had also recombined so most of the matching bimorphisms were in allele 2 and there were 114 polymorphisms. M is a smaller gene and not as genetically diverse, so there were only 29 polymorphisms and most that matched the pandemic gene were in Allele 2. NP was somewhat unusual. There were 63 polymorphisms that were evenly divided, but there were 61 additional polymorphisms that were not in either allele, but were already in Group E which was defined by an NP gene that was novel to the other isolates in Hong Kong.

The genotype data presented in FIG. 1 also revealed more limited recombination, which could be seen in the paired isolates. In PA, the chicken isolates YU822.2 were from Group A and matched Allele 1. However, the two sequences diverged between positions 933 and 1143. There were 18 bimorphisms in this region and only two positions were the same in both isolates. For the 16 positions that diverged, the mouse brain isolate matched allele 2 at all 16 positions. A similar crossing over event was seen in the PB1 gene. NT873.3 was in Group E and matched allele 2. However, the two sequences diverged between positions 1374 and 1509. The were 10 bimorphisms and NT873.3 matched the consensus sequence for allele 1 at all 10 positions, identifying another cross-over event that had caused a short region of allele 1 to be detected in allele 2.

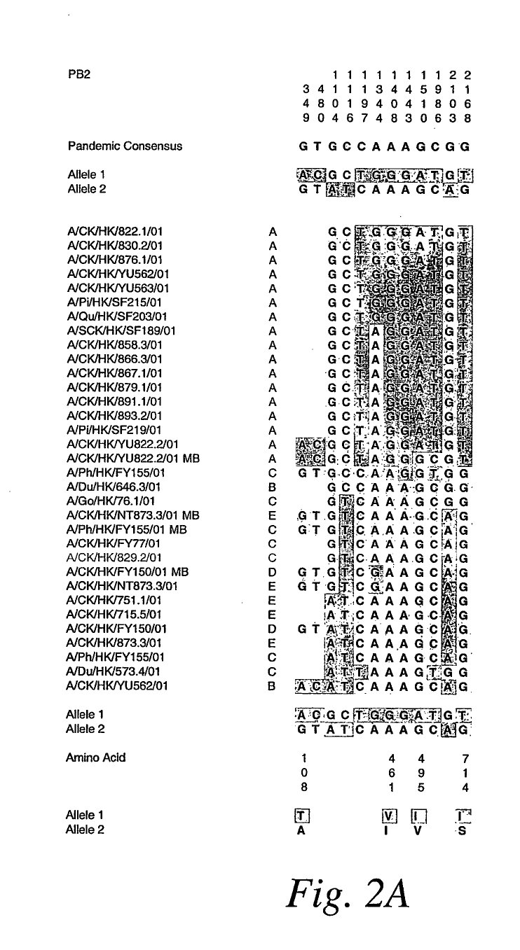

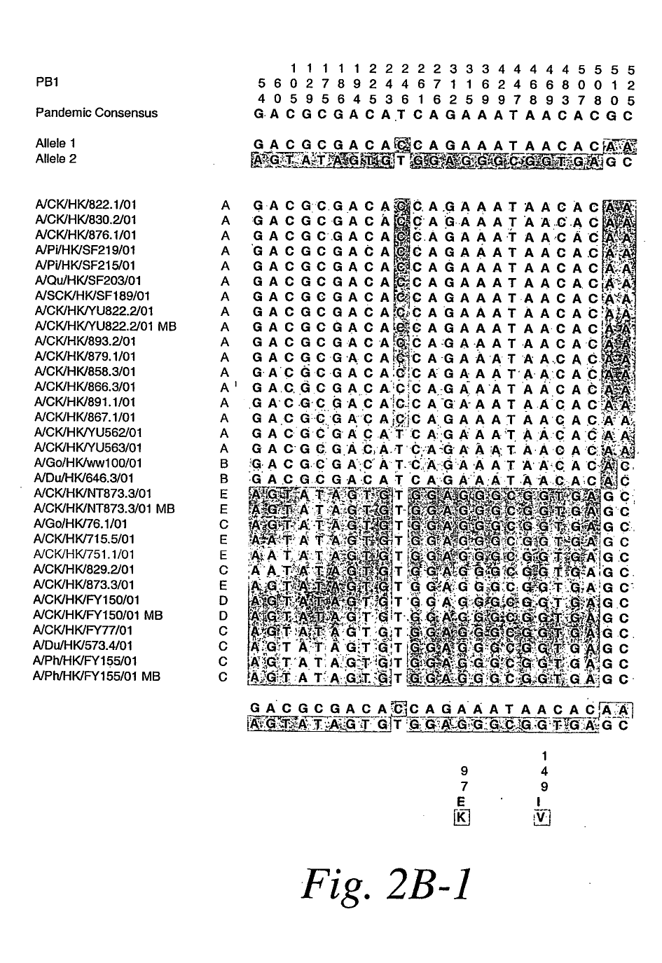

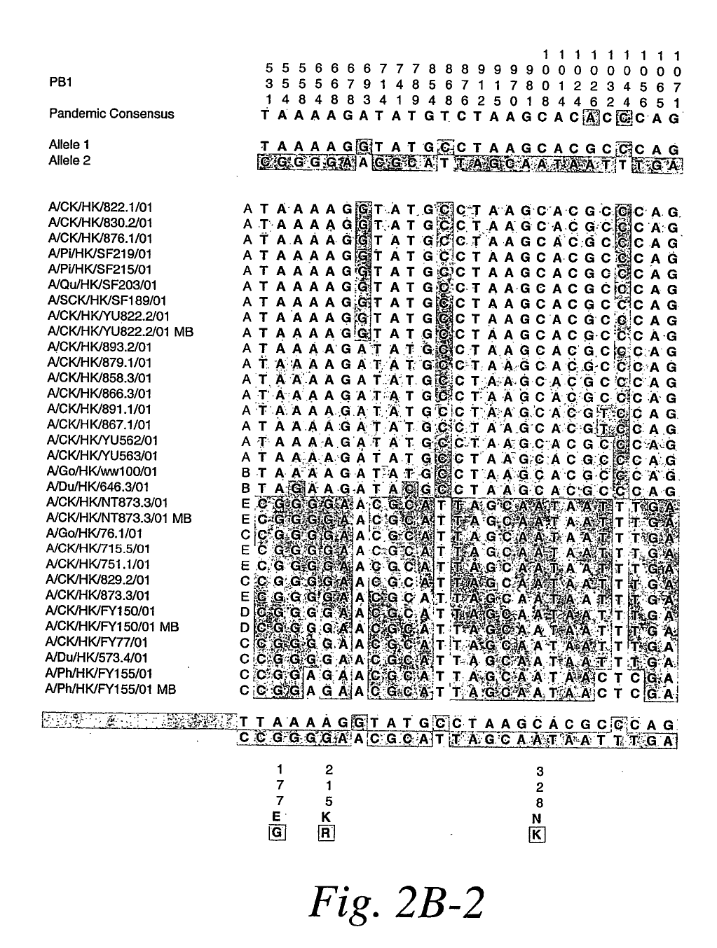

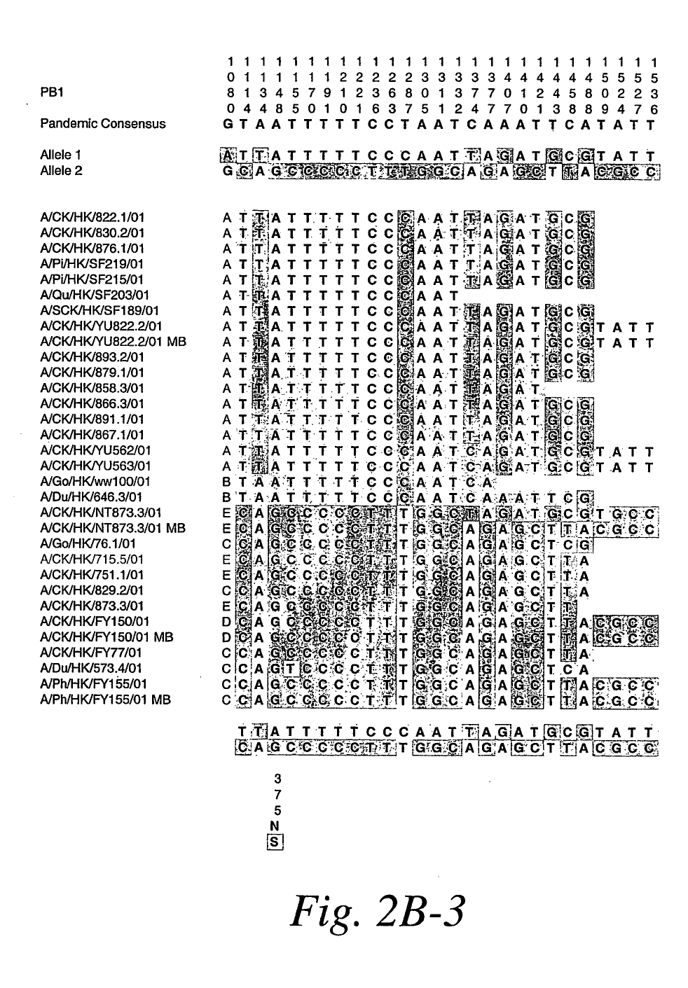

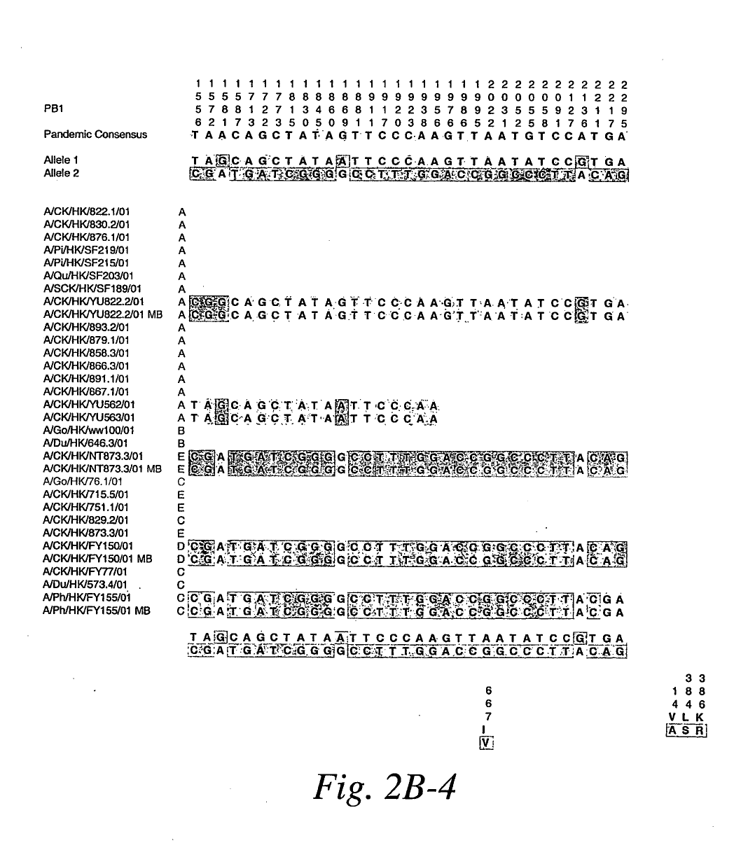

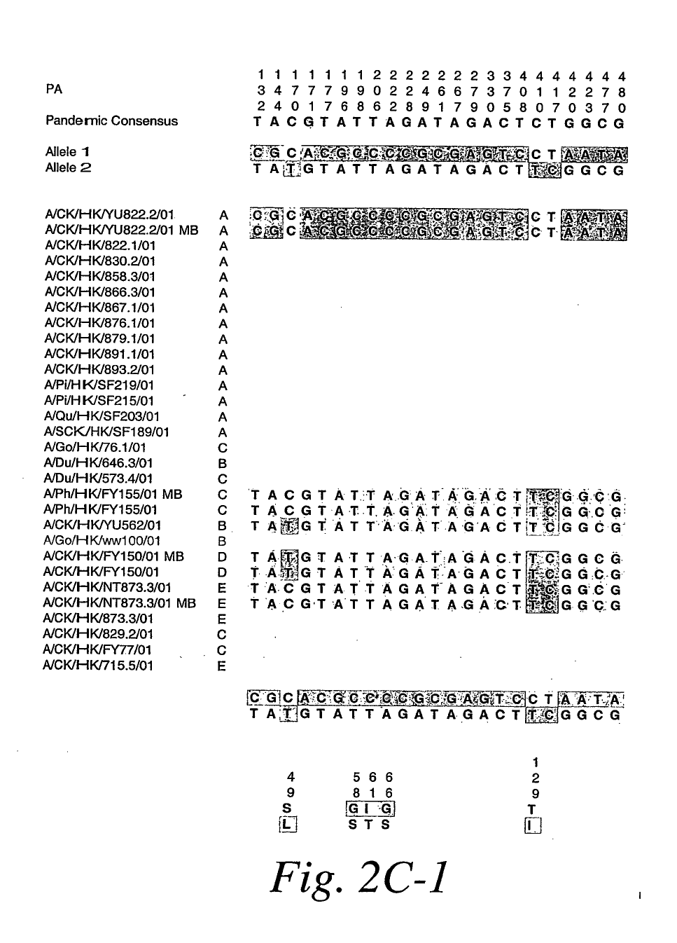

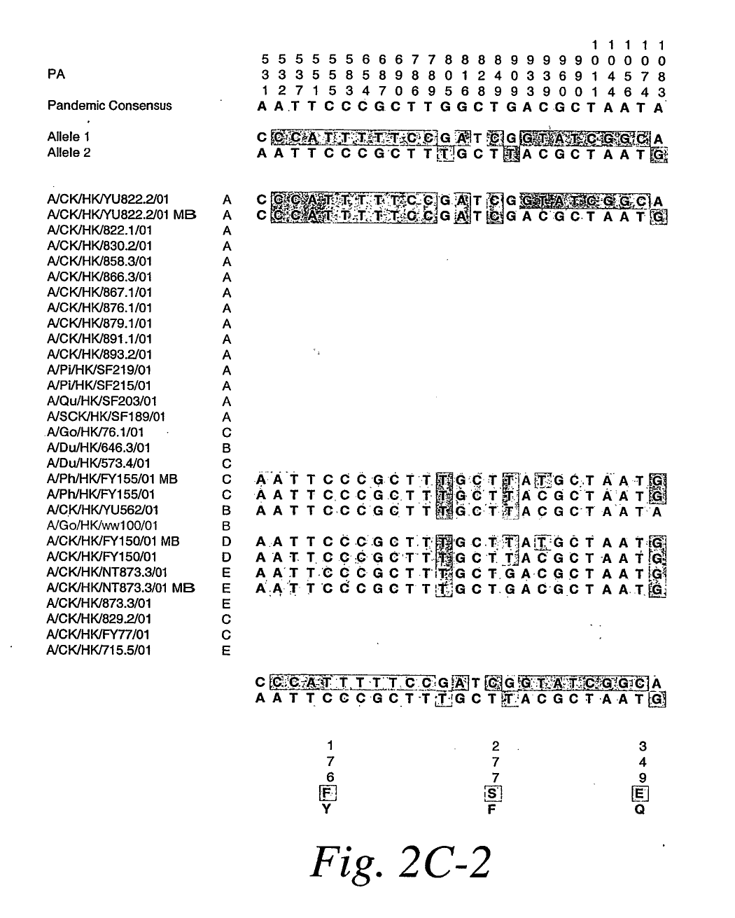

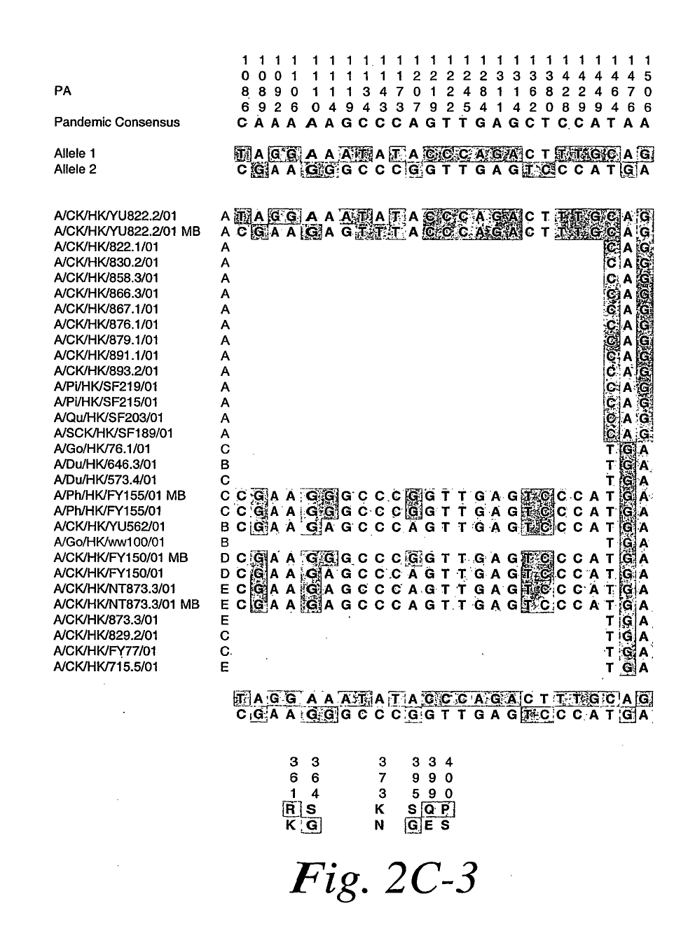

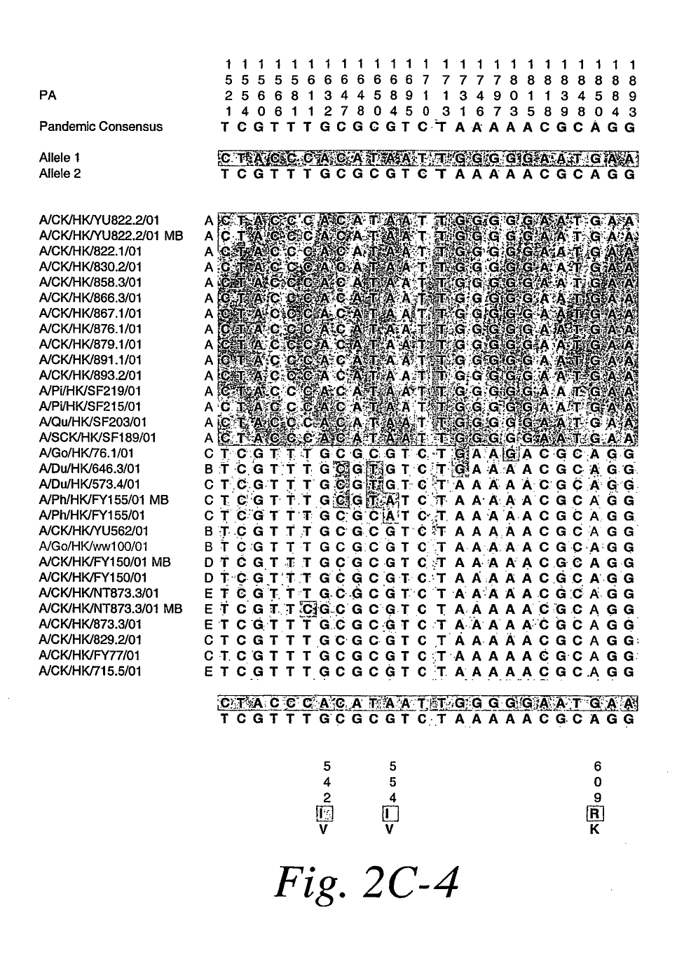

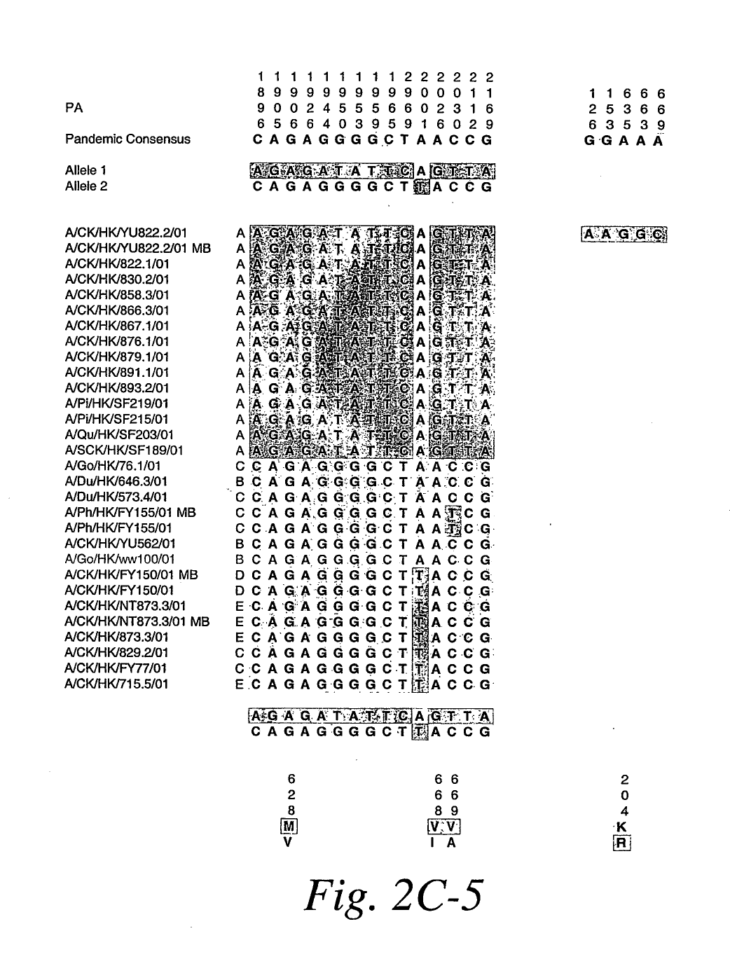

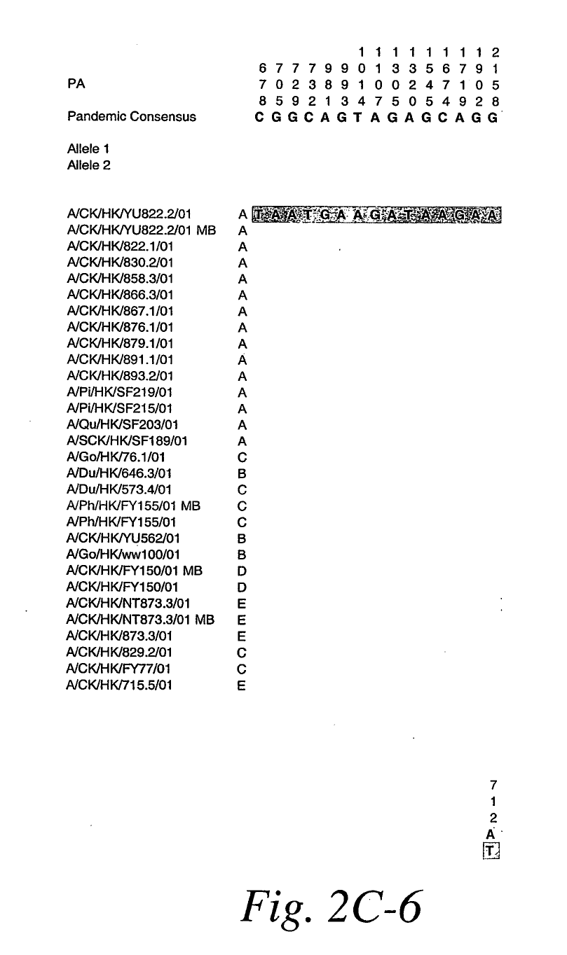

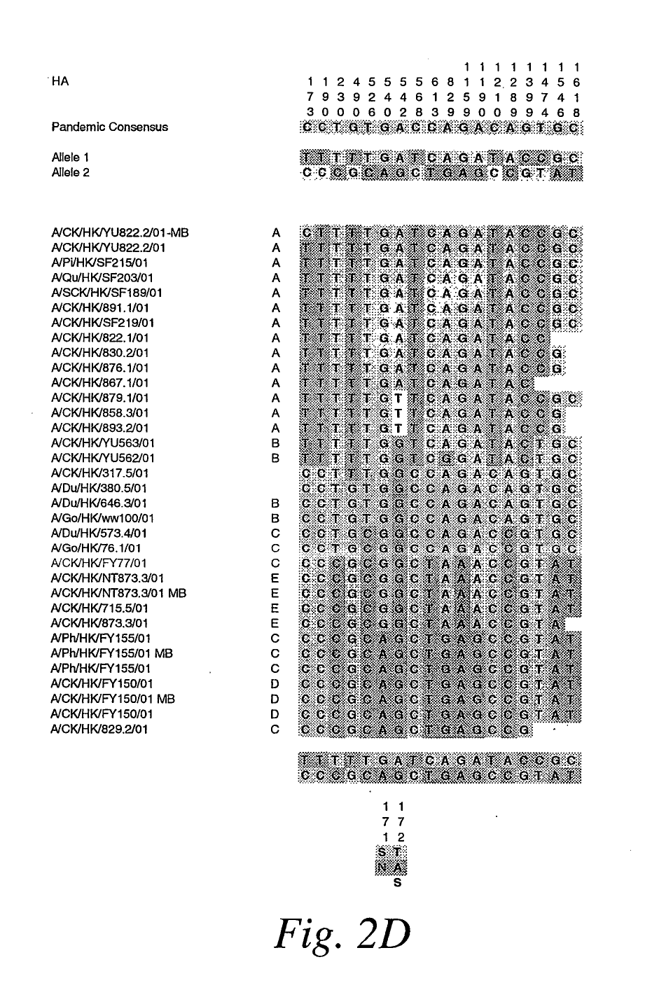

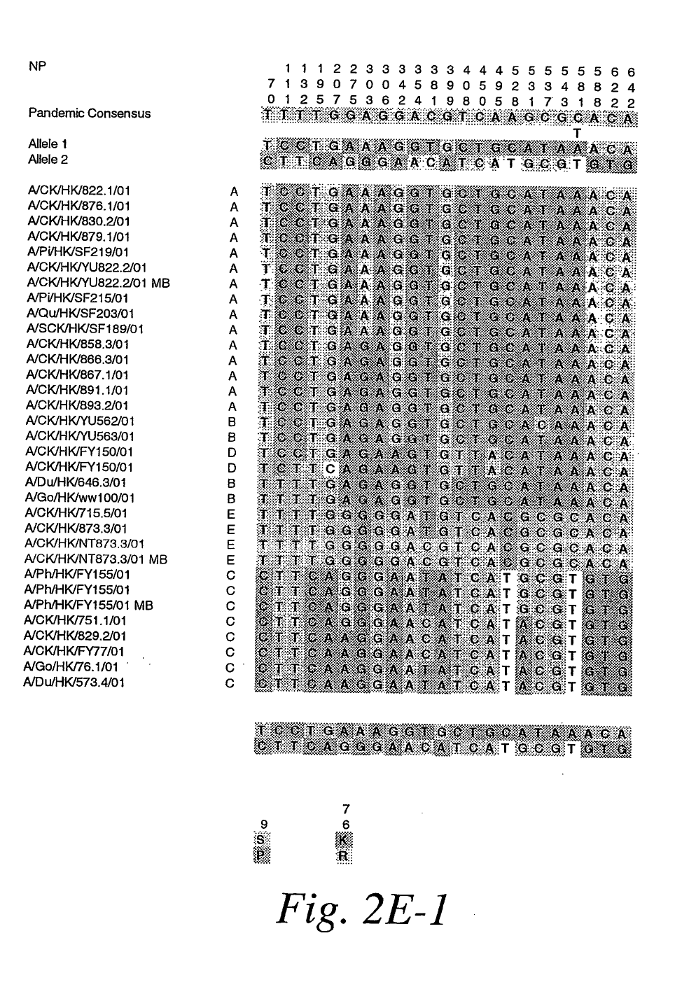

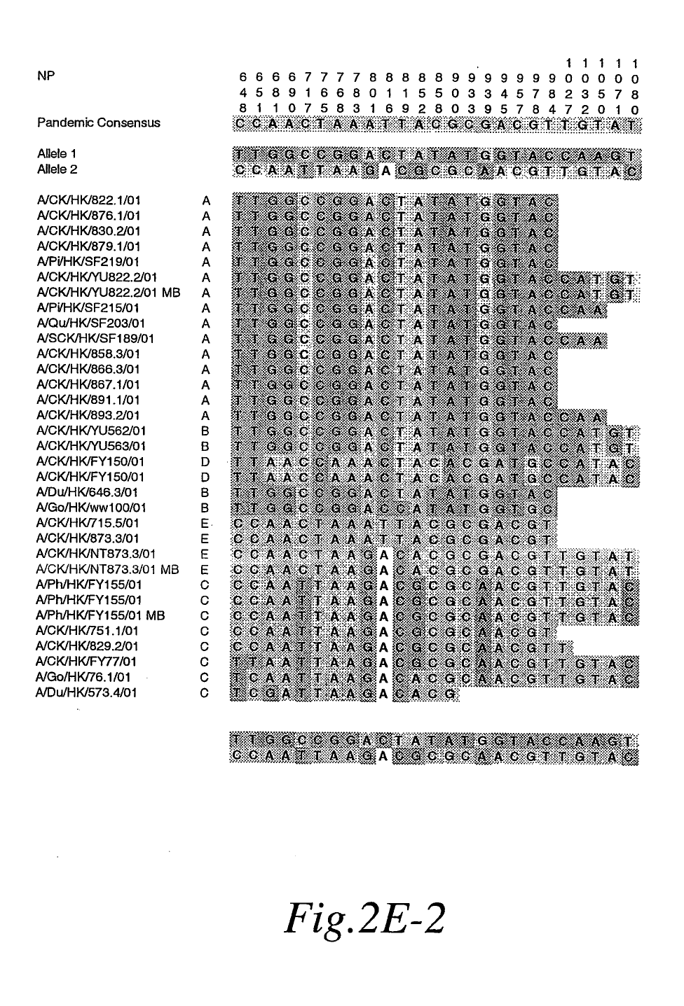

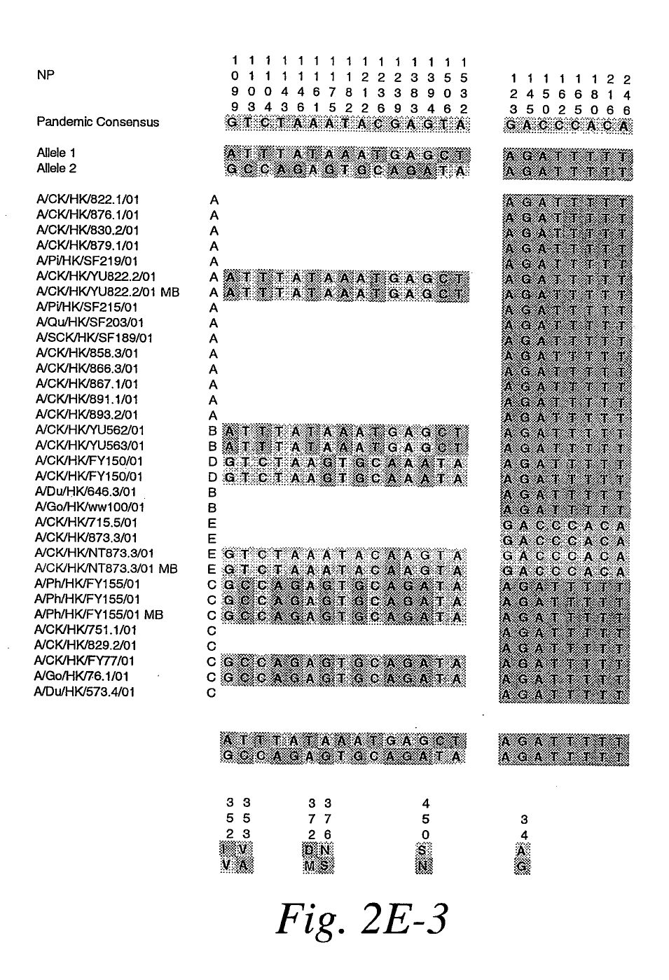

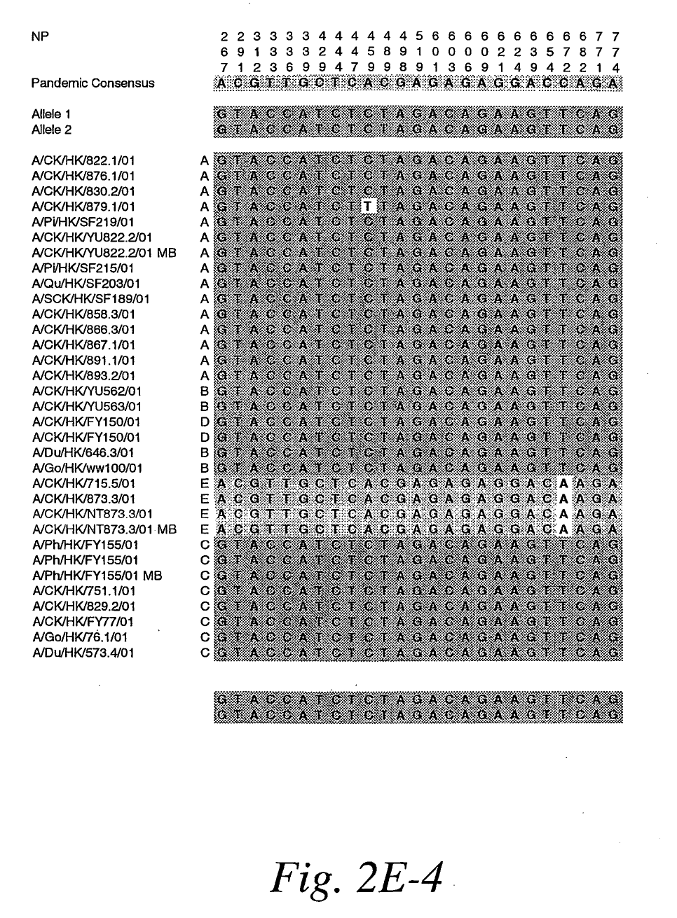

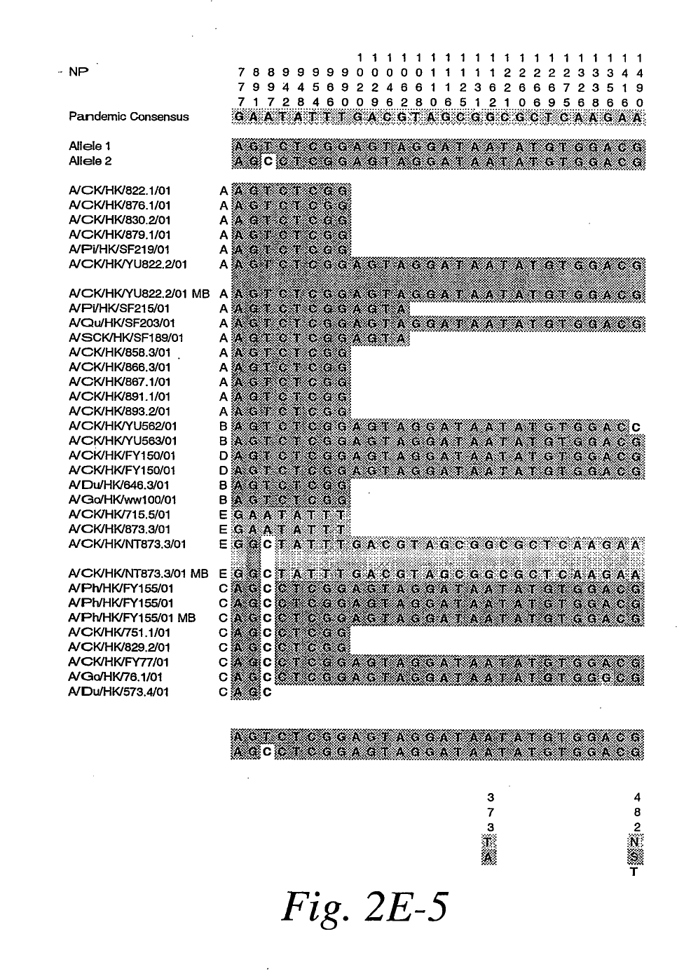

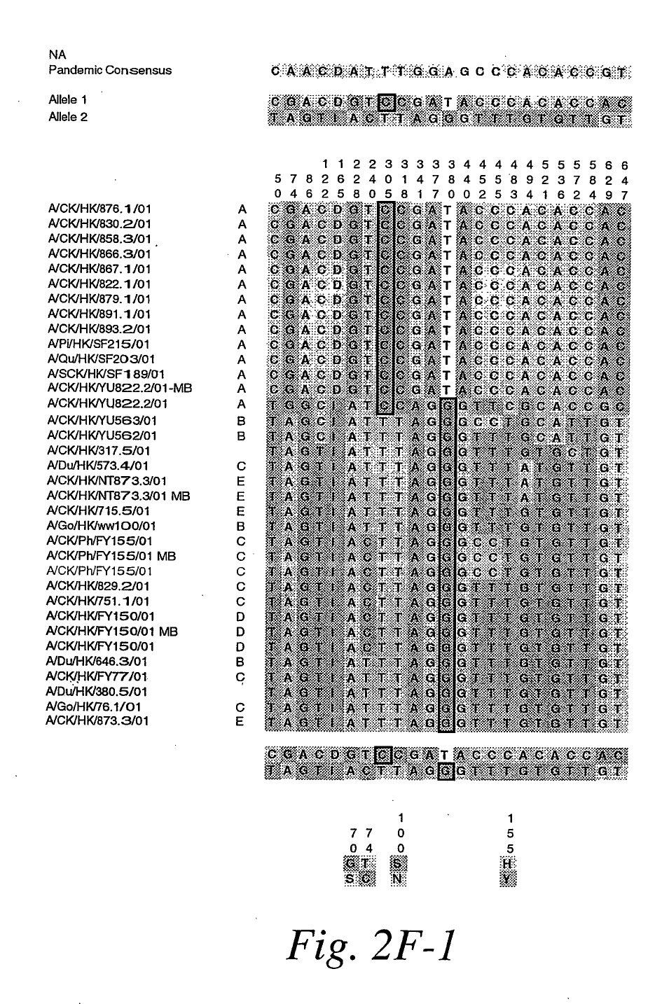

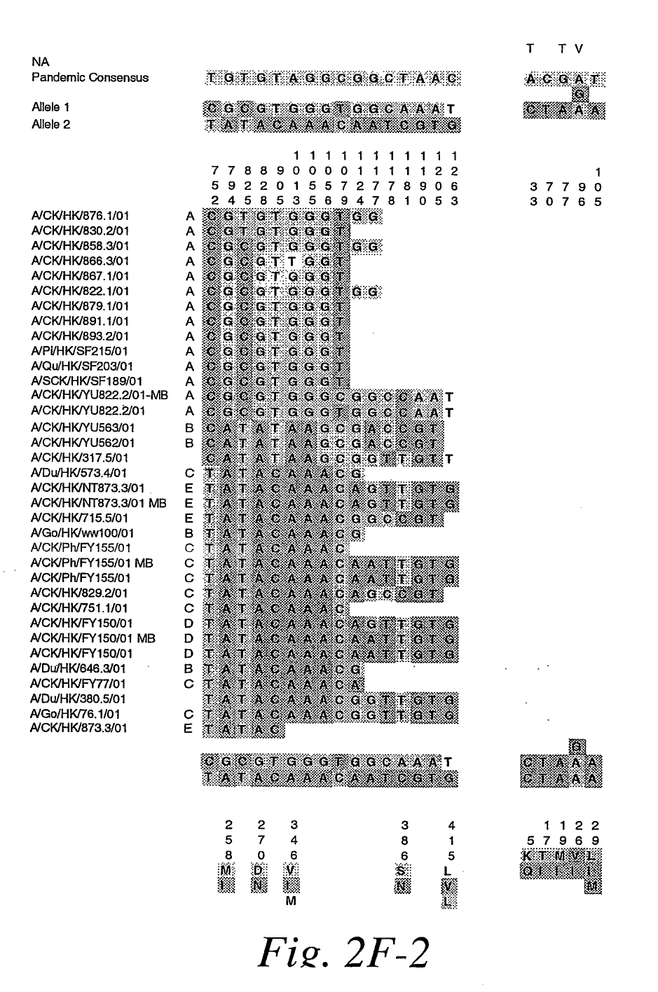

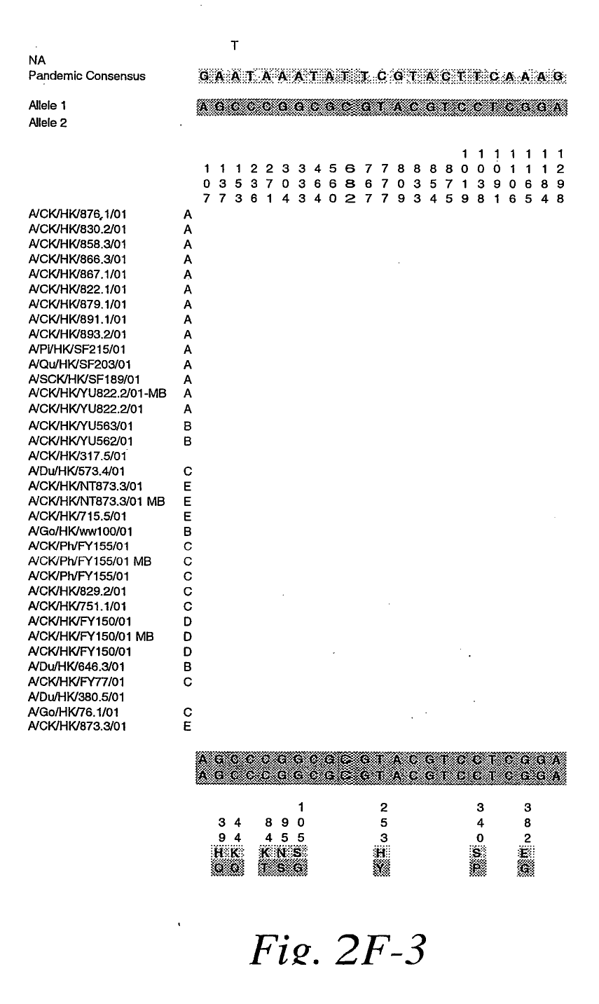

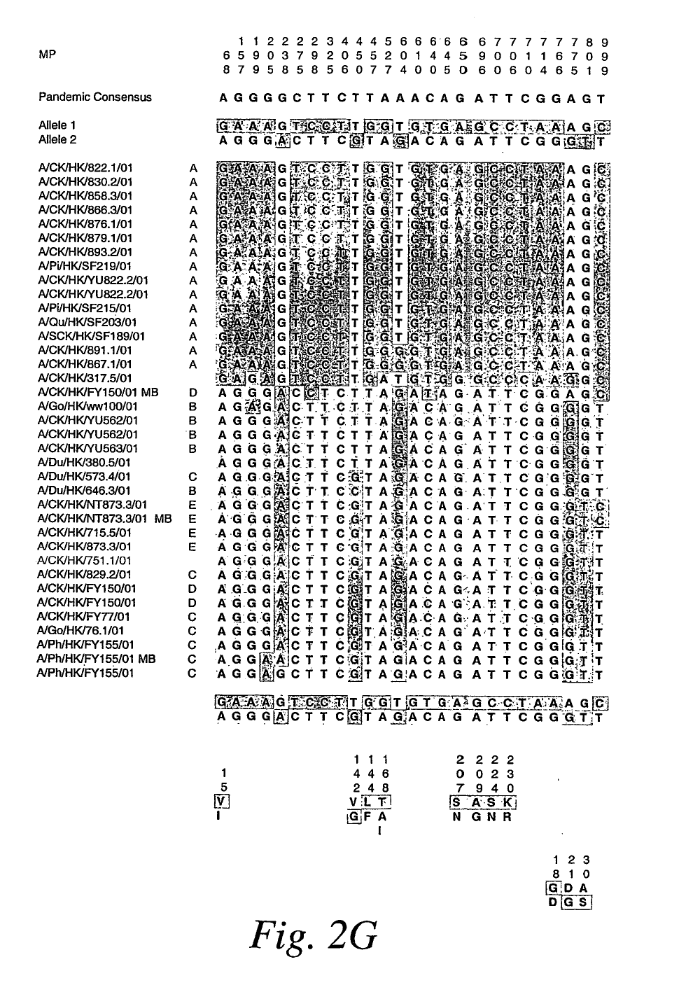

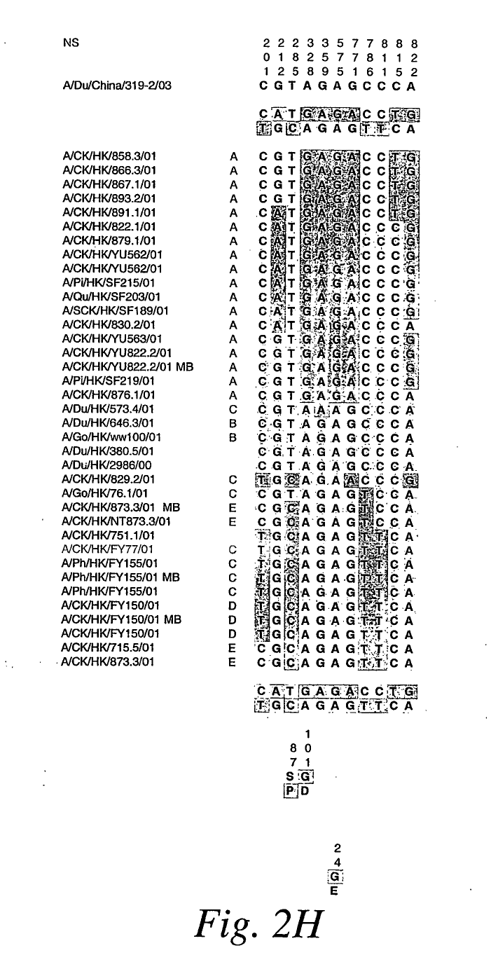

FIG. 2 shows the result of much more extensive recombination seen in the Hong Kong isolates from 2002 and 2003 (Strum-Ramirez et al 2004). Panel 1 contains the same bimorphisms from FIG. 1. However, the more recent isolates have recombined the polymorphisms from allele 1 and 2 to create a new allele that is highly homologous to the pandemic strain. This recombination was present in all 8 genes. FIG. 2 also contains the bimorphisms that were present in the pandemic strain but were missing from the Hong Kong isolates from 2001. The genomes recombined in the bimorphisms in Panel A also had most of the polymorphisms not present in Hong Kong in 2001 (Panel B). Thus, not only were the bimorphisms on allele 1 and 2 combined, but the genes now contained most of the missing bimorphisms, which were likely acquired outside of Hong Kong. Panel C lists the bimorphisms not present in the 2002 and 2003 Hong Kong isolates. These bimorphisms can be found in more distantly related mammalian isolates (HLN in preparation). However, FIG. 2 shows that the bimorphisms from FIG. 1 recombined and added most of the bimorphisms that were not present in Hong Kong in 2001.

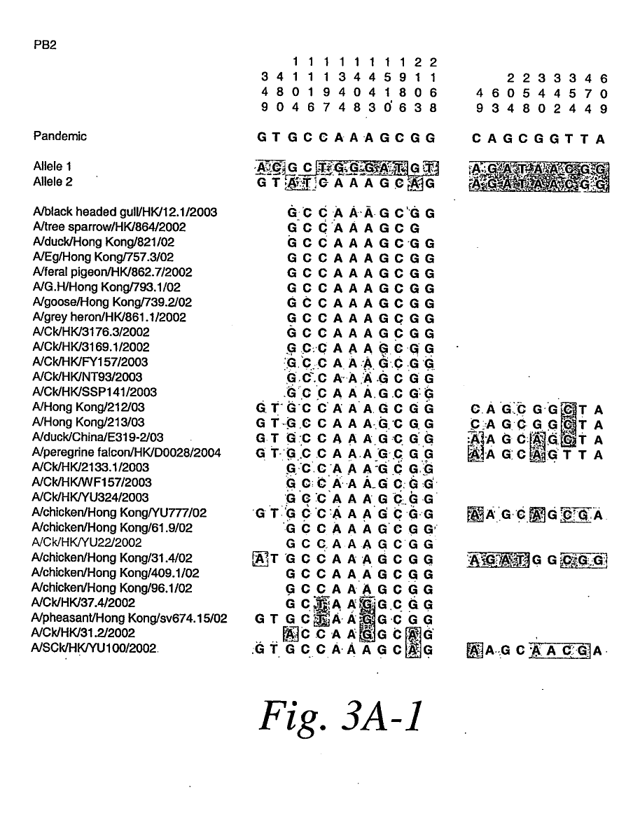

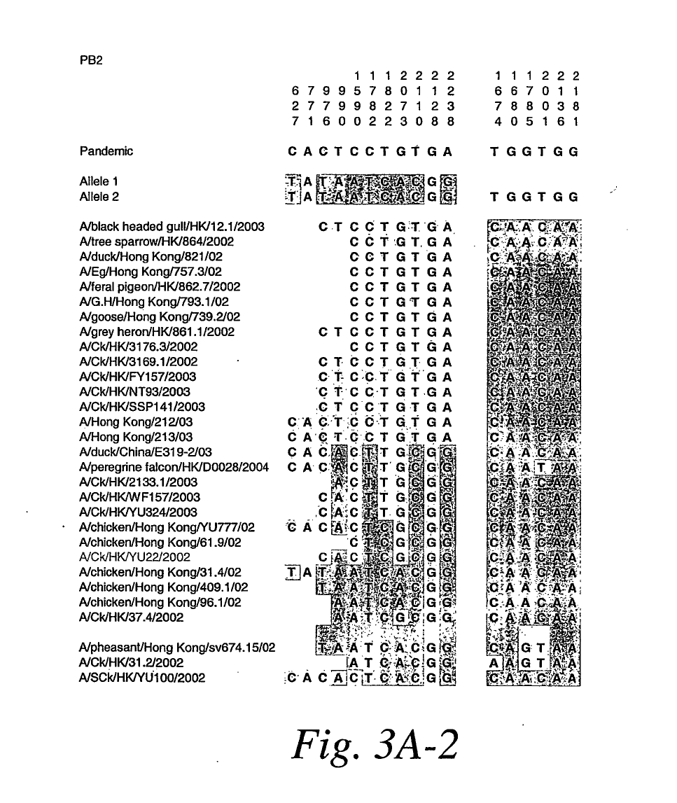

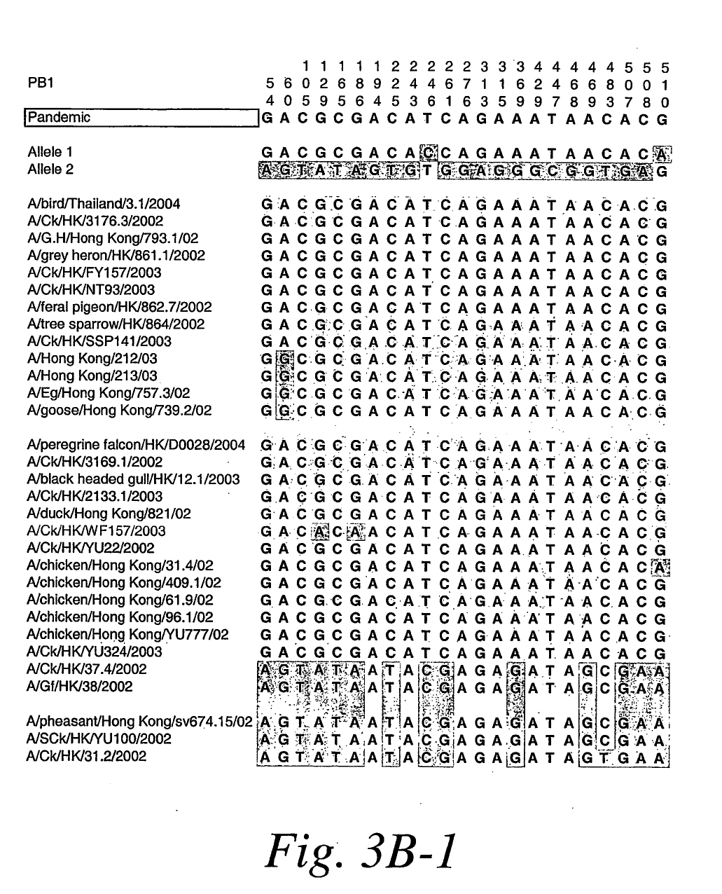

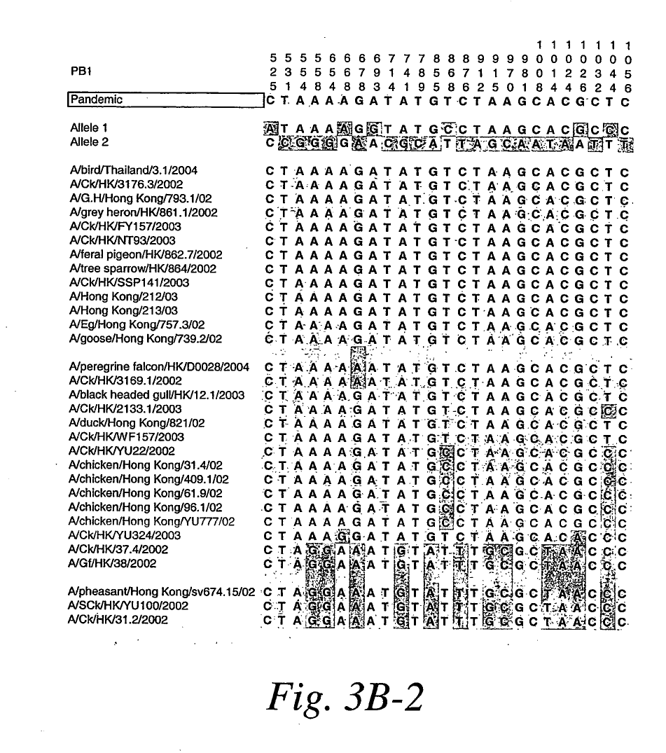

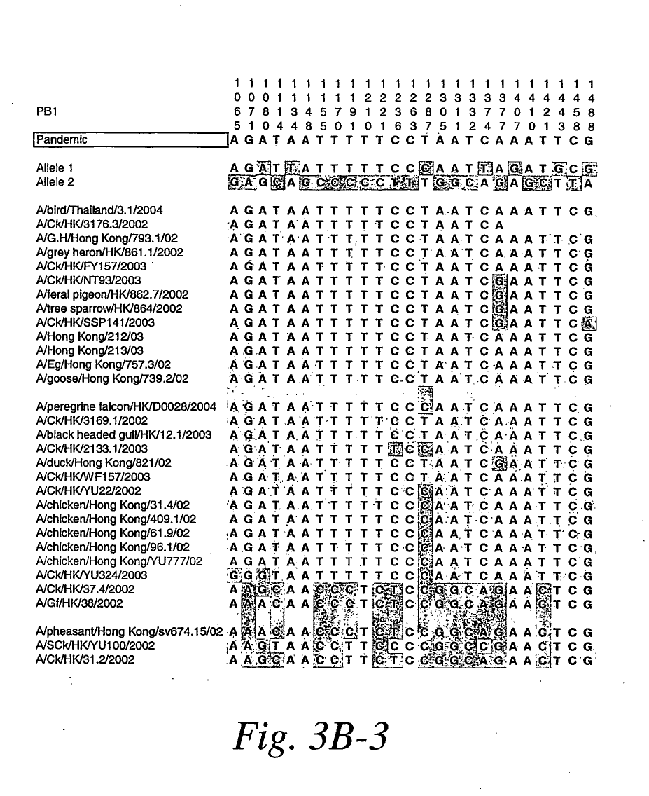

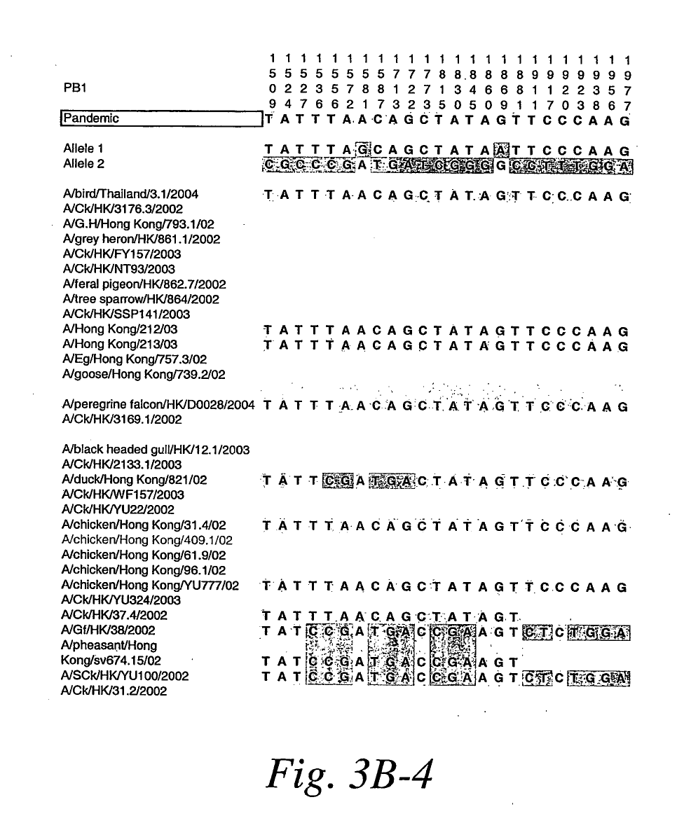

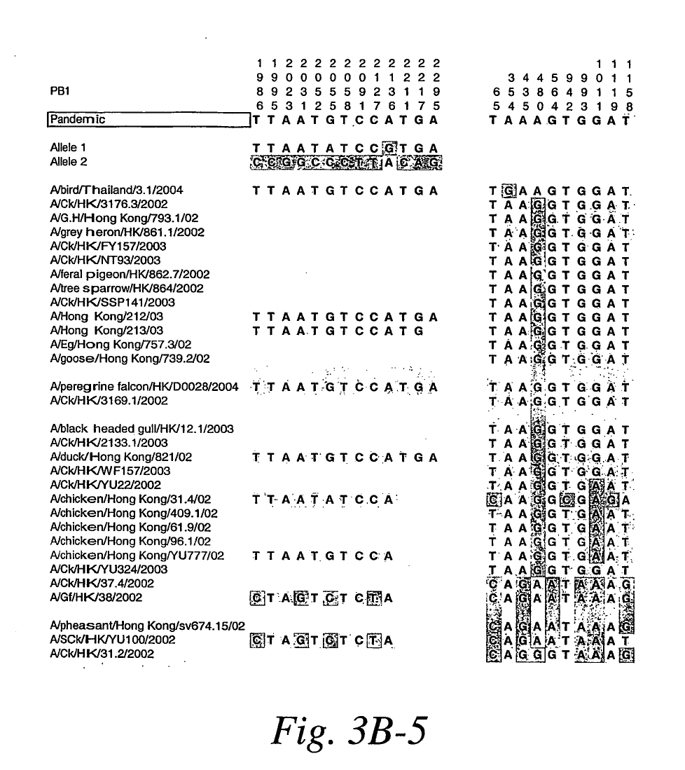

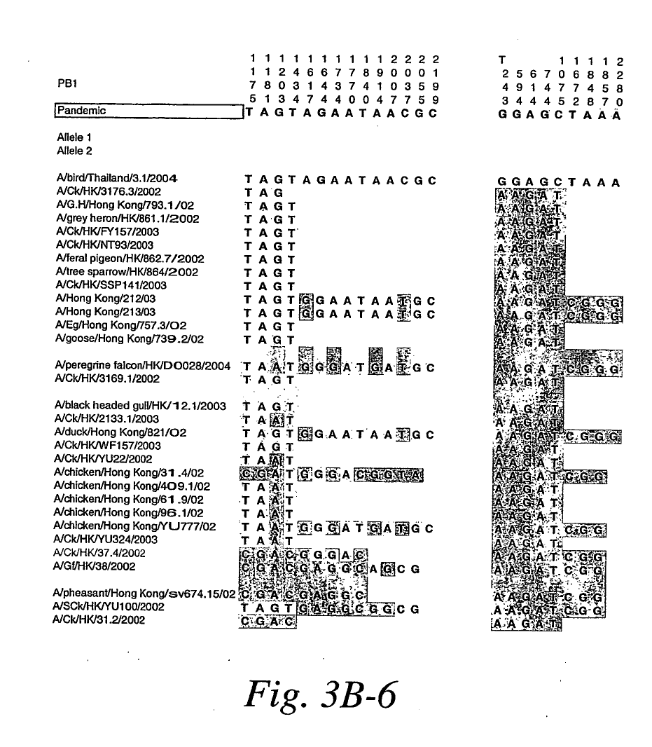

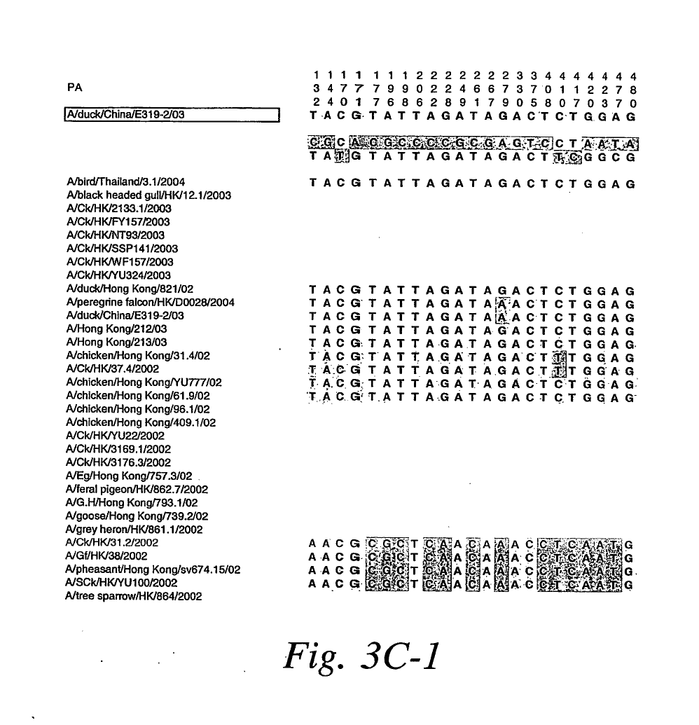

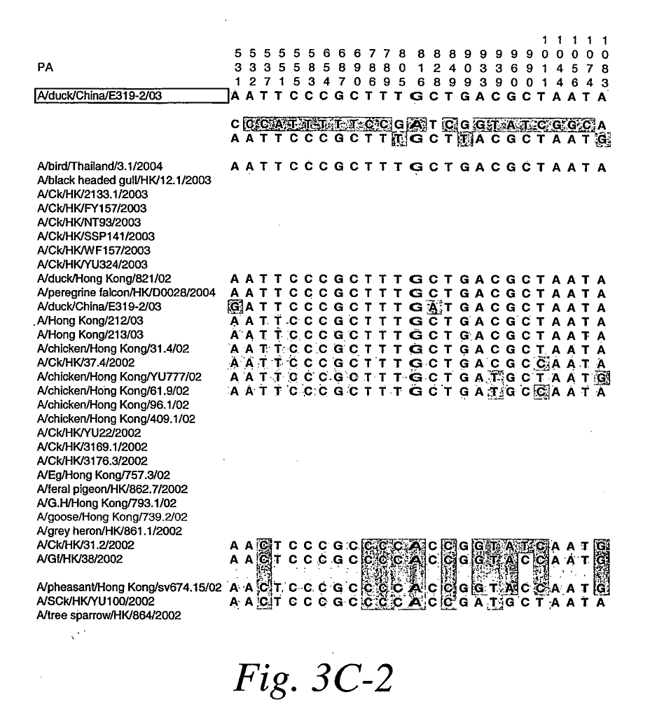

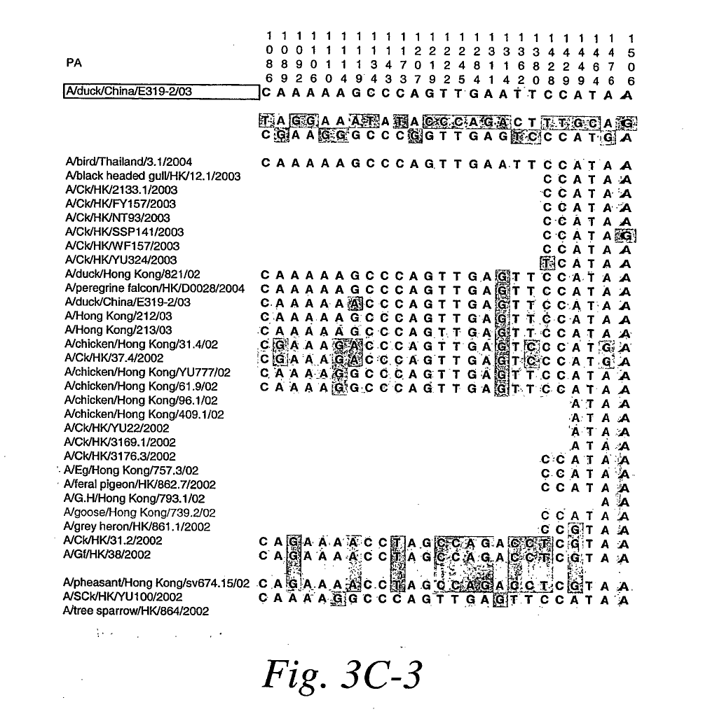

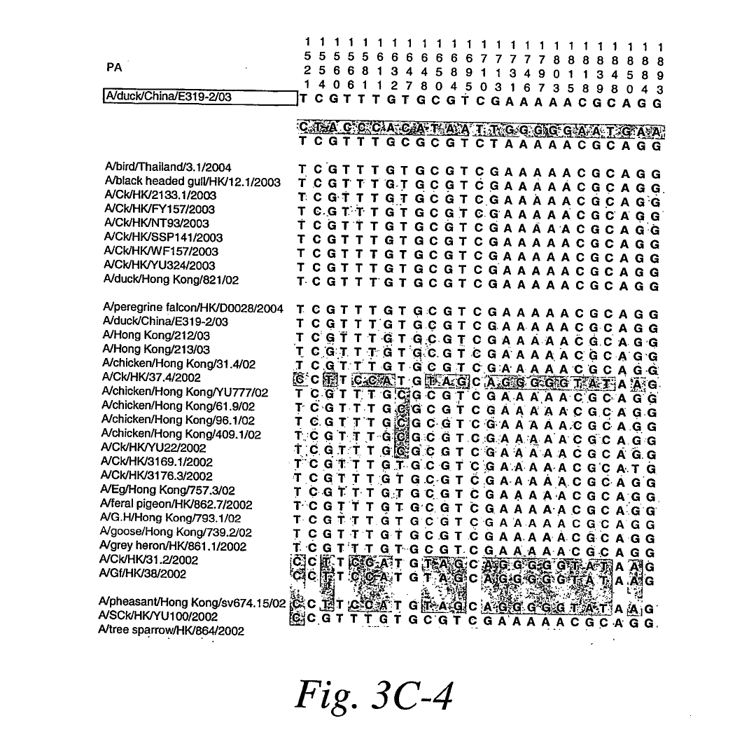

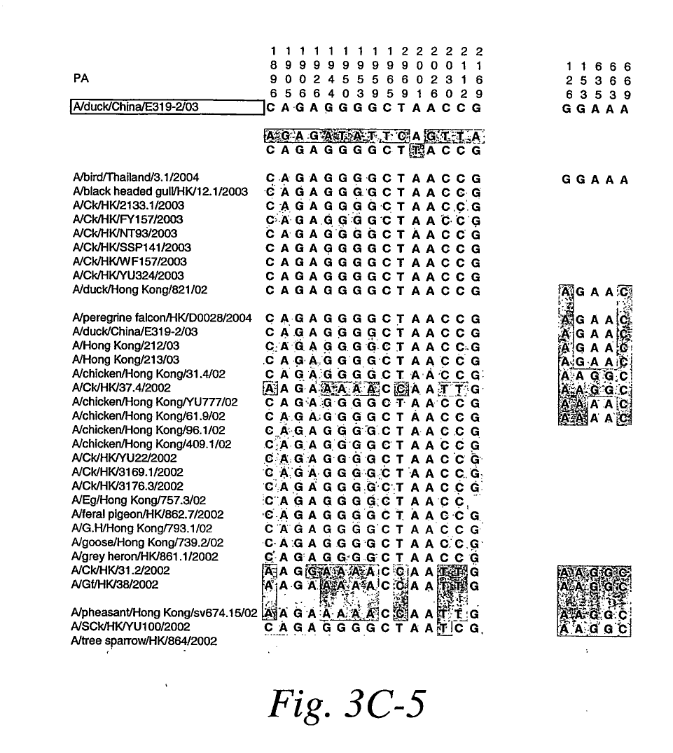

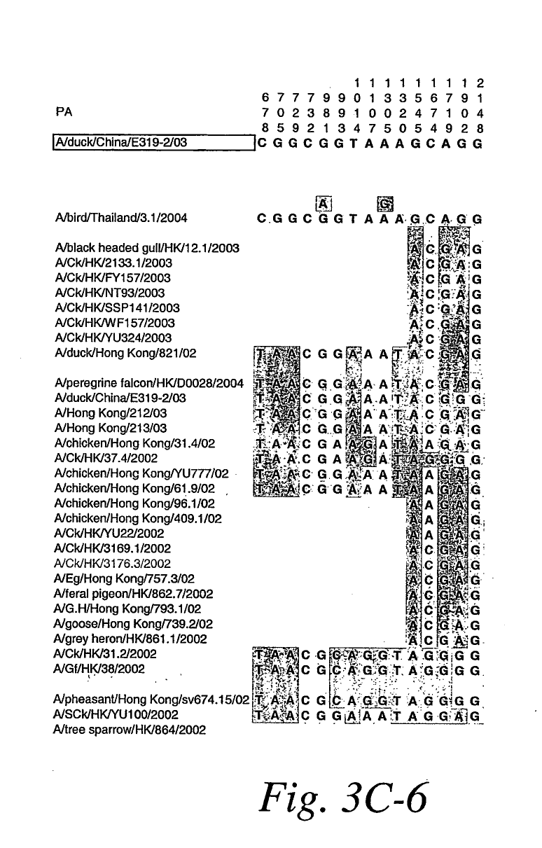

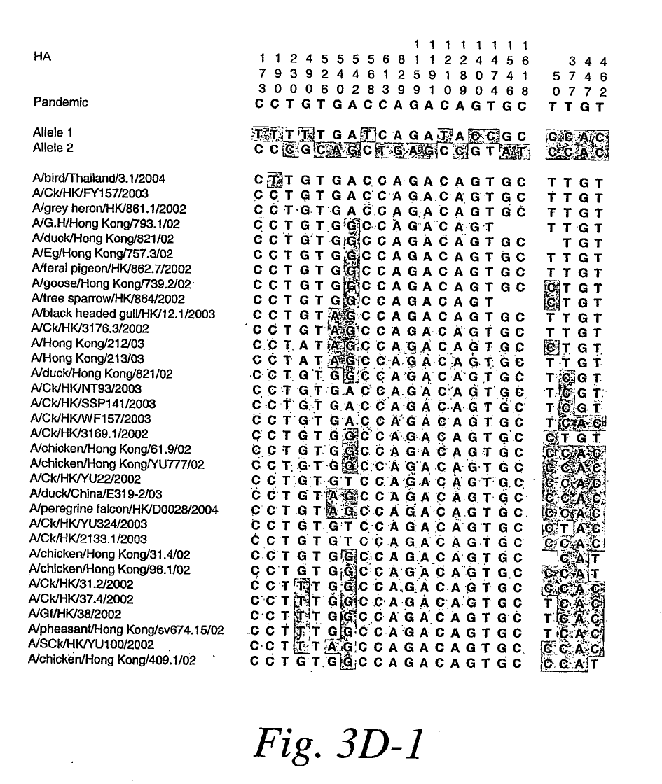

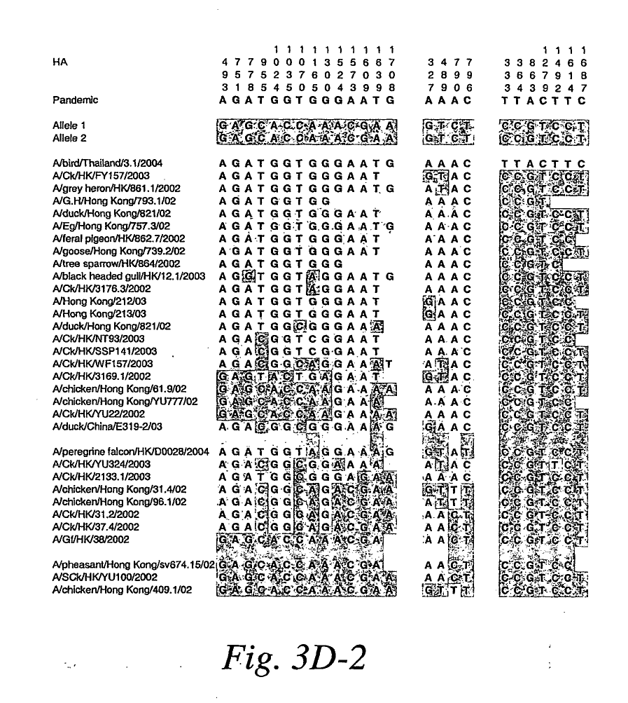

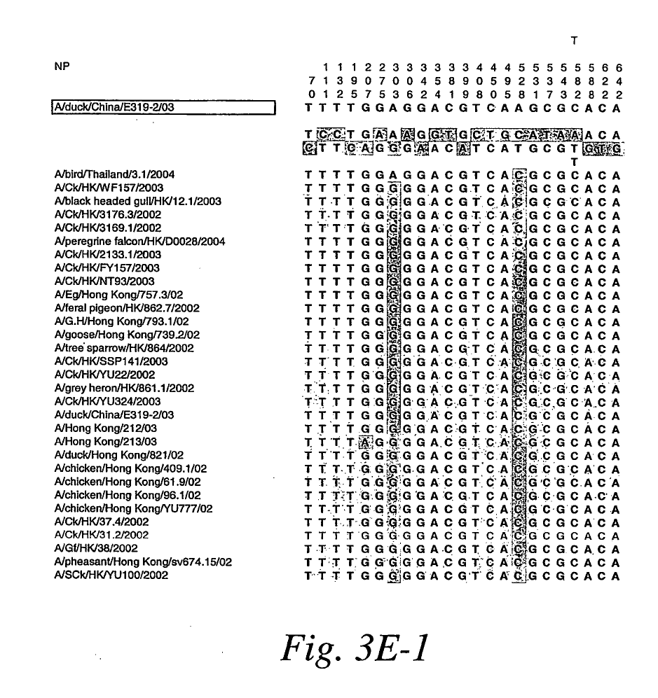

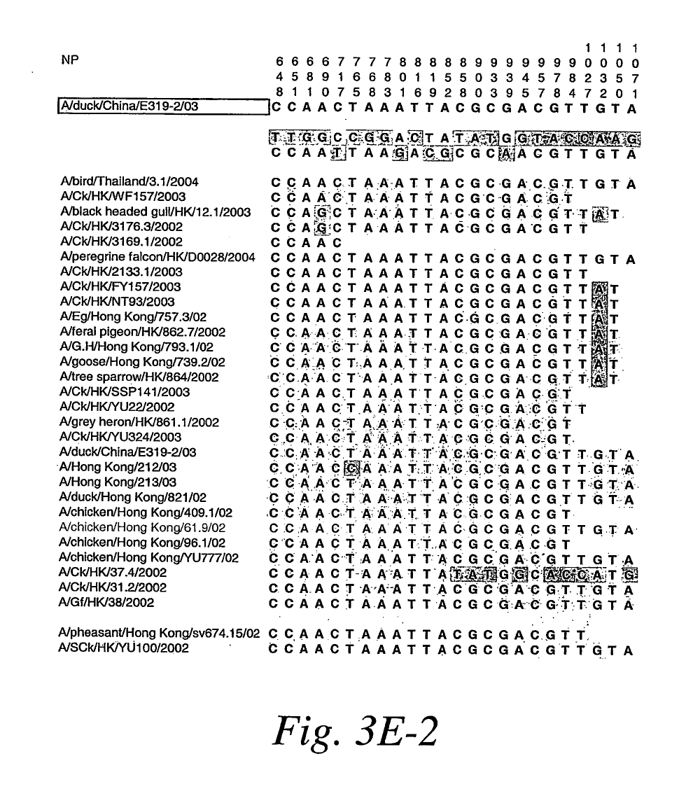

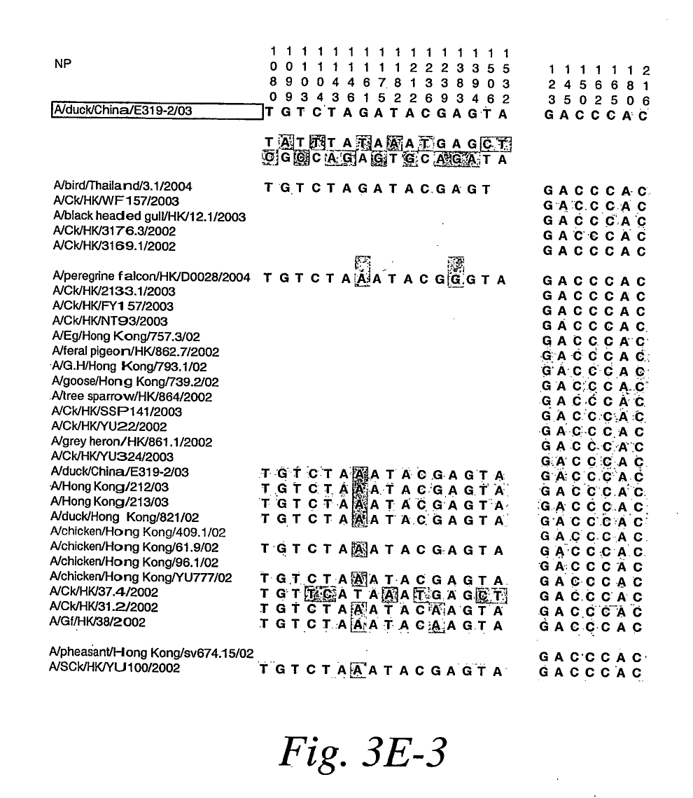

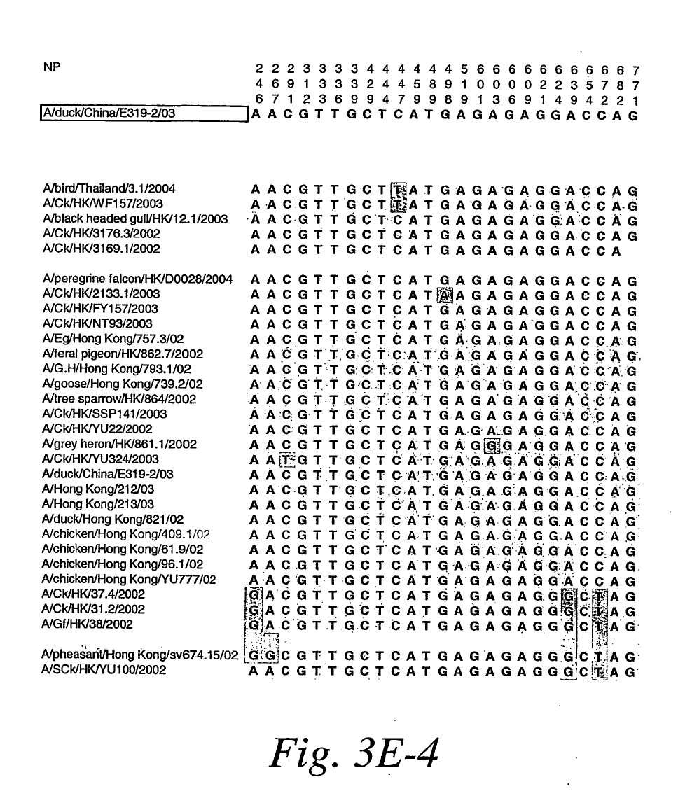

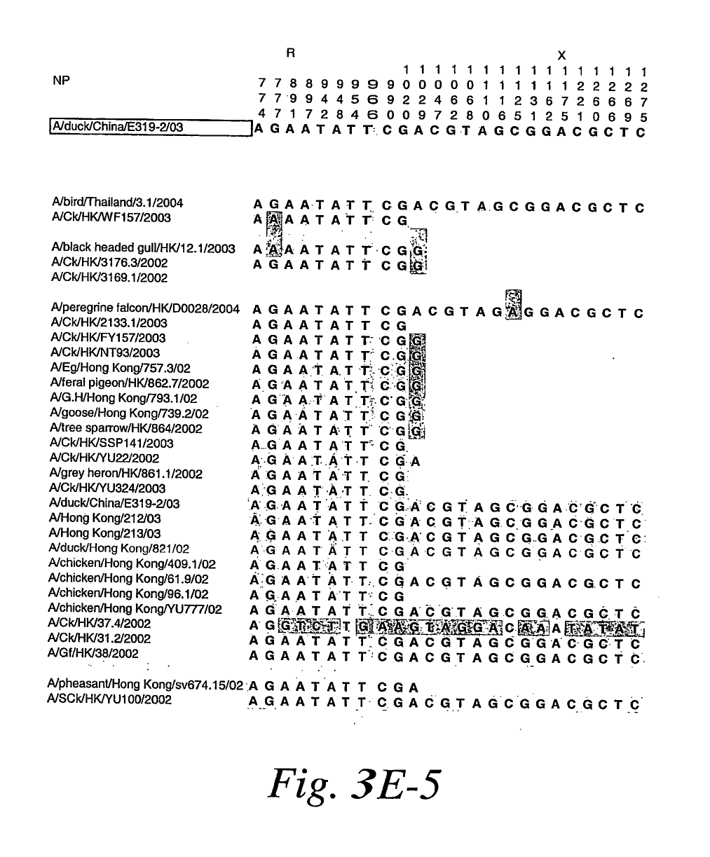

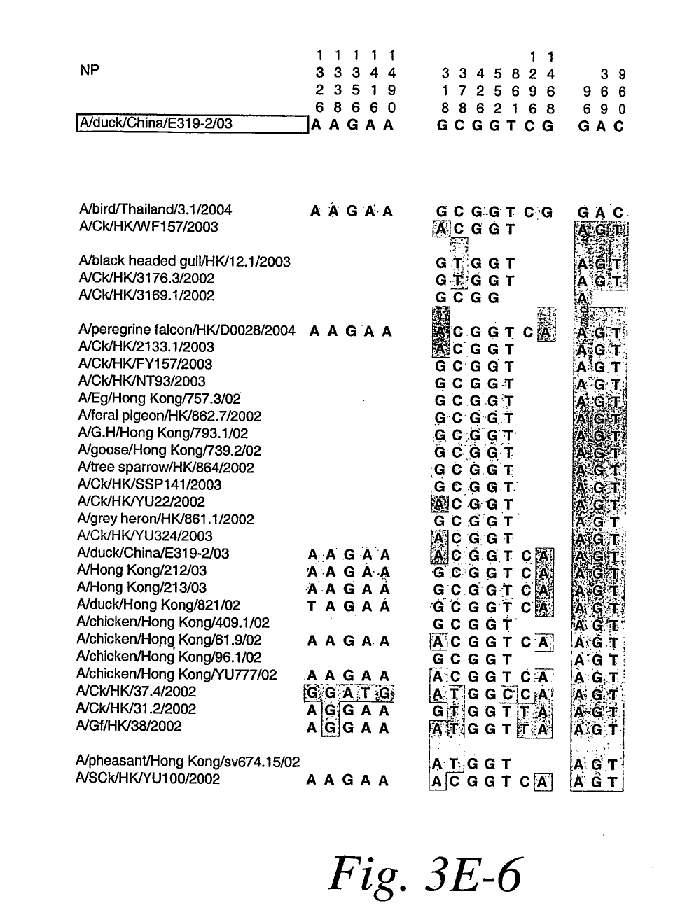

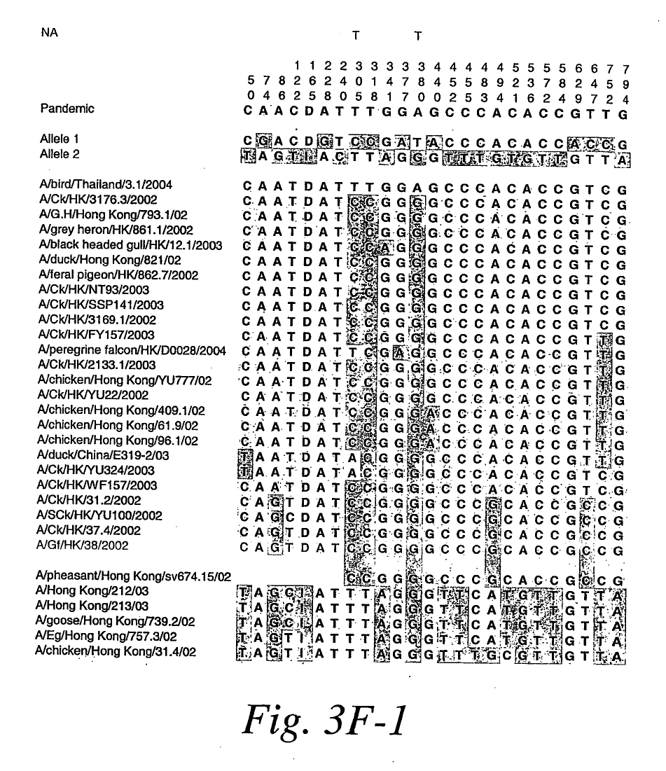

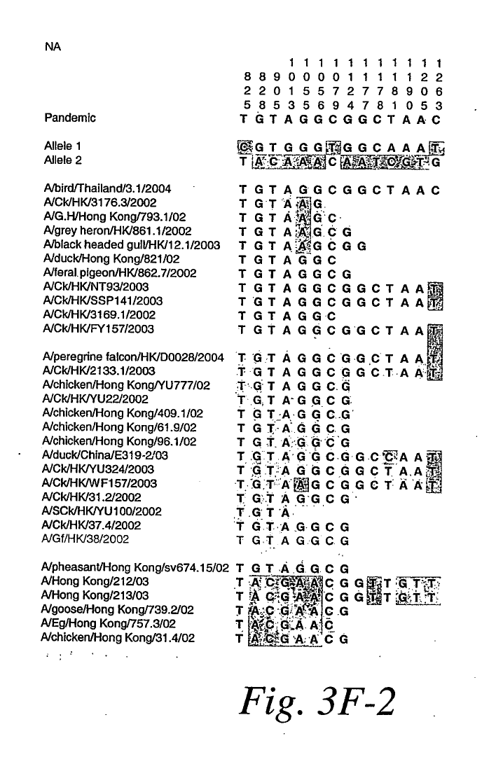

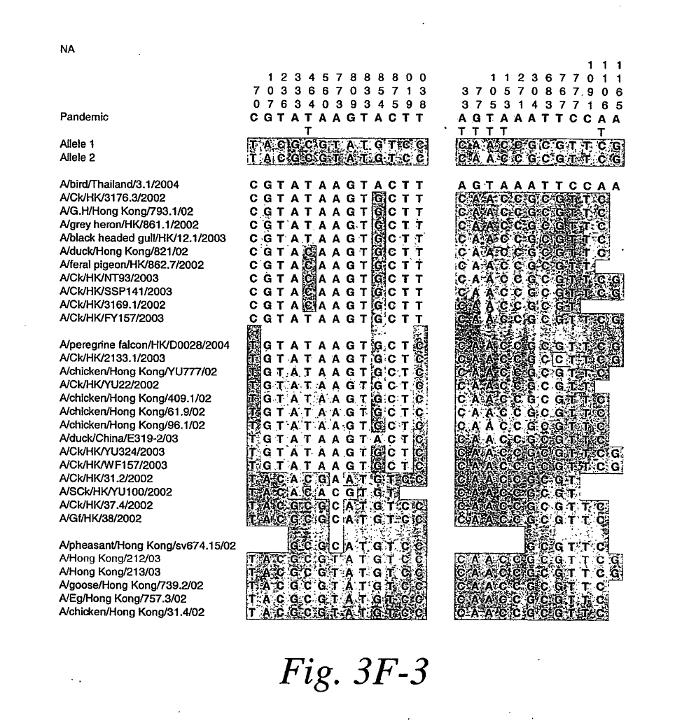

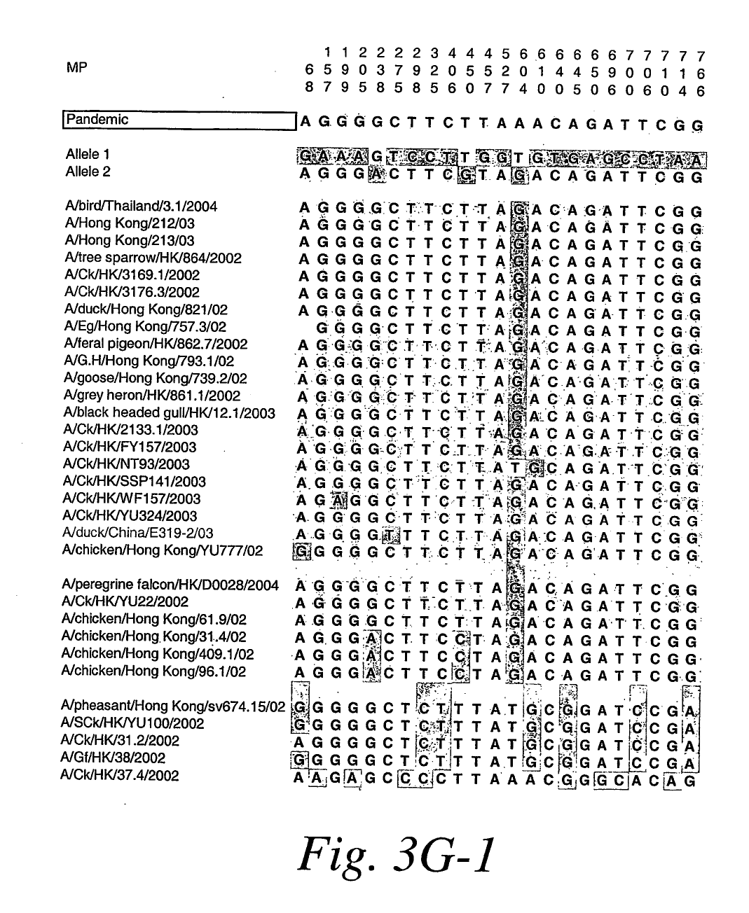

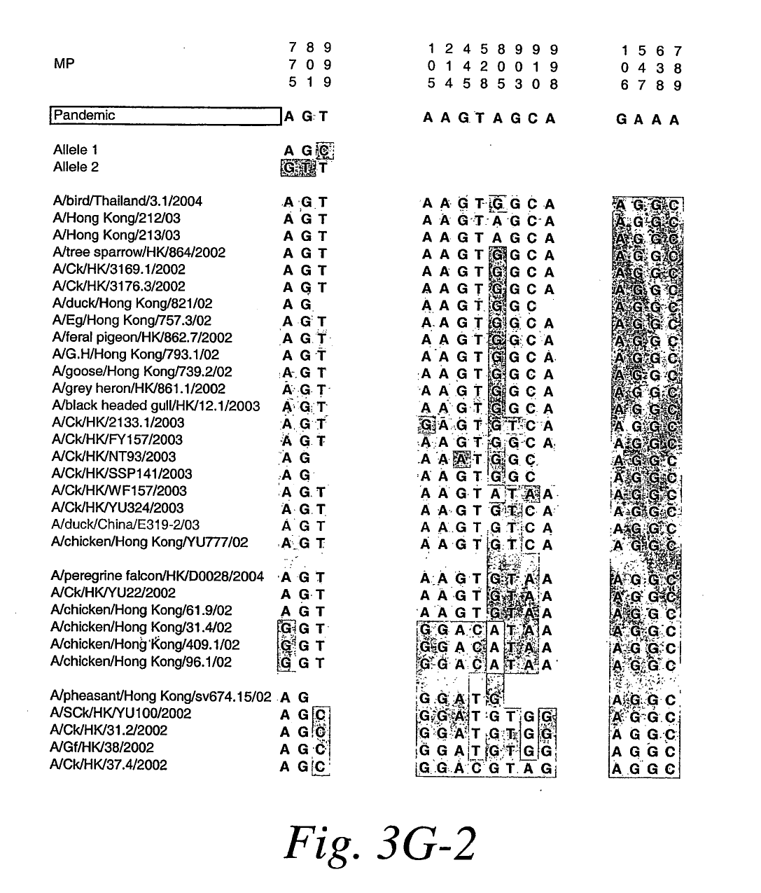

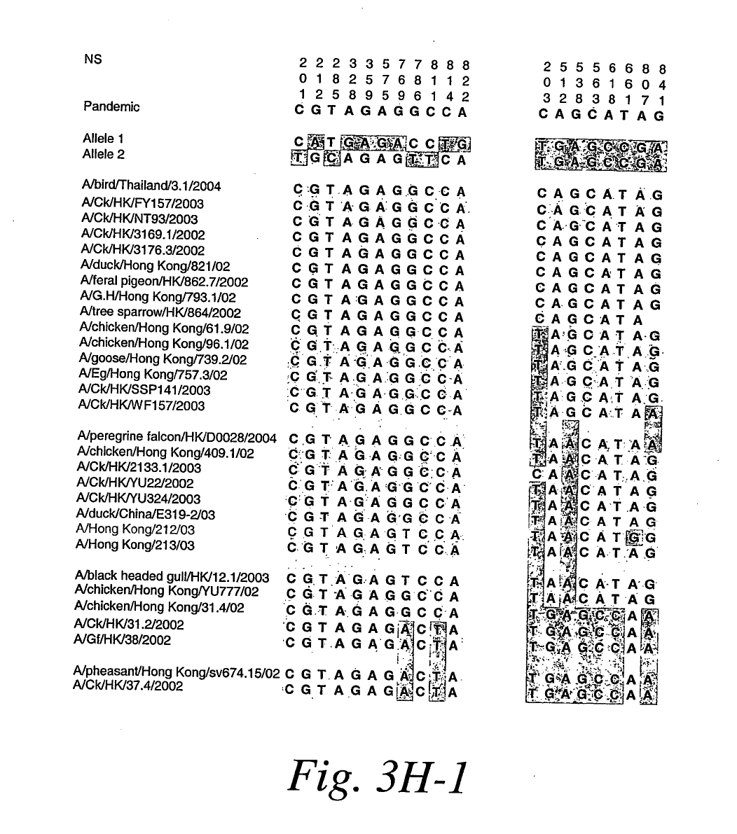

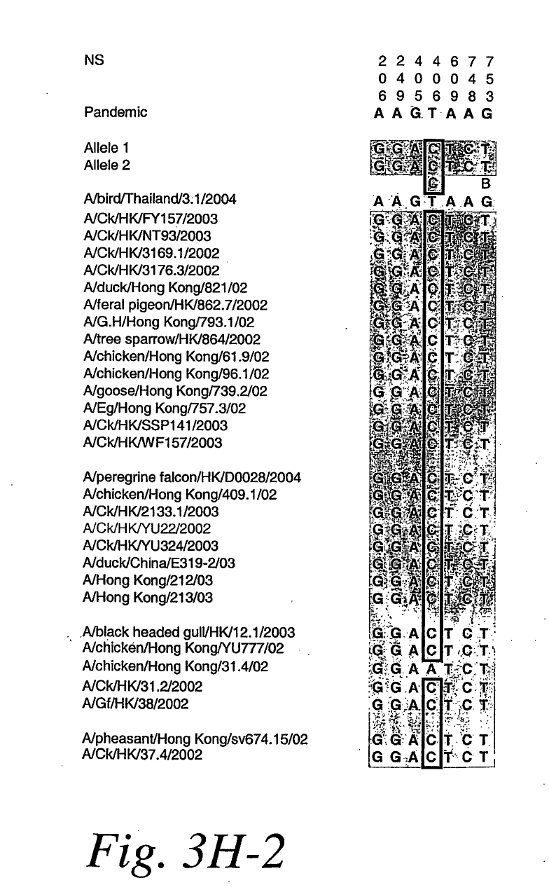

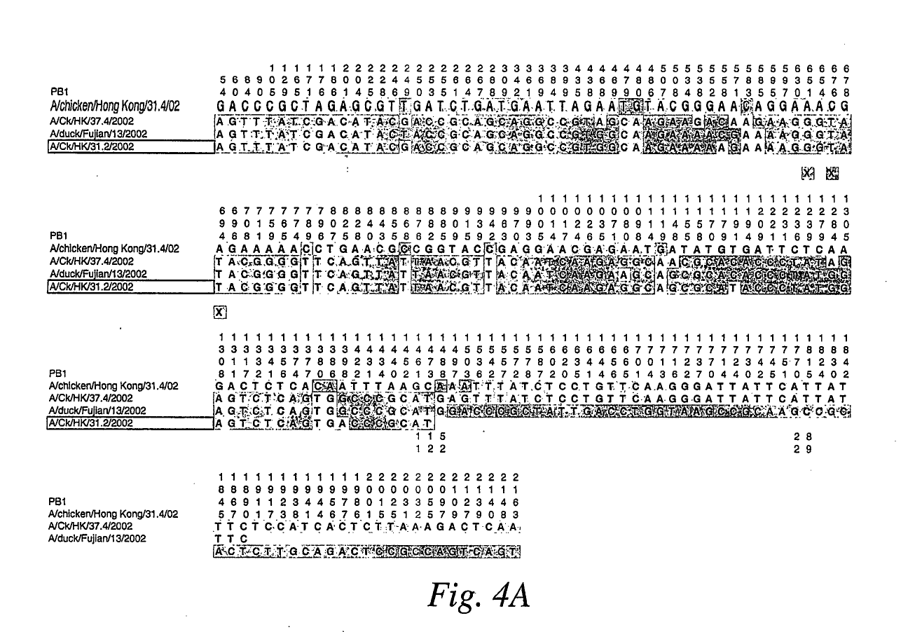

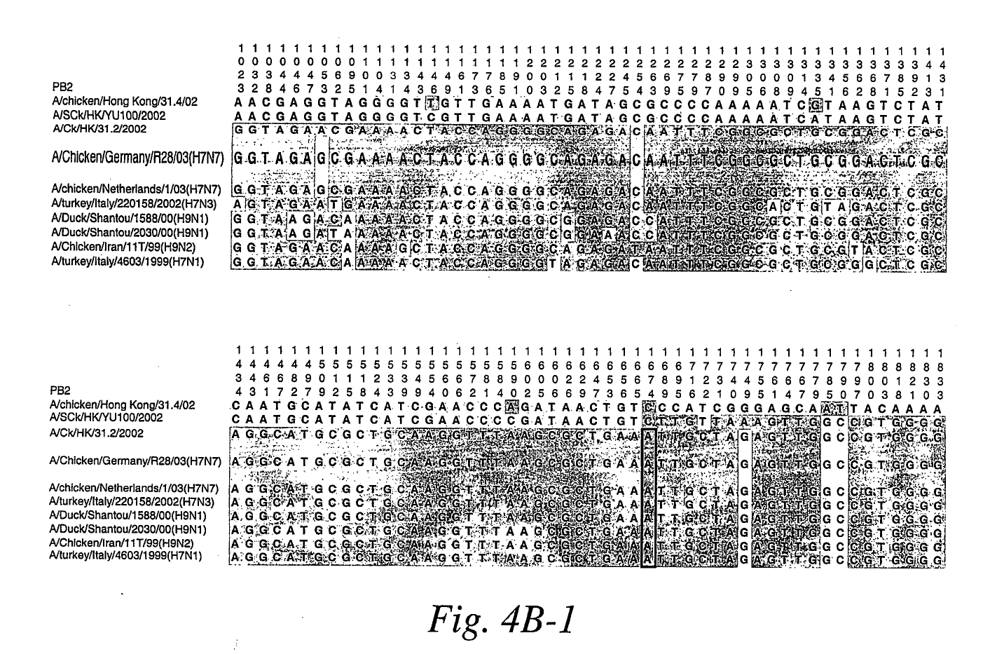

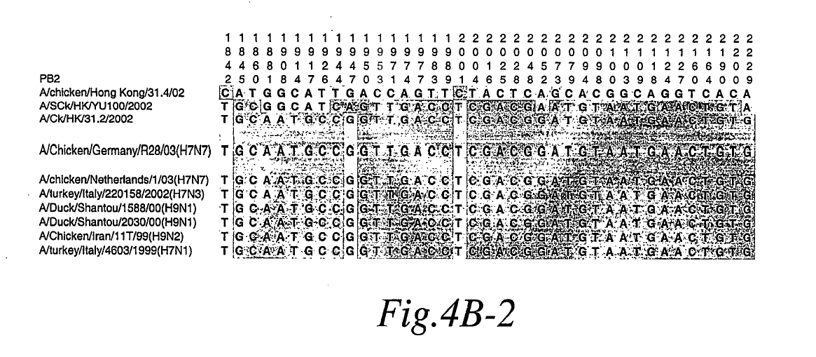

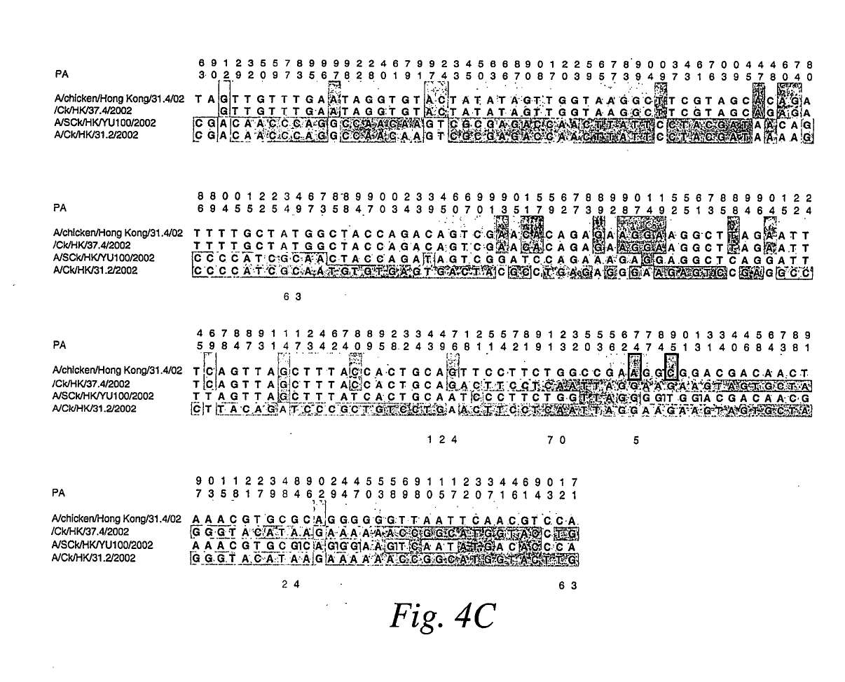

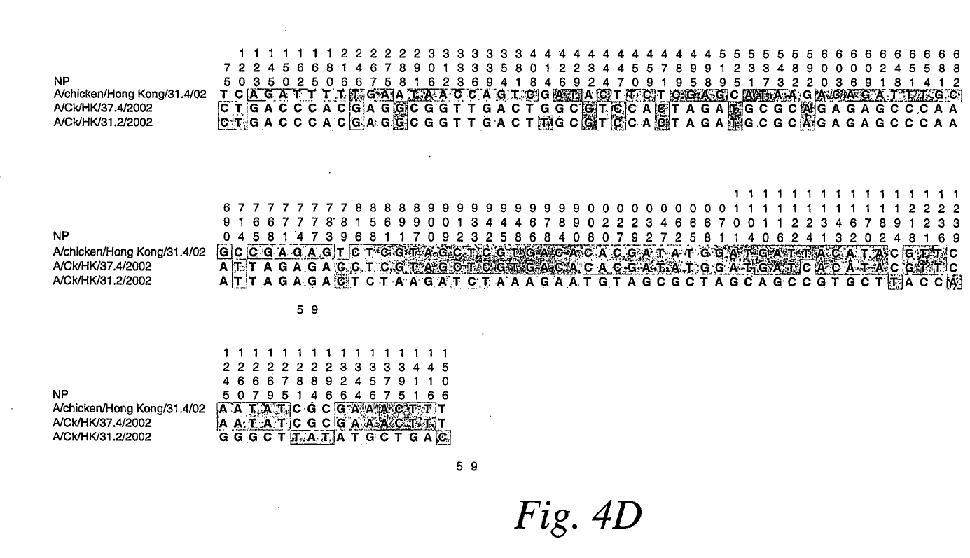

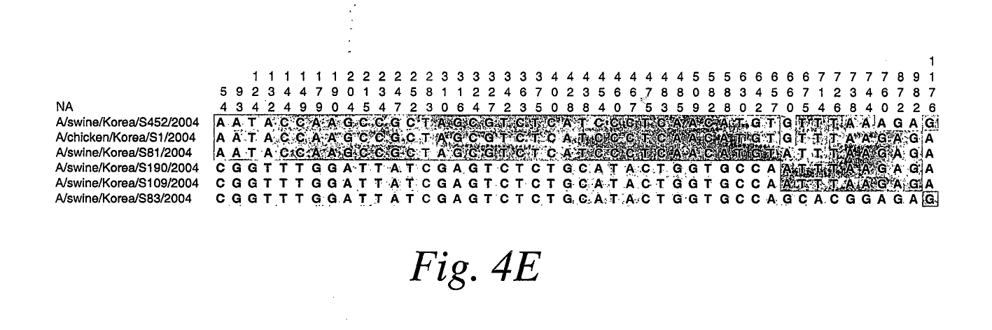

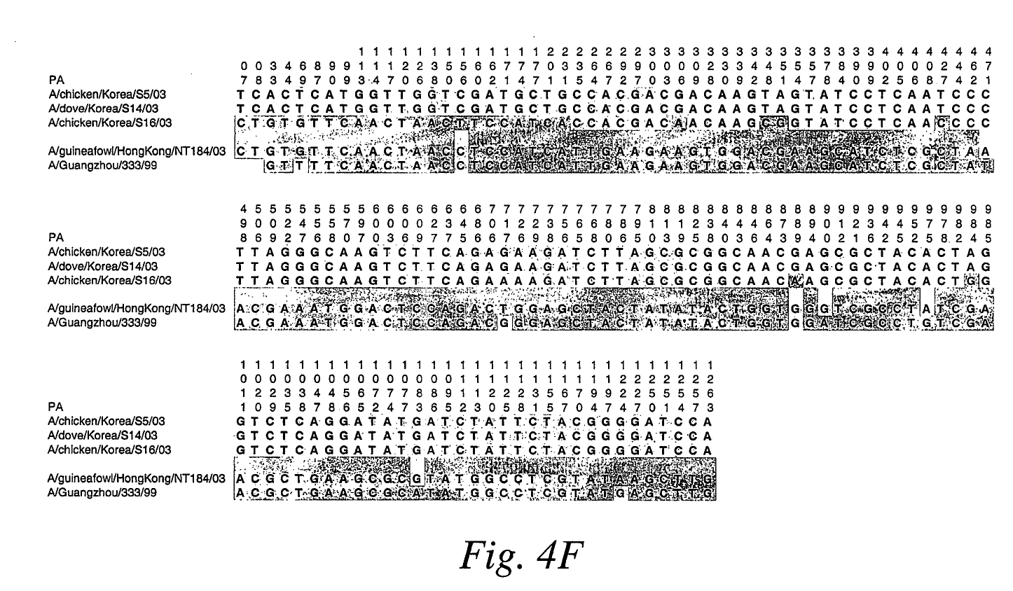

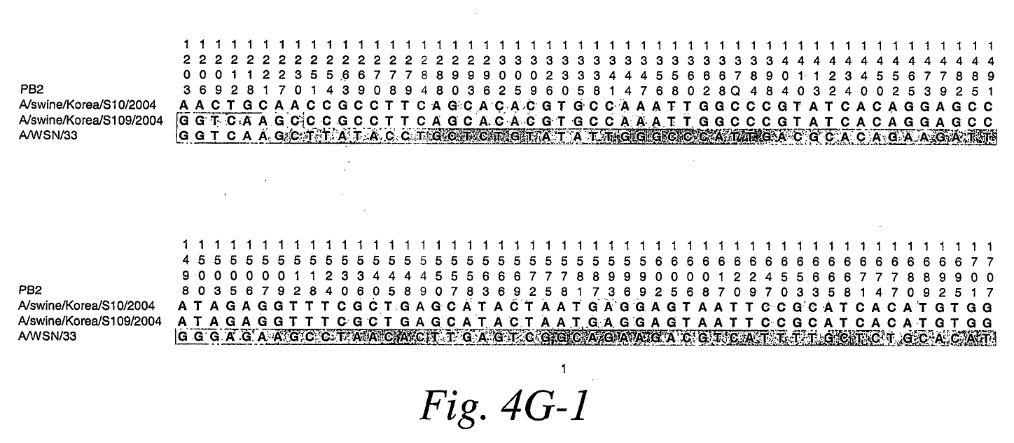

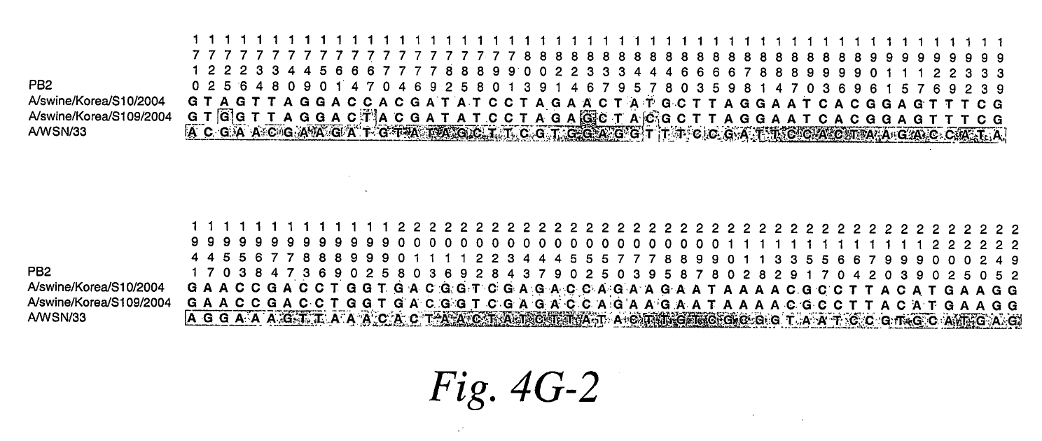

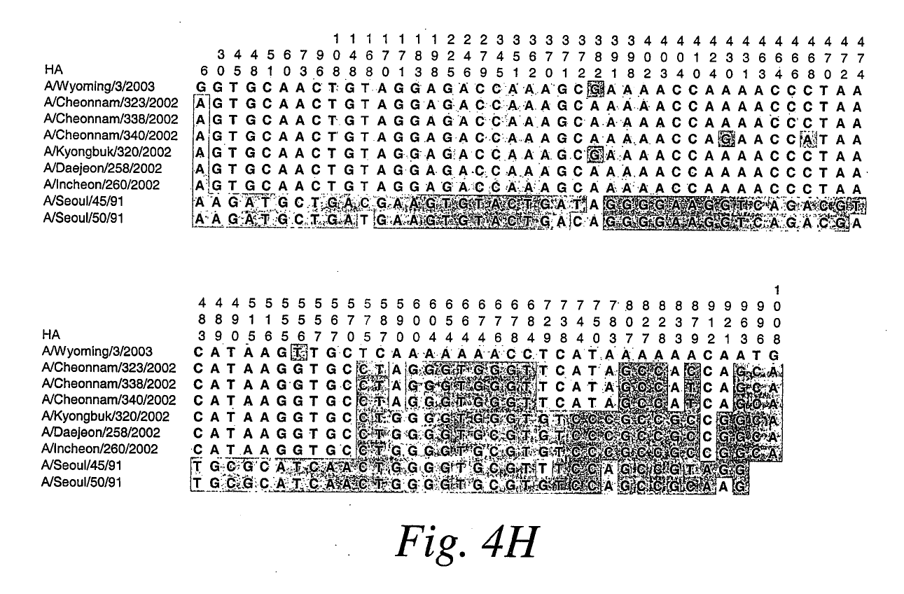

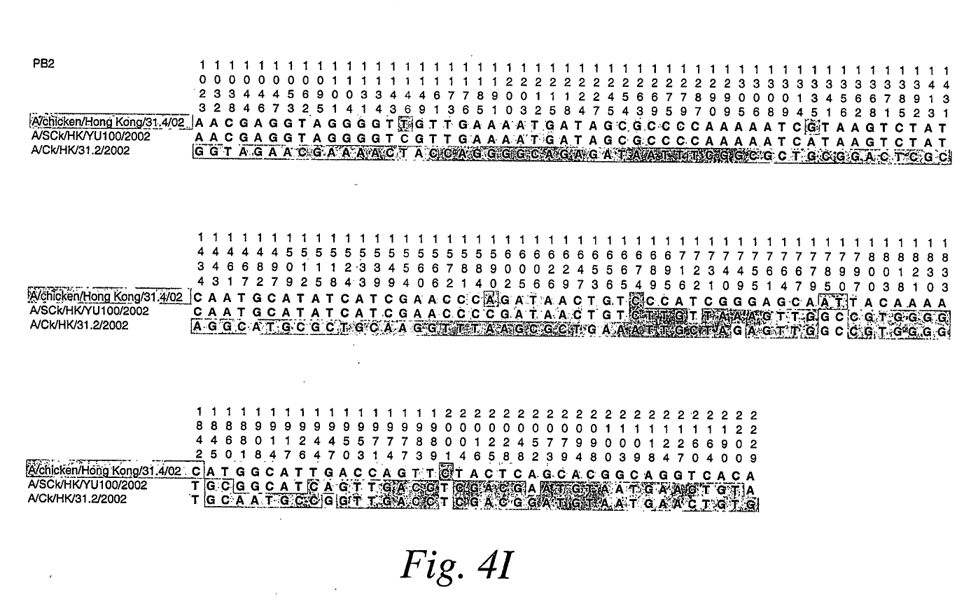

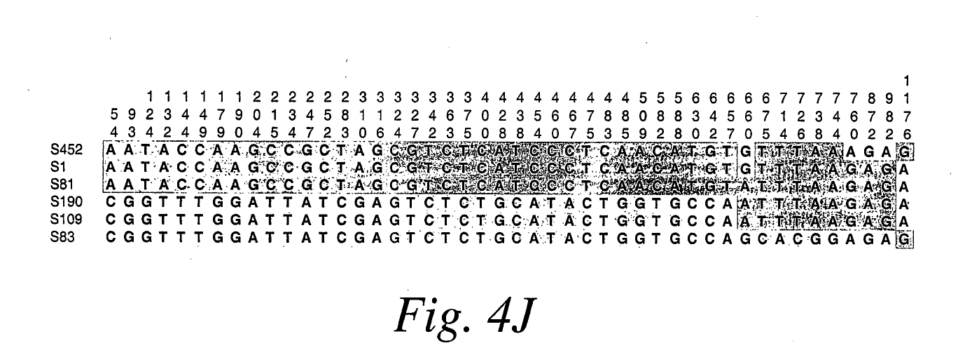

FIG. 3 displays additional examples of recombination. In the first four panels (FIGS. 3A-3D), polymorphisms for the four genes for the replication complex are listed. These bimorphisms were defined by two isolates from the same chicken in Hong Kong, 31.2 and 31.4. The large number of polymorphisms is due to the complementary relationship between 31.2 and 31.4. 31.2 has recombined and is closely related to the pandemic strain. In contrast, the corresponding opposite purine or pyrimidine is found in the 31.4 sequence. These two complementary sequences act as parents of additional recombinants found in Hong Kong live markets, as displayed in the first four panels.

The number of bimorphisms was high for each of the four genes and the two parental genes were homologous or non-homologous to the pandemic genes. For PB1 (FIG. 3A) there were 181 bimorphisms. The recombinant 37.4 was virtually identical to 31.2 through position 1418. The remainder of the available sequence (through position 1890) matched the pandemic strain. Included in panel A is an H5N1 isolate from Fujian province, which is homologous to the 31.2 sequence. The Fujian sequence is complete and was used to define the polymorphisms in the position of the sequence absent for 31.2. The Fujian sequence is one of many sequences outside of Hong Kong that is homologous to 31.2.

For PB2 (FIG. 3B), only a partial sequence was available, but in the 3′ half of the gene beginning at position 1023, there were 144 bimorphisms. For this gene, YU100 is the recombinant and it was virtually identical to the pandemic gene through position 1665 and then matched 31.4 for the remainder of the gene. For comparison, homologous genes from other serotypes (H7N7, H7N3, H9N1, H9N2) from around the world were aligned, and demonstrated that the sequences with the alternate purine or pyrimidine were widespread and not limited to a novel gene found in Hong Kong.

For PA (FIG. 3C), there were 191 bimorphisms and both YU100 and 37.4 were recombinants on this gene. YU100 matched 31.4 through position 1449 and then switched to the 31.2 sequence. In contrast, YU100 matched 31.2 through position 873 and then switched to the 31.4 sequence.

For NP (FIG. 3D), the relationship between the two Hong Kong parental strains had switched. 31.2 was highly homologous to the pandemic strain, to which 31.4 was distantly related. 37.4 was a recombinant in NP, sharing bimorphisms with 31.2 through position 789 and then matching 31.4 for the 3′ half of the gene.

Thus, FIGS. 3A-D display the two parental sequences found in chicken 31, as well as one or two recombinants that have a single crossover point. The two parental sequences in chicken 31 are common. The pandemic version was found in genotype Z isolates (Guan et al, 2004) throughout Asia and the sequences with the opposite purine or pyrimidine were found in H5N1 isolates throughout Asia, as well as other serotypes throughout the world. Thus, recombination produced two genes, which differed significantly at the nucleotide level, but were highly homologous at the protein level.

Recombination was not limited to the internal genes. Evidence of recombination in NA is shown in FIG. 3E. These sequences were from H9N2 isolates in Korea. In the 5′ half of the gene, two swine (S452 and S81) shared sequences with a chicken sequence (S1), while the remaining 3 swine sequences (S83, S109, S190) formed the opposite bimorphic sequence. However, at position 660, two of these swine sequences switched to the alternate sequence.

A Korean avian isolate (S16) was a recombinant in PA. The sequence for the first 231 bp was virtually identical to two H9N2 isolates from Hong Kong/Guangzhou. There was only one bp difference with a 2003 avian isolate, and only 2 bp differences with a human H9N2 isolate from 1999. These homologies suggested dual infections between the Korean avian isolate and isolates from Hong Kong. This association was increased by the sequence of the M gene in S16. It was an exact match with a H9N2 1998 swine sequence (10) or differed by a single bp with a second 1998 swine isolate (9).

Recombination could also be found in human flu genes. Two Korean swine isolates displayed PB2 recombinants between the human lab strain WSN/33 and the 2004 Korean swine isolates. FIG. 3G shows the match at the 5′ end of the Genbank sequence of S109 with WSN/33. This match extended to the 5′ end of the gene (Sang Seo, personal communication) and a similar match was found between WSN/33 and S81 (Sang Seo, personal communication).

Human/human recombination is demonstrated in FIG. 3H. The 5′ half of HA in 2002 Korean isolates matched current H3N2 isolates, represented by the Wyoming vaccine sequence. These Korean isolates matched Korean isolates from 1991 at the 3′ portion of the gene. Sequences closely related to the 1991 Seoul sequences were identified worldwide in the late 1980's, but were absent from HA sequences at Genbank from 1991 to 2002, only to re-emerge as recombinants.

Discussion

Influenza evolution has been described as a series of drifts and shifts. Drifting was thought to be driven by point mutations generated by an error prone polymerase, while shifting was linked to the reassortment of the 8 sub-genomic influenza RNAs. However, the present invention shows drifts and shifts occur by recombination, and has provided a mechanism for the genetic diversity seen in viruses and other gene systems, and in particular influenza.

Reassortment between influenza subtype H5N1 and other subtypes has been observed previously. The H5N1 1997 isolates from patients in Hong Kong were reassortants (Guan et al 1999). Internal genes were closely related to genes found in H9N2 and H6N1 isolates. However, this constellation of genes was not seen after H5N1 culling in Hong Kong in 1997. H5N1 was again isolated from humans in Hong Kong in 2003 and these isolates had a very different constellation that was called the Z genotype (Guan et al 2004; Chen et al 2004). Later that year, a related constellation was designated Z+ and this group was found throughout Asia by the beginning of 2004. However, these isolates had regional specific polymorphisms and isolates from patients in Vietnam and Thailand contained polymorphisms unique to those two countries or uniquely shared between those two countries. These polymorphisms will be described elsewhere, but in general the polymorphisms were not found in other H5N1 isolates, but serotypes commonly found in mammals. The biological differences between the Z+ genotypes were due to the polymorphisms, not reassortment. Similarly, the differences between isolates from mouse brains and the parent virus were due to polymorphism, and not reassortment. Some of the differences were clearly generated by short stretches of recombination.

Although other polymorphisms looked like point mutations, the polymorphisms were not due to recent mutations. They could be found in mammalian serotypes. Similarly, many of the polymorphisms found in the H5N1 pandemic strain could be found in previously-circulating H5N1 isolates. However, as shown herein, these polymorphisms merged via recombination, frequently involving co-circulating haplotypes. These polymorphisms were largely bimorphisms, which were merged via recombination. This process created two viruses simultaneously, which differed in third base codon positions. Since most of these differences were transitions, most of the protein changes were synonymous because transitions in the third base position of 60 of the 64 codons create a synonymous change.

In Hong Kong in 2001 there were two major closely related genotypes circulating. In genes that had not recently recombined, the new recombined bimorphisms looked like point mutations. Paired samples demonstrated short segments of recombination. Some of the genes had already recombined which led to one haplotype with a large number of bimorphisms that matched the pandemic strain and an opposite version that had a large number of bimorphisms that did not match. Further recombination between these two dissimilar haplotypes created new genes that were chimeras with one end of the gene from one parent and the other half from another.

In one mechanism, a larger number of crossover events happen in a dual infection in one host; in another mechanism, the multiple crossover events accumulate via a series of dual infections. Some of the recombinants were generated by a single cross over near the center of the gene, while others involved a short region of a few hundred bp. The number of reassortments and recombinations identified in subsequent isolates is much less than the theoretical number that could be generated via a dual infection.

Recombination is not limited to H5N1 or avian genes. Human genes evolve using the same mechanism and in the Korean swine sequences, genes that were half human and half avian were found. In the example given above, the recombination was with viruses that had been widespread in the late 80's and early 90's, but had disappeared from sequences at GenBank for 10 years. Thus, the sequences can be quite stable and reappear within the populations at a later date. These data show that in the absence of recombination, the fidelity of replication is exceedingly high. This conservation of sequence identity can also be found in highly evolving environments. In a 2003 Korean isolate, there was evidence of dual infections with isolates from Hong Kong, a region that has produced rapid genetic change. However, exact copies of the M gene from a 1998 swine isolate in Hong Kong could be found in a Korean avian isolate five years later. These data indicated that the polymerase is not error-prone, or errors are corrected. The diversity seen every year in avian or human isolates is not due to recent mutations. Instead, the changes are clustered bimorphisms, which are distributed via recombination.

The evolution of virus via recombination is stable. The recombinations seen in 2001 strains in Hong Kong are still present in the 2004 pandemic strains. Hong Kong isolates provided a window on the recombination process, but the same types of recombination were seen elsewhere, including isolates that had recombined genes at an earlier date. Comparison of human H1N1 flu isolated in the 1930's, such as WSN/33, with classical swine H1N1 also isolated in the 1930's, identified complementary bimorphisms found in the 1918 pandemic strain. These relationships will be described elsewhere.

These data have several practical applications. Since the newly-formed recombinants were generated by crossovers between the parental genes, new recombinants could be predicted based on the prevalent genomes in the area. The recombinants align between the two dominant genotypes and complementary versions of the recombinants can be generated. The bimorphisms can be used to determine the origins of the recombinants and can also be used to predict the sequence of the complementary version, since the changes are largely transitions. Therefore, one version predicts the sequence of the complementary version. These rules can be used to identify both future and past haplotypes, which have applications in vaccine development.

The applications of the rules described herein extend well beyond influenza evolution. Viral sequences at GenBank display high rates of transitions at the codon third base position. The other viruses also use bimorphisms for rapid evolution and complementary versions of viruses are abundant. These complementary relationships can be seen for coronaviruses including SARS (Mara et al 2003; Rota et al 2003), NL63 (va der Hoek et al 2004) and HKU-1 (Woo et al 2005). These relationships will be described elsewhere.

These relationships can also be found in other systems and can explain the gene repair of higher organisms such as that recently described for Arabidopsis HTH genes. Eleven polymorphisms for the Arabidopsis HTH genes were characterized (Lolle et al 2004), and all eleven were transitions. However, these transitions were selected from mutagenized stocks, so they were not third base transitions and the changes were non-synonymous (Krolikowski et al 2003). However, like influenza, the changes were template-driven and specific. They were found at higher frequencies and under various conditions (Lolle et al 2004). Thus recombination in double-stranded RNA versions of genes can explain the non-mendelian inheritance observed after self fertilization in F3 progeny of Arbidopsis.

The high frequency recombination in the viral genomes is driven by dual infections. However, dual infections can also play a role in more dramatic evolution involving unrelated genomes. An 18 bp region of H5N1 HA can be found in the Ebola spike gene (HLN in preparation). This particular region contains regional specific bimorphisms in both genomes. Thus, the isolates from Vietnam and Thailand have a specific bimorphism that is not found in earlier H5N1 isolates. Moreover, a second polymorphism generates the HA sequence found in the 1918 pandemic HA. Thus, the high frequency of homologous recombination seen in hosts infected by the same class of virus also extends to viruses of different classes leading to sharing of sequences which can be linked to similar clinical manifestation such as excessive hemorrhaging seen in humans or animals infected with Ebola, H5N1, or H1N1. Details of these relationships will be presented elsewhere.

Recombination is a strong driver of rapid evolution. In influenza, recombination can produce both drifts and shifts and the same mechanisms have been adopted universally for rapid evolutionary change.

### Example 2

### Copy-Choice Recombination Between Distinct Viruses

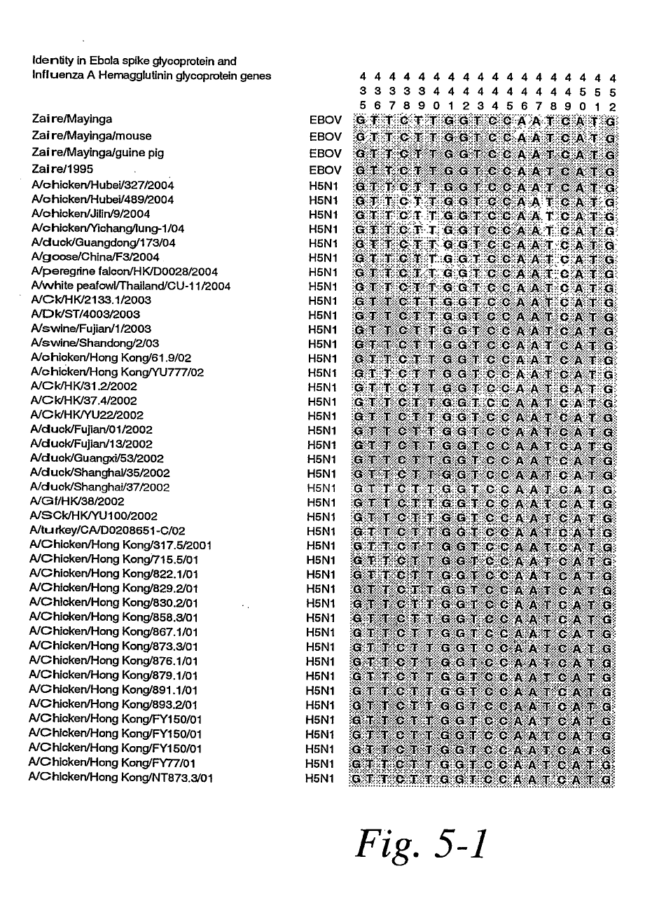

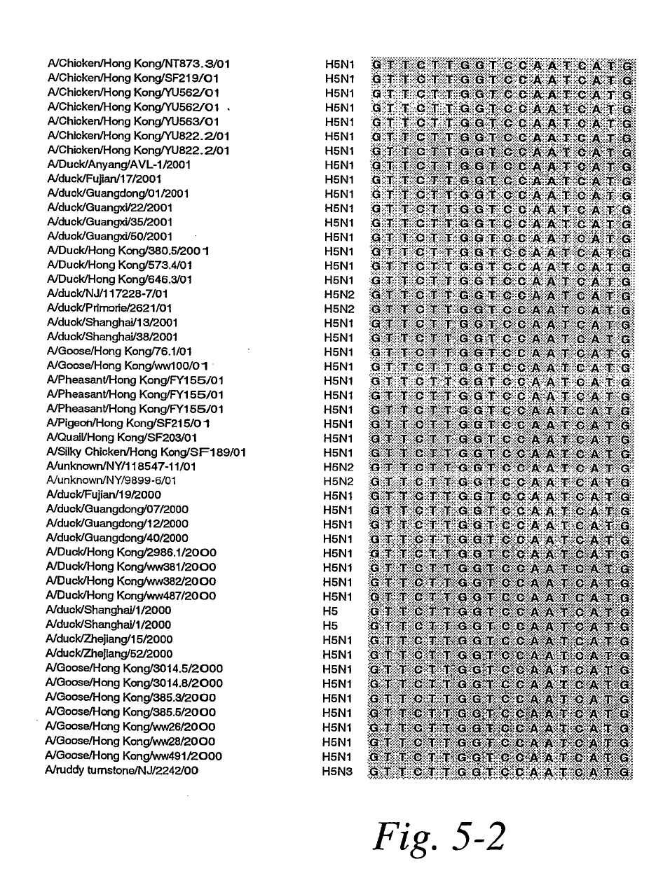

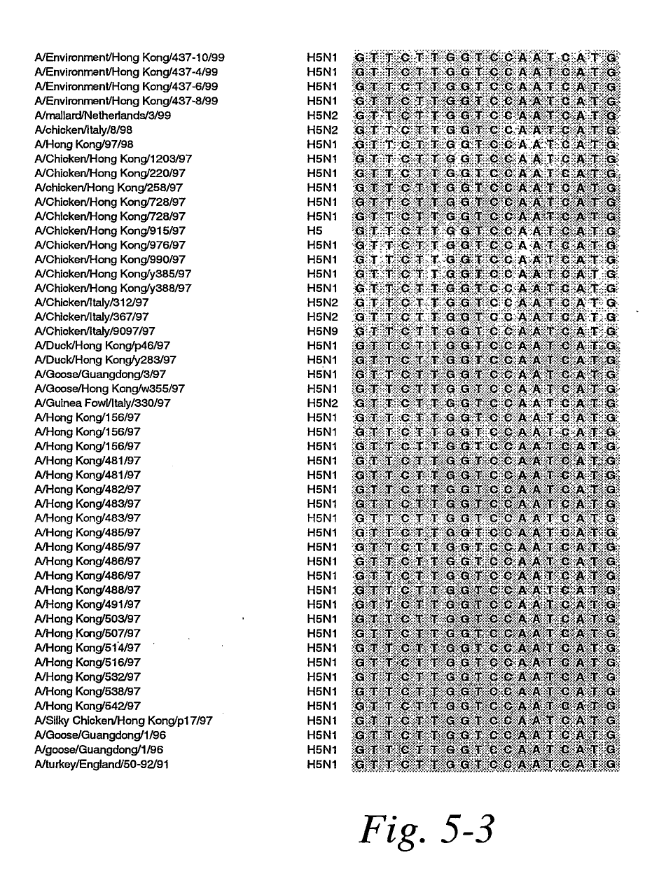

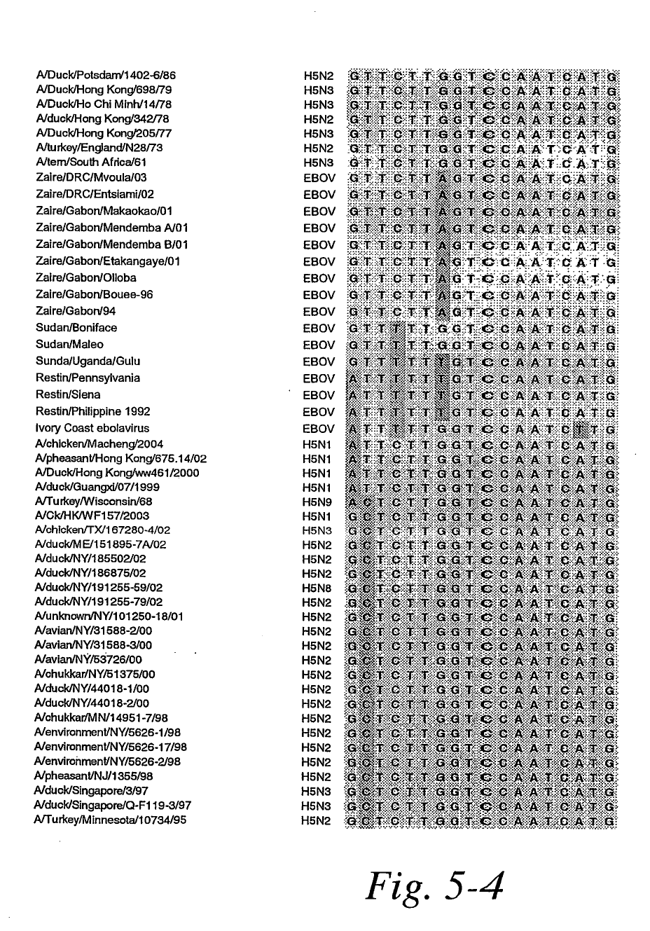

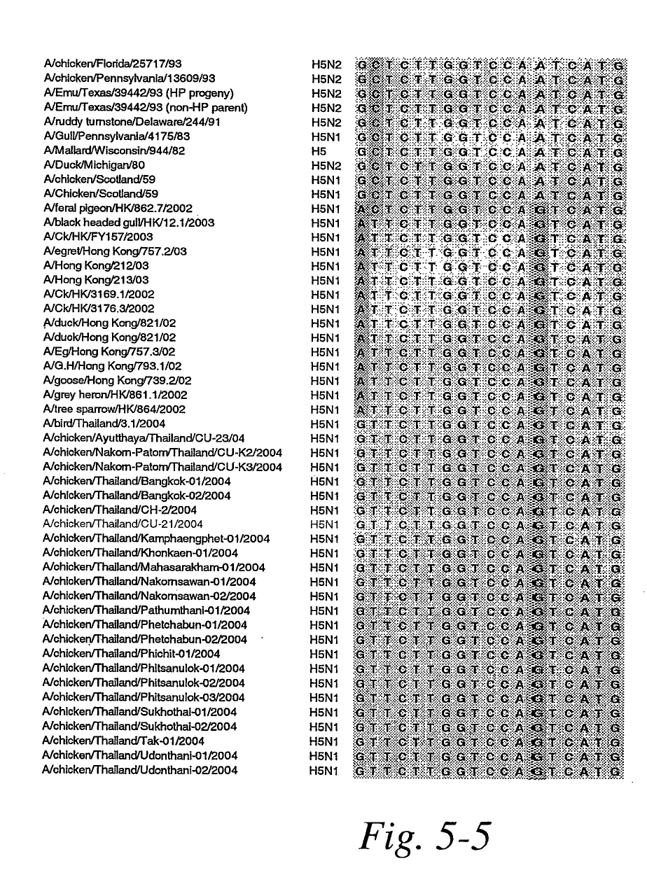

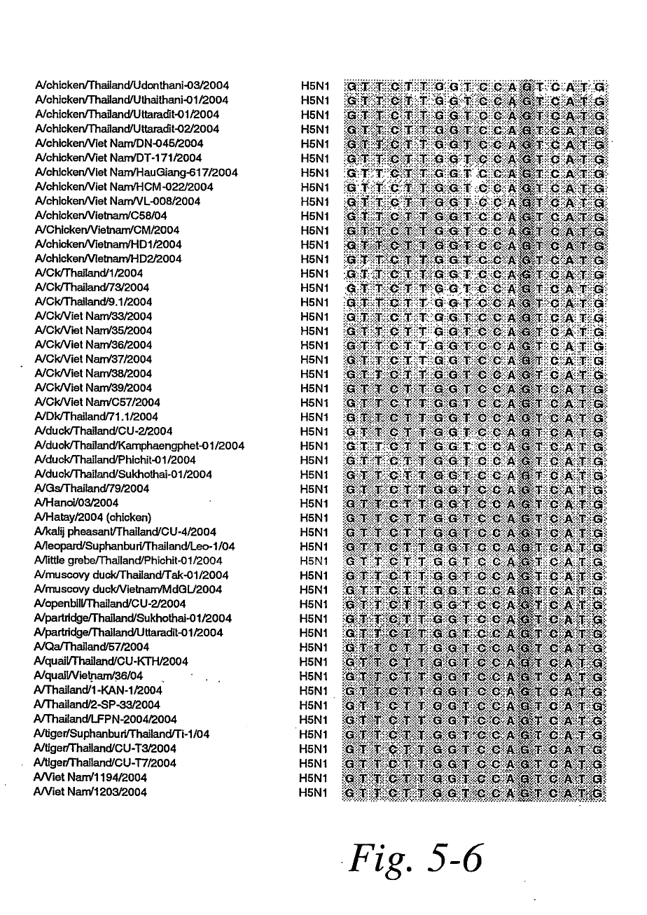

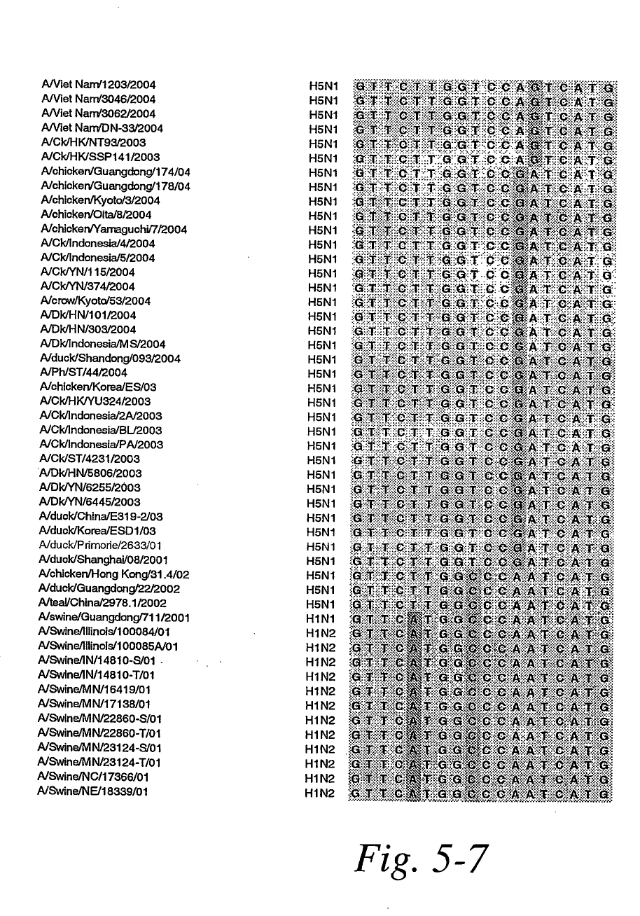

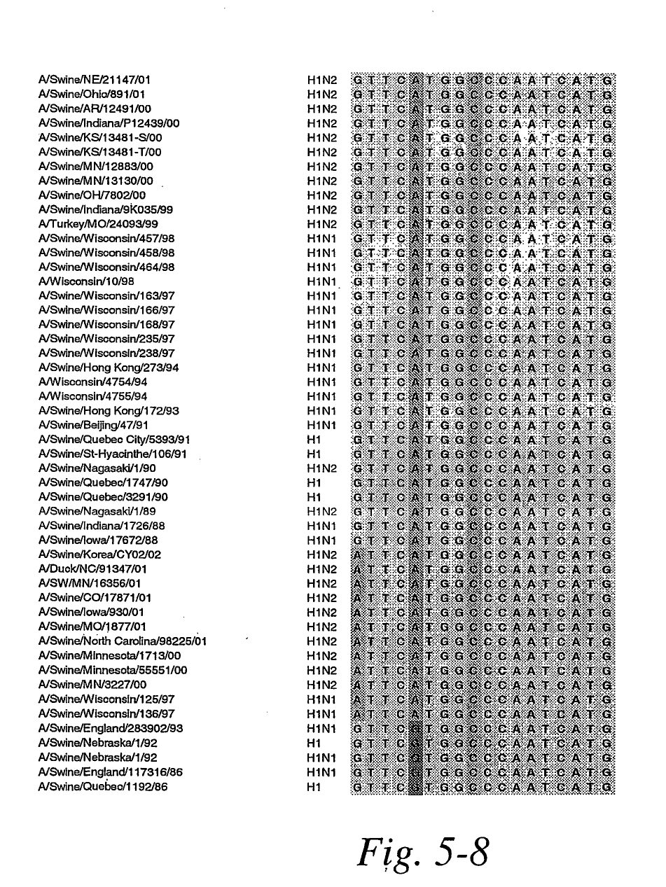

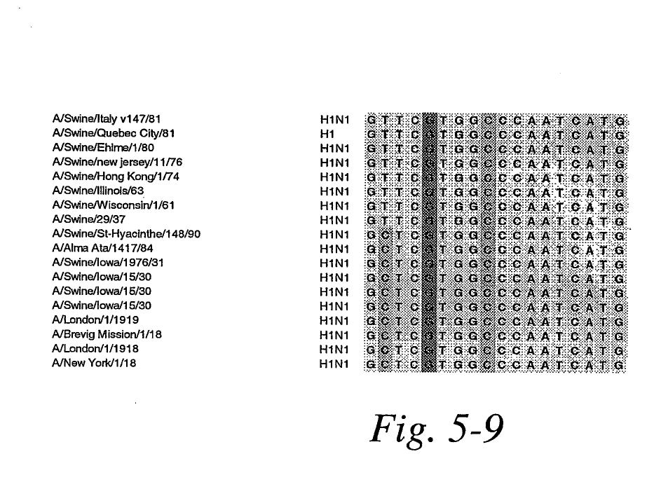

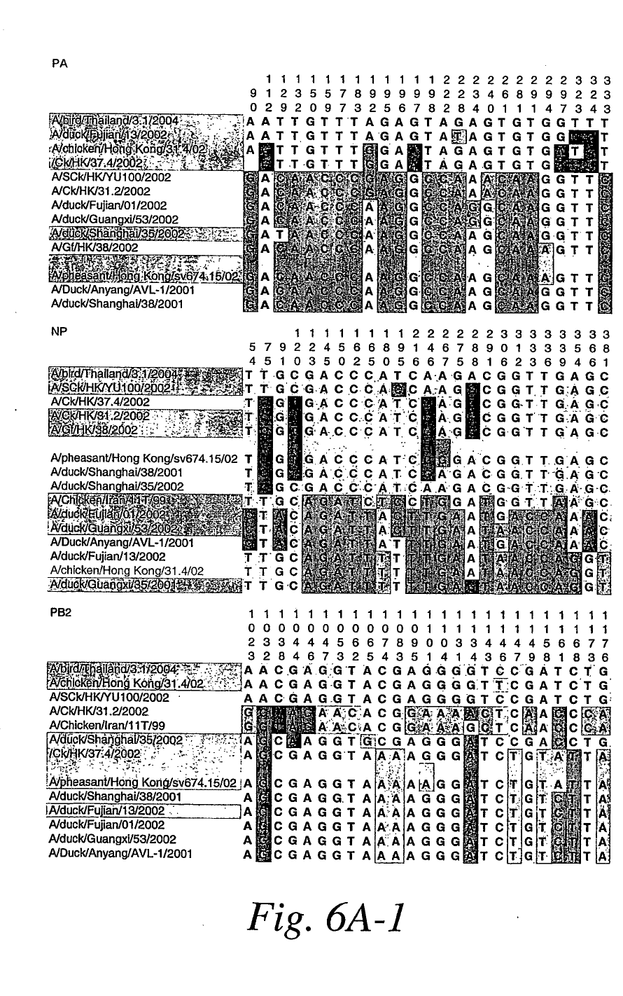

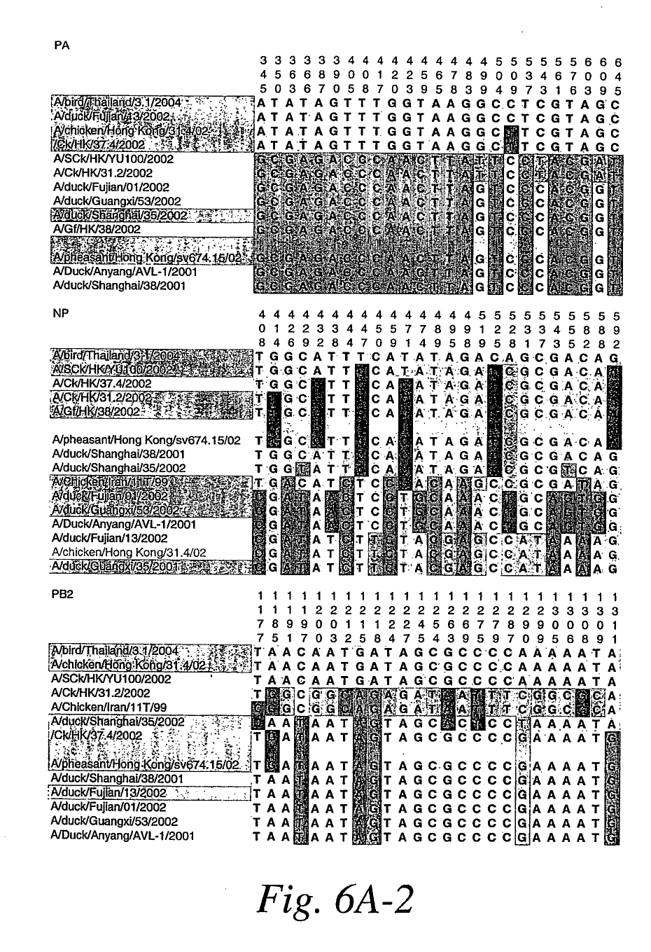

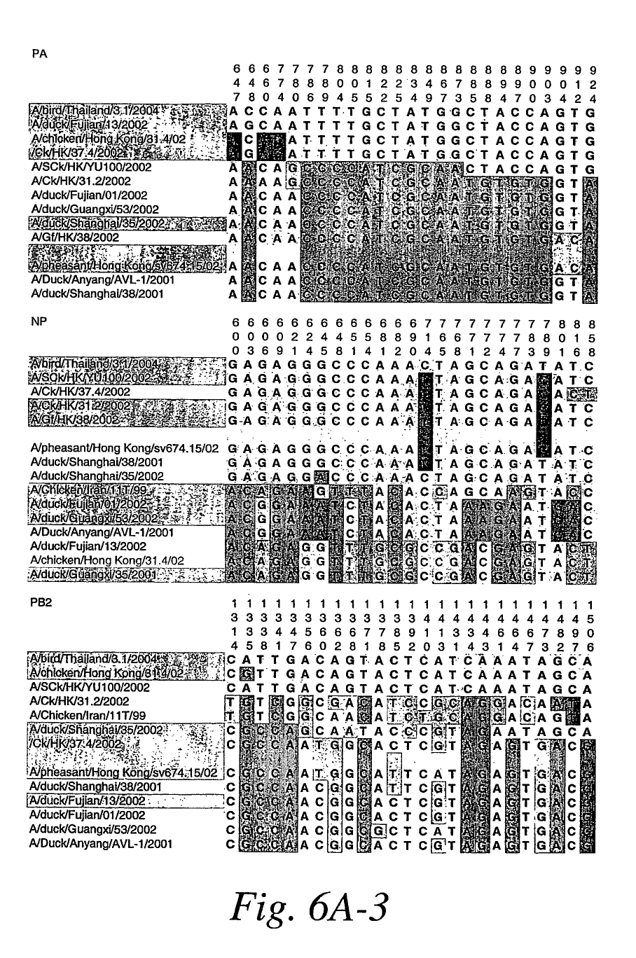

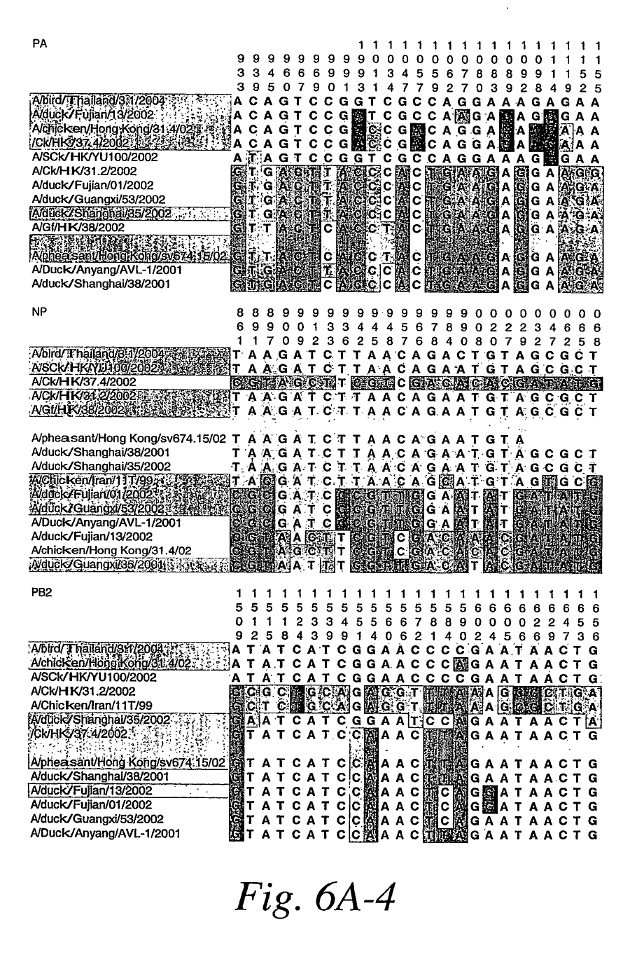

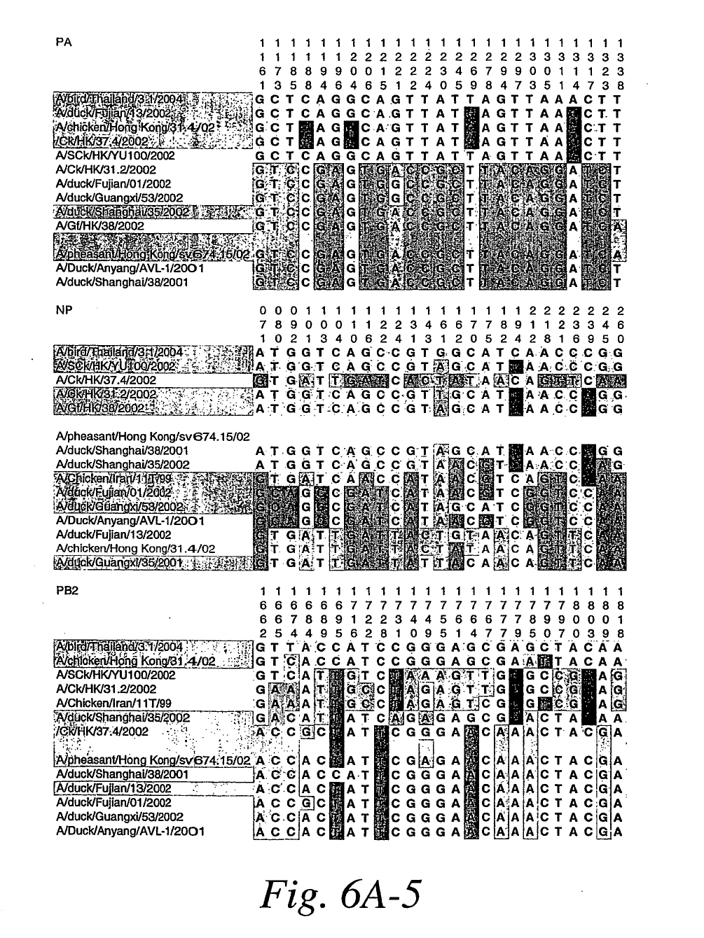

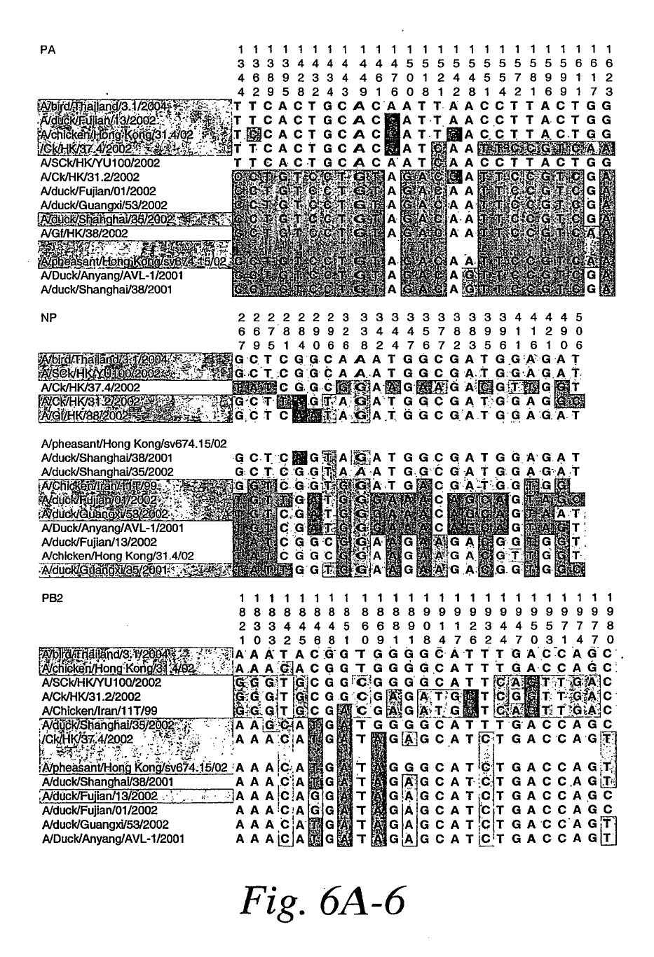

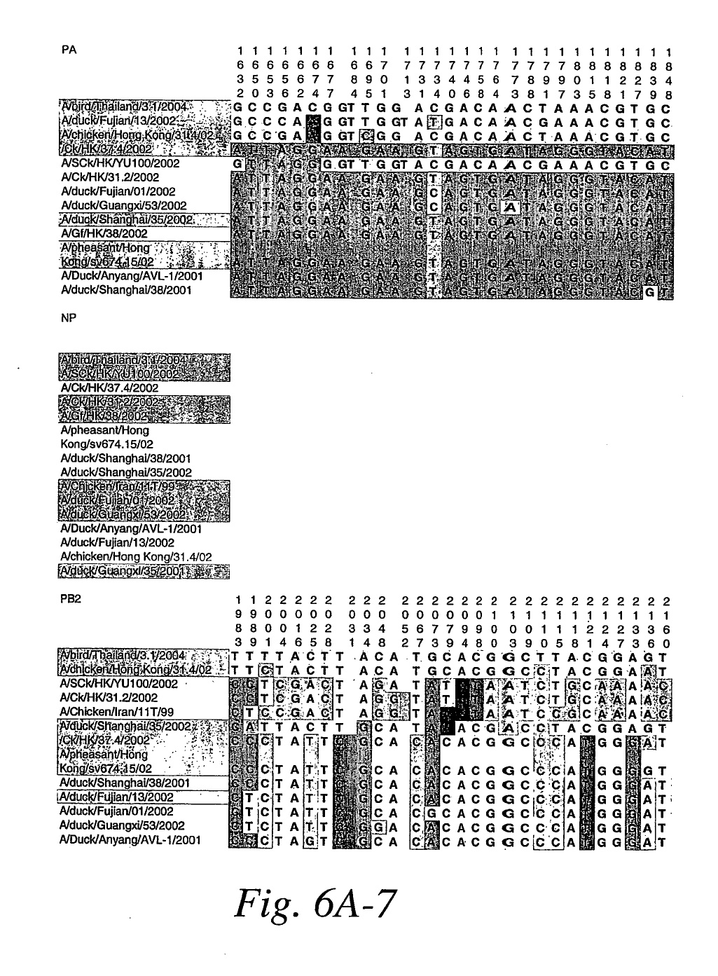

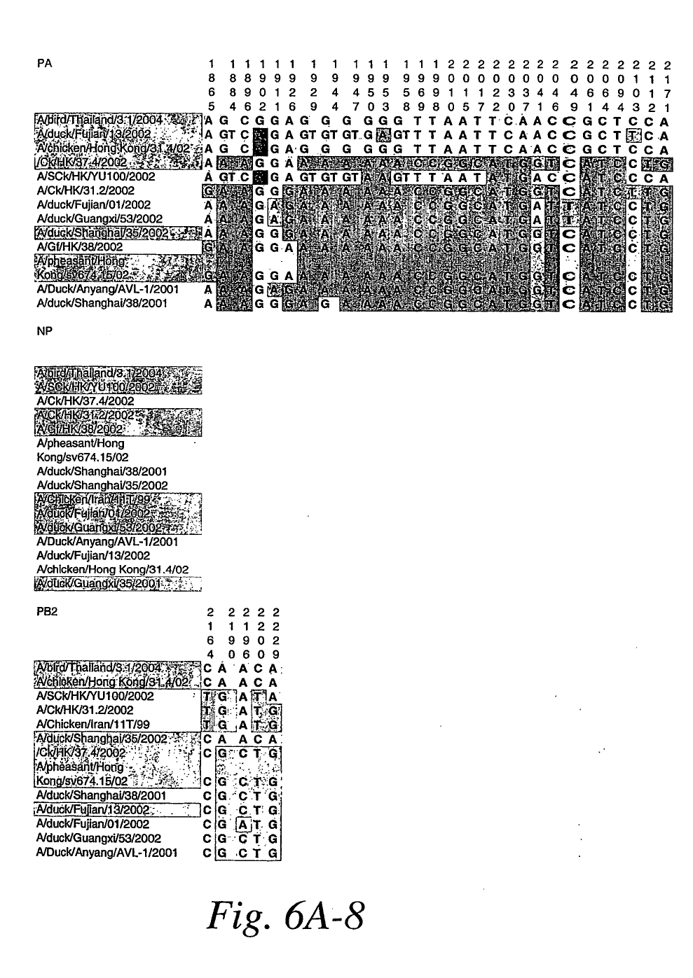

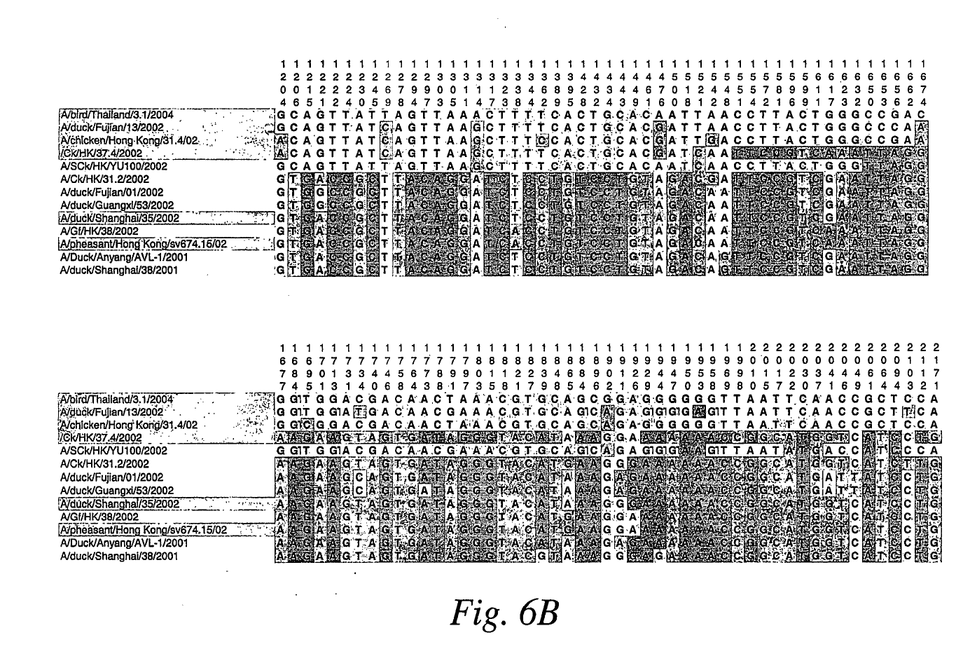

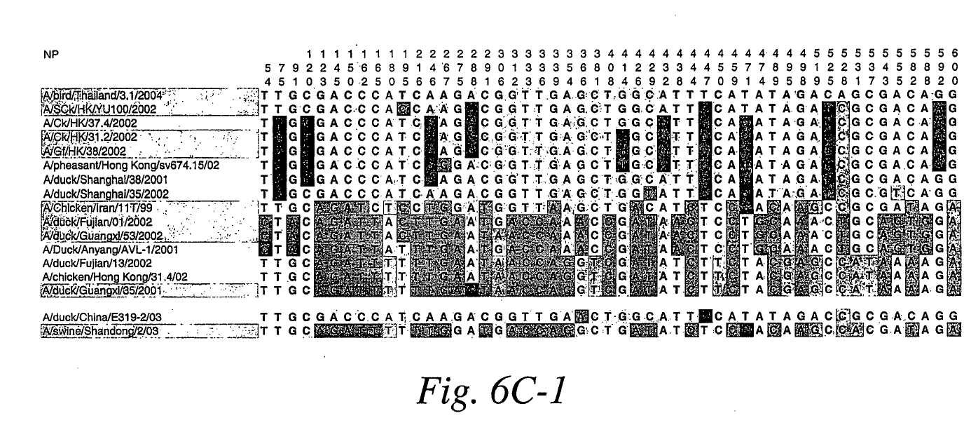

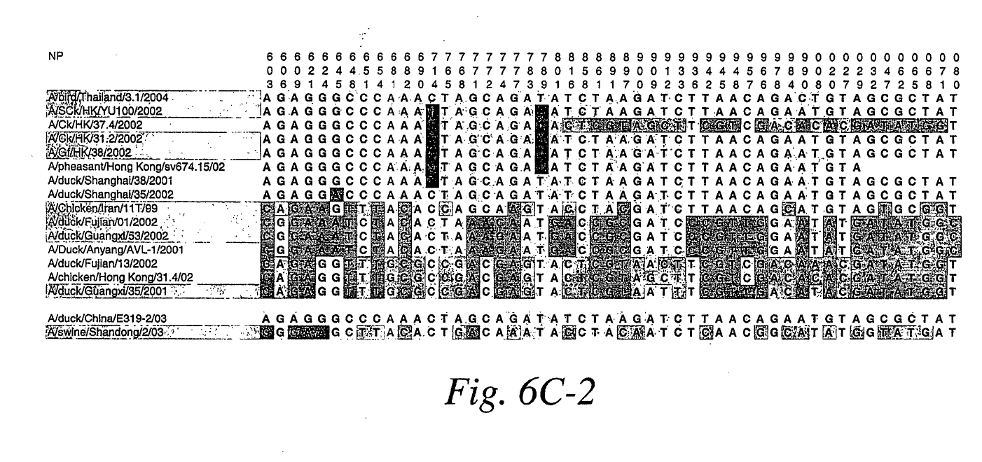

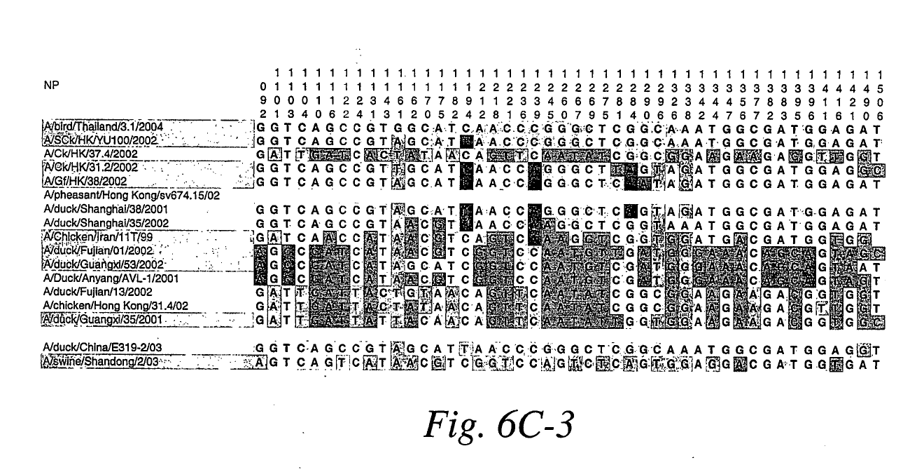

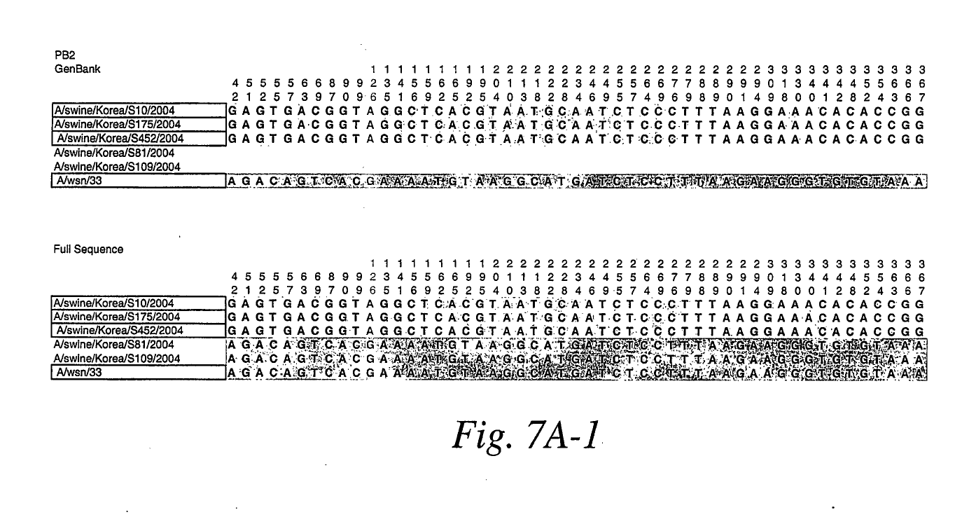

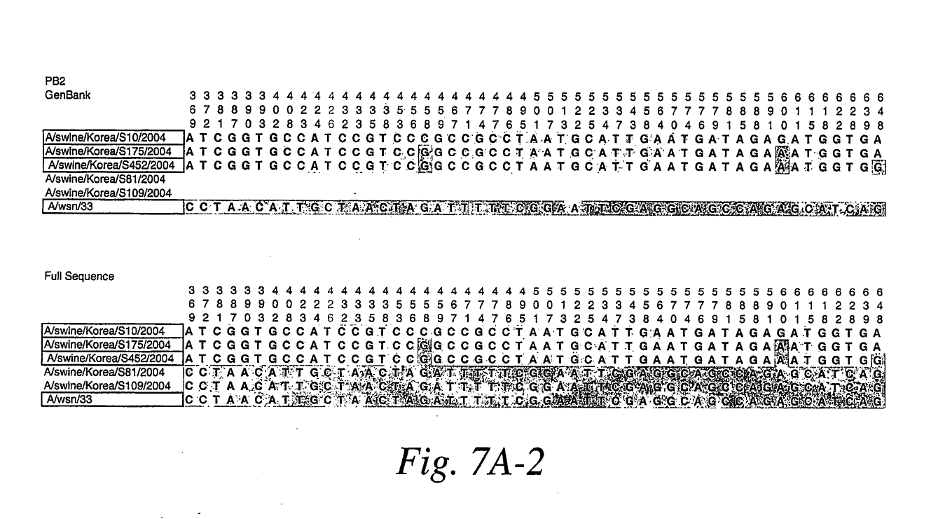

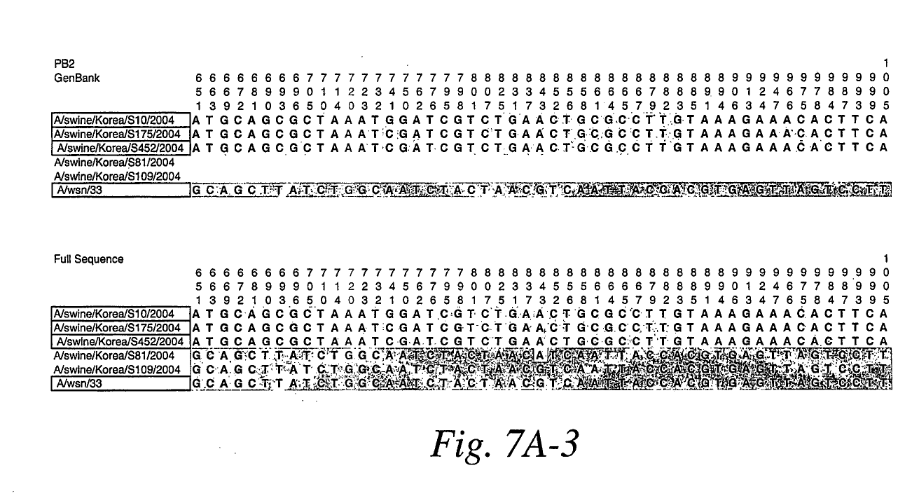

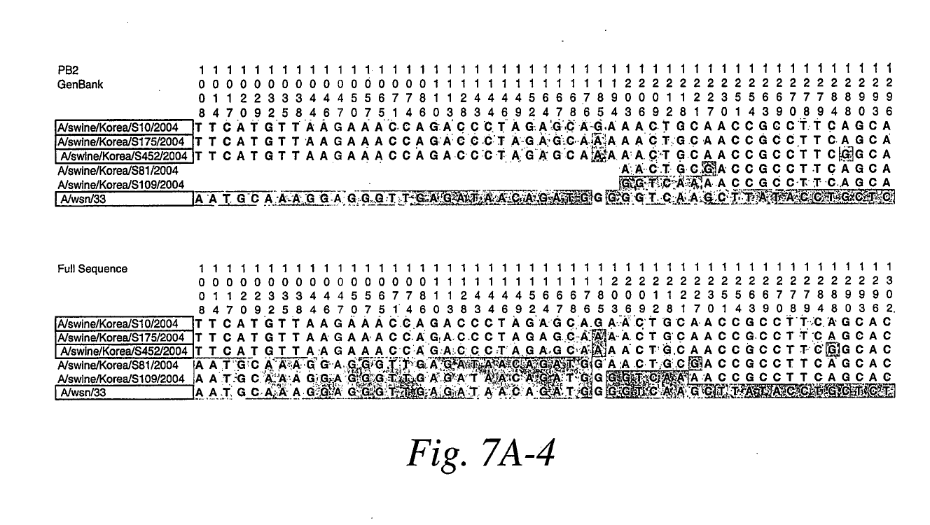

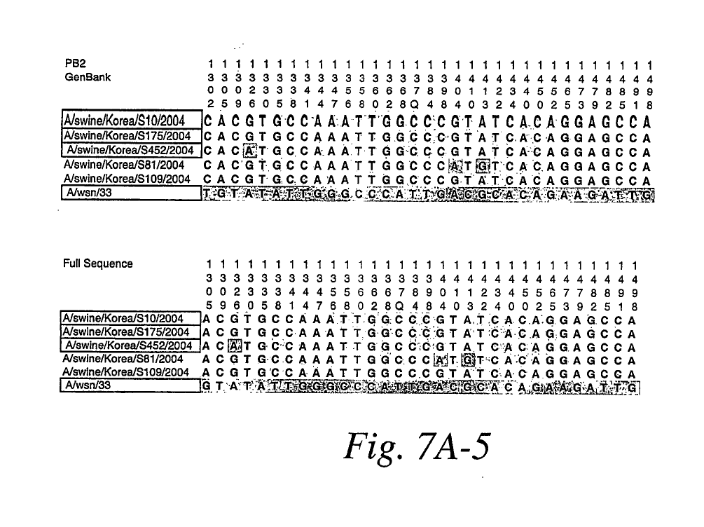

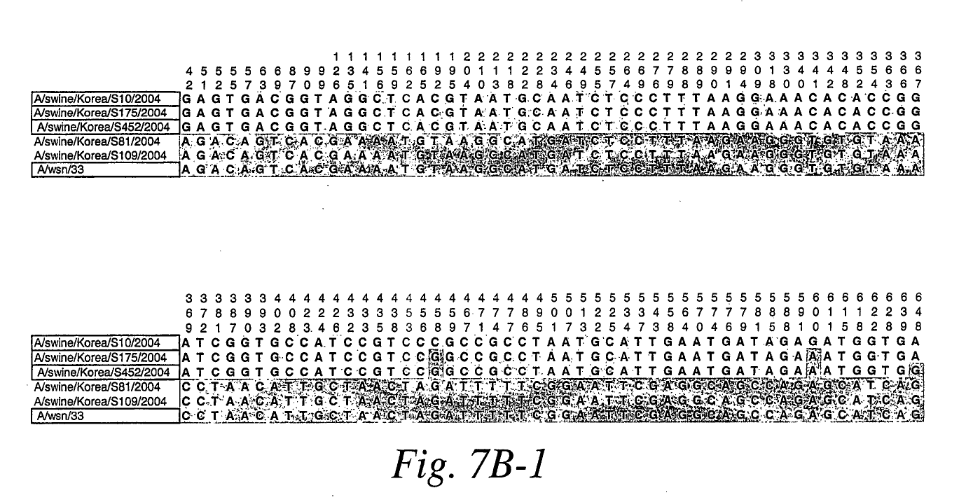

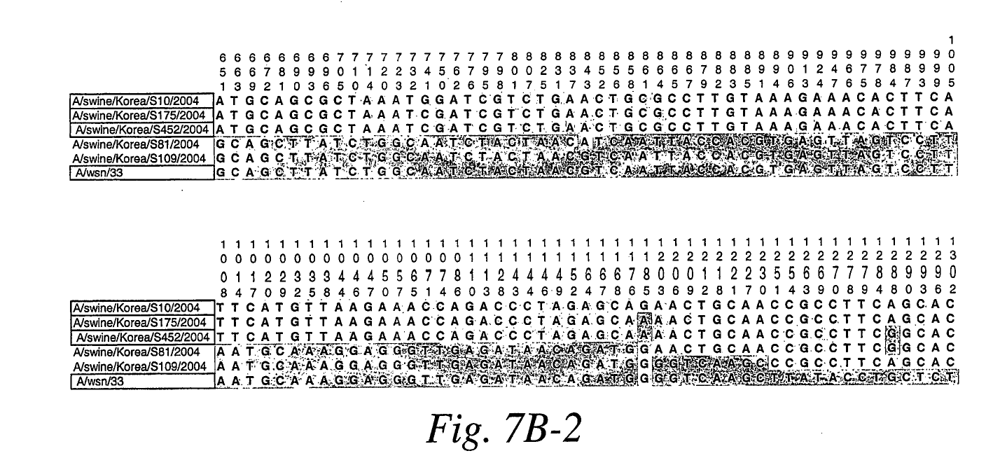

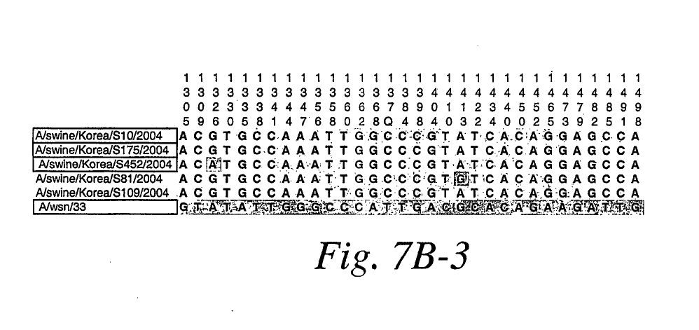

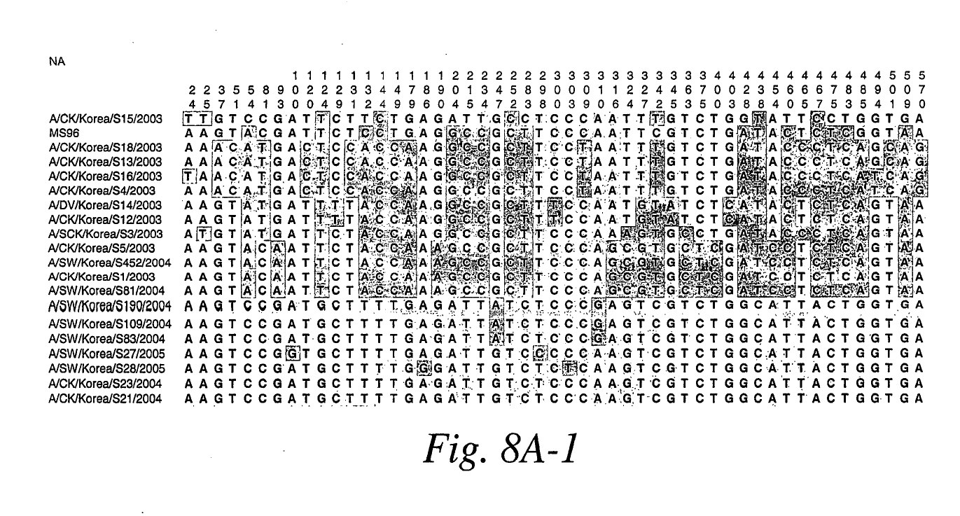

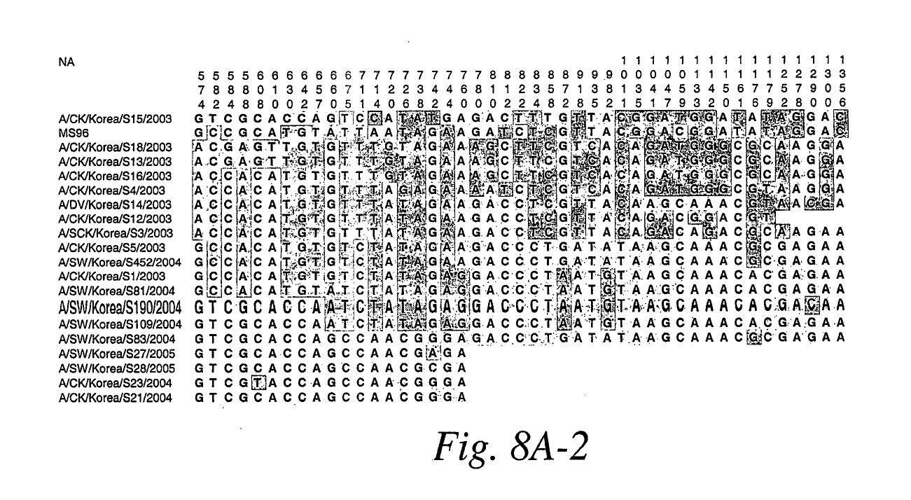

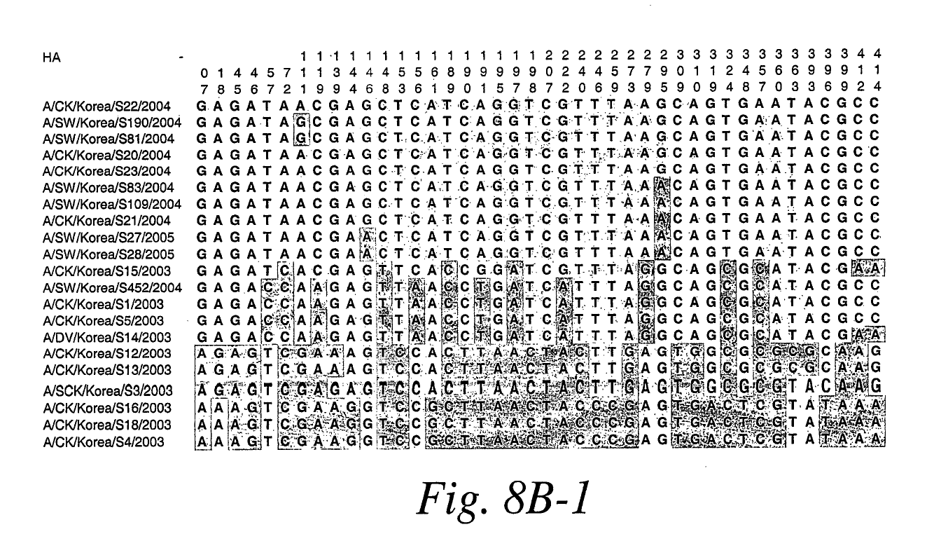

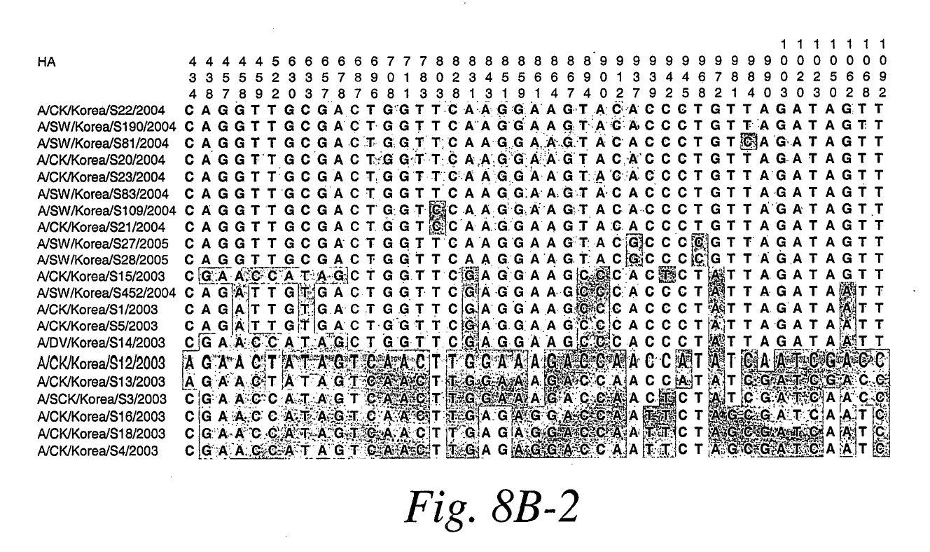

The signature of copy-choice recombination can be observed in the mutant progeny of distinct types of parental viruses. In FIG. 5, a tract of 18 nucleotides in length has been conserved between Ebola in Africa and the 1918 flu pandemic, connected through intermediate mutant progeny strains of H5 influenza strains. Copy-choice recombination, when combined with selective pressure, can therefore act to conserve blocks of sequence between distinct parental viruses. The conserved block of 18 nucleotides shown in FIG. 5 likely encodes a small RNA, e.g., a miRNA, possessing functional activity. Similar recombination of sequences from distinct viruses has been observed for SARS, IBV and astroviruses (where a conserved 3′ stem loop structure is shared); foot and mouth disease and Newcastle disesase; and HIV and coronavirus.

### Example 3

### Copy-Choice Recombination Applied to Bacteria

The ability to predict the composition of mutant progeny sequences of bacteria that are likely to arise from combination of parental bacterial sequences can be used to enhance prediction and identification of mutant progeny bacteria(e) that may possess a given phenotypic trait (e.g., drug resistance). Two parental bacterial sequences can be combined in vitro, in vivo, or in silico, with the rules of the present invention allowing for enhanced prediction of which mutant progeny bacteria(e) will exhibit a monitored trait. The present invention can therefore be applied, e.g., to drug screening approaches.

### REFERENCES

J S Peiris, W C Yu, C W Leung, C Y Cheung, W F Ng, J M Nicholls, T K Ng, K H Chan, S T Lai, W L Lim, K Y Yuen, and Y Guan. Re-emergence of fatal human influenza A subtype H5N1 disease. Lancet, February 2004; 363(9409): 617-9.
Ron Fouchier¹, Thijs Kuiken¹, Guus Rimmelzwaan¹ and Albert Osterhaus¹ Global task force for influenza. Nature 435, 419-420 (26 May 2005)|doi: 10.1038/435419a.
Michael T. Osterholm. Preparing for the Next Pandemic. N. Engl. J. Med., May 2005; 352: 1839-1842.
Y. Guan, J. S. M. Peiris, A. S. Lipatov, T. M. Ellis, K. C. Dyrting, S. Krauss, L. J. Zhang, R. G. Webster, and K. F. Shortridge. Emergence of multiple genotypes of H5N1 avian influenza viruses in Hong Kong SAR. PNAS, June 2002; 99: 8950-8955.
Tran Tinh Hien, Nguyen Thanh Liem, Nguyen Thi Dung, Luong Thi San, Pham Phuong Mai, Nguyen van Vinh Chau, Pham Thi Suu, Vo Cong Dong, Le Thi Quynh Mai, Ngo Thi Thi, Dao Bach Khoa, Le Phuc Phat, Nguyen Thanh Truong, Hoang Thuy Long, Cao Viet Tung, Le Truong Giang, Nguyen Dac Tho, Le Hong Nga, Nguyen Thi Kim Tien, Le Hoang San, Le Van Tuan, Christiane Dolecek, Tran Tan Thanh, Menno de Jong, Constance Schultsz, Peter Cheng, Wilina Lim, Peter Horby, the World Health Organization International Avian Influenza Investigative Team, and Jeremy Farrar. Avian Influenza A (H5N1) in 10 Patients in Vietnam. N. Engl. J. Med., March 2004; 350: 1179-1188.
US CDC. Cases of Influenza A (H5N1)—Thailand, 2004. JAMA, March 2004; 291: 1059-1060.
Y Guan, M Peiris, K F Kong, K C Dyrting, T M Ellis, T Sit, L J Zhang, and K F Shortridge. H5N1 influenza viruses isolated from geese in Southeastern China: evidence for genetic reassortment and interspecies transmission to ducks. Virology, January 2002; 292(1): 16-23.
Robert G. Webster, Yi Guan, Malik Peiris, David Walker, Scott Krauss, Nan Nan Zhou, Elena A. Govorkova, Trevor M. Ellis, K. C. Dyrting, Thomas Sit, Daniel R. Perez, and Kennedy F. Shortridge. Characterization of H5N1 Influenza Viruses That Continue To Circulate in Geese in Southeastern China. J. Virol., January 2002; 76: 118-126.
Aleksandr S. Lipatov, Scott Krauss, Yi Guan, Malik Peiris, Jerold E. Rehg, Daniel R. Perez, and Robert G. Webster. Neurovirulence in Mice of H5N1 Influenza Virus Genotypes Isolated from Hong Kong Poultry in 2001. J. Virol., March 2003; 77: 3816-3823.
R G Webster, W J Bean, O T Gorman, T M Chambers, and Y Kawaoka. Evolution and ecology of influenza A viruses. Microbiol. Rev., March 1992; 56: 152-179.
Katharine M. Sturm-Ramirez, Trevor Ellis, Barry Bousfield, Lucy Bissett, Kitman Dyrting, Jerold E. Rehg, Leo Poon, Yi Guan, Malik Peiris, and Robert G. Webster. Reemerging H5N1 Influenza Viruses in Hong Kong in 2002 Are Highly Pathogenic to Ducks. J. Virol., May 2004; 78: 4892-4901.
Li K S, Guan Y, Wang J, Smith G J, Xu K M, Duan L, Rahardjo A P, Puthavathana P, Buranathai C, Nguyen T D, Estoepangestie A T, Chaisingh A, Auewarakul P, Long H T, Hanh N T, Webby R J, Poon L L, Chen H, Shortridge K F, Yuen K Y, Webster R G, Peiris J S. Genesis of a highly pathogenic and potentially pandemic H5N1 influenza virus in eastern Asia. Nature. 2004 Jul. 8; 430(6996):209-13.
Y. Guan, L. L. M. Poon, C. Y. Cheung, T. M. Ellis, W. Lim, A. S. Lipatov, K. H. Chan, K. M. Sturm-Ramirez, C. L. Cheung, Y. H. C. Leung, K. Y. Yuen, R. G. Webster, and J. S. M. Peiris. H5N1 influenza: A protean pandemic threat. PNAS, May 2004; 101: 8156-8161.
Choi Y K, Seo S H, Kim J A, Webby R J, Webster R G. Avian influenza viruses in Korean live poultry markets and their pathogenic potential. Virology. 2005 Feb. 20; 332(2):529-37.
Peiris J S, Guan Y, Markwell D, Ghose P, Webster R G, Shortridge K F. Cocirculation of avian H9N2 and contemporary “human” H3N2 influenza A viruses in pigs in southeastern China: potential for genetic reassortment? J Virol. 2001 October; 75(20):9679-86.
Chen H, Deng G, Li Z, Tian G, Li Y, Jiao P, Zhang L, Liu Z, Webster R G, Yu K. The evolution of H5N1 influenza viruses in ducks in southern China. Proc Natl Acad Sci USA. 2004 Jul. 13; 101(28):10452-7. Epub 2004 Jul. 2.
Kanta Subbarao, * Alexander Klimov, Jacqueline Katz, Helen Regnery, Wilina Lim, Henrietta Hall, Michael Perdue, David Swayne, Catherine Bender, Jing Huang, Mark Hemphill, Thomas Rowe, Michael Shaw, Xiyan Xu, Keiji Fukuda, Nancy Cox. Characterization of an Avian Influenza A (H5N1) Virus Isolated from a Child with a Fatal Respiratory Illness. Science, Vol 279, Issue 5349, 393-396, 16 January 1998 [DOI: 10.1126/science.279.5349.393].
E C Claas, A D Osterhaus, R van Beek, J C De Jong, G F Rimmelzwaan, D A Senne, S Krauss, K F Shortridge, and R G Webster. Human influenza A H5N1 virus related to a highly pathogenic avian influenza virus. Lancet, February 1998; 351(9101): 472-7.
Yi Guan, Kennedy F. Shortridge, Scott Krauss, and Robert G. Webster. Molecular characterization of H9N2 influenza viruses: Were they the donors of the “internal” genes of H5N1 viruses in Hong Kong? PNAS, August 1999; 96: 9363-9367.
Marra M A, Jones S J, Astell C R, Holt R A, Brooks-Wilson A, Butterfield Y S, Khattra J, Asano J K, Barber S A, Chan S Y, Cloutier A, Coughlin S M, Freeman D, Girn N, Griffith O L, Leach S R, Mayo M, McDonald H, Montgomery S B, Pandoh P K, Petrescu A S, Robertson A G, Schein J E, Siddiqui A, Smailus D E, Stott J M, Yang G S, Plummer F, Andonov A, Artsob H, Bastien N, Bernard K, Booth T F, Bowness D, Czub M, Drebot M, Fernando L, Flick R, Garbutt M, Gray M, Grolla A, Jones S, Feldmann H, Meyers A, Kabani A, Li Y, Normand S, Stroher U, Tipples G A, Tyler S, Vogrig R, Ward D, Watson B, Brunham R C, Krajden M, Petric M, Skowronski D M, Upton C, Roper R L. The Genome sequence of the SARS-associated coronavirus. Science. 2003 May 30; 300(5624):1399-404. Epub 2003 May 1.
Rota P A, Oberste M S, Monroe S S, Nix W A, Campagnoli R, Icenogle J P, Penaranda S, Bankamp B, Maher K, Chen M H, Tong S, Tamin A, Lowe L, Frace M, DeRisi J L, Chen Q, Wang D, Erdman D D, Peret T C, Burns C, Ksiazek T G, Rollin P E, Sanchez A, Liffick S, Holloway B, Limor J, McCaustland K, Olsen-Rasmussen M, Fouchier R, Gunther S, Osterhaus A D, Drosten C, Pallansch M A, Anderson L J, Bellini W J. Characterization of a novel coronavirus associated with severe acute respiratory syndrome. Science. 2003 May 30; 300(5624):1394-9. Epub 2003 May 1.
der Hoek L, Pyrc K, Jebbink M F, Vermeulen-Oost W, Berkhout R J, Wolthers K C, Wertheim-van Dillen P M, Kaandorp J, Spaargaren J, Berkhout B. Identification of a new human coronavirus. Nat. Med. 2004 April; 10(4):368-73. Epub 2004 Mar. 21.
Woo P C, Lau S K, Chu C M, Chan K H, Tsoi H W, Huang Y, Wong B H, Poon R W, Cai J J, Luk W K, Poon L L, Wong S S, Guan Y, Peiris J S, Yuen K Y. Characterization and complete genome sequence of a novel coronavirus, coronavirus HKU1, from patients with pneumonia. J Virol. 2005 January; 79(2):884-95.
S J Lolle, J L Victor, J M Young, and R E Pruitt. Genome-wide non-mendelian inheritance of extra-genomic information in Arabidopsis. Nature, March 2005; 434(7032): 505-9.
Krolikowski K A, Victor J L, Wagler T N, Lolle S J, Pruitt R E. Isolation and characterization of the Arabidopsis organ fusion gene HOTHEAD. Plant J. 2003 August; 35(4):501-11.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for identifying parental viral strains in a population of viruses, wherein the population comprises parental viral strains and mutant progeny viral strains, comprising the steps of:
a) obtaining the nucleic acid or polypeptide sequence of one or more viral genes from a number of isolated viral strains from the population, the number sufficient to allow for identification of the viral strains prevalent in the population, the viral strains having sequence divergence in the population, or both;
b) identifying the viral strains prevalent in the population, or viral strains with sequence divergence in the population, or both;
wherein the prevalent viral sequences, or the viral sequences with sequence divergence are identified as the parental viral strains.

2. The method of claim 1, wherein the prevalent viral sequences, or the viral sequences with sequence divergence are determined by aligning multiple nucleic acid or polypeptide sequences.

3. The method of claim 1, wherein the mutant progeny viral strains are formed by recombination according to a copy-choice mechanism.

4. The method of claim 1, wherein the parental viral strains are the two most prevalent sequences in the population.

5. The method of claim 1, wherein the parental strains are the two strains with greatest sequence divergence.

6. The method of claim 1, wherein the viruses are RNA viruses.

7. The method of claim 6, wherein the RNA viruses are single-stranded RNA viruses.

8. The method of claim 7 wherein the single-stranded RNA viruses are positive-sense RNA viruses.

9. The method of claim 7, wherein the single-stranded RNA viruses are negative-sense RNA viruses.

10. The method of claim 6, wherein the RNA viruses are double-stranded RNA viruses.

11. The method of claim 10, wherein the double-stranded RNA viruses are positive-strand RNA viruses.

12. The method of claim 10, wherein the double-stranded RNA viruses are negative-strand RNA viruses.

13. The method of claim 1, wherein the viruses are DNA viruses.

14. The method of claim 13, wherein the DNA viruses are single-stranded DNA viruses.

15. The method of claim 13, wherein the DNA viruses are double-stranded DNA viruses.

16. The method of claim 9, wherein the viruses are influenza viruses.

17. The method of claim 9, wherein the viruses are coronavirus viruses.

18. The method of claim 1, wherein the protein or nucleic acid sequences are from influenza viruses.

19. The method of claim 18, wherein the influenza nucleic acid or polypeptide sequences are selected from the group consisting of: HA, NA, NP, PA, PB1, PB2, MP, and NS, or combinations thereof.

20. The method of claim 1, wherein the nucleic acid or polypeptide sequences are obtained by sequencing the isolated viral strains.

21. The method of claim 1, wherein the nucleic acid or polypeptide sequences are obtained from a publicly available database.

22. The method of claim 1, wherein the sufficient number is five or more viral sequences.

23. The method of claim 1, wherein the sufficient number is 15 or more viral sequences.

24. The method of claim 1, wherein the one or more viral genes is at least two genes.

25. The method of claim 1, wherein the one or more viral genes is at least three viral genes.

26. The method of claim 1, wherein the one or more viral genes is at least four viral genes.

27. A method of producing a viral vaccine, comprising:
infecting a host animal with the parental viral strains identified according to claim 1; and
isolating mutant progeny viral strains from the host animal.

28. A method of producing a viral vaccine, comprising:
infecting a cell line with the parental viral strains identified according to claim 1; and
isolating mutant progeny viral strains from the host animal.

29. A method of producing a viral vaccine, comprising:
infecting an egg with the parental viral strains identified according to claim 1; and
isolating mutant progeny viral strains from the host animal.

30. A method of producing a viral vaccine, comprising:
infecting a insect cell with the parental viral strains identified according to claim 1; and
isolating mutant progeny viral strains from the host animal.

31. A method of producing a viral vaccine, comprising:
infecting a bacterial cell with the parental viral strains identified according to claim 1; and
isolating mutant progeny viral strains from the host animal.

32. A method of producing a viral vaccine, comprising:
infecting a cell or cell extract capable of supporting viral replication with the parental viral strains identified according to claim 1; and
isolating mutant progeny viral strains from the host animal.

33. The method of any one of claims 27-32 further comprising attenuating the mutant progeny viral strains to make an attenuated viral vaccine.

34. The method of any one of claims 27-32, further comprising killing the mutant progeny viral strains to make a killed viral vaccine.

35. The method of any one of claims 27-32 further comprising isolating viral antigens, or portions thereof, from the mutant progeny viral strains to make a subunit viral vaccine.

36. The method of any one of claims 27-32 wherein the viruses are RNA viruses.

37. The method of claim 36, wherein the RNA viruses are single-stranded RNA viruses.

38. The method of claim 37, wherein the single-stranded RNA viruses are positive-sense RNA viruses.

39. The method of claim 37, wherein the single-stranded RNA viruses are negative-sense RNA viruses.

40. The method of claim 36 wherein the RNA viruses are double-stranded RNA viruses.

41. The method of claim 40, wherein the double-stranded RNA viruses are positive-strand RNA viruses.

42. The method of claim 40, wherein the double-stranded RNA viruses are negative-strand RNA viruses.

43. The method of claim 42 wherein the viruses are DNA viruses.

44. The method of claim 43, wherein the DNA viruses are single-stranded DNA viruses.

45. The method of claim 43, wherein the DNA viruses are double-stranded DNA viruses.

46. A method of immunizing a subject against a virus comprising:
administering to the subject the attenuated virus vaccine of claim 43 in an amount sufficient to immunize the subject.

47. A method of immunizing a subject against a virus comprising:
administering to the subject the killed virus vaccine of claim 44 in an amount sufficient to immunize the subject.

48. A method of immunizing a subject against a virus comprising administering to the subject the subunit virus vaccine of claim 46 in an amount sufficient to immunize the subject.

49. The method of claim 39, wherein the parental strains are influenza viral strains.

50. The method of claim 39, wherein the parental strains are corona virus viral strains.

51. A method of immunizing a subject against a virus comprising:
administering to the subject a first virus representing the first parental viral strain and a second virus representing a second parental viral strain, the first and second parental viral strains identified according to the method of claim 1, in an amount sufficient to immunize the subject.

52. The method of claim 51, wherein the viruses are RNA viruses.

53. The method of claim 52, wherein the RNA viruses are single-stranded RNA viruses.

54. The method of claim 53, wherein the single-stranded RNA viruses are positive-sense RNA viruses.

55. The method of claim 53, wherein the single-stranded RNA viruses are negative-sense RNA viruses.

56. The method of claim 52, wherein the RNA viruses are double-stranded RNA viruses.

57. The method of claim 51, wherein the double-stranded RNA viruses are positive-strand RNA viruses.

58. The method of claim 51, wherein the double-stranded RNA viruses are negative-strand RNA viruses.

59. The method of claim 51, wherein the viruses are DNA viruses.

60. The method of claim 59, wherein the DNA viruses are single-stranded DNA viruses.

61. The method of claim 59, wherein the DNA viruses are double-stranded DNA viruses.

62. The method of claim 51, further comprising attenuating the parental viral strains prior to administering to the subject.

63. The method of claim 51, further comprising killing the parental viral strains prior to administering to the subject.

64. The method of claim 51, further comprising isolating viral antigens, or portions thereof, from the parental viral strains to make a subunit viral vaccine prior to administering to the subject.

65. The method of claim 51, wherein the parental strains are influenza viral strains.

66. The method of claim 51, wherein the parental strains are corona virus viral strains.

67. A viral vaccine composition comprising the parental viral strains identified according to claim 1, or antigens, or portions of antigens, therefrom.

68. The viral vaccine of claim 67, further comprising mutant progeny viral strains derived from the parental viral strains, or antigens, or portions of antigens, therefrom.

69. The viral vaccine of claim 67, wherein the vaccine comprises two viral strains, or antigens, or portions of antigens from two viral strains.

70. A vaccine composition, comprising mutant progeny viral strains, or antigens or portions of antigens therefrom, made by recombination according to a copy-choice mechanism of two viral strains whose genomes are made up of non-identical nucleic acid sequences.

71. The vaccine composition of claim 70, wherein two viral strains are parental viral strains identified according to the method of claim 1.

72. The vaccine composition of claim 70, wherein the mutant progeny viral strains are produced by recombination according to a copy-choice mechanism in a host animal or cell thereof.

73. The vaccine composition of claim 70, wherein the mutant progeny viral strains are produced by recombination according to a copy-choice mechanism in cell culture or extract thereof.

74. The vaccine composition of claim 70, wherein the viruses are RNA viruses.

75. The vaccine composition of claim 69, wherein the RNA viruses are single-stranded RNA viruses.

76. The vaccine composition of claim 70, wherein the single-stranded RNA viruses are positive-sense RNA viruses.

77. The vaccine composition of claim 70, wherein the single-stranded RNA viruses are negative-sense RNA viruses.

78. The vaccine composition of claim 70, wherein the RNA viruses are double-stranded RNA viruses.

79. The vaccine composition of claim 78 wherein the double-stranded RNA viruses are positive-strand RNA viruses.

80. The vaccine composition of claim 78, wherein the double-stranded RNA viruses are negative-strand RNA viruses.

81. The vaccine composition of claim 70 wherein the viruses are DNA viruses.

82. The vaccine composition of claim 81, wherein the DNA viruses are single-stranded DNA viruses.

83. The vaccine composition of claim 81, wherein the DNA viruses are double-stranded DNA viruses.

84. A method of identifying the stability of a genome in a population of viruses, comprising:
obtaining the nucleic acid or polypeptide sequence of one or more viral genes from a sufficient number of isolated viruses from the population;
comparing the number of recombinant viral sequences in the isolated viruses;
wherein the greater the number of distinct viral sequences, the greater the instability of the viral genome.

85. A method of identifying the stability of a genome in a population of viruses, comprising:
obtaining the nucleic acid or polypeptide sequence of one or more viral genes from a sufficient number of isolated viruses from the population;
comparing the diversity of viral sequences in the isolated viruses;
wherein the greater the diversity, the greater the instability of the viral genome.

86. A method of immunizing a subject against a virus comprising:
administering to the subject mutant progeny viral strains, or antigens or portions of antigens therefrom, made by recombination according to a copy-choice mechanism of two viral strains whose genomes are made up of non-identical nucleic acid sequences.

87. A method of immunizing a subject against a virus comprising:
determining the parental viral strains in a population of viruses;
allowing the parental viral strains to recombine according to a copy-choice mechanism to produce mutant progeny viral strains;
administering the parental viral strains, or mutant progeny viral strains, or antigens or portions of antigens therefrom, in an amount sufficient to immunize the subject.

88. A method for identifying parental influenza strains in a population of influenza viruses, wherein the population comprises parental influenza strains and mutant progeny influenza strains, comprising the steps of:
a) obtaining the nucleic acid or polypeptide sequence of one or more influenza genes from a number of isolated influenza strains from the population, the number sufficient to allow for identification of the influenza strains prevalent in the population, the influenza strains having sequence divergence in the population, or both;
b) identifying the influenza strains prevalent in the population, or influenza strains with sequence divergence in the population, or both;
wherein the influenza sequences, or the influenza sequences with sequence divergence are the parental influenza strains.

89. A method of producing an influenza vaccine, comprising:
infecting a host animal with the parental influenza strains identified according to claim 88; and
isolating mutant progeny influenza strains from the host animal.

90. A method of immunizing a subject against an influenza virus comprising:
administering to the subject a first influenza virus representing the first parental influenza strain and a second influenza virus representing a second parental influenza strain, the first and second parental influenza strains identified according to the method of claim 88, in an amount sufficient to immunize the subject.

91. A vaccine composition, comprising mutant progeny influenza strains, or antigens or portions of antigens therefrom, made by recombination according to a copy-choice mechanism of two influenza strains whose genomes are made up of non-identical nucleic acid sequences.

92. A method for identifying parental coronavirus strains in a population of coronavirus viruses, wherein the population comprises parental coronavirus strains and mutant progeny coronavirus strains, comprising the steps of:
a) obtaining the nucleic acid or polypeptide sequence of one or more coronavirus genes from a number of isolated coronavirus strains from the population, the number sufficient to allow for identification of the coronavirus strains prevalent in the population, the coronavirus strains having sequence divergence in the population, or both;
b) identifying the coronavirus strains prevalent in the population, or coronavirus strains with sequence divergence in the population, or both;
wherein the prevalent coronavirus sequences, or the coronavirus sequences with sequence divergence are the parental coronavirus strains.

93. A method of producing a coronavirus vaccine, comprising:
infecting a host animal cell, cell line, egg cell, insect cell, bacterial cell, or cell extract capable of supporting viral replication with the parental coronavirus strains identified according to claim 92; and
isolating mutant progeny coronavirus strains from the cell or cell extract.

94. A method of immunizing a subject against an coronavirus virus comprising:
administering to the subject a first coronavirus virus representing the first parental coronavirus strain and a second coronavirus virus representing a second parental coronavirus strain, the first and second parental coronavirus strains identified according to the method of claim 88, in an amount sufficient to immunize the subject.

95. A vaccine composition, comprising mutant progeny coronavirus strains, or antigens or portions of antigens therefrom, made by recombination according to a copy-choice mechanism of two coronavirus strains whose genomes are made up of non-identical nucleic acid sequences.

96. A method of producing mutant progeny viral strains for the manufacture of a viral vaccine comprising;
infecting a cell or animal with two non-identical viral strains;
allowing for recombination of the non-identical viral strains according to a copy-choice mechanism;
thereby producing mutant progeny viral strains.

97. The method of claim 96, further comprising isolating the mutant progeny viral strains from the host cell or animal.

98. The method of claim 96, wherein the non-identical viral strains are from different viruses.

99. The method of claim 98, wherein the non-identical viral strains from different viruses are from influenza and Ebola.

100. A method of determining the efficacy of a vaccine comprising:
obtaining the nucleic acid or polypeptide sequence of one or more viral genes from a number of isolated viral strains from a population that has been treated with a viral vaccine, the number sufficient to allow for number of mutant progeny viral strains in the population;
wherein, the lower the number of different mutant progeny viral strain sequences, the greater the efficacy of the vaccine.

101. A method of determining the efficacy of a viral vaccine by monitoring the genetic diversity in a population of viruses, comprising:
obtaining the nucleic acid or polypeptide sequence of one or more viral genes from a sufficient number of isolated viruses from the population before and after treatment with a viral vaccine;
comparing the number of mutant viral progeny strains in the viruses isolated before treatment, to the number of mutant viral progeny strains isolated after treatment;
wherein a decrease in the genetic diversity of the sequences is indicative of the efficacy of the treatment.

102. A method of producing a library of viral strains, comprising:
infecting a cell or host animal with two or more viral strains;
allowing the viral strains to recombine by the copy choice mechanism;
thereby creating a library of viral strains.

103. The method of claim 102, further comprising the step of isolating the library of viral strains from the cell or host animal.

104. A method of predicting the genotype of antiviral resistant viruses comprising:
infecting a cell or host animal having been treated with an antiviral compound with two or more parental viral strains;
allowing said viral strains to produce mutant viral progeny strains by a copy choice mechanism;
isolating the mutant progeny viral strains from the cell or host animal;
wherein the isolated mutant progeny viral strains are indicative of the antiviral resistant viral strains that will be produced in a population of infected subjects treated with the antiviral compound.

105. A method for identifying parental viral strains in a population of viruses, wherein the population comprises parental viral strains and mutant progeny viral strains produced from the parental viral strains via a copy-choice recombination mechanism, comprising the steps of:
a) obtaining the nucleic acid or polypeptide sequence of one or more viral genes from a number of isolated viral strains from the population, the number sufficient to allow for identification of the viral strains prevalent in the population, the viral strains having sequence divergence in the population, or both;
b) identifying the viral strains prevalent in the population, or viral strains with sequence divergence in the population, or both;
wherein the prevalent viral sequences, or the viral sequences with sequence divergence are the parental viral strains.

106. A method of producing mutant progeny viral strains for the manufacture of a viral vaccine comprising;
infecting a cell or animal with two non-identical viral strains;
allowing for recombination of the non-identical viral strains according to a copy-choice mechanism;
selecting mutant progeny viral strains from the population;
thereby producing mutant progeny viral strains for the manufacture of a viral vaccine.

107. The method of claim 106, wherein mutant progeny viral strains are isolated based on a determinable phenotype or genotype.

108. The method of claim 107, wherein said observable phenotype is selected from the group consisting of: recombination, virulence, and tropism.

109. A method of predicting the sequence of one or more genes in a mutant progeny viral strain comprising:
obtaining the sequence of the one of more genes from the parental viral strains;
determining the location of possible recombination events;
thereby predicting the sequence of one or more genes in a mutant progeny viral strain.

110. The method of claim 109, wherein the viral strain is selected from the group consisting of an influenza viral strain, a corona viral strain, and an HIV viral strain.

111. The method of claim 109, further comprising using the predicted sequence of the mutant progeny viral strain to develop a vaccine against said virus.

112. A method of producing mutant progeny viral strains comprising;
infecting a cell or animal with two non-identical viral strains;
allowing for recombination of the non-identical viral strains according to a copy-choice mechanism;
thereby producing mutant progeny viral strains.

113. The method of claim 112, further comprising isolating said mutant progeny viral strains.

114. A method of producing a population of recombinant genes comprising:
introducing into a cell two or more non-identical copies of a gene;
allowing for recombination of the genes;
thereby producing a population of recombinant genes.

115. The method of claim 114, wherein the recombination occurs via a copy-choice mechanism.

116. The method of claim 114, further comprising isolating the one or more members of the population of recombinant genes.

117. A method of producing a recombined DNA molecule comprising:
introducing two non-identical copies of a gene into a cell,
allowing the genes to recombine via copy-choice recombination such that the resulting recombined DNA molecule has a portion of the DNA sequence derived from each of the two non-identical copies of the gene.

118. The method of claim 117, wherein said gene is a plant gene.

119. The method of claim 117, wherein gene is an animal gene.

120. The method of claim 117, wherein said gene is a viral gene.

121. The method of any of claims 117-120, wherein said gene is a heterologous gene or fragment thereof.

122. A viral vaccine effective in immunizing an animal to a number of recombinant viral progeny strains.

123. A viral vaccine effective against a recombinant viral progeny strain.

124. A viral vaccine effective against a recombinant viral progeny strain, wherein said recombinant viral progeny strain derives from parental viral strains with different serotypes.

125. The viral vaccine of claim 124, wherein parental viral strain serotypes are selected from the group consisting of H1N1, H1N2, H2N2, H3N2, H5N1, H7N2, H7N3, H7N7, H9N2, and H3N8.

126. A method for predicting progeny viral strain sequences from sequences of two or more viral strains, comprising:
a) selecting two or more non-identical viral strain sequences, and
b) predicting progeny viral strain sequences produced from the selected strain sequences via a copy-choice recombination mechanism,
such that progeny viral strain sequences are predicted.

127. The method of claim 126, wherein the method further comprises raising a vaccine to at least one progeny viral strain.

128. The method of claim 127, wherein the method further comprises administering the vaccine to a subject.

129. A method for predicting the sequence of a recombined DNA molecule, comprising:
a) selecting two non-identical copies of a gene, and
b) modeling recombination of the selected genes via a copy-choice recombination mechanism,
such that the sequence of a recombined DNA molecule is predicted.

130. A method of identifying recombinant progeny viral strain sequences from sequences of two or more viral strains, comprising:
a) selecting two or more viral strain sequences, and
b) identifying progeny viral strain sequences produced via a copy-choice recombination,
such that recombinant progeny viral strain sequences are identified.


SEQUENCE LISTING (1422 sequences)

SEQ ID NO: 1 - DNA, 12 bp, Influenza virus
gtgccaaagcgg

SEQ ID NO: 2 - DNA, 12 bp, Influenza virus
acgctgggatgt

SEQ ID NO: 3 - DNA, 12 bp, Influenza virus
gtatcaaagcag

SEQ ID NO: 4 - DNA, 10 bp, Influenza virus
gctgggatgt

SEQ ID NO: 5 - DNA, 10 bp, Influenza virus
gctgggatgt

SEQ ID NO: 6 - DNA, 10 bp, Influenza virus
gctgggatgt

SEQ ID NO: 7 - DNA, 10 bp, Influenza virus
gctgggatgt

SEQ ID NO: 8 - DNA, 10 bp, Influenza virus
gctgggatgt

SEQ ID NO: 9 - DNA, 10 bp, Influenza virus
gctgggatgt

SEQ ID NO: 10 - DNA, 10 bp, Influenza virus
gctgggatgt

SEQ ID NO: 11 - DNA, 10 bp, Influenza virus
gctaggatgt

SEQ ID NO: 12 - DNA, 10 bp, Influenza virus
gctaggatgt

SEQ ID NO: 13 - DNA, 10 bp, Influenza virus
gctaggatgt

SEQ ID NO: 14 - DNA, 10 bp, Influenza virus
gctaggatgt

SEQ ID NO: 15 - DNA, 10 bp, Influenza virus
gctaggatgt

SEQ ID NO: 16 - DNA, 10 bp, Influenza virus
gctaggatgt

SEQ ID NO: 17 - DNA, 10 bp, Influenza virus
gctaggatgt

SEQ ID NO: 18 - DNA, 10 bp, Influenza virus
gctaggatgt

SEQ ID NO: 19 - DNA, 12 bp, Influenza virus
acgctaggatgt

SEQ ID NO: 20 - DNA, 12 bp, Influenza virus
acgctagggcgt

SEQ ID NO: 21 - DNA, 12 bp, Influenza virus
gtgccaaggtgg

SEQ ID NO: 22 - DNA, 10 bp, Influenza virus
gccaaagcgg

SEQ ID NO: 23 - DNA, 10 bp, Influenza virus
gtcaaagcgg

SEQ ID NO: 24 - DNA, 12 bp, Influenza virus
gtgtcaaagcag

SEQ ID NO: 25 - DNA, 12 bp, Influenza virus
gtgtcaaagcag

SEQ ID NO: 26 - DNA, 10 bp, Influenza virus
gtcaaagcag

SEQ ID NO: 27 - DNA, 10 bp, Influenza virus
gtcaaagcag

SEQ ID NO: 28 - DNA, 12 bp, Influenza virus
gtgtcgaagcag

SEQ ID NO: 29 - DNA, 12 bp, Influenza virus
gtgtcgaagcag

SEQ ID NO: 30 - DNA, 10 bp, Influenza virus
atcaaagcag

SEQ ID NO: 31 - DNA, 10 bp, Influenza virus
atcaaagcag

SEQ ID NO: 32 - DNA, 12 bp, Influenza virus
gtatcaaagcag

SEQ ID NO: 33 - DNA, 10 bp, Influenza virus
atcaaagcag

SEQ ID NO: 34 - DNA, 10 bp, Influenza virus
atcaaagcag

SEQ ID NO: 35 - DNA, 10 bp, Influenza virus
attaaagtgg

SEQ ID NO: 36 - DNA, 12 bp, Influenza virus
acatcaaagcag

SEQ ID NO: 37 - DNA, 12 bp, Influenza virus
acgctgggatgt

SEQ ID NO: 38 - DNA, 12 bp, Influenza virus
gtatcaaagcag

SEQ ID NO: 39 - DNA, 110 bp, Influenza virus
gacgcgacatcagaaataacacgctaaaagatatgtctaagcacacccaggtaatttttcctaatcaaattcatatttaacagctatagttcccaagttaatgtccatga

SEQ ID NO: 40 - DNA, 110 bp, Influenza virus
gacgcgacaccagaaataacacaataaaaggtatgcctaagcacgcccagattatttttcccaattagatgcgtatttagcagctataattcccaagttaatatccgtga

SEQ ID NO: 41 - DNA, 110 bp, Influenza virus
agtatagtgtggagggcggtgagccggggaacgcattagcaataatttgagcagccccctttggcagagcttacgcccgatgatcggggcctttggaccggcccttacag

SEQ ID NO: 42 - DNA, 73 bp, Influenza virus
gacgcgacaccagaaataacacaataaaaggtatgcctaagcacgcccagattatttttcccaattagatgcg

SEQ ID NO: 43 - DNA, 73 bp, Influenza virus; identical to SEQ ID NO: 42

SEQ ID NO: 44 - DNA, 73 bp, Influenza virus; identical to SEQ ID NO: 42

SEQ ID NO: 45 - DNA, 73 bp, Influenza virus; identical to SEQ ID NO: 42

SEQ ID NO: 46 - DNA, 73 bp, Influenza virus; identical to SEQ ID NO: 42

SEQ ID NO: 47 - DNA, 65 bp, Influenza virus
gacgcgacaccagaaataacacaataaaaggtatgcctaagcacgcccagattatttttcccaat

SEQ ID NO: 48 - DNA, 73 bp, Influenza virus; identical to SEQ ID NO: 42

SEQ ID NO: 49 - DNA, 110 bp, Influenza virus
gacgcgacaccagaaataacacaataaaaggtatgcctaagcacgcccagattatttttcccaattagatgcgtattcggcagctatagttcccaagttaatatccgtga

SEQ ID NO: 50 - DNA, 110 bp, Influenza virus; identical to SEQ ID NO: 49

SEQ ID NO: 51 - DNA, 73 bp, Influenza virus
gacgcgacaccagaaataacacaataaaagatatgcctaagcacgcccagattatttttcccaattagatgcg

SEQ ID NO: 52 - DNA, 73 bp, Influenza virus; identical to SEQ ID NO: 51

SEQ ID NO: 53 - DNA, 70 bp, Influenza virus
gacgcgacaccagaaataacacaataaaagatatgcctaagcacgcccagattatttttcccaattagat

SEQ ID NO: 54 - DNA, 73 bp, Influenza virus; identical to SEQ ID NO: 51

SEQ ID NO: 55 - DNA, 73 bp, Influenza virus
gacgcgacaccagaaataacacaataaaagatatgcctaagcacgtccagattatttttcccaattagatgcg

SEQ ID NO: 56 - DNA, 73 bp, Influenza virus; identical to SEQ ID NO: 55

SEQ ID NO: 57 - DNA, 96 bp, Influenza virus
gacgcgacatcagaaataacacaataaaagatatgcctaagcacgcccagattatttttcccaatcagatgcgtatttagcagctataattcccaa

SEQ ID NO: 58 - DNA, 96 bp, Influenza virus; identical to SEQ ID NO: 57

SEQ ID NO: 59 - DNA, 67 bp, Influenza virus
gacgcgacatcagaaataacacactaaaagatatgcctaagcacgcccagataatttttcccaatca

SEQ ID NO: 60 - DNA, 73 bp, Influenza virus
gacgcgacatcagaaataacacactagaagatacgcctaagcacgcccagataatttttcccaatcaaattcg

SEQ ID NO: 61 - DNA, 110 bp, Influenza virus
agtatagtgtggagggcggtgagccggggaacgcattagcaataatttgagcagccccctttggctagatgcgtgcccgatgatcggggcctttggaccggcccttacag

SEQ ID NO: 62 - DNA, 110 bp, Influenza virus; identical to SEQ ID NO: 41

SEQ ID NO: 63 - DNA, 73 bp, Influenza virus
agtatagtgtggagggcggtgagccggggaacgcattagcaataatttgagcagccccctttggcagagctcg

SEQ ID NO: 64 - DNA, 73 bp, Influenza virus
aatatagtgtggagggcggtgagccggggaacgcattagcaataatttgagcagccccctttggcagagctta

SEQ ID NO: 65 - DNA, 73 bp, Influenza virus; identical to SEQ ID NO: 64

SEQ ID NO: 66 - DNA, 73 bp, Influenza virus; identical to SEQ ID NO: 64

SEQ ID NO: 67 - DNA, 72 bp, Influenza virus
agtatagtgtggagggcggtgagccggggaacgcattagcaataatttgagcagccccctttggcagagctt

SEQ ID NO: 68 - DNA, 110 bp, Influenza virus; identical to SEQ ID NO: 41

SEQ ID NO: 69 - DNA, 110 bp, Influenza virus; identical to SEQ ID NO: 41

SEQ ID NO: 70 - DNA, 73 bp, Influenza virus
agtatagtgtggagggcggtgagccggggaacgcattagcaataatttgagcagccccctttggcagagctta

SEQ ID NO: 71 - DNA, 73 bp, Influenza virus
agtatagtgtggagggcggtgagccggggaacgcattagcaataatttgagcagtcccctttggcagagctca

SEQ ID NO: 72 - DNA, 110 bp, Influenza virus
agtatagtgtggagggcggtgagccggagaacgcattagcaataactcgagcagccccctttggcagagcttacgcccgatgatcggggcctttggaccggcccttacga

SEQ ID NO: 73 - DNA, 110 bp, Influenza virus; identical to SEQ ID NO: 72

SEQ ID NO: 74 - DNA, 110 bp, Influenza virus; identical to SEQ ID NO: 40

SEQ ID NO: 75 - DNA, 110 bp, Influenza virus; identical to SEQ ID NO: 41

SEQ ID NO: 76 - DNA, 113 bp, Influenza virus
tacgtattagatagactctggcgaattcccgcttggctgacgctaatacaaaaagcccagttgagctccataatcgtttgcgcgtctaaaaacgcaggcagaggggctaaccg

SEQ ID NO: 77 - DNA, 113 bp, Influenza virus
cgcacgccccgcgagtcctaatacccatttttccgatcggtatcggcataggaaatatacccagactttgcagctacccacataattgggggaatgaaagagatattcagtta

SEQ ID NO: 78 - DNA, 113 bp, Influenza virus
tatgtattagatagacttcggcgaattcccgctttgcttacgctaatgcgaagggcccggttgagtcccatgatcgtttgcgcgtctaaaaacgcaggcagaggggcttaccg

SEQ ID NO: 79 - DNA, 113 bp, Influenza virus; identical to SEQ ID NO: 77

SEQ ID NO: 80 - DNA, 113 bp, Influenza virus
cgcacgccccgcgagtcctaatacccatttttccgatcgacgctaatgcgaagagtttacccagactttgcagctacccacataattgggggaatgaaagagatattcagtta

SEQ ID NO: 81 - DNA, 43 bp, Influenza virus
cagctacccacataattgggggaatgaaagagatattcagtta

SEQ ID NO: 82 - DNA, 43 bp, Influenza virus; identical to SEQ ID NO: 81

SEQ ID NO: 83 - DNA, 43 bp, Influenza virus; identical to SEQ ID NO: 81

SEQ ID NO: 84 - DNA, 43 bp, Influenza virus; identical to SEQ ID NO: 81

SEQ ID NO: 85 - DNA, 43 bp, Influenza virus; identical to SEQ ID NO: 81

SEQ ID NO: 86 - DNA, 43 bp, Influenza virus; identical to SEQ ID NO: 81

SEQ ID NO: 87 - DNA, 43 bp, Influenza virus; identical to SEQ ID NO: 81

SEQ ID NO: 88 - DNA, 43 bp, Influenza virus; identical to SEQ ID NO: 81

SEQ ID NO: 89 - DNA, 43 bp, Influenza virus; identical to SEQ ID NO: 81

SEQ ID NO: 90 - DNA, 43 bp, Influenza virus; identical to SEQ ID NO: 81

SEQ ID NO: 91 - DNA, 43 bp, Influenza virus; identical to SEQ ID NO: 81

SEQ ID NO: 92 - DNA, 43 bp, Influenza virus; identical to SEQ ID NO: 81

SEQ ID NO: 93 - DNA, 43 bp, Influenza virus; identical to SEQ ID NO: 81

SEQ ID NO: 94 - DNA, 43 bp, Influenza virus
tgatcgtttgcgcgtctgaagacgcaggcagaggggctaaccg

SEQ ID NO: 95 - DNA, 43 bp, Influenza virus
tgatcgtttgcgtgtctgaaaacgcaggcagaggggctaaccg

SEQ ID NO: 96 - DNA, 43 bp, Influenza virus
tgatcgtttgcgtgtctaaaaacgcaggcagaggggctaaccg

SEQ ID NO: 97 - DNA, 113 bp, Influenza virus
tacgtattagatagacttcggcgaattcccgctttgcttatgctaatgcgaagggcccggttgagtcccatgatcgtttgcgtatctaaaaacgcaggcagaggggctaatcg

SEQ ID NO: 98 - DNA, 113 bp, Influenza virus
tacgtattagatagacttcggcgaattcccgctttgcttacgctaatgcgaagggcccggttgagtcccatgatcgtttgcgcatctaaaaacgcaggcagaggggctaatcg

SEQ ID NO: 99 - DNA, 113 bp, Influenza virus
tatgtattagatagacttcggcgaattcccgctttgcttacgctaatacgaagagcccagttgagtcccatgatcgtttgcgcgtctaaaaacgcaggcagaggggctaaccg

SEQ ID NO: 100 - DNA, 43 bp, Influenza virus
tgatcgtttgcgcgtctaaaaacgcaggcagaggggctaaccg

SEQ ID NO: 101 - DNA, 113 bp, Influenza virus
tatgtattagatagacttcggcgaattcccgctttgcttatgctaatgcgaagggcccggttgagtcccatgatcgtttgcgcgtctaaaaacgcaggcagaggggcttaccg

SEQ ID NO: 102 - DNA, 113 bp, Influenza virus
tatgtattagatagacttcggcgaattcccgctttgcttacgctaatgcgaagagcccagttgagtcccatgatcgtttgcgcgtctaaaaacgcaggcagaggggcttaccg

SEQ ID NO: 103 - DNA, 113 bp, Influenza virus
tacgtattagatagacttcggcgaattcccgctttgctgacgctaatgcgaagagcccagttgagtcccatgatcgtttgcgcgtctaaaaacgcaggcagaggggcttaccg

SEQ ID NO: 104 - DNA, 113 bp, Influenza virus
tacgtattagatagacttcggcgaattcccgctttgctgacgctaatgcgaagagcccagttgagtcccatgatcgttcgcgcgtctaaaaacgcaggcagaggggcttaccg

SEQ ID NO: 105 - DNA, 43 bp, Influenza virus
tgatcgtttgcgcgtctaaaaacgcaggcagaggggcttaccg

SEQ ID NO: 106 - DNA, 43 bp, Influenza virus; identical to SEQ ID NO: 105

SEQ ID NO: 107 - DNA, 43 bp, Influenza virus; identical to SEQ ID NO: 105

SEQ ID NO: 108 - DNA, 43 bp, Influenza virus; identical to SEQ ID NO: 105

SEQ ID NO: 109 - DNA, 113 bp, Influenza virus; identical to SEQ ID NO: 77

SEQ ID NO: 110 - DNA, 113 bp, Influenza virus; identical to SEQ ID NO: 78

SEQ ID NO: 111 - DNA, 20 bp, Influenza virus
ggaaacggcagtagagcagg

SEQ ID NO: 112 - DNA, 20 bp, Influenza virus
aaggctaatgaagataagaa

SEQ ID NO: 113 - DNA, 18 bp, Influenza virus
cctgtgaccagacagtgc

SEQ ID NO: 114 - DNA, 18 bp, Influenza virus
tttttgatcagataccgc

SEQ ID NO: 115 - DNA, 18 bp, Influenza virus
cccgcagctgagccgtat

SEQ ID NO: 116 - DNA, 18 bp, Influenza virus
cttttgatcagataccgc

SEQ ID NO: 117 - DNA, 18 bp, Influenza virus
tttttgatcagataccgc

SEQ ID NO: 118 - DNA, 18 bp, Influenza virus
tttttgatcagataccgc

SEQ ID NO: 119 - DNA, 18 bp, Influenza virus
tttttgatcagataccgc

SEQ ID NO: 120 - DNA, 18 bp, Influenza virus
tttttgatcagataccgc

SEQ ID NO: 121 - DNA, 18 bp, Influenza virus
tttttgatcagataccgc

SEQ ID NO: 122 - DNA, 18 bp, Influenza virus
tttttgatcagataccgc

SEQ ID NO: 123 - DNA, 16 bp, Influenza virus
tttttgatcagatacc

SEQ ID NO: 124 - DNA, 17 bp, Influenza virus
tttttgatcagataccg

SEQ ID NO: 125 - DNA, 17 bp, Influenza virus
tttttgatcagataccg

SEQ ID NO: 126 - DNA, 15 bp, Influenza virus
tttttgatcagatac

SEQ ID NO: 127 - DNA, 18 bp, Influenza virus
tttttgttcagataccgc

SEQ ID NO: 128 - DNA, 17 bp, Influenza virus
tttttgttcagataccg

SEQ ID NO: 129 - DNA, 17 bp, Influenza virus
tttttgttcagataccg

SEQ ID NO: 130 - DNA, 18 bp, Influenza virus
tttttggtcagatactgc

SEQ ID NO: 131 - DNA, 18 bp, Influenza virus
tttttggtcggatactgc

SEQ ID NO: 132 - DNA, 18 bp, Influenza virus
cctttggccagacagtgc

SEQ ID NO: 133 - DNA, 18 bp, Influenza virus
cctgtggccagacagtgc

SEQ ID NO: 134 - DNA, 18 bp, Influenza virus
cctgtggccagacagtgc

SEQ ID NO: 135 - DNA, 18 bp, Influenza virus
cctgtggccagacagtgc

SEQ ID NO: 136 - DNA, 18 bp, Influenza virus
cctgcggccagaccgtgc

SEQ ID NO: 137 - DNA, 18 bp, Influenza virus
cctgcggccagaccgtgc

SEQ ID NO: 138 - DNA, 18 bp, Influenza virus
cccgcggctaaaccgtat

SEQ ID NO: 139 - DNA, 18 bp, Influenza virus
cccgcggctaaaccgtat

SEQ ID NO: 140 - DNA, 18 bp, Influenza virus
cccgcggctaaaccgtat

SEQ ID NO: 141 - DNA, 18 bp, Influenza virus
cccgcggctaaaccgtat

SEQ ID NO: 142 - DNA, 17 bp, Influenza virus
cccgcggctaaaccgta

SEQ ID NO: 143 - DNA, 18 bp, Influenza virus
cccgcagctgagccgtat

SEQ ID NO: 144 - DNA, 18 bp, Influenza virus
cccgcagctgagccgtat

SEQ ID NO: 145 - DNA, 18 bp, Influenza virus
cccgcagctgagccgtat

SEQ ID NO: 146 - DNA, 18 bp, Influenza virus
cccgcagctgagccgtat

SEQ ID NO: 147 - DNA, 18 bp, Influenza virus
cccgcagctgagccgtat

SEQ ID NO: 148 - DNA, 18 bp, Influenza virus
cccgcagctgagccgtat

SEQ ID NO: 149 - DNA, 15 bp, Influenza virus
cccgcagctgagccg

SEQ ID NO: 150 - DNA, 18 bp, Influenza virus
tttttgatcagataccgc

SEQ ID NO: 151 - DNA, 18 bp, Influenza virus
cccgcagctgagccgtat

SEQ ID NO: 152 - DNA, 63 bp, Influenza virus
ttttggaggacgtcaagcgcacaccaactaaattacgcgacgttgtatgtctaaatacgagta

SEQ ID NO: 153 - DNA, 63 bp, Influenza virus
tcctgaaaggtgctgcataaacattggccggactatatggtaccaagtatttataaatgagct

SEQ ID NO: 154 - DNA, 63 bp, Influenza virus
cttcagggaacatcatgcgtgtgccaattaagacgcgcaacgttgtacgccagagtgcagata

SEQ ID NO: 155 - DNA, 43 bp, Influenza virus
tcctgaaaggtgctgcataaacattggccggactatatggtac

SEQ ID NO: 156 - DNA, 43 bp, Influenza virus; identical to SEQ ID NO: 155

SEQ ID NO: 157 - DNA, 43 bp, Influenza virus; identical to SEQ ID NO: 155

SEQ ID NO: 158 - DNA, 43 bp, Influenza virus; identical to SEQ ID NO: 155

SEQ ID NO: 159 - DNA, 43 bp, Influenza virus; identical to SEQ ID NO: 155

SEQ ID NO: 160 - DNA, 63 bp, Influenza virus
tcctgaaaggtgctgcataaacattggccggactatatggtaccatgtatttataaatgagct

SEQ ID NO: 161 - DNA, 63 bp, Influenza virus; identical to SEQ ID NO: 160

SEQ ID NO: 162 - DNA, 46 bp, Influenza virus
tcctgaaaggtgctgcataaacattggccggactatatggtaccaa

SEQ ID NO: 163 - DNA, 43 bp, Influenza virus; identical to SEQ ID NO: 155

SEQ ID NO: 164 - DNA, 46 bp, Influenza virus; identical to SEQ ID NO: 162

SEQ ID NO: 165 - DNA, 43 bp, Influenza virus
tcctgagaggtgctgcataaacattggccggactatatggtac

SEQ ID NO: 166 - DNA, 43 bp, Influenza virus; identical to SEQ ID NO: 165

SEQ ID NO: 167 - DNA, 43 bp, Influenza virus; identical to SEQ ID NO: 165

SEQ ID NO: 168 - DNA, 43 bp, Influenza virus; identical to SEQ ID NO: 165

SEQ ID NO: 169 - DNA, 46 bp, Influenza virus
tcctgagaggtgctgcataaacattggccggactatatggtaccaa

SEQ ID NO: 170 - DNA, 63 bp, Influenza virus
tcctgagaggtgctgcacaaacattggccggactatatggtaccatgtatttataaatgagct

SEQ ID NO: 171 - DNA, 63 bp, Influenza virus
tcctgagaggtgctgcataaacattggccggactatatggtaccatgtatttataaatgagct

SEQ ID NO: 172 - DNA, 63 bp, Influenza virus
tcctgagaagtgttacataaacattaaccaaactacacgatgccatacgtctaagtgcaaata

SEQ ID NO: 173 - DNA, 63 bp, Influenza virus
tcttcagaagtgttacataaacattaaccaaactacacgatgccatacgtctaagtgcaaata

SEQ ID NO: 174 - DNA, 43 bp, Influenza virus
ttttgagaggtgctgcataaacattggccggactatatggtac

SEQ ID NO: 175 - DNA, 43 bp, Influenza virus
ttttgagaggtgctgcataaacattggccggaccatatggtgc

SEQ ID NO: 176 - DNA, 43 bp, Influenza virus
ttttgggggatgtcacgcgcacaccaactaaattacgcgacgt

SEQ ID NO: 177 - DNA, 43 bp, Influenza virus; identical to SEQ ID NO: 176

SEQ ID NO: 178 - DNA, 63 bp, Influenza virus
ttttgggggacgtcacgcgcacaccaactaagacacgcgacgttgtatgtctaaatacaagta

SEQ ID NO: 179 - DNA, 63 bp, Influenza virus; identical to SEQ ID NO: 178

SEQ ID NO: 180 - DNA, 63 bp, Influenza virus
cttcagggaatatcatgcgtgtgccaattaagacgcgcaacgttgtacgccagagtgcagata

SEQ ID NO: 181 - DNA, 63 bp, Influenza virus; identical to SEQ ID NO: 180

SEQ ID NO: 182 - DNA, 63 bp, Influenza virus; identical to SEQ ID NO: 180

SEQ ID NO: 183 - DNA, 43 bp, Influenza virus
cttcagggaacatcatacgtgtgccaattaagacgcgcaacgt

SEQ ID NO: 184 - DNA, 44 bp, Influenza virus
cttcaaggaacatcatacgtgtgccaattaagacgcgcaacgtt

SEQ ID NO: 185 - DNA, 63 bp, Influenza virus
cttcaaggaacatcatacgtgtgttaattaagacgcgcaacgttgtacgccagagtgcagata

SEQ ID NO: 186 - DNA, 63 bp, Influenza virus
cttcaaggaatatcatacgtgtgtcaattaagacacgcaacgttgtacgccagagtgcagata

SEQ ID NO: 187 - DNA, 37 bp, Influenza virus
cttcaaggaatatcatacgtgtgtcgattaagacacg

SEQ ID NO: 188 - DNA, 63 bp, Influenza virus; identical to SEQ ID NO: 153

SEQ ID NO: 189 - DNA, 63 bp, Influenza virus; identical to SEQ ID NO: 154

SEQ ID NO: 190 - DNA, 61 bp, Influenza virus
gacccacaacgttgctcacgagagaggaccagagaatatttgacgtagcggcgctcaagaa

SEQ ID NO: 191 - DNA, 61 bp, Influenza virus
agatttttgtaccatctctagacagaagttcagagtctcggagtaggataatatgtggacg

SEQ ID NO: 192 - DNA, 61 bp, Influenza virus
agatttttgtaccatctctagacagaagttcagagcctcggagtaggataatatgtggacg

SEQ ID NO: 193 - DNA, 41 bp, Influenza virus
agatttttgtaccatctctagacagaagttcagagtctcgg

SEQ ID NO: 194 - DNA, 41 bp, Influenza virus; identical to SEQ ID NO: 193

SEQ ID NO: 195 - DNA, 41 bp, Influenza virus; identical to SEQ ID NO: 193

SEQ ID NO: 196 - DNA, 41 bp, Influenza virus
agatttttgtaccatctttagacagaagttcagagtctcgg

SEQ ID NO: 197 - DNA, 41 bp, Influenza virus; identical to SEQ ID NO: 193

SEQ ID NO: 198 - DNA, 61 bp, Influenza virus; identical to SEQ ID NO: 191

SEQ ID NO: 199 - DNA, 61 bp, Influenza virus; identical to SEQ ID NO: 191

SEQ ID NO: 200 - DNA, 45 bp, Influenza virus
agatttttgtaccatctctagacagaagttcagagtctcggagta

SEQ ID NO: 201 - DNA, 61 bp, Influenza virus; identical to SEQ ID NO: 191

SEQ ID NO: 202 - DNA, 45 bp, Influenza virus; identical to SEQ ID NO: 200

SEQ ID NO: 203 - DNA, 41 bp, Influenza virus; identical to SEQ ID NO: 193

SEQ ID NO: 204 - DNA, 41 bp, Influenza virus; identical to SEQ ID NO: 193

SEQ ID NO: 205 - DNA, 41 bp, Influenza virus; identical to SEQ ID NO: 193

SEQ ID NO: 206 - DNA, 41 bp, Influenza virus; identical to SEQ ID NO: 193

SEQ ID NO: 207 - DNA, 41 bp, Influenza virus; identical to SEQ ID NO: 193

SEQ ID NO: 208 - DNA, 61 bp, Influenza virus
agatttttgtaccatctctagacagaagttcagagtctcggagtaggataatatgtggacc

SEQ ID NO: 209 - DNA, 61 bp, Influenza virus; identical to SEQ ID NO: 191

SEQ ID NO: 210 - DNA, 61 bp, Influenza virus; identical to SEQ ID NO: 191

SEQ ID NO: 211 - DNA, 61 bp, Influenza virus; identical to SEQ ID NO: 191

SEQ ID NO: 212 - DNA, 41 bp, Influenza virus; identical to SEQ ID NO: 193

SEQ ID NO: 213 - DNA, 41 bp, Influenza virus; identical to SEQ ID NO: 193

SEQ ID NO: 214 - DNA, 41 bp, Influenza virus
gacccacaacgttgctcacgagagaggacaagagaatattt

SEQ ID NO: 215 - DNA, 41 bp, Influenza virus; identical to SEQ ID NO: 214

SEQ ID NO: 216 - DNA, 61 bp, Influenza virus
gacccacaacgttgctcacgagagaggacaagaggctatttgacgtagcggcgctcaagaa

SEQ ID NO: 217 - DNA, 61 bp, Influenza virus; identical to SEQ ID NO: 216

SEQ ID NO: 218 - DNA, 61 bp, Influenza virus; identical to SEQ ID NO: 192

SEQ ID NO: 219 - DNA, 61 bp, Influenza virus; identical to SEQ ID NO: 192

SEQ ID NO: 220 - DNA, 61 bp, Influenza virus; identical to SEQ ID NO: 192

SEQ ID NO: 221 - DNA, 41 bp, Influenza virus
agatttttgtaccatctctagacagaagttcagagcctcgg

SEQ ID NO: 222 - DNA, 41 bp, Influenza virus; identical to SEQ ID NO: 221

SEQ ID NO: 223 - DNA, 61 bp, Influenza virus; identical to SEQ ID NO: 192

SEQ ID NO: 224 - DNA, 61 bp, Influenza virus
agatttttgtaccatctctagacagaagttcagagcctcggagtaggataatatgtgggcg

SEQ ID NO: 225 - DNA, 36 bp, Influenza virus
agatttttgtaccatctctagacagaagttcagagc

SEQ ID NO: 226 - DNA, 61 bp, Influenza virus; identical to SEQ ID NO: 191

SEQ ID NO: 227 - DNA, 61 bp, Influenza virus; identical to SEQ ID NO: 192

SEQ ID NO: 228 - DNA, 39 bp, Influenza virus
caacdatttggagcccacaccgttgtgtaggcggctaac

SEQ ID NO: 229 - DNA, 39 bp, Influenza virus
cgacdgtccgatacccacaccaccgcgtgggtggcaaat

SEQ ID NO: 230 - DNA, 39 bp, Influenza virus
  modified_base (5): Inosine
tagtnacttagggtttgtgttgttatacaaacaatcgtg

SEQ ID NO: 231 - DNA, 34 bp, Influenza virus
cgacdgtccgatacccacaccaccgtgtgggtgg

SEQ ID NO: 232 - DNA, 32 bp, Influenza virus
cgacdgtccgatacccacaccaccgtgtgggt

SEQ ID NO: 233 - DNA, 34 bp, Influenza virus
cgacdgtccgatacccacaccaccgcgtgggtgg

SEQ ID NO: 234 - DNA, 32 bp, Influenza virus
cgacdgtccgatacccacaccaccgcgttggt

SEQ ID NO: 235 - DNA, 32 bp, Influenza virus
cgacdgtccgatacccacaccaccgcgtgggt

SEQ ID NO: 236 - DNA, 34 bp, Influenza virus; identical to SEQ ID NO: 233

SEQ ID NO: 237 - DNA, 32 bp, Influenza virus; identical to SEQ ID NO: 235

SEQ ID NO: 238 - DNA, 32 bp, Influenza virus; identical to SEQ ID NO: 235

SEQ ID NO: 239 - DNA, 32 bp, Influenza virus; identical to SEQ ID NO: 235

SEQ ID NO: 240 - DNA, 32 bp, Influenza virus; identical to SEQ ID NO: 235

SEQ ID NO: 241 - DNA, 32 bp, Influenza virus; identical to SEQ ID NO: 235

SEQ ID NO: 242 - DNA, 32 bp, Influenza virus; identical to SEQ ID NO: 235

SEQ ID NO: 243 - DNA, 39 bp, Influenza virus
cgacdgtccgatacccacaccaccgcgtgggcggccaat

SEQ ID NO: 244 - DNA, 39 bp, Influenza virus
  modified_base (5): Inosine
tggcnatccagggttcgcaccgccgcgtgggtggccaat

SEQ ID NO: 245 - DNA, 38 bp, Influenza virus
  modified_base (5): Inosine
tagcnatttagggcctgcattgtcatataagcgaccgt

SEQ ID NO: 246 - DNA, 38 bp, Influenza virus
  modified_base (5): Inosine
tagcnatttagggtttgcattgtcatataagcgaccgt

SEQ ID NO: 247 - DNA, 39 bp, Influenza virus
  modified_base (5): Inosine
tagtnatttagggtttgtgctgtcatataagcggttgtt

SEQ ID NO: 248 - DNA, 33 bp, Influenza virus
  modified_base (5): Inosine
tagtnatttagggtttatgttgttatacaaacg

SEQ ID NO: 249 - DNA, 39 bp, Influenza virus
  modified_base (5): Inosine
tagtnatttagggtttatgttgttatacaaacagttgtg

SEQ ID NO: 250 - DNA, 39 bp, Influenza virus; identical to SEQ ID NO: 249
  modified_base (5): Inosine

SEQ ID NO: 251 - DNA, 38 bp, Influenza virus
  modified_base (5): Inosine
tagtnatttagggtttgtgttgttatacaaacggccgt

SEQ ID NO: 252 - DNA, 33 bp, Influenza virus
  modified_base (5): Inosine
tagtnatttagggtttgtgttgttatacaaacg

SEQ ID NO: 253 - DNA, 32 bp, Influenza virus
  modified_base (5): Inosine
tagtnacttagggcctgtgttgttatacaaac

SEQ ID NO: 254 - DNA, 39 bp, Influenza virus
  modified_base (5): Inosine
tagtnacttagggcctgtgttgttatacaaacaattgtg

SEQ ID NO: 255 - DNA, 39 bp, Influenza virus; identical to SEQ ID NO: 254
  modified_base (5): Inosine

SEQ ID NO: 256 - DNA, 38 bp, Influenza virus
  modified_base (5): Inosine
tagtnacttagggtttgtgttgttatacaaacagccgt

SEQ ID NO: 257 - DNA, 32 bp, Influenza virus
  modified_base (5): Inosine
tagtnacttagggtttgtgttgttatacaaac

SEQ ID NO: 258 - DNA, 39 bp, Influenza virus
  modified_base (5): Inosine
tagtnacttagggtttgtgttgttatacaaacagttgtg

SEQ ID NO: 259 - DNA, 39 bp, Influenza virus
  modified_base (5): Inosine
tagtnacttagggtttgtgttgttatacaaacaattgtg

SEQ ID NO: 260 - DNA, 39 bp, Influenza virus; identical to SEQ ID NO: 259
  modified_base (5): Inosine

SEQ ID NO: 261 - DNA, 33 bp, Influenza virus; identical to SEQ ID NO: 252
  modified_base (5): Inosine

SEQ ID NO: 262 - DNA, 33 bp, Influenza virus
  modified_base (5): Inosine
tagtnatttagggtttgtgttgttatacaaaca

SEQ ID NO: 263 - DNA, 39 bp, Influenza virus
  modified_base (5): Inosine
tagtnatttagggtttgtgttgttatacaaacggttgtg

SEQ ID NO: 264 - DNA, 39 bp, Influenza virus; identical to SEQ ID NO: 263
  modified_base (5): Inosine

SEQ ID NO: 265 - DNA, 28 bp, Influenza virus
  modified_base (5): Inosine
tagtnatttagggtttgtgttgttatac

SEQ ID NO: 266 - DNA, 39 bp, Influenza virus; identical to SEQ ID NO: 229

SEQ ID NO: 267 - DNA, 39 bp, Influenza virus; identical to SEQ ID NO: 230
  modified_base (5): Inosine

SEQ ID NO: 268 - DNA, 28 bp, Influenza virus
acgatgaataaatattcgtacttcaaag

SEQ ID NO: 269 - DNA, 28 bp, Influenza virus
ctaaaagcccggcgcgtacgtcctcgga

SEQ ID NO: 270 - DNA, 28 bp, Influenza virus
ctaaaagcccggcgcgtacgtcctcgga

SEQ ID NO: 271 - DNA, 28 bp, Influenza virus
ctaaaagcccggcgcgtacgtcctcgga

SEQ ID NO: 272 - DNA, 26 bp, Influenza virus
aggggcttcttaaacagattcggagt

SEQ ID NO: 273 - DNA, 26 bp, Influenza virus
gaaagtccttggtgtgagcctaaagc

SEQ ID NO: 274 - DNA, 26 bp, Influenza virus
agggacttcgtagacagattcgggtt

SEQ ID NO: 275 - DNA, 26 bp, Influenza virus
gaaagtccttggtgtgagcctaaagc

SEQ ID NO: 276 - DNA, 26 bp, Influenza virus
gaaagtccttggtgtgagcctaaagc

SEQ ID NO: 277 - DNA, 26 bp, Influenza virus
gaaagtccttggtgtgagcctaaagc

SEQ ID NO: 278 - DNA, 26 bp, Influenza virus
gaaagtccttggtgtgagcctaaagc

SEQ ID NO: 279 - DNA, 26 bp, Influenza virus
gaaagtccttggtgtgagcctaaagc

SEQ ID NO: 280 - DNA, 26 bp, Influenza virus
gaaagtccttggtgtgagcctaaagc

SEQ ID NO: 281 - DNA, 26 bp, Influenza virus
gaaagtccttggtgtgagcctaaagc

SEQ ID NO: 282 - DNA, 26 bp, Influenza virus
gaaagtccttggtgtgagcctaaagc

SEQ ID NO: 283 - DNA, 26 bp, Influenza virus
gaaagtccttggtgtgagcctaaagc

SEQ ID NO: 284 - DNA, 26 bp, Influenza virus
gaaagtccttggtgtgagcctaaagc

SEQ ID NO: 285 - DNA, 26 bp, Influenza virus
gaaagtccttggtgtgagcctaaagc

SEQ ID NO: 286 - DNA, 26 bp, Influenza virus
gaaagtccttggtgtgagcctaaagc

SEQ ID NO: 287 - DNA, 26 bp, Influenza virus
gaaagtccttggtgtgagcctaaagc

SEQ ID NO: 288 - DNA, 26 bp, Influenza virus
gaaagtccttggggtgagcctaaagc

SEQ ID NO: 289 - DNA, 26 bp, Influenza virus
gaaagtccttggggtgagcctaaagc

SEQ ID NO: 290 - DNA, 26 bp, Influenza virus
gagagtccttgatgtgggcccaaggc

SEQ ID NO: 291 - DNA, 26 bp, Influenza virus
agggacctcttagatagattcggagc

SEQ ID NO: 292 - DNA, 26 bp, Influenza virus
agagacttcttagacagattcggggt

SEQ ID NO: 293 - DNA, 26 bp, Influenza virus
agggacttcttagacagattcggggt

SEQ ID NO: 294 - DNA, 26 bp, Influenza virus
agggacttcttagacagattcggggt

SEQ ID NO: 295 - DNA, 26 bp, Influenza virus
agggacttcttagacagattcggggt

SEQ ID NO: 296 - DNA, 26 bp, Influenza virus
agggacttcttagacagattcggggt

SEQ ID NO: 297 - DNA, 26 bp, Influenza virus
agggacttcgtagacagattcggggt

SEQ ID NO: 298 - DNA, 26 bp, Influenza virus
agggacttcctagacagattcggggt

SEQ ID NO: 299 - DNA, 26 bp, Influenza virus
agggacttcgtagacagattcgggtc

SEQ ID NO: 300 - DNA, 26 bp, Influenza virus
agggacttcgtagacagattcgggtc

SEQ ID NO: 301 - DNA, 26 bp, Influenza virus
agggacttcgtagacagattcgggtt

SEQ ID NO: 302 - DNA, 26 bp, Influenza virus
agggacttcgtagacagattcgggtt

SEQ ID NO: 303 - DNA, 26 bp, Influenza virus
agggacttcgtagacagattcgggtt

SEQ ID NO: 304 - DNA, 26 bp, Influenza virus
agggacttcgtagacagattcgggtt

SEQ ID NO: 305 - DNA, 26 bp, Influenza virus
agggacttcgtagacagattcgggtt

SEQ ID NO: 306 - DNA, 26 bp, Influenza virus
agggacttcgtagacagattcgggtt

SEQ ID NO: 307 - DNA, 26 bp, Influenza virus
agggacttcgtagacagattcgggtt

SEQ ID NO: 308 - DNA, 26 bp, Influenza virus
agggacttcgtagacagattcgggtt

SEQ ID NO: 309 - DNA, 26 bp, Influenza virus
agggacttcgtagacagattcgggtt

SEQ ID NO: 310 - DNA, 26 bp, Influenza virus
aggaacttcgtagacagattcgggtt

SEQ ID NO: 311 - DNA, 26 bp, Influenza virus
aggagcttcgtagacagattcgggtt

SEQ ID NO: 312 - DNA, 26 bp, Influenza virus
gaaagtccttggtgtgagcctaaagc

SEQ ID NO: 313 - DNA, 26 bp, Influenza virus
agggacttcgtagacagattcgggtt

SEQ ID NO: 314 - DNA, 11 bp, Influenza virus
cgtagagccca

SEQ ID NO: 315 - DNA, 11 bp, Influenza virus
catgagacctg

SEQ ID NO: 316 - DNA, 11 bp, Influenza virus
tgcagagttca

SEQ ID NO: 317 - DNA, 11 bp, Influenza virus
cgtgagacctg

SEQ ID NO: 318 - DNA, 11 bp, Influenza virus
cgtgagacctg

SEQ ID NO: 319 - DNA, 11 bp, Influenza virus
cgtgagacctg

SEQ ID NO: 320 - DNA, 11 bp, Influenza virus
cgtgagacctg

SEQ ID NO: 321 - DNA, 11 bp, Influenza virus
catgagacctg

SEQ ID NO: 322 - DNA, 11 bp, Influenza virus
catgagacccg

SEQ ID NO: 323 - DNA, 11 bp, Influenza virus
catgagacccg

SEQ ID NO: 324 - DNA, 11 bp, Influenza virus
catgagacccg

SEQ ID NO: 325 - DNA, 11 bp, Influenza virus
catgagacccg

SEQ ID NO: 326 - DNA, 11 bp, Influenza virus
catgagacccg

SEQ ID NO: 327 - DNA, 11 bp, Influenza virus
catgagacccg

SEQ ID NO: 328 - DNA, 11 bp, Influenza virus
catgagacccg

SEQ ID NO: 329 - DNA, 11 bp, Influenza virus
catgagaccca

SEQ ID NO: 330 - DNA, 11 bp, Influenza virus
cgtgagacccg

SEQ ID NO: 331 - DNA, 11 bp, Influenza virus
cgtgagacccg

SEQ ID NO: 332 - DNA, 11 bp, Influenza virus
cgtgagacccg

SEQ ID NO: 333 - DNA, 11 bp, Influenza virus
cgtgagacccg

SEQ ID NO: 334 - DNA, 11 bp, Influenza virus
cgtgagaccca

SEQ ID NO: 335 - DNA, 11 bp, Influenza virus
cgtaaagccca

SEQ ID NO: 336 - DNA, 11 bp, Influenza virus
cgtagagccca

SEQ ID NO: 337 - DNA, 11 bp, Influenza virus
cgtagagccca

SEQ ID NO: 338 - DNA, 11 bp, Influenza virus
cgtagagccca

SEQ ID NO: 339 - DNA, 11 bp, Influenza virus
cgtagagccca

SEQ ID NO: 340 - DNA, 11 bp, Influenza virus
tgcagaacccg

SEQ ID NO: 341 - DNA, 11 bp, Influenza virus
cgtagagtcca

SEQ ID NO: 342 - DNA, 11 bp, Influenza virus
cgcagagtcca

SEQ ID NO: 343 - DNA, 11 bp, Influenza virus
cgcagagtcca

SEQ ID NO: 344 - DNA, 11 bp, Influenza virus
tgcagagttca

SEQ ID NO: 345 - DNA, 11 bp, Influenza virus
tgcagagttca

SEQ ID NO: 346 - DNA, 11 bp, Influenza virus
tgcagagttca

SEQ ID NO: 347 - DNA, 11 bp, Influenza virus
tgcagagttca

SEQ ID NO: 348 - DNA, 11 bp, Influenza virus
tgcagagttca

SEQ ID NO: 349 - DNA, 11 bp, Influenza virus
tgcagagttca

SEQ ID NO: 350 - DNA, 11 bp, Influenza virus
tgcagagttca

SEQ ID NO: 351 - DNA, 11 bp, Influenza virus
tgcagagttca

SEQ ID NO: 352 - DNA, 11 bp, Influenza virus
cgcagagttca

SEQ ID NO: 353 - DNA, 11 bp, Influenza virus
cgcagagttca

SEQ ID NO: 354 - DNA, 11 bp, Influenza virus
catgagacctg

SEQ ID NO: 355 - DNA, 11 bp, Influenza virus
tgcagagttca

SEQ ID NO: 356 - DNA, 12 bp, Influenza virus
gtgccaaagcgg

SEQ ID NO: 357 - DNA, 12 bp, Influenza virus
acgctgggatgt

SEQ ID NO: 358 - DNA, 12 bp, Influenza virus
gtatcaaagcag

SEQ ID NO: 359 - DNA, 10 bp, Influenza virus
gccaaagcgg

SEQ ID NO: 360 - DNA, 9 bp, Influenza virus
gccaaagcg

SEQ ID NO: 361 - DNA, 10 bp, Influenza virus
gccaaagcgg

SEQ ID NO: 362 - DNA, 10 bp, Influenza virus
gccaaagcgg

SEQ ID NO: 363 - DNA, 10 bp, Influenza virus
gccaaagcgg

SEQ ID NO: 364 - DNA, 10 bp, Influenza virus
gccaaagcgg

SEQ ID NO: 365 - DNA, 10 bp, Influenza virus
gccaaagcgg

SEQ ID NO: 366 - DNA, 10 bp, Influenza virus
gccaaagcgg

SEQ ID NO: 367 - DNA, 10 bp, Influenza virus
gccaaagcgg

SEQ ID NO: 368 - DNA, 10 bp, Influenza virus
gccaaagcgg

SEQ ID NO: 369 - DNA, 10 bp, Influenza virus
gccaaagcgg

SEQ ID NO: 370 - DNA, 10 bp, Influenza virus
gccaaagcgg

SEQ ID NO: 371 - DNA, 10 bp, Influenza virus
gccaaagcgg

SEQ ID NO: 372 - DNA, 12 bp, Influenza virus
gtgccaaagcgg

SEQ ID NO: 373 - DNA, 12 bp, Influenza virus
gtgccaaagcgg

SEQ ID NO: 374 - DNA, 12 bp, Influenza virus
gtgccaaagcgg

SEQ ID NO: 375 - DNA, 12 bp, Influenza virus
gtgccaaagcgg

SEQ ID NO: 376 - DNA, 10 bp, Influenza virus
gccaaagcgg

SEQ ID NO: 377 - DNA, 10 bp, Influenza virus
gccaaagcgg

SEQ ID NO: 378 - DNA, 10 bp, Influenza virus
gccaaagcgg

SEQ ID NO: 379 - DNA, 12 bp, Influenza virus
gtgccaaagcgg

SEQ ID NO: 380 - DNA, 10 bp, Influenza virus
gccaaagcgg

SEQ ID NO: 381 - DNA, 10 bp, Influenza virus
gccaaagcgg

SEQ ID NO: 382 - DNA, 12 bp, Influenza virus
atgccaaagcgg

SEQ ID NO: 383 - DNA, 10 bp, Influenza virus
gccaaagcgg

SEQ ID NO: 384 - DNA, 10 bp, Influenza virus
gccaaagcgg

SEQ ID NO: 385 - DNA, 10 bp, Influenza virus
gctaaggcgg

SEQ ID NO: 386 - DNA, 12 bp, Influenza virus
gtgctaaggcgg

SEQ ID NO: 387 - DNA, 10 bp, Influenza virus
accaaggcag

SEQ ID NO: 388 - DNA, 12 bp, Influenza virus
gtgccaaagcag

SEQ ID NO: 389 - DNA, 20 bp, Influenza virus
cagcggttacactcctgtga

SEQ ID NO: 390 - DNA, 20 bp, Influenza virus
agataacggtataatcacgg

SEQ ID NO: 391 - DNA, 20 bp, Influenza virus
agataacggtataatcacgg

SEQ ID NO: 392 - DNA, 20 bp, Influenza virus
cagcggctacactcctgtga

SEQ ID NO: 393 - DNA, 20 bp, Influenza virus
cagcggctacactcctgtga

SEQ ID NO: 394 - DNA, 20 bp, Influenza virus
aagcagctacacacttgcgg

SEQ ID NO: 395 - DNA, 20 bp, Influenza virus
aagcagttacacacttgcgg

SEQ ID NO: 396 - DNA, 20 bp, Influenza virus
aagcagcgacacactcgcgg

SEQ ID NO: 397 - DNA, 20 bp, Influenza virus
agatggcggtataatcacgg

SEQ ID NO: 398 - DNA, 20 bp, Influenza virus
aagcaacgacacactcacgg

SEQ ID NO: 399 - DNA, 110 bp, Influenza virus
gacgcgacatcagaaataacacgctaaaagatatgtctaagcacgctcagataatttttcctaatcaaattcgtatttaacagctatagttcccaagttaatgtccatga

SEQ ID NO: 400 - DNA, 110 bp, Influenza virus; identical to SEQ ID NO: 40

SEQ ID NO: 401 - DNA, 110 bp, Influenza virus; identical to SEQ ID NO: 41

SEQ ID NO: 402 - DNA, 110 bp, Influenza virus; identical to SEQ ID NO: 399

SEQ ID NO: 403 - DNA, 67 bp, Influenza virus
gacgcgacatcagaaataacacgctaaaagatatgtctaagcacgctcagataatttttcctaatca

SEQ ID NO: 404 - DNA, 73 bp, Influenza virus
gacgcgacatcagaaataacacgctaaaagatatgtctaagcacgctcagataatttttcctaatcaaattcg

SEQ ID NO: 405 - DNA, 73 bp, Influenza virus; identical to SEQ ID NO: 404

SEQ ID NO: 406 - DNA, 73 bp, Influenza virus; identical to SEQ ID NO: 404

SEQ ID NO: 407 - DNA, 73 bp, Influenza virus
gacgcgacatcagaaataacacgctaaaagatatgtctaagcacgctcagataatttttcctaatcgaattcg

SEQ ID NO: 408 - DNA, 73 bp, Influenza virus; identical to SEQ ID NO: 407

SEQ ID NO: 409 - DNA, 73 bp, Influenza virus; identical to SEQ ID NO: 407

SEQ ID NO: 410 - DNA, 73 bp, Influenza virus
gacgcgacatcagaaataacacgctaaaagatatgtctaagcacgctcagataatttttcctaatcgaattca

SEQ ID NO: 411 - DNA, 110 bp, Influenza virus
ggcgcgacatcagaaataacacgctaaaagatatgtctaagcacgctcagataatttttcctaatcaaattcgtatttaacagctatagttcccaagttaatgtccatga

SEQ ID NO: 412 - DNA, 109 bp, Influenza virus
ggcgcgacatcagaaataacacgctaaaagatatgtctaagcacgctcagataatttttcctaatcaaattcgtatttaacagctatagttcccaagttaatgtccatg

SEQ ID NO: 413 - DNA, 73 bp, Influenza virus
ggcgcgacatcagaaataacacgctaaaagatatgtctaagcacgctcagataatttttcctaatcaaattcg

SEQ ID NO: 414 - DNA, 73 bp, Influenza virus; identical to SEQ ID NO: 413

SEQ ID NO: 415 - DNA, 110 bp, Influenza virus
gacgcgacatcagaaataacacgctaaaaaatatgtctaagcacgctcagataatttttcccaatcaaattcgtatttaacagctatagttcccaagttaatgtccatga

SEQ ID NO: 416 - DNA, 73 bp, Influenza virus
gacgcgacatcagaaataacacgctaaaaaatatgtctaagcacgctcagataatttttcctaatcaaattcg

SEQ ID NO: 417 - DNA, 73 bp, Influenza virus; identical to SEQ ID NO: 404

SEQ ID NO: 418 - DNA, 73 bp, Influenza virus
gacgcgacatcagaaataacacgctaaaagatatgtctaagcacgcccagataattttttccaatcaaattcg

SEQ ID NO: 419 - DNA, 110 bp, Influenza virus
gacgcgacatcagaaataacacgctaaaagatatgtctaagcacgctcagataatttttcctaatcgaattcgtattcgatgactatagttcccaagttaatgtccatga

SEQ ID NO: 420 - DNA, 73 bp, Influenza virus
gacacaacatcagaaataacacgctaaaagatatgtctaagcacgctcagataatttttcctaatcaaattcg

SEQ ID NO: 421 - DNA, 73 bp, Influenza virus
gacgcgacatcagaaataacacgctaaaagatatgcctaagcacgcccagataatttttcccaatcaaattcg

SEQ ID NO: 422 - DNA, 107 bp, Influenza virus
gacgcgacatcagaaataacacactaaaagatatgcctaagcacgcccagataatttttcccaatcaaattcgtatttaacagctatagttcccaagttaatatcca

SEQ ID NO: 423 - DNA, 73 bp, Influenza virus; identical to SEQ ID NO: 421

SEQ ID NO: 424 - DNA, 73 bp, Influenza virus; identical to SEQ ID NO: 421

SEQ ID NO: 425 - DNA, 73 bp, Influenza virus; identical to SEQ ID NO: 421

SEQ ID NO: 426 - DNA, 107 bp, Influenza virus
gacgcgacatcagaaataacacgctaaaagatatgcctaagcacgcccagataatttttcccaatcaaattcgtatttaacagctatagttcccaagttaatgtcca

SEQ ID NO: 427 - DNA, 73 bp, Influenza virus
gacgcgacatcagaaataacacgctaaaggatatgtctaagcacacccgggtaatttttcccaatcaaattcg

SEQ ID NO: 428 - DNA, 90 bp, Influenza virus
agtataatacgagagatagcgaactaggaaatgtatttgcgctaacccaagcaaccctctccggcagaactcgtatttaacagctatagt

SEQ ID NO: 429 - DNA, 107 bp, Influenza virus
agtataatacgagagatagcgaactaggaaatgtatttgcgctaacccaaacaaccctctccggcagaactcgtatccgatgaccgaagtctctggactagtctcta

SEQ ID NO: 430 - DNA, 90 bp, Influenza virus
agtataatacgagagatagcgaactaggaaatgtatttgcgctaacccaaacaaccctctccggcagaactcgtatccgatgaccgaagt

SEQ ID NO: 431 - DNA, 107 bp, Influenza virus
agtataatacgagagatagcgaactaggaaatgtatttgcgctaacccaagtaaccttccccggccgaactcgtatccgatgaccgaagtctctggactagtctcta

SEQ ID NO: 432 - DNA, 73 bp, Influenza virus
agtataatacgagagatagtgaactaggaaatgtatttgcgctaacccaagcaaccttctccggcagaactcg

SEQ ID NO: 433 - DNA, 24 bp, Influenza virus
taaagtggattagtagaataacgc

SEQ ID NO: 434 - DNA, 24 bp, Influenza virus
tgaagtggattagtagaataacgc

SEQ ID NO: 435 - DNA, 13 bp, Influenza virus
taaggtggattag

SEQ ID NO: 436 - DNA, 14 bp, Influenza virus
taaggtggattagt

SEQ ID NO: 437 - DNA, 14 bp, Influenza virus
taaggtggattagt

SEQ ID NO: 438 - DNA, 14 bp, Influenza virus
taaggtggattagt

SEQ ID NO: 439 - DNA, 14 bp, Influenza virus
taaggtggattagt

SEQ ID NO: 440 - DNA, 14 bp, Influenza virus
taaggtggattagt

SEQ ID NO: 441 - DNA, 14 bp, Influenza virus
taaggtggattagt

SEQ ID NO: 442 - DNA, 14 bp, Influenza virus
taaggtggattagt

SEQ ID NO: 443 - DNA, 24 bp, Influenza virus
taaggtggattagtggaataatgc

SEQ ID NO: 444 - DNA, 24 bp, Influenza virus
taaggtggattagtggaataatgc

SEQ ID NO: 445 - DNA, 14 bp, Influenza virus
taaggtggattagt

SEQ ID NO: 446 - DNA, 14 bp, Influenza virus
taaggtggattagt

SEQ ID NO: 447 - DNA, 24 bp, Influenza virus
taaggtggattaatgggatgatgc

SEQ ID NO: 448 - DNA, 14 bp, Influenza virus
taaggtggattagt

SEQ ID NO: 449 - DNA, 14 bp, Influenza virus
taaggtggattagt

SEQ ID NO: 450 - DNA, 14 bp, Influenza virus
taaggtggattaat

SEQ ID NO: 451 - DNA, 24 bp, Influenza virus
taaggtggattagtggaataatgc

SEQ ID NO: 452 - DNA, 14 bp, Influenza virus
taaggtggattagt

SEQ ID NO: 453 - DNA, 14 bp, Influenza virus
taaggtgaattaat

SEQ ID NO: 454 - DNA, 23 bp, Influenza virus
caaggcgagacgatgggacggta

SEQ ID NO: 455 - DNA, 14 bp, Influenza virus
taaggtgaattaat

SEQ ID NO: 456 - DNA, 14 bp, Influenza virus
taaggtgaattaat

SEQ ID NO: 457 - DNA, 14 bp, Influenza virus
taaggtgaattaat

SEQ ID NO: 458 - DNA, 24 bp, Influenza virus
taaggtgaattaatgggatgatgc

SEQ ID NO: 459 - DNA, 14 bp, Influenza virus
taaggtggattaat

SEQ ID NO: 460 - DNA, 19 bp, Influenza virus
cagaataaagcgacgggac

SEQ ID NO: 461 - DNA, 23 bp, Influenza virus
cagaataaagcgacgaggcagcg

SEQ ID NO: 462 - DNA, 19 bp, Influenza virus
cagaataaagcgacgaggc

SEQ ID NO: 463 - DNA, 23 bp, Influenza virus
cagaataaattagtgaggcggcg

SEQ ID NO: 464 - DNA, 14 bp, Influenza virus
cagggtaaagcgac

SEQ ID NO: 465 - DNA, 113 bp, Influenza virus
tacgtattagatagactctggagaattcccgctttgctgacgctaatacaaaaagcccagttgaattccataatcgtttgtgcgtcgaaaaacgcaggcagaggggctaaccg

SEQ ID NO: 466 - DNA, 113 bp, Influenza virus; identical to SEQ ID NO: 77

SEQ ID NO: 467 - DNA, 113 bp, Influenza virus; identical to SEQ ID NO: 78

SEQ ID NO: 468 - DNA, 113 bp, Influenza virus; identical to SEQ ID NO: 465

SEQ ID NO: 469 - DNA, 46 bp, Influenza virus
ccataatcgtttgtgcgtcgaaaaacgcaggcagaggggctaaccg

SEQ ID NO: 470 - DNA, 46 bp, Influenza virus; identical to SEQ ID NO: 469

SEQ ID NO: 471 - DNA, 46 bp, Influenza virus; identical to SEQ ID NO: 469

SEQ ID NO: 472 - DNA, 46 bp, Influenza virus; identical to SEQ ID NO: 469

SEQ ID NO: 473 - DNA, 46 bp, Influenza virus
ccatagtcgtttgtgcgtcgaaaaacgcaggcagaggggctaaccg

SEQ ID NO: 474 - DNA, 46 bp, Influenza virus; identical to SEQ ID NO: 469

SEQ ID NO: 475 - DNA, 46 bp, Influenza virus
tcataatcgtttgtgcgtcgaaaaacgcaggcagaggggctaaccg

SEQ ID NO: 476 - DNA, 113 bp, Influenza virus
tacgtattagatagactctggagaattcccgctttgctgacgctaatacaaaaagcccagttgagttccataatcgtttgtgcgtcgaaaaacgcaggcagaggggctaaccg

SEQ ID NO: 477 - DNA, 113 bp, Influenza virus
tacgtattagataaactctggagaattcccgctttgctgacgctaatacaaaaagcccagttgagttccataatcgtttgtgcgtcgaaaaacgcaggcagaggggctaaccg

SEQ ID NO: 478 - DNA, 113 bp, Influenza virus
tacgtattagataaactctggaggattcccgctttgatgacgctaatacaaaaaacccagttgagttccataatcgtttgtgcgtcgaaaaacgcaggcagaggggctaaccg

SEQ ID NO: 479 - DNA, 113 bp, Influenza virus; identical to SEQ ID NO: 476

SEQ ID NO: 480 - DNA, 113 bp, Influenza virus; identical to SEQ ID NO: 476

SEQ ID NO: 481 - DNA, 113 bp, Influenza virus
tacgtattagatagactttggagaattcccgctttgctgacgctaatacgaaagacccagttgagtcccatgatcgtttgtgcgtcgaaaaacgcaggcagaggggctaaccg

SEQ ID NO: 482 - DNA, 113 bp, Influenza virus
tacgtattagatagactttggagaattcccgctttgctgacgccaatacgaaagacccagttgagtcccatgaccttccatgtagcagggggtataagaagaaaaaccaattg

SEQ ID NO: 483 - DNA, 113 bp, Influenza virus
tacgtattagatagactctggagaattcccgctttgctgatgctaatgcaaaaggcccagttgagttccataatcgtttgcgcgtcgaaaaacgcaggcagaggggctaaccg

SEQ ID NO: 484 - DNA, 113 bp, Influenza virus
tacgtattagatagactctggagaattcccgctttgctgatgccaatacaaaaggcccagttgagttccataatcgtttgcgcgtcgaaaaacgcaggcagaggggctaaccg

SEQ ID NO: 485 - DNA, 44 bp, Influenza virus
ataatcgtttgcgcgtcgaaaaacgcaggcagaggggctaaccg

SEQ ID NO: 486 - DNA, 44 bp, Influenza virus; identical to SEQ ID NO: 485

SEQ ID NO: 487 - DNA, 44 bp, Influenza virus; identical to SEQ ID NO: 485

SEQ ID NO: 488 - DNA, 44 bp, Influenza virus
ataatcgtttgtgcgtcgaaaaacgcatgcagaggggctaaccg

SEQ ID NO: 489 - DNA, 46 bp, Influenza virus; identical to SEQ ID NO: 469

SEQ ID NO: 490 - DNA, 45 bp, Influenza virus
ccataatcgtttgtgcgtcgaaaaacgcaggcagaggggctaacc

SEQ ID NO: 491 - DNA, 46 bp, Influenza virus; identical to SEQ ID NO: 469

SEQ ID NO: 492 - DNA, 42 bp, Influenza virus
aatcgtttgtgcgtcgaaaaacgcaggcagaggggctaaccg

SEQ ID NO: 493 - DNA, 46 bp, Influenza virus; identical to SEQ ID NO: 469

SEQ ID NO: 494 - DNA, 46 bp, Influenza virus
ccgtaatcgtttgtgcgtcgaaaaacgcaggcagaggggctaaccg

SEQ ID NO: 495 - DNA, 113 bp, Influenza virus
aacgcgctcaacaaacctcaatgaactcccgccccaccggtatcaatgcagaaaacctagccagacctcgtaaccttccatgtagcagggggtataagaaggaaaaccaattg

SEQ ID NO: 496 - DNA, 113 bp, Influenza virus
aacgcgctcaacaaacctcaatgaactcccgccccaccggtaccaatgcagaaaacctagccagacctcgtaaccttccatgtagcagggggtataagaagaaaaaccaattg

SEQ ID NO: 497 - DNA, 113 bp, Influenza virus; identical to SEQ ID NO: 496

SEQ ID NO: 498 - DNA, 113 bp, Influenza virus
aacgcgctcaacaaacctcaatgaactcccgccccaccgatgctaatacaaaaggcccagttgagttccataaccgtttgtgcgtcgaaaaacgcaggcagaggggctaatcg

SEQ ID NO: 499 - DNA, 20 bp, Influenza virus
ggaaacggcggtaaagcagg

SEQ ID NO: 500 - DNA, 20 bp, Influenza virus
ggaaacggcggtaaagcagg

SEQ ID NO: 501 - DNA, 20 bp, Influenza virus
agaactaacggaaatacgag

SEQ ID NO: 502 - DNA, 20 bp, Influenza virus
agaactaacggaaatacgag

SEQ ID NO: 503 - DNA, 20 bp, Influenza virus
agaactaacggaaatacggg

SEQ ID NO: 504 - DNA, 20 bp, Influenza virus
agaactaacggaaatacgag

SEQ ID NO: 505 - DNA, 20 bp, Influenza virus
agaactaacggaaatacgag

SEQ ID NO: 506 - DNA, 20 bp, Influenza virus
aaggctaacgaagataagag

SEQ ID NO: 507 - DNA, 20 bp, Influenza virus
aaggctaacgaagatagggg

SEQ ID NO: 508 - DNA, 20 bp, Influenza virus
aaaactaacggaaataagag

SEQ ID NO: 509 - DNA, 20 bp, Influenza virus
aaaactaacggaaataagag

SEQ ID NO: 510 - DNA, 20 bp, Influenza virus
aaggctaacgcaggtagggg

SEQ ID NO: 511 - DNA, 20 bp, Influenza virus
aaggctaacgcaggtagggg

SEQ ID NO: 512 - DNA, 20 bp, Influenza virus
aaggctaacgcaggtagggg

SEQ ID NO: 513 - DNA, 20 bp, Influenza virus
aaggctaacggaaataggag

SEQ ID NO: 514 - DNA, 18 bp, Influenza virus
cctgtgaccagacagtgc

SEQ ID NO: 515 - DNA, 18 bp, Influenza virus
tttttgatcagataccgc

SEQ ID NO: 516 - DNA, 18 bp, Influenza virus
cccgcagctgagccgtat

SEQ ID NO: 517 - DNA, 18 bp, Influenza virus
cttgtgaccagacagtgc

SEQ ID NO: 518 - DNA, 18 bp, Influenza virus
cctgtgaccagacagtgc

SEQ ID NO: 519 - DNA, 18 bp, Influenza virus
cctgtgaccagacagtgc

SEQ ID NO: 520 - DNA, 16 bp, Influenza virus
cctgtggccagacagt

SEQ ID NO: 521 - DNA, 18 bp, Influenza virus
cctgtggccagacagtgc

SEQ ID NO: 522 - DNA, 18 bp, Influenza virus
cctgtggccagacagtgc

SEQ ID NO: 523 - DNA, 18 bp, Influenza virus
cctgtggccagacagtgc

SEQ ID NO: 524 - DNA, 18 bp, Influenza virus
cctgtggccagacagtgc

SEQ ID NO: 525 - DNA, 16 bp, Influenza virus
cctgtggccagacagt

SEQ ID NO: 526 - DNA, 18 bp, Influenza virus
cctgtagccagacagtgc

SEQ ID NO: 527 - DNA, 18 bp, Influenza virus
cctgtagccagacagtgc

SEQ ID NO: 528 - DNA, 18 bp, Influenza virus
cctatagccagacagtgc

SEQ ID NO: 529 - DNA, 18 bp, Influenza virus
cctatagccagacagtgc

SEQ ID NO: 530 - DNA, 18 bp, Influenza virus
cctgtggccagacagtgc

SEQ ID NO: 531 - DNA, 18 bp, Influenza virus
cctgtgaccagacagtgc

SEQ ID NO: 532 - DNA, 18 bp, Influenza virus
cctgtgaccagacagtgc

SEQ ID NO: 533 - DNA, 18 bp, Influenza virus
cctgtgaccagacagtgc

SEQ ID NO: 534 - DNA, 18 bp, Influenza virus
cctgtggccagacagtgc

SEQ ID NO: 535 - DNA, 18 bp, Influenza virus
cctgtggccagacagtgc

SEQ ID NO: 536 - DNA, 18 bp, Influenza virus
cctgtggccagacagtgc

SEQ ID NO: 537 - DNA, 18 bp, Influenza virus
cctgtgtccagacagtgc

SEQ ID NO: 538 - DNA, 18 bp, Influenza virus
cctgtagccagacagtgc

SEQ ID NO: 539 - DNA, 18 bp, Influenza virus
cctgtagccagacagtgc

SEQ ID NO: 540 - DNA, 18 bp, Influenza virus
cctgtgtccagacagtgc

SEQ ID NO: 541 - DNA, 18 bp, Influenza virus
cctgtgtccagacagtgc

SEQ ID NO: 542 - DNA, 18 bp, Influenza virus
cctgtggccagacagtgc

SEQ ID NO: 543 - DNA, 18 bp, Influenza virus
cctgtggccagacagtgc

SEQ ID NO: 544 - DNA, 18 bp, Influenza virus
cctttggccagacagtgc

SEQ ID NO: 545 - DNA, 18 bp, Influenza virus
cctttggccagacagtgc

SEQ ID NO: 546 - DNA, 18 bp, Influenza virus
cctttggccagacagtgc

SEQ ID NO: 547 - DNA, 18 bp, Influenza virus
cctttggccagacagtgc

SEQ ID NO: 548 - DNA, 18 bp, Influenza virus
cctttagccagacagtgc

SEQ ID NO: 549 - DNA, 18 bp, Influenza virus
cctgtggccagacagtgc

SEQ ID NO: 550 - DNA, 18 bp, Influenza virus
ttgtagatggtgggaatg

SEQ ID NO: 551 - DNA, 18 bp, Influenza virus
ccacgagcaccaaacgaa

SEQ ID NO: 552 - DNA, 18 bp, Influenza virus
ccacgagcaccaaacgaa

SEQ ID NO: 553 - DNA, 18 bp, Influenza virus
ttgtagatggtgggaatg

SEQ ID NO: 554 - DNA, 17 bp, Influenza virus
ttgtagatggtgggaat

SEQ ID NO: 555 - DNA, 18 bp, Influenza virus
ttgtagatggtgggaatg

SEQ ID NO: 556 - DNA, 13 bp, Influenza virus
ttgtagatggtgg

SEQ ID NO: 557 - DNA, 16 bp, Influenza virus
tgtagatggtgggaat

SEQ ID NO: 558 - DNA, 18 bp, Influenza virus
ttgtagatggtgggaatg

SEQ ID NO: 559 - DNA, 17 bp, Influenza virus
ttgtagatggtgggaat

SEQ ID NO: 560 - DNA, 17 bp, Influenza virus
ctgtagatggtgggaat

SEQ ID NO: 561 - DNA, 14 bp, Influenza virus
ctgtagatggtggg

SEQ ID NO: 562 - DNA, 18 bp, Influenza virus
ttgtaggtggtaggaatg

SEQ ID NO: 563 - DNA, 17 bp, Influenza virus
ttgtagatggtaggaat

SEQ ID NO: 564 - DNA, 17 bp, Influenza virus
ctgtagatggtgggaat

SEQ ID NO: 565 - DNA, 17 bp, Influenza virus
ttgtagatggtgggaat

SEQ ID NO: 566 - DNA, 17 bp, Influenza virus
tcgtagatggcgggaaa

SEQ ID NO: 567 - DNA, 17 bp, Influenza virus
tcgtagacggtcggaat

SEQ ID NO: 568 - DNA, 17 bp, Influenza virus
tcgtagacggtcggaat

SEQ ID NO: 569 - DNA, 18 bp, Influenza virus
tcacagacggcaggaaat

SEQ ID NO: 570 - DNA, 17 bp, Influenza virus
ctgtgagtactgagaat

SEQ ID NO: 571 - DNA, 18 bp, Influenza virus
ccacgagcaccaagaaaa

SEQ ID NO: 572 - DNA, 17 bp, Influenza virus
ccacgagcaccaagaaa

SEQ ID NO: 573 - DNA, 18 bp, Influenza virus
ccacgagcaccaagaaaa

SEQ ID NO: 574 - DNA, 18 bp, Influenza virus
ccacagacggcgggaaag

SEQ ID NO: 575 - DNA, 18 bp, Influenza virus
ccacagatggtaggaaag

SEQ ID NO: 576 - DNA, 17 bp, Influenza virus
ctacagacggcggaaaa

SEQ ID NO: 577 - DNA, 18 bp, Influenza virus
ccacagatggcgggagaa

SEQ ID NO: 578 - DNA, 17 bp, Influenza virus
catagacggcagacgaa

SEQ ID NO: 579 - DNA, 18 bp, Influenza virus
ccatagacggcagacgaa

SEQ ID NO: 580 - DNA, 18 bp, Influenza virus
ccacagacggcagacgaa

SEQ ID NO: 581 - DNA, 18 bp, Influenza virus
tcacagacggcagacgaa

SEQ ID NO: 582 - DNA, 17 bp, Influenza virus
tcacgagcaccaaacga

SEQ ID NO: 583 - DNA, 17 bp, Influenza virus
tcacgagcaccaaacga

SEQ ID NO: 584 - DNA, 18 bp, Influenza virus
ccacgagcaccaaacgaa

SEQ ID NO: 585 - DNA, 18 bp, Influenza virus
ccatgagcaccaaacgaa

SEQ ID NO: 586 - DNA, 63 bp, Influenza virus
ttttggaggacgtcaagcgcacaccaactaaattacgcgacgttgtatgtctagatacgagta

SEQ ID NO: 587 - DNA, 63 bp, Influenza virus; identical to SEQ ID NO: 153

SEQ ID NO: 588 - DNA, 63 bp, Influenza virus; identical to SEQ ID NO: 154

SEQ ID NO: 589 - DNA, 62 bp, Influenza virus
ttttggaggacgtcacgcgcacaccaactaaattacgcgacgttgtatgtctagatacgagt

SEQ ID NO: 590 - DNA, 43 bp, Influenza virus
ttttgggggacgtcacgcgcacaccaactaaattacgcgacgt

SEQ ID NO: 591 - DNA, 46 bp, Influenza virus
ttttgggggacgtcacgcgcacaccagctaaattacgcgacgttat

SEQ ID NO: 592 - DNA, 44 bp, Influenza virus
ttttgggggacgtcacgcgcacaccagctaaattacgcgacgtt

SEQ ID NO: 593 - DNA, 28 bp, Influenza virus
ttttgggggacgtcacgcgcacaccaac

SEQ ID NO: 594 - DNA, 63 bp, Influenza virus
ttttgggggacgtcacgcgcacaccaactaaattacgcgacgttgtatgtctaaatacgggta

SEQ ID NO: 595 - DNA, 44 bp, Influenza virus
ttttgggggacgtcacgcgcacaccaactaaattacgcgacgtt

SEQ ID NO: 596 - DNA, 46 bp, Influenza virus
ttttgggggacgtcacgcgcacaccaactaaattacgcgacgttat

SEQ ID NO: 597 - DNA, 46 bp, Influenza virus; identical to SEQ ID NO: 596

SEQ ID NO: 598 - DNA, 46 bp, Influenza virus; identical to SEQ ID NO: 596

SEQ ID NO: 599 - DNA, 46 bp, Influenza virus; identical to SEQ ID NO: 596

SEQ ID NO: 600 - DNA, 46 bp, Influenza virus; identical to SEQ ID NO: 596

SEQ ID NO: 601 - DNA, 46 bp, Influenza virus; identical to SEQ ID NO: 596

SEQ ID NO: 602 - DNA, 46 bp, Influenza virus; identical to SEQ ID NO: 596

SEQ ID NO: 603 - DNA, 43 bp, Influenza virus; identical to SEQ ID NO: 590

SEQ ID NO: 604 - DNA, 44 bp, Influenza virus; identical to SEQ ID NO: 595

SEQ ID NO: 605 - DNA, 43 bp, Influenza virus; identical to SEQ ID NO: 590

SEQ ID NO: 606 - DNA, 43 bp, Influenza virus; identical to SEQ ID NO: 590

SEQ ID NO: 607 - DNA, 63 bp, Influenza virus
ttttgggggacgtcacgcgcacaccaactaaattacgcgacgttgtatgtctaaatacgagta

SEQ ID NO: 608 - DNA, 63 bp, Influenza virus
ttttgggggacgtcacgcgcacaccaaccaaattacgcgacgttgtatgtctaaatacgagta

SEQ ID NO: 609 - DNA, 63 bp, Influenza virus
ttttaggggacgtcacgcgcacaccaactaaattacgcgacgttgtatgtctaaatacgagta

SEQ ID NO: 610 - DNA, 63 bp, Influenza virus; identical to SEQ ID NO: 607

SEQ ID NO: 611 - DNA, 43 bp, Influenza virus; identical to SEQ ID NO: 590

SEQ ID NO: 612 - DNA, 63 bp, Influenza virus; identical to SEQ ID NO: 607

SEQ ID NO: 613 - DNA, 43 bp, Influenza virus; identical to SEQ ID NO: 590

SEQ ID NO: 614 - DNA, 63 bp, Influenza virus; identical to SEQ ID NO: 607

SEQ ID NO: 615 - DNA, 63 bp, Influenza virus
ttttgggggacgtcacgcgcacaccaactaaattatatggcaccatgtgttcataaatgagct

SEQ ID NO: 616 - DNA, 63 bp, Influenza virus
ttttgggggacgtcacgcgcacaccaactaaattacgcgacgttgtatgtctaaatacaagta

SEQ ID NO: 617 - DNA, 63 bp, Influenza virus; identical to SEQ ID NO: 616

SEQ ID NO: 618 - DNA, 44 bp, Influenza virus; identical to SEQ ID NO: 595

SEQ ID NO: 619 - DNA, 63 bp, Influenza virus; identical to SEQ ID NO: 607

SEQ ID NO: 620 - DNA, 62 bp, Influenza virus
gacccacaacgttgctcatgagagaggaccagagaatattcgacgtagcggacgctcaagaa

SEQ ID NO: 621 - DNA, 62 bp, Influenza virus
gacccacaacgttgcttatgagagaggaccagagaatattcgacgtagcggacgctcaagaa

SEQ ID NO: 622 - DNA, 42 bp, Influenza virus
gacccacaacgttgcttatgagagaggaccagaaaatattcg

SEQ ID NO: 623 - DNA, 43 bp, Influenza virus
gacccacaacgttgctcatgagagaggaccagaaaatattcgg

SEQ ID NO: 624 - DNA, 43 bp, Influenza virus
gacccacaacgttgctcatgagagaggaccagagaatattcgg

SEQ ID NO: 625 - DNA, 31 bp, Influenza virus
gacccacaacgttgctcatgagagaggacca

SEQ ID NO: 626 - DNA, 62 bp, Influenza virus
gacccacaacgttgctcatgagagaggaccagagaatattcgacgtagaggacgctcaagaa

SEQ ID NO: 627 - DNA, 42 bp, Influenza virus
gacccacaacgttgctcataagagaggaccagagaatattcg

SEQ ID NO: 628 - DNA, 43 bp, Influenza virus; identical to SEQ ID NO: 624

SEQ ID NO: 629 - DNA, 43 bp, Influenza virus; identical to SEQ ID NO: 624

SEQ ID NO: 630 - DNA, 43 bp, Influenza virus; identical to SEQ ID NO: 624

SEQ ID NO: 631 - DNA, 43 bp, Influenza virus; identical to SEQ ID NO: 624

SEQ ID NO: 632 - DNA, 43 bp, Influenza virus; identical to SEQ ID NO: 624

SEQ ID NO: 633 - DNA, 43 bp, Influenza virus; identical to SEQ ID NO: 624

SEQ ID NO: 634 - DNA, 43 bp, Influenza virus; identical to SEQ ID NO: 624

SEQ ID NO: 635 - DNA, 42 bp, Influenza virus
gacccacaacgttgctcatgagagaggaccagagaatattcg

SEQ ID NO: 636 - DNA, 43 bp, Influenza virus
gacccacaacgttgctcatgagagaggaccagagaatattcga

SEQ ID NO: 637 - DNA, 42 bp, Influenza virus
gacccacaacgttgctcatgagggaggaccagagaatattcg

SEQ ID NO: 638 - DNA, 42 bp, Influenza virus
gacccacaatgttgctcatgagagaggaccagagaatattcg

SEQ ID NO: 639 - DNA, 62 bp, Influenza virus; identical to SEQ ID NO: 620

SEQ ID NO: 640 - DNA, 62 bp, Influenza virus; identical to SEQ ID NO: 620

SEQ ID NO: 641 - DNA, 62 bp, Influenza virus; identical to SEQ ID NO: 620

SEQ ID NO: 642 - DNA, 62 bp, Influenza virus
gacccacaacgttgctcatgagagaggaccagagaatattcgacgtagcggacgctctagaa

SEQ ID NO: 643 - DNA, 42 bp, Influenza virus; identical to SEQ ID NO: 635

SEQ ID NO: 644 - DNA, 62 bp, Influenza virus; identical to SEQ ID NO: 620

SEQ ID NO: 645 - DNA, 42 bp, Influenza virus; identical to SEQ ID NO: 635

SEQ ID NO: 646 - DNA, 62 bp, Influenza virus; identical to SEQ ID NO: 620

SEQ ID NO: 647 - DNA, 62 bp, Influenza virus
gacccacgacgttgctcatgagagagggctagaggtcttgaagtaggacaaatatatggatg

SEQ ID NO: 648 - DNA, 62 bp, Influenza virus
gacccacgacgttgctcatgagagagggctagagaatattcgacgtagcggacgctcaggaa

SEQ ID NO: 649 - DNA, 62 bp, Influenza virus; identical to SEQ ID NO: 648

SEQ ID NO: 650 - DNA, 43 bp, Influenza virus
gacccacggcgttgctcatgagagagggctagagaatattcga

SEQ ID NO: 651 - DNA, 62 bp, Influenza virus
gacccacaacgttgctcatgagagagggctagagaatattcgacgtagcggacgctcaagaa

SEQ ID NO: 652 - DNA, 39 bp, Influenza virus; identical to SEQ ID NO: 228

SEQ ID NO: 653 - DNA, 39 bp, Influenza virus; identical to SEQ ID NO: 229

SEQ ID NO: 654 - DNA, 39 bp, Influenza virus; identical to SEQ ID NO: 230
  modified_base (5): Inosine

SEQ ID NO: 655 - DNA, 39 bp, Influenza virus
caatdatttggagcccacaccgtcgtgtaggcggctaac

SEQ ID NO: 656 - DNA, 31 bp, Influenza virus
caatdatccggggcccacaccgtcgtgtaag

SEQ ID NO: 657 - DNA, 32 bp, Influenza virus
caatdatccggggcccacaccgtcgtgtaagc

SEQ ID NO: 658 - DNA, 33 bp, Influenza virus
caatdatccggggcccacaccgtcgtgtaagcg

SEQ ID NO: 659 - DNA, 34 bp, Influenza virus
caatdatccagggcccacaccgtcgtgtaagcgg

SEQ ID NO: 660 - DNA, 32 bp, Influenza virus
caatdatccggggcccacaccgtcgtgtaggc

SEQ ID NO: 661 - DNA, 33 bp, Influenza virus
caatdatccggggcccacaccgtcgtgtaggcg

SEQ ID NO: 662 - DNA, 39 bp, Influenza virus
caatdatccggggcccacaccgtcgtgtaggcggctaat

SEQ ID NO: 663 - DNA, 39 bp, Influenza virus; identical to SEQ ID NO: 662

SEQ ID NO: 664 - DNA, 32 bp, Influenza virus; identical to SEQ ID NO: 660

SEQ ID NO: 665 - DNA, 39 bp, Influenza virus
caatdatccggggcccacaccgttgtgtaggcggctaat

SEQ ID NO: 666 - DNA, 39 bp, Influenza virus
caatdattcgaggcccacaccgttgtgtaggcggctaat

SEQ ID NO: 667 - DNA, 39 bp, Influenza virus; identical to SEQ ID NO: 665

SEQ ID NO: 668 - DNA, 33 bp, Influenza virus
caatdatccggggcccacaccgttgtgtaggcg

SEQ ID NO: 669 - DNA, 33 bp, Influenza virus; identical to SEQ ID NO: 668

SEQ ID NO: 670 - DNA, 33 bp, Influenza virus
caatdatccgggacccacaccgttgtgtaggcg

SEQ ID NO: 671 - DNA, 33 bp, Influenza virus; identical to SEQ ID NO: 670

SEQ ID NO: 672 - DNA, 33 bp, Influenza virus; identical to SEQ ID NO: 670

SEQ ID NO: 673 - DNA, 39 bp, Influenza virus
taatdatacggggcccacaccgttgtgtaggcggccaat

SEQ ID NO: 674 - DNA, 39 bp, Influenza virus
taatdatacggggcccacaccgtcgtgtaggcggctaat

SEQ ID NO: 675 - DNA, 39 bp, Influenza virus
caatdatccggggcccacaccgtcgtgtaagcggctaat

SEQ ID NO: 676 - DNA, 33 bp, Influenza virus
cagtdatccggggcccgcaccgccgtgtaggcg

SEQ ID NO: 677 - DNA, 29 bp, Influenza virus
cagcdatccggggcccgcaccgccgtgta

SEQ ID NO: 678 - DNA, 33 bp, Influenza virus; identical to SEQ ID NO: 676

SEQ ID NO: 679 - DNA, 33 bp, Influenza virus; identical to SEQ ID NO: 676

SEQ ID NO: 680 - DNA, 26 bp, Influenza virus
ccggggcccgcaccgccgtgtaggcg

SEQ ID NO: 681 - DNA, 39 bp, Influenza virus
  modified_base (5): Inosine
tagcnatttagggttcatgttgttatacgaacggttgtt

SEQ ID NO: 682 - DNA, 39 bp, Influenza virus; identical to SEQ ID NO: 681
  modified_base (5): Inosine

SEQ ID NO: 683 - DNA, 33 bp, Influenza virus
  modified_base (5): Inosine
tagcnatttagggttcatgttgttatacgaacg

SEQ ID NO: 684 - DNA, 32 bp, Influenza virus
  modified_base (5): Inosine
tagtnatttagggttcatgttgttatacgaac

SEQ ID NO: 685 - DNA, 33 bp, Influenza virus
  modified_base (5): Inosine
tagtnatttagggtttgcgttgttatacgaacg

SEQ ID NO: 686 - DNA, 13 bp, Influenza virus
cgtataagtactt

SEQ ID NO: 687 - DNA, 13 bp, Influenza virus
tacgcgtatgtcc

SEQ ID NO: 688 - DNA, 13 bp, Influenza virus
tacgcgtatgtcc

SEQ ID NO: 689 - DNA, 13 bp, Influenza virus
cgtataagtactt

SEQ ID NO: 690 - DNA, 13 bp, Influenza virus
cgtataagtgctt

SEQ ID NO: 691 - DNA, 13 bp, Influenza virus
cgtataagtgctt

SEQ ID NO: 692 - DNA, 13 bp, Influenza virus
cgtataagtgctt

SEQ ID NO: 693 - DNA, 13 bp, Influenza virus
cgtataagtgctt

SEQ ID NO: 694 - DNA, 13 bp, Influenza virus
cgtacaagtgctt

SEQ ID NO: 695 - DNA, 13 bp, Influenza virus
cgtacaagtgctt

SEQ ID NO: 696 - DNA, 13 bp, Influenza virus
cgtacaagtgctt

SEQ ID NO: 697 - DNA, 13 bp, Influenza virus
cgtacaagtgctt

SEQ ID NO: 698 - DNA, 13 bp, Influenza virus
cgtacaagtgctt

SEQ ID NO: 699 - DNA, 13 bp, Influenza virus
cgtataagtgctt

SEQ ID NO: 700 - DNA, 13 bp, Influenza virus
tgtataagtgctc

SEQ ID NO: 701 - DNA, 13 bp, Influenza virus
tgtataagtgctc

SEQ ID NO: 702 - DNA, 13 bp, Influenza virus
tgtataagtgctc

SEQ ID NO: 703 - DNA, 13 bp, Influenza virus
tgtataagtgctc

SEQ ID NO: 704 - DNA, 13 bp, Influenza virus
tgtataagtgctc

SEQ ID NO: 705 - DNA, 13 bp, Influenza virus
tgtataagtgctc

SEQ ID NO: 706 - DNA, 13 bp, Influenza virus
tgtataagtgctc

SEQ ID NO: 707 - DNA, 13 bp, Influenza virus
tgtataagtactc

SEQ ID NO: 708 - DNA, 13 bp, Influenza virus
tgtataagtgctc

SEQ ID NO: 709 - DNA, 13 bp, Influenza virus
tgtataagtgctc

SEQ ID NO: 710 - DNA, 13 bp, Influenza virus
tacacgaatgtcc

SEQ ID NO: 711 - DNA, 11 bp, Influenza virus
tacacacgtgt

SEQ ID NO: 712 - DNA, 13 bp, Influenza virus
tacgcgcatgtcc

SEQ ID NO: 713 - DNA, 13 bp, Influenza virus
tacgcgcatgtcc

SEQ ID NO: 714 - DNA, 10 bp, Influenza virus
gcgcatgtcc

SEQ ID NO: 715 - DNA, 13 bp, Influenza virus
tacgcgtatgtcc

SEQ ID NO: 716 - DNA, 13 bp, Influenza virus
tacgcgtatgtcc

SEQ ID NO: 717 - DNA, 13 bp, Influenza virus
tacgcgtatgtcc

SEQ ID NO: 718 - DNA, 13 bp, Influenza virus
tacgcgtatgtcc

SEQ ID NO: 719 - DNA, 13 bp, Influenza virus
tacgcgtatgtcc

SEQ ID NO: 720 - DNA, 12 bp, Influenza virus
agtaaattccaa

SEQ ID NO: 721 - DNA, 12 bp, Influenza virus
caaccgcgttcg

SEQ ID NO: 722 - DNA, 12 bp, Influenza virus
caaccgcgttcg

SEQ ID NO: 723 - DNA, 12 bp, Influenza virus
agtaaattccaa

SEQ ID NO: 724 - DNA, 11 bp, Influenza virus
caaccgcgttc

SEQ ID NO: 725 - DNA, 11 bp, Influenza virus
caaccgcgttc

SEQ ID NO: 726 - DNA, 11 bp, Influenza virus
caaccgcgttc

SEQ ID NO: 727 - DNA, 11 bp, Influenza virus
caaccgcgttc

SEQ ID NO: 728 - DNA, 11 bp, Influenza virus
caaccgcgttc

SEQ ID NO: 729 - DNA, 10 bp, Influenza virus
caaccgcgtt

SEQ ID NO: 730 - DNA, 12 bp, Influenza virus
caaccgcgttcg

SEQ ID NO: 731 - DNA, 12 bp, Influenza virus
caaccgcgttcg

SEQ ID NO: 732 - DNA, 9 bp, Influenza virus
caaccgcgt

SEQ ID NO: 733 - DNA, 12 bp, Influenza virus
caaccgcgttcg

SEQ ID NO: 734 - DNA, 12 bp, Influenza virus
caaccgcgttcg

SEQ ID NO: 735 - DNA, 12 bp, Influenza virus
caaccgccttcg

SEQ ID NO: 736 - DNA, 11 bp, Influenza virus
caaccgcgttc

SEQ ID NO: 737 - DNA, 10 bp, Influenza virus
caaccgcgtt

SEQ ID NO: 738 - DNA, 11 bp, Influenza virus
caaccgcgttc

SEQ ID NO: 739 - DNA, 11 bp, Influenza virus
caaccgcgttc

SEQ ID NO: 740 - DNA, 11 bp, Influenza virus
caaccgcgttc

SEQ ID NO: 741 - DNA, 11 bp, Influenza virus
caaccgcgttc

SEQ ID NO: 742 - DNA, 12 bp, Influenza virus
caaccgcgttcg

SEQ ID NO: 743 - DNA, 12 bp, Influenza virus
caaccgcgttcg

SEQ ID NO: 744 - DNA, 9 bp, Influenza virus
caaccgcgt

SEQ ID NO: 745 - DNA, 9 bp, Influenza virus
caaccgcgt

SEQ ID NO: 746 - DNA, 11 bp, Influenza virus
caaccgcgttc

SEQ ID NO: 747 - DNA, 11 bp, Influenza virus
caaccgcgttc

SEQ ID NO: 748 - DNA, 6 bp, Influenza virus
gcgttc

SEQ ID NO: 749 - DNA, 12 bp, Influenza virus
caaccgcgttcg

SEQ ID NO: 750 - DNA, 12 bp, Influenza virus
caaccgcgttcg

SEQ ID NO: 751 - DNA, 11 bp, Influenza virus
caaccgcgttc

SEQ ID NO: 752 - DNA, 11 bp, Influenza virus
caaccgcgttc

SEQ ID NO: 753 - DNA, 11 bp, Influenza virus
caaccgcgttc

SEQ ID NO: 754 - DNA, 26 bp, Influenza virus
aggggcttcttaaacagattcggagt

SEQ ID NO: 755 - DNA, 26 bp, Influenza virus
gaaagtccttggtgtgagcctaaagc

SEQ ID NO: 756 - DNA, 26 bp, Influenza virus
agggacttcgtagacagattcgggtt

SEQ ID NO: 757 - DNA, 26 bp, Influenza virus
aggggcttcttagacagattcggagt

SEQ ID NO: 758 - DNA, 26 bp, Influenza virus
aggggcttcttagacagattcggagt

SEQ ID NO: 759 - DNA, 26 bp, Influenza virus
aggggcttcttagacagattcggagt

SEQ ID NO: 760 - DNA, 26 bp, Influenza virus
aggggcttcttagacagattcggagt

SEQ ID NO: 761 - DNA, 26 bp, Influenza virus
aggggcttcttagacagattcggagt

SEQ ID NO: 762 - DNA, 26 bp, Influenza virus
aggggcttcttagacagattcggagt

SEQ ID NO: 763 - DNA, 25 bp, Influenza virus
aggggcttcttagacagattcggag

SEQ ID NO: 764 - DNA, 25 bp, Influenza virus
ggggcttcttagacagattcggagt

SEQ ID NO: 765 - DNA, 26 bp, Influenza virus
aggggcttcttagacagattcggagt

SEQ ID NO: 766 - DNA, 26 bp, Influenza virus
aggggcttcttagacagattcggagt

SEQ ID NO: 767 - DNA, 26 bp, Influenza virus
aggggcttcttagacagattcggagt

SEQ ID NO: 768 - DNA, 26 bp, Influenza virus
aggggcttcttagacagattcggagt

SEQ ID NO: 769 - DNA, 26 bp, Influenza virus
aggggcttcttagacagattcggagt

SEQ ID NO: 770 - DNA, 26 bp, Influenza virus
aggggcttcttagacagattcggagt

SEQ ID NO: 771 - DNA, 26 bp, Influenza virus
aggggcttcttagacagattcggagt

SEQ ID NO: 772 - DNA, 25 bp, Influenza virus
aggggcttcttatgcagattcggag

SEQ ID NO: 773 - DNA, 25 bp, Influenza virus
aggggcttcttagacagattcggag

SEQ ID NO: 774 - DNA, 26 bp, Influenza virus
agaggcttcttagacagattcggagt

SEQ ID NO: 775 - DNA, 26 bp, Influenza virus
aggggcttcttagacagattcggagt

SEQ ID NO: 776 - DNA, 26 bp, Influenza virus
aggggtttcttagacagattcggagt

SEQ ID NO: 777 - DNA, 26 bp, Influenza virus
gggggcttcttagacagattcggagt

SEQ ID NO: 778 - DNA, 26 bp, Influenza virus
aggggcttcttagacagattcggagt

SEQ ID NO: 779 - DNA, 26 bp, Influenza virus
aggggcttcttagacagattcggagt

SEQ ID NO: 780 - DNA, 26 bp, Influenza virus
aggggcttcttagacagattcggagt

SEQ ID NO: 781 - DNA, 26 bp, Influenza virus
agggacttcctagacagattcggggt

SEQ ID NO: 782 - DNA, 26 bp, Influenza virus
agggacttcctagacagattcggggt

SEQ ID NO: 783 - DNA, 26 bp, Influenza virus
agggacttcctagacagattcggggt

SEQ ID NO: 784 - DNA, 25 bp, Influenza virus
gggggctctttatgcggatccgaag

SEQ ID NO: 785 - DNA, 26 bp, Influenza virus
gggggctctttatgcggatccgaagc

SEQ ID NO: 786 - DNA, 26 bp, Influenza virus
aggggctctttatgcggatccgaagc

SEQ ID NO: 787 - DNA, 26 bp, Influenza virus
gggggctctttatgcggatccgaagc

SEQ ID NO: 788 - DNA, 26 bp, Influenza virus
aagagccccttaaacgggcacagagc

SEQ ID NO: 789 - DNA, 11 bp, Influenza virus
cgtagaggcca

SEQ ID NO: 790 - DNA, 11 bp, Influenza virus
catgagacctg

SEQ ID NO: 791 - DNA, 11 bp, Influenza virus
tgcagagttca

SEQ ID NO: 792 - DNA, 11 bp, Influenza virus
cgtagaggcca

SEQ ID NO: 793 - DNA, 11 bp, Influenza virus
cgtagaggcca

SEQ ID NO: 794 - DNA, 11 bp, Influenza virus
cgtagaggcca

SEQ ID NO: 795 - DNA, 11 bp, Influenza virus
cgtagaggcca

SEQ ID NO: 796 - DNA, 11 bp, Influenza virus
cgtagaggcca

SEQ ID NO: 797 - DNA, 11 bp, Influenza virus
cgtagaggcca

SEQ ID NO: 798 - DNA, 11 bp, Influenza virus
cgtagaggcca

SEQ ID NO: 799 - DNA, 11 bp, Influenza virus
cgtagaggcca

SEQ ID NO: 800 - DNA, 11 bp, Influenza virus
cgtagaggcca

SEQ ID NO: 801 - DNA, 11 bp, Influenza virus
cgtagaggcca

SEQ ID NO: 802 - DNA, 11 bp, Influenza virus
cgtagaggcca

SEQ ID NO: 803 - DNA, 11 bp, Influenza virus
cgtagaggcca

SEQ ID NO: 804 - DNA, 11 bp, Influenza virus
cgtagaggcca

SEQ ID NO: 805 - DNA, 11 bp, Influenza virus
cgtagaggcca

SEQ ID NO: 806 - DNA, 11 bp, Influenza virus
cgtagaggcca

SEQ ID NO: 807 - DNA, 11 bp, Influenza virus
cgtagaggcca

SEQ ID NO: 808 - DNA, 11 bp, Influenza virus
cgtagaggcca

SEQ ID NO: 809 - DNA, 11 bp, Influenza virus
cgtagaggcca

SEQ ID NO: 810 - DNA, 11 bp, Influenza virus
cgtagaggcca

SEQ ID NO: 811 - DNA, 11 bp, Influenza virus
cgtagaggcca

SEQ ID NO: 812 - DNA, 11 bp, Influenza virus
cgtagaggcca

SEQ ID NO: 813 - DNA, 11 bp, Influenza virus
cgtagagtcca

SEQ ID NO: 814 - DNA, 11 bp, Influenza virus
cgtagagtcca

SEQ ID NO: 815 - DNA, 11 bp, Influenza virus
cgtagagtcca

SEQ ID NO: 816 - DNA, 11 bp, Influenza virus
cgtagaggcca

SEQ ID NO: 817 - DNA, 11 bp, Influenza virus
cgtagaggcca

SEQ ID NO: 818 - DNA, 11 bp, Influenza virus
cgtagagacta

SEQ ID NO: 819 - DNA, 11 bp, Influenza virus
cgtagagacta

SEQ ID NO: 820 - DNA, 11 bp, Influenza virus
cgtagagacta

SEQ ID NO: 821 - DNA, 11 bp, Influenza virus
cgtagagacta

SEQ ID NO: 822 - DNA, 181 bp, Influenza virus
gacccgctagagcgttgatctgatgaattagaatgtacgggaacaggaaacgagaaaaacctgaacgccggtaccgaggaacgagaatgatatgtgattctcaagactctcacaatttaagcaaatttatctcctgttcaagggattattcattatttctccatcactcttaaagactcaa

SEQ ID NO: 823 - DNA, 159 bp, Influenza virus
agtttatcgacatacgaccgcagcaggccgtagcaagaagacaagaagggtatacggggttcagttattaacgttacaatcaagaggcaacgcacaccctatagagtctcagtggcccgcatgagtttatctcctgttcaagggattattcattatttc

SEQ ID NO: 824 - DNA, 181 bp, Influenza virus
agtttatcgacatactaccgcagcaggccgtggcaagaaaacgaaaagggtatacggggttcagttattaacgttacaatcaagaagcagcgcacaccctatggagtctcagtggcccgcatggacccgctattgacctggtaagccgcaagccgcactcttgcagactccgccagtcagt

SEQ ID NO: 825 - DNA, 122 bp, Influenza virus
agtttatcgacatacgaccgcagcaggccgtggcaagaaaaagaaaagggtatacggggttcagttattaacgttacaatcaagaggcagcgcataccctatggagtctcagtgacccgcat

SEQ ID NO: 826 - DNA, 143 bp, Influenza virus
aacgaggtaggggttgttgaaaatgatagcgccccaaaaatcgtaagtctatcaatgcatatcatcgaacccagataactgtcccatcgggagcaattacaaaacatggcattgaccagttctactcagcacggcaggtcaca

SEQ ID NO: 827 - DNA, 143 bp, Influenza virus
aacgaggtaggggtcgttgaaaatgatagcgccccaaaaatcataagtctatcaatgcatatcatcgaaccccgataactgtcttgttaaagttggccgtggggtgcggcatcagttgacctcgacgaatgtaatgaactgta

SEQ ID NO: 828 - DNA, 143 bp, Influenza virus
ggtagaacgaaaactaccaggggcagagacaatttcggcgctgcggactcgcaggcatgcgctgcaaggtttaagcgctgaaattgctagagttggccgtggggtgcaatgccggttgacctcgacggatgtaatgaactgtg

SEQ ID NO: 829 - DNA, 143 bp, Influenza virus
ggtagagcgaaaactaccaggggcagagacaatttcggcgctgcggactcgcaggcatgcgctgcaaggtttaagcgctgaaattgctagagttggccgtggggtgcaatgccggttgacctcgacggatgtaatgaactgtg

SEQ ID NO: 830 - DNA, 143 bp, Influenza virus; identical to SEQ ID NO: 829

SEQ ID NO: 831 - DNA, 143 bp, Influenza virus
agtagaatgaaaactaccaggggcagagacaatttcggcactgtagactcgcaggcatgcgctgcaaggtttaagcgctgaaattgctagagttggccgtggggtgcaatgccggttgacctcgacggatgtaatgaactgtg

SEQ ID NO: 832 - DNA, 143 bp, Influenza virus
ggtaagacaaaaactaccaggggcggagaccatttcggcgctgcggactcgcaggcatgcgctgcaaggtttaagcgctgaaattgctagagttggccgtggggtgcaatgccggttgacctcgacggatgtaatgaactgtg

SEQ ID NO: 833 - DNA, 143 bp, Influenza virus
ggtaagataaaaactaccaggggcggaaaccatttcggcgctgcggactcgcaggcatgcgctgcaaggtttaagcgctgaaattgctagagttggccgtggggtgcaatgccggttgacctcgacggatgtaatgaactgtg

SEQ ID NO: 834 - DNA, 143 bp, Influenza virus
ggtagaacaaaagctaccaggggcagagataatttcggcgctgcgtactcgcaggcatgcgctgcaaggtttaagcgctgaaattgctagagttggccgtggggtgcaatgccggttgacctcgacggatgtaatgaactgtg

SEQ ID NO: 835 - DNA, 143 bp, Influenza virus
ggtagaacaaaaactaccaggggtagagacaatttcggcgctgcgggctcgcaggcatgcgctgcaaggtttaagcgctgaaattgctagagttggccgtggggtgcaatgccggttgacctcgacggatgtaatgaactgtg

SEQ ID NO: 836 - DNA, 190 bp, Influenza virus
tagttgtttgaataggtgtactatatagtttggtaaggcttcgtagcacagattttgctatggctaccagacagtcgaacgacagagaaggaaggcttagaatttcagttagctttaccactgcagttccttactggccgaaggtcggacgacaactaaacgtgcgcaggggggttaattcaacgctcca

SEQ ID NO: 837 - DNA, 187 bp, Influenza virus
gttgtttgaataggtgtactatatagtttggtaaggcttcgtagcagagattttgctatggctaccagacagtcgaacgacagagaaggaaggcttagaatttcagttagctttaccactgcagacttccgtcaattaggaagaagtagtgctagggtacataagaaaaaaccggcatggtatcctg

SEQ ID NO: 838 - DNA, 196 bp, Influenza virus
cgacaacccaggccaacaagtcgcgagacccaacttattcctacgataacagccccatcgcaactaccagatagtcggatgccagaaagaggaggctcaggattttagttagctttatcactgcaatcccttactggttaggggttggtacgacaacgaaacgtgcgtcagtgtgtaagttcaatatgacatccca

SEQ ID NO: 839 - DNA, 189 bp, Influenza virus
cgacaacccaggccaacaagtcgcgagacccaacttattcctacgataaaagccccatcgcaatgtgtgagtgactacgcactgagagggaagagtccgaggcccttacagatcccgctgtcctgaacttccgtcaattaggaagaagtagtgctagggtacataagaaaaaaccggcatggtatcttg

SEQ ID NO: 840 - DNA, 118 bp, Influenza virus
tcagatttttgaataaccagtcgatacttctcgagcataagacagatttgcgccgagagtctcgtagctcgtgacacacgatatggatgattacatacgttcaatatcgcgaaacttt

SEQ ID NO: 841 - DNA, 118 bp, Influenza virus
ctgacccacgaggcggttgactggcgtccactagatgcgcagagagcccaaattagagacctcgtagctcgtgacacacgatatggatgatcacatacgttcaatatcgcgaaacttt

SEQ ID NO: 842 - DNA, 118 bp, Influenza virus
ctgacccacgaggcggttgacttgcgtccactagatgcgcagagagcccaaattagagactctaagatctaaagaatgtagcgctagcagccgtgcttaccagggcttatatgctgac

SEQ ID NO: 843 - DNA, 46 bp, Influenza virus
aataccaagccgctagcgtctcatccctcaacatgtgtttaaagag

SEQ ID NO: 844 - DNA, 46 bp, Influenza virus
aataccaagccgctagcgtctcatccctcaacatgtgtttaagaga

SEQ ID NO: 845 - DNA, 46 bp, Influenza virus
aataccaagccgctagcgtctcatccctcaacatgtatttaagaga

SEQ ID NO: 846 - DNA, 46 bp, Influenza virus
cggtttggattatcgagtctctgcatactggtgccaatttaagaga

SEQ ID NO: 847 - DNA, 46 bp, Influenza virus; identical to SEQ ID NO: 846

SEQ ID NO: 848 - DNA, 46 bp, Influenza virus
cggtttggattatcgagtctctgcatactggtgccagcacggagag

SEQ ID NO: 849 - DNA, 137 bp, Influenza virus
tcactcatggttggtcgatgctgccacgacgacaagtagtatcctcaatcccttagggcaagtcttcagagaagatcttagcgcggcaacgagcgctacactaggtctcaggatatgatctattctacggggatcca

SEQ ID NO: 850 - DNA, 137 bp, Influenza virus; identical to SEQ ID NO: 849

SEQ ID NO: 851 - DNA, 137 bp, Influenza virus
ctgtgttcaactaacttccatcaccacgacaacaagcggtatcctcaaccccttagggcaagtcttcagaaaagatcttagcgcggcaacaagcgctacactgggtctcaggatatgatctattctacggggatcca

SEQ ID NO: 852 - DNA, 137 bp, Influenza virus
ctgtgttcaactaacctccatcattgaagaagtggacgaagcatctcgctaaacgaaatggactccagactggagctactatatactggtgggtcgcctatcgaacgctgaagcgcgtatggcctcgtataagcttg

SEQ ID NO: 853 - DNA, 135 bp, Influenza virus
gttttcaactaacctccatcattgaagaagtggacgaagcatctcgctatacgaaatggactccagacgggagctactatatactggtggatcgcctgtcgaacgctgaagcgcatatggcctcgtatgagcttg

SEQ ID NO: 854 - DNA, 209 bp, Influenza virus
aactgcaaccgccttcagcacacgtgccaaattggcccgtatcacaggagccatagaggtttcgctgagcatactaatgaggagtaattccgcatcacatgtgggtagttaggaccacgatatcctagaactatgcttaggaatcacggagtttcggaaccgacctggtgacggtcgagaccagaagaataaaacgccttacatgaagg

SEQ ID NO: 855 - DNA, 209 bp, Influenza virus
ggtcaagcccgccttcagcacacgtgccaaattggcccgtatcacaggagccatagaggtttcgctgagcatactaatgaggagtaattccgcatcacatgtgggtggttaggactacgatatcctagagctacgcttaggaatcacggagtttcggaaccgacctggtgacggtcgagaccagaagaataaaacgccttacatgaagg

SEQ ID NO: 856 - DNA, 209 bp, Influenza virus
ggtcaagcttatacctgctctgtatattgggcccattgacgcacagaagattgggagaagcctaacacttgagtcggcagaagacgtcattttgctctgcacatacgaacgaagatgtatagcttcgtggaggtttccgattccactaagaccataaggaaagttaaacactaactatcttatacttgtcgcggtaatccgtgcatgag

SEQ ID NO: 857 - DNA, 77 bp, Influenza virus
ggtgcaactgtaggagaccaaagcgaaaaccaaaaccctaacataagttgctcaaaaaaacctcataaaaaacaatg

SEQ ID NO: 858 - DNA, 77 bp, Influenza virus
agtgcaactgtaggagaccaaagcaaaaaccaaaaccctaacataaggtgcctagggtgggttcatagccaccagca

SEQ ID NO: 859 - DNA, 77 bp, Influenza virus
agtgcaactgtaggagaccaaagcaaaaaccaaaaccctaacataaggtgcctagggtgggttcatagccatcagca

SEQ ID NO: 860 - DNA, 77 bp, Influenza virus
agtgcaactgtaggagaccaaagcaaaaaccagaaccataacataaggtgcctagggtgggttcatagccatcagca

SEQ ID NO: 861 - DNA, 77 bp, Influenza virus
agtgcaactgtaggagaccaaagcgaaaaccaaaaccctaacataaggtgcctggggtgggtgtcccgccgccggca

SEQ ID NO: 862 - DNA, 77 bp, Influenza virus
agtgcaactgtaggagaccaaagcaaaaaccaaaaccctaacataaggtgcctggggtgcgtgtcccgccgccggca

SEQ ID NO: 863 - DNA, 77 bp, Influenza virus; identical to SEQ ID NO: 862

SEQ ID NO: 864 - DNA, 75 bp, Influenza virus
aagatgctgacgaagtgtactgataggggaaggtcagacgttgcgcatcaactggggtgcgtttccagccgtagg

SEQ ID NO: 865 - DNA, 75 bp, Influenza virus
aagatgctgatgaagtgtactgacaggggaaggtcagacgatgcgcatcaactggggtgcgtgtccagccgcaag

SEQ ID NO: 866 - DNA, 143 bp, Influenza virus; identical to SEQ ID NO: 826

SEQ ID NO: 867 - DNA, 143 bp, Influenza virus; identical to SEQ ID NO: 827

SEQ ID NO: 868 - DNA, 143 bp, Influenza virus
ggtagaacgaaaactaccaggggcagagataatttcggcgctgcggactcgcaggcatgcgctgcaaggtttaagcgctgaaattgctagagttggccgtggggtgcaatgccggttgacctcgacggatgtaatgaactgtg

SEQ ID NO: 869 - DNA, 46 bp, Influenza virus; identical to SEQ ID NO: 843

SEQ ID NO: 870 - DNA, 46 bp, Influenza virus; identical to SEQ ID NO: 844

SEQ ID NO: 871 - DNA, 46 bp, Influenza virus; identical to SEQ ID NO: 845

SEQ ID NO: 872 - DNA, 46 bp, Influenza virus; identical to SEQ ID NO: 846

SEQ ID NO: 873 - DNA, 46 bp, Influenza virus; identical to SEQ ID NO: 846

SEQ ID NO: 874 - DNA, 46 bp, Influenza virus; identical to SEQ ID NO: 848

SEQ ID NO: 875 - DNA, 18 bp, Ebola virus
gttcttggtccaatcatg

SEQ ID NO: 876 - DNA, 18 bp, Ebola virus
gttcttggtccaatcatg

SEQ ID NO: 877 - DNA, 18 bp, Ebola virus
gttcttggtccaatcatg

SEQ ID NO: 878 - DNA, 18 bp, Ebola virus
gttcttggtccaatcatg

SEQ ID NO: 879 - DNA, 18 bp, Influenza virus
gttcttggtccaatcatg

SEQ ID NO: 880 - DNA, 18 bp, Influenza virus
gttcttggtccaatcatg

SEQ ID NO: 881 - DNA, 18 bp, Influenza virus
gttcttggtccaatcatg

SEQ ID NO: 882 - DNA, 18 bp, Influenza virus
gttcttggtccaatcatg

SEQ ID NO: 883 - DNA, 18 bp, Influenza virus
gttcttggtccaatcatg

SEQ ID NO: 884 - DNA, 18 bp, Influenza virus
gttcttggtccaatcatg

SEQ ID NO: 885 - DNA, 18 bp, Influenza virus
gttcttggtccaatcatg

SEQ ID NO: 886 - DNA, 18 bp, Influenza virus
gttcttggtccaatcatg

SEQ ID NO: 887 - DNA, 18 bp, Influenza virus
gttcttggtccaatcatg

SEQ ID NO: 888 - DNA, 18 bp, Influenza virus
gttcttggtccaatcatg

SEQ ID NO: 889 - DNA, 18 bp, Influenza virus
gttcttggtccaatcatg

SEQ ID NO: 890 - DNA, 18 bp, Influenza virus
gttcttggtccaatcatg

SEQ ID NO: 891 - DNA, 18 bp, Influenza virus
gttcttggtccaatcatg

SEQ ID NO: 892 - DNA, 18 bp, Influenza virus
gttcttggtccaatcatg

SEQ ID NO: 893 - DNA, 18 bp, Influenza virus
gttcttggtccaatcatg

SEQ ID NO: 894 - DNA, 18 bp, Influenza virus
gttcttggtccaatcatg

SEQ ID NO: 895 - DNA, 18 bp, Influenza virus
gttcttggtccaatcatg

SEQ ID NO: 896 - DNA, 18 bp, Influenza virus
gttcttggtccaatcatg

SEQ ID NO: 897 - DNA, 18 bp, Influenza virus
gttcttggtccaatcatg

SEQ ID NO: 898 - DNA, 18 bp, Influenza virus
gttcttggtccaatcatg

SEQ ID NO: 899 - DNA, 18 bp, Influenza virus
gttcttggtccaatcatg

SEQ ID NO: 900 - DNA, 18 bp, Influenza virus
gttcttggtccaatcatg

SEQ ID NO: 901 - DNA, 18 bp, Influenza virus
gttcttggtccaatcatg

SEQ ID NO: 902 - DNA, 18 bp, Influenza virus
gttcttggtccaatcatg

SEQ ID NO: 903 - DNA, 18 bp, Influenza virus
gttcttggtccaatcatg

SEQ ID NO: 904 - DNA, 18 bp, Influenza virus
gttcttggtccaatcatg

SEQ ID NO: 905 - DNA, 18 bp, Influenza virus
gttcttggtccaatcatg

SEQ ID NO: 906 - DNA, 18 bp, Influenza virus
gttcttggtccaatcatg

SEQ ID NO: 907 - DNA, 18 bp, Influenza virus
gttcttggtccaatcatg

SEQ ID NO: 908 - DNA, 18 bp, Influenza virus
gttcttggtccaatcatg

SEQ ID NO: 909 - DNA, 18 bp, Influenza virus
gttcttggtccaatcatg

SEQ ID NO: 910 - DNA, 18 bp, Influenza virus
gttcttggtccaatcatg

SEQ ID NO: 911 - DNA, 18 bp, Influenza virus
gttcttggtccaatcatg

SEQ ID NO: 912 - DNA, 18 bp, Influenza virus
gttcttggtccaatcatg

SEQ ID NO: 913 - DNA, 18 bp, Influenza virus
gttcttggtccaatcatg

SEQ ID NO: 914 - DNA, 18 bp, Influenza virus
gttcttggtccaatcatg

SEQ ID NO: 915 - DNA, 18 bp, Influenza virus
gttcttggtccaatcatg

SEQ ID NO: 916 - DNA, 18 bp, Influenza virus
gttcttggtccaatcatg

SEQ ID NO: 917 - DNA, 18 bp, Influenza virus
gttcttggtccaatcatg

SEQ ID NO: 918 - DNA, 18 bp, Influenza virus
gttcttggtccaatcatg

SEQ ID NO: 919 - DNA, 18 bp, Influenza virus
gttcttggtccaatcatg

SEQ ID NO: 920 - DNA, 18 bp, Influenza virus
gttcttggtccaatcatg

SEQ ID NO: 921 - DNA, 18 bp, Influenza virus
gttcttggtccaatcatg

SEQ ID NO: 922 - DNA, 18 bp, Influenza virus
gttcttggtccaatcatg

SEQ ID NO: 923 - DNA, 18 bp, Influenza virus
gttcttggtccaatcatg

SEQ ID NO: 924 - DNA, 18 bp, Influenza virus
gttcttggtccaatcatg

SEQ ID NO: 925 - DNA, 18 bp, Influenza virus
gttcttggtccaatcatg

SEQ ID NO: 926 - DNA, 18 bp, Influenza virus
gttcttggtccaatcatg

SEQ ID NO: 927 - DNA, 18 bp, Influenza virus
gttcttggtccaatcatg

SEQ ID NO: 928 - DNA, 18 bp, Influenza virus
gttcttggtccaatcatg

SEQ ID NO: 929 - DNA, 18 bp, Influenza virus
gttcttggtccaatcatg

SEQ ID NO: 930 - DNA, 18 bp, Influenza virus
gttcttggtccaatcatg

SEQ ID NO: 931 - DNA, 18 bp, Influenza virus
gttcttggtccaatcatg

SEQ ID NO: 932 - DNA, 18 bp, Influenza virus
gttcttggtccaatcatg

SEQ ID NO: 933 - DNA, 18 bp, Influenza virus
gttcttggtccaatcatg

SEQ ID NO: 934 - DNA, 18 bp, Influenza virus
gttcttggtccaatcatg

SEQ ID NO: 935 - DNA, 18 bp, Influenza virus
gttcttggtccaatcatg

SEQ ID NO: 936 - DNA, 18 bp, Influenza virus
gttcttggtccaatcatg

SEQ ID NO: 937 - DNA, 18 bp, Influenza virus
gttcttggtccaatcatg

SEQ ID NO: 938 - DNA, 18 bp, Influenza virus
gttcttggtccaatcatg

SEQ ID NO: 939 - DNA, 18 bp, Influenza virus
gttcttggtccaatcatg

SEQ ID NO: 940 - DNA, 18 bp, Influenza virus
gttcttggtccaatcatg

SEQ ID NO: 941 - DNA, 18 bp, Influenza virus
gttcttggtccaatcatg

SEQ ID NO: 942 - DNA, 18 bp, Influenza virus
gttcttggtccaatcatg

SEQ ID NO: 943 - DNA, 18 bp, Influenza virus
gttcttggtccaatcatg

SEQ ID NO: 944 - DNA, 18 bp, Influenza virus
gttcttggtccaatcatg

SEQ ID NO: 945 - DNA, 18 bp, Influenza virus
gttcttggtccaatcatg

SEQ ID NO: 946 - DNA, 18 bp, Influenza virus
gttcttggtccaatcatg

SEQ ID NO: 947 - DNA, 18 bp, Influenza virus
gttcttggtccaatcatg

SEQ ID NO: 948 - DNA, 18 bp, Influenza virus
gttcttggtccaatcatg

SEQ ID NO: 949 - DNA, 18 bp, Influenza virus
gttcttggtccaatcatg

SEQ ID NO: 950 - DNA, 18 bp, Influenza virus
gttcttggtccaatcatg

SEQ ID NO: 951 - DNA, 18 bp, Influenza virus
gttcttggtccaatcatg

SEQ ID NO: 952 - DNA, 18 bp, Influenza virus
gttcttggtccaatcatg

SEQ ID NO: 953 - DNA, 18 bp, Influenza virus
gttcttggtccaatcatg

SEQ ID NO: 954 - DNA, 18 bp, Influenza virus
gttcttggtccaatcatg

SEQ ID NO: 955 - DNA, 18 bp, Influenza virus
gttcttggtccaatcatg

SEQ ID NO: 956 - DNA, 18 bp, Influenza virus
gttcttggtccaatcatg

SEQ ID NO: 957 - DNA, 18 bp, Influenza virus
gttcttggtccaatcatg

SEQ ID NO: 958 - DNA, 18 bp, Influenza virus
gttcttggtccaatcatg

SEQ ID NO: 959 - DNA, 18 bp, Influenza virus
gttcttggtccaatcatg

SEQ ID NO: 960 - DNA, 18 bp, Influenza virus
gttcttggtccaatcatg

SEQ ID NO: 961 - DNA, 18 bp, Influenza virus
gttcttggtccaatcatg

SEQ ID NO: 962 - DNA, 18 bp, Influenza virus
gttcttggtccaatcatg

SEQ ID NO: 963 - DNA, 18 bp, Influenza virus
gttcttggtccaatcatg

SEQ ID NO: 964 - DNA, 18 bp, Influenza virus
gttcttggtccaatcatg

SEQ ID NO: 965 - DNA, 18 bp, Influenza virus
gttcttggtccaatcatg

SEQ ID NO: 966 - DNA, 18 bp, Influenza virus
gttcttggtccaatcatg

SEQ ID NO: 967 - DNA, 18 bp, Influenza virus
gttcttggtccaatcatg

SEQ ID NO: 968 - DNA, 18 bp, Influenza virus
gttcttggtccaatcatg

SEQ ID NO: 969 - DNA, 18 bp, Influenza virus
gttcttggtccaatcatg

SEQ ID NO: 970 - DNA, 18 bp, Influenza virus
gttcttggtccaatcatg

SEQ ID NO: 971 - DNA, 18 bp, Influenza virus
gttcttggtccaatcatg

SEQ ID NO: 972 - DNA, 18 bp, Influenza virus
gttcttggtccaatcatg

SEQ ID NO: 973 - DNA, 18 bp, Influenza virus
gttcttggtccaatcatg

SEQ ID NO: 974 - DNA, 18 bp, Influenza virus
gttcttggtccaatcatg

SEQ ID NO: 975 - DNA, 18 bp, Influenza virus
gttcttggtccaatcatg

SEQ ID NO: 976 - DNA, 18 bp, Influenza virus
gttcttggtccaatcatg

SEQ ID NO: 977 - DNA, 18 bp, Influenza virus
gttcttggtccaatcatg

SEQ ID NO: 978 - DNA, 18 bp, Influenza virus
gttcttggtccaatcatg

SEQ ID NO: 979 - DNA, 18 bp, Influenza virus
gttcttggtccaatcatg

SEQ ID NO: 980 - DNA, 18 bp, Influenza virus
gttcttggtccaatcatg

SEQ ID NO: 981 - DNA, 18 bp, Influenza virus
gttcttggtccaatcatg

SEQ ID NO: 982 - DNA, 18 bp, Influenza virus
gttcttggtccaatcatg

SEQ ID NO: 983 - DNA, 18 bp, Influenza virus
gttcttggtccaatcatg

SEQ ID NO: 984 - DNA, 18 bp, Influenza virus
gttcttggtccaatcatg

SEQ ID NO: 985 - DNA, 18 bp, Influenza virus
gttcttggtccaatcatg

SEQ ID NO: 986 - DNA, 18 bp, Influenza virus
gttcttggtccaatcatg

SEQ ID NO: 987 - DNA, 18 bp, Influenza virus
gttcttggtccaatcatg

SEQ ID NO: 988 - DNA, 18 bp, Influenza virus
gttcttggtccaatcatg

SEQ ID NO: 989 - DNA, 18 bp, Influenza virus
gttcttggtccaatcatg

SEQ ID NO: 990 - DNA, 18 bp, Influenza virus
gttcttggtccaatcatg

SEQ ID NO: 991 - DNA, 18 bp, Influenza virus
gttcttggtccaatcatg

SEQ ID NO: 992 - DNA, 18 bp, Influenza virus
gttcttggtccaatcatg

SEQ ID NO: 993 - DNA, 18 bp, Influenza virus
gttcttggtccaatcatg

SEQ ID NO: 994 - DNA, 18 bp, Influenza virus
gttcttggtccaatcatg

SEQ ID NO: 995 - DNA, 18 bp, Influenza virus
gttcttggtccaatcatg

SEQ ID NO: 996 - DNA, 18 bp, Influenza virus
gttcttggtccaatcatg

SEQ ID NO: 997 - DNA, 18 bp, Influenza virus
gttcttggtccaatcatg

SEQ ID NO: 998 - DNA, 18 bp, Influenza virus
gttcttggtccaatcatg

SEQ ID NO: 999 - DNA, 18 bp, Influenza virus
gttcttggtccaatcatg

SEQ ID NO: 1000 - DNA, 18 bp, Influenza virus
gttcttggtccaatcatg

SEQ ID NO: 1001 - DNA, 18 bp, Influenza virus
gttcttggtccaatcatg

SEQ ID NO: 1002 - DNA, 18 bp, Influenza virus
gttcttggtccaatcatg

SEQ ID NO: 1003 - DNA, 18 bp, Influenza virus
gttcttggtccaatcatg

SEQ ID NO: 1004 - DNA, 18 bp, Influenza virus
gttcttggtccaatcatg

SEQ ID NO: 1005 - DNA, 18 bp, Influenza virus
gttcttggtccaatcatg

SEQ ID NO: 1006 - DNA, 18 bp, Influenza virus
gttcttggtccaatcatg

SEQ ID NO: 1007 - DNA, 18 bp, Influenza virus
gttcttggtccaatcatg

SEQ ID NO: 1008 - DNA, 18 bp, Influenza virus
gttcttggtccaatcatg

SEQ ID NO: 1009 - DNA, 18 bp, Influenza virus
gttcttggtccaatcatg

SEQ ID NO: 1010 - DNA, 18 bp, Influenza virus
gttcttggtccaatcatg

SEQ ID NO: 1011 - DNA, 18 bp, Influenza virus
gttcttggtccaatcatg

SEQ ID NO: 1012 - DNA, 18 bp, Influenza virus
gttcttggtccaatcatg

SEQ ID NO: 1013 - DNA, 18 bp, Influenza virus
gttcttggtccaatcatg

SEQ ID NO: 1014 - DNA, 18 bp, Influenza virus
gttcttggtccaatcatg

SEQ ID NO: 1015 - DNA, 18 bp, Influenza virus
gttcttggtccaatcatg

SEQ ID NO: 1016 - DNA, 18 bp, Influenza virus
gttcttggtccaatcatg

SEQ ID NO: 1017 - DNA, 18 bp, Influenza virus
gttcttggtccaatcatg

SEQ ID NO: 1018 - DNA, 18 bp, Influenza virus
gttcttggtccaatcatg

SEQ ID NO: 1019 - DNA, 18 bp, Influenza virus
gttcttggtccaatcatg

SEQ ID NO: 1020 - DNA, 18 bp, Influenza virus
gttcttggtccaatcatg

SEQ ID NO: 1021 - DNA, 18 bp, Influenza virus
gttcttggtccaatcatg

SEQ ID NO: 1022 - DNA, 18 bp, Influenza virus
gttcttggtccaatcatg

SEQ ID NO: 1023 - DNA, 18 bp, Influenza virus
gttcttggtccaatcatg

SEQ ID NO: 1024 - DNA, 18 bp, Influenza virus
gttcttggtccaatcatg

SEQ ID NO: 1025 - DNA, 18 bp, Influenza virus
gttcttggtccaatcatg

SEQ ID NO: 1026 - DNA, 18 bp, Influenza virus
gttcttggtccaatcatg

SEQ ID NO: 1027 - DNA, 18 bp, Influenza virus
gttcttggtccaatcatg

SEQ ID NO: 1028 - DNA, 18 bp, Ebola virus
gttcttagtccaatcatg

SEQ ID NO: 1029 - DNA, 18 bp, Ebola virus
gttcttagtccaatcatg

SEQ ID NO: 1030 - DNA, 18 bp, Ebola virus
gttcttagtccaatcatg

SEQ ID NO: 1031 - DNA, 18 bp, Ebola virus
gttcttagtccaatcatg

SEQ ID NO: 1032 - DNA, 18 bp, Ebola virus
gttcttagtccaatcatg

SEQ ID NO: 1033 - DNA, 18 bp, Ebola virus
gttcttagtccaatcatg

SEQ ID NO: 1034 - DNA, 18 bp, Ebola virus
gttcttagtccaatcatg

SEQ ID NO: 1035 - DNA, 18 bp, Ebola virus
gttcttagtccaatcatg

SEQ ID NO: 1036 - DNA, 18 bp, Ebola virus
gttcttagtccaatcatg

SEQ ID NO: 1037 - DNA, 18 bp, Ebola virus
gtttttggtccaatcatg

SEQ ID NO: 1038 - DNA, 18 bp, Ebola virus
gtttttggtccaatcatg

SEQ ID NO: 1039 - DNA, 18 bp, Ebola virus
gttttttgtccaatcatg

SEQ ID NO: 1040 - DNA, 18 bp, Ebola virus
attttttgtccaatcatg

SEQ ID NO: 1041 - DNA, 18 bp, Ebola virus
attttttgtccaatcatg

SEQ ID NO: 1042 - DNA, 18 bp, Ebola virus
attttttgtccaatcatg

SEQ ID NO: 1043 - DNA, 18 bp, Ebola virus
atttttggtccaatcttg

SEQ ID NO: 1044 - DNA, 18 bp, Influenza virus
attcttggtccaatcatg

SEQ ID NO: 1045 - DNA, 18 bp, Influenza virus
attcttggtccaatcatg

SEQ ID NO: 1046 - DNA, 18 bp, Influenza virus
attcttggtccaatcatg

SEQ ID NO: 1047 - DNA, 18 bp, Influenza virus
attcttggtccaatcatg

SEQ ID NO: 1048 - DNA, 18 bp, Influenza virus
actcttggtccaatcatg

SEQ ID NO: 1049 - DNA, 18 bp, Influenza virus
gctcttggtccaatcatg

SEQ ID NO: 1050 - DNA, 18 bp, Influenza virus
gctcttggtccaatcatg

SEQ ID NO: 1051 - DNA, 18 bp, Influenza virus
gctcttggtccaatcatg

SEQ ID NO: 1052 - DNA, 18 bp, Influenza virus
gctcttggtccaatcatg

SEQ ID NO: 1053 - DNA, 18 bp, Influenza virus
gctcttggtccaatcatg

SEQ ID NO: 1054 - DNA, 18 bp, Influenza virus
gctcttggtccaatcatg

SEQ ID NO: 1055 - DNA, 18 bp, Influenza virus
gctcttggtccaatcatg

SEQ ID NO: 1056 - DNA, 18 bp, Influenza virus
gctcttggtccaatcatg

SEQ ID NO: 1057 - DNA, 18 bp, Influenza virus
gctcttggtccaatcatg

SEQ ID NO: 1058 - DNA, 18 bp, Influenza virus
gctcttggtccaatcatg

SEQ ID NO: 1059 - DNA, 18 bp, Influenza virus
gctcttggtccaatcatg

SEQ ID NO: 1060 - DNA, 18 bp, Influenza virus
gctcttggtccaatcatg

SEQ ID NO: 1061 - DNA, 18 bp, Influenza virus
gctcttggtccaatcatg

SEQ ID NO: 1062 - DNA, 18 bp, Influenza virus
gctcttggtccaatcatg

SEQ ID NO: 1063 - DNA, 18 bp, Influenza virus
gctcttggtccaatcatg

SEQ ID NO: 1064 - DNA, 18 bp, Influenza virus
gctcttggtccaatcatg

SEQ ID NO: 1065 - DNA, 18 bp, Influenza virus
gctcttggtccaatcatg

SEQ ID NO: 1066 - DNA, 18 bp, Influenza virus
gctcttggtccaatcatg

SEQ ID NO: 1067 - DNA, 18 bp, Influenza virus
gctcttggtccaatcatg

SEQ ID NO: 1068 - DNA, 18 bp, Influenza virus
gctcttggtccaatcatg

SEQ ID NO: 1069 - DNA, 18 bp, Influenza virus
gctcttggtccaatcatg

SEQ ID NO: 1070 - DNA, 18 bp, Influenza virus
gctcttggtccaatcatg

SEQ ID NO: 1071 - DNA, 18 bp, Influenza virus
gctcttggtccaatcatg

SEQ ID NO: 1072 - DNA, 18 bp, Influenza virus
gctcttggtccaatcatg

SEQ ID NO: 1073 - DNA, 18 bp, Influenza virus
gctcttggtccaatcatg

SEQ ID NO: 1074 - DNA, 18 bp, Influenza virus
gctcttggtccaatcatg

SEQ ID NO: 1075 - DNA, 18 bp, Influenza virus
gctcttggtccaatcatg

SEQ ID NO: 1076 - DNA, 18 bp, Influenza virus
gctcttggtccaatcatg

SEQ ID NO: 1077 - DNA, 18 bp, Influenza virus
gctcttggtccaatcatg

SEQ ID NO: 1078 - DNA, 18 bp, Influenza virus
gctcttggtccaatcatg

SEQ ID NO: 1079 - DNA, 18 bp, Influenza virus
gctcttggtccaatcatg

SEQ ID NO: 1080 - DNA, 18 bp, Influenza virus
gctcttggtccaatcatg

SEQ ID NO: 1081 - DNA, 18 bp, Influenza virus
actcttggtccagtcatg

SEQ ID NO: 1082 - DNA, 18 bp, Influenza virus
attcttggtccagtcatg

SEQ ID NO: 1083 - DNA, 18 bp, Influenza virus
attcttggtccagtcatg

SEQ ID NO: 1084 - DNA, 18 bp, Influenza virus
attcttggtccagtcatg

SEQ ID NO: 1085 - DNA, 18 bp, Influenza virus
attcttggtccagtcatg

SEQ ID NO: 1086 - DNA, 18 bp, Influenza virus
attcttggtccagtcatg

SEQ ID NO: 1087 - DNA, 18 bp, Influenza virus
attcttggtccagtcatg

SEQ ID NO: 1088 - DNA, 18 bp, Influenza virus
attcttggtccagtcatg

SEQ ID NO: 1089 - DNA, 18 bp, Influenza virus
attcttggtccagtcatg

SEQ ID NO: 1090 - DNA, 18 bp, Influenza virus
attcttggtccagtcatg

SEQ ID NO: 1091 - DNA, 18 bp, Influenza virus
attcttggtccagtcatg

SEQ ID NO: 1092 - DNA, 18 bp, Influenza virus
attcttggtccagtcatg

SEQ ID NO: 1093 - DNA, 18 bp, Influenza virus
attcttggtccagtcatg

SEQ ID NO: 1094 - DNA, 18 bp, Influenza virus
attcttggtccagtcatg

SEQ ID NO: 1095 - DNA, 18 bp, Influenza virus
attcttggtccagtcatg

SEQ ID NO: 1096 - DNA, 18 bp, Influenza virus
gttcttggtccagtcatg

SEQ ID NO: 1097 - DNA, 18 bp, Influenza virus
gttcttggtccagtcatg

SEQ ID NO: 1098 - DNA, 18 bp, Influenza virus
gttcttggtccagtcatg

SEQ ID NO: 1099 - DNA, 18 bp, Influenza virus
gttcttggtccagtcatg

SEQ ID NO: 1100 - DNA, 18 bp, Influenza virus
gttcttggtccagtcatg

SEQ ID NO: 1101 - DNA, 18 bp, Influenza virus
gttcttggtccagtcatg

SEQ ID NO: 1102 - DNA, 18 bp, Influenza virus
gttcttggtccagtcatg

SEQ ID NO: 1103 - DNA, 18 bp, Influenza virus
gttcttggtccagtcatg

SEQ ID NO: 1104 - DNA, 18 bp, Influenza virus
gttcttggtccagtcatg

SEQ ID NO: 1105 - DNA, 18 bp, Influenza virus
gttcttggtccagtcatg

SEQ ID NO: 1106 - DNA, 18 bp, Influenza virus
gttcttggtccagtcatg

SEQ ID NO: 1107 - DNA, 18 bp, Influenza virus
gttcttggtccagtcatg

SEQ ID NO: 1108 - DNA, 18 bp, Influenza virus
gttcttggtccagtcatg

SEQ ID NO: 1109 - DNA, 18 bp, Influenza virus
gttcttggtccagtcatg

SEQ ID NO: 1110 - DNA, 18 bp, Influenza virus
gttcttggtccagtcatg

SEQ ID NO: 1111 - DNA, 18 bp, Influenza virus
gttcttggtccagtcatg

SEQ ID NO: 1112 - DNA, 18 bp, Influenza virus
gttcttggtccagtcatg

SEQ ID NO: 1113 - DNA, 18 bp, Influenza virus
gttcttggtccagtcatg

SEQ ID NO: 1114 - DNA, 18 bp, Influenza virus
gttcttggtccagtcatg

SEQ ID NO: 1115 - DNA, 18 bp, Influenza virus
gttcttggtccagtcatg

SEQ ID NO: 1116 - DNA, 18 bp, Influenza virus
gttcttggtccagtcatg

SEQ ID NO: 1117 - DNA, 18 bp, Influenza virus
gttcttggtccagtcatg

SEQ ID NO: 1118 - DNA, 18 bp, Influenza virus
gttcttggtccagtcatg

SEQ ID NO: 1119 - DNA, 18 bp, Influenza virus
gttcttggtccagtcatg

SEQ ID NO: 1120 - DNA, 18 bp, Influenza virus
gttcttggtccagtcatg

SEQ ID NO: 1121 - DNA, 18 bp, Influenza virus
gttcttggtccagtcatg

SEQ ID NO: 1122 - DNA, 18 bp, Influenza virus
gttcttggtccagtcatg

SEQ ID NO: 1123 - DNA, 18 bp, Influenza virus
gttcttggtccagtcatg

SEQ ID NO: 1124 - DNA, 18 bp, Influenza virus
gttcttggtccagtcatg

SEQ ID NO: 1125 - DNA, 18 bp, Influenza virus
gttcttggtccagtcatg

SEQ ID NO: 1126 - DNA, 18 bp, Influenza virus
gttcttggtccagtcatg

SEQ ID NO: 1127 - DNA, 18 bp, Influenza virus
gttcttggtccagtcatg

SEQ ID NO: 1128 - DNA, 18 bp, Influenza virus
gttcttggtccagtcatg

SEQ ID NO: 1129 - DNA, 18 bp, Influenza virus
gttcttggtccagtcatg

SEQ ID NO: 1130 - DNA, 18 bp, Influenza virus
gttcttggtccagtcatg

SEQ ID NO: 1131 - DNA, 18 bp, Influenza virus
gttcttggtccagtcatg

SEQ ID NO: 1132 - DNA, 18 bp, Influenza virus
gttcttggtccagtcatg

SEQ ID NO: 1133 - DNA, 18 bp, Influenza virus
gttcttggtccagtcatg

SEQ ID NO: 1134 - DNA, 18 bp, Influenza virus
gttcttggtccagtcatg

SEQ ID NO: 1135 - DNA, 18 bp, Influenza virus
gttcttggtccagtcatg

SEQ ID NO: 1136 - DNA, 18 bp, Influenza virus
gttcttggtccagtcatg

SEQ ID NO: 1137 - DNA, 18 bp, Influenza virus
gttcttggtccagtcatg

SEQ ID NO: 1138 - DNA, 18 bp, Influenza virus
gttcttggtccagtcatg

SEQ ID NO: 1139 - DNA, 18 bp, Influenza virus
gttcttggtccagtcatg

SEQ ID NO: 1140 - DNA, 18 bp, Influenza virus
gttcttggtccagtcatg

SEQ ID NO: 1141 - DNA, 18 bp, Influenza virus
gttcttggtccagtcatg

SEQ ID NO: 1142 - DNA, 18 bp, Influenza virus
gttcttggtccagtcatg

SEQ ID NO: 1143 - DNA, 18 bp, Influenza virus
gttcttggtccagtcatg

SEQ ID NO: 1144 - DNA, 18 bp, Influenza virus
gttcttggtccagtcatg

SEQ ID NO: 1145 - DNA, 18 bp, Influenza virus
gttcttggtccagtcatg

SEQ ID NO: 1146 - DNA, 18 bp, Influenza virus
gttcttggtccagtcatg

SEQ ID NO: 1147 - DNA, 18 bp, Influenza virus
gttcttggtccagtcatg

SEQ ID NO: 1148 - DNA, 18 bp, Influenza virus
gttcttggtccagtcatg

SEQ ID NO: 1149 - DNA, 18 bp, Influenza virus
gttcttggtccagtcatg

SEQ ID NO: 1150 - DNA, 18 bp, Influenza virus
gttcttggtccagtcatg

SEQ ID NO: 1151 - DNA, 18 bp, Influenza virus
gttcttggtccagtcatg

SEQ ID NO: 1152 - DNA, 18 bp, Influenza virus
gttcttggtccagtcatg

SEQ ID NO: 1153 - DNA, 18 bp, Influenza virus
gttcttggtccagtcatg

SEQ ID NO: 1154 - DNA, 18 bp, Influenza virus
gttcttggtccagtcatg

SEQ ID NO: 1155 - DNA, 18 bp, Influenza virus
gttcttggtccagtcatg

SEQ ID NO: 1156 - DNA, 18 bp, Influenza virus
gttcttggtccagtcatg

SEQ ID NO: 1157 - DNA, 18 bp, Influenza virus
gttcttggtccagtcatg

SEQ ID NO: 1158 - DNA, 18 bp, Influenza virus
gttcttggtccagtcatg

SEQ ID NO: 1159 - DNA, 18 bp, Influenza virus
gttcttggtccagtcatg

SEQ ID NO: 1160 - DNA, 18 bp, Influenza virus
gttcttggtccagtcatg

SEQ ID NO: 1161 - DNA, 18 bp, Influenza virus
gttcttggtccagtcatg

SEQ ID NO: 1162 - DNA, 18 bp, Influenza virus
gttcttggtccagtcatg

SEQ ID NO: 1163 - DNA, 18 bp, Influenza virus
gttcttggtccagtcatg

SEQ ID NO: 1164 - DNA, 18 bp, Influenza virus
gttcttggtccagtcatg

SEQ ID NO: 1165 - DNA, 18 bp, Influenza virus
gttcttggtccagtcatg

SEQ ID NO: 1166 - DNA, 18 bp, Influenza virus
gttcttggtccagtcatg

SEQ ID NO: 1167 - DNA, 18 bp, Influenza virus
gttcttggtccagtcatg

SEQ ID NO: 1168 - DNA, 18 bp, Influenza virus
gttcttggtccagtcatg

SEQ ID NO: 1169 - DNA, 18 bp, Influenza virus
gttcttggtccagtcatg

SEQ ID NO: 1170 - DNA, 18 bp, Influenza virus
gttcttggtccagtcatg

SEQ ID NO: 1171 - DNA, 18 bp, Influenza virus
gttcttggtccagtcatg

SEQ ID NO: 1172 - DNA, 18 bp, Influenza virus
gttcttggtccagtcatg

SEQ ID NO: 1173 - DNA, 18 bp, Influenza virus
gttcttggtccagtcatg

SEQ ID NO: 1174 - DNA, 18 bp, Influenza virus
gttcttggtccagtcatg

SEQ ID NO: 1175 - DNA, 18 bp, Influenza virus
gttcttggtccagtcatg

SEQ ID NO: 1176 - DNA, 18 bp, Influenza virus
gttcttggtccagtcatg

SEQ ID NO: 1177 - DNA, 18 bp, Influenza virus
gttcttggtccgatcatg

SEQ ID NO: 1178 - DNA, 18 bp, Influenza virus
gttcttggtccgatcatg

SEQ ID NO: 1179 - DNA, 18 bp, Influenza virus
gttcttggtccgatcatg

SEQ ID NO: 1180 - DNA, 18 bp, Influenza virus
gttcttggtccgatcatg

SEQ ID NO: 1181 - DNA, 18 bp, Influenza virus
gttcttggtccgatcatg

SEQ ID NO: 1182 - DNA, 18 bp, Influenza virus
gttcttggtccgatcatg

SEQ ID NO: 1183 - DNA, 18 bp, Influenza virus
gttcttggtccgatcatg

SEQ ID NO: 1184 - DNA, 18 bp, Influenza virus
gttcttggtccgatcatg

SEQ ID NO: 1185 - DNA, 18 bp, Influenza virus
gttcttggtccgatcatg

SEQ ID NO: 1186 - DNA, 18 bp, Influenza virus
gttcttggtccgatcatg

SEQ ID NO: 1187 - DNA, 18 bp, Influenza virus
gttcttggtccgatcatg

SEQ ID NO: 1188 - DNA, 18 bp, Influenza virus
gttcttggtccgatcatg

SEQ ID NO: 1189 - DNA, 18 bp, Influenza virus
gttcttggtccgatcatg

SEQ ID NO: 1190 - DNA, 18 bp, Influenza virus
gttcttggtccgatcatg

SEQ ID NO: 1191 - DNA, 18 bp, Influenza virus
gttcttggtccgatcatg

SEQ ID NO: 1192 - DNA, 18 bp, Influenza virus
gttcttggtccgatcatg

SEQ ID NO: 1193 - DNA, 18 bp, Influenza virus
gttcttggtccgatcatg

SEQ ID NO: 1194 - DNA, 18 bp, Influenza virus
gttcttggtccgatcatg

SEQ ID NO: 1195 - DNA, 18 bp, Influenza virus
gttcttggtccgatcatg

SEQ ID NO: 1196 - DNA, 18 bp, Influenza virus
gttcttggtccgatcatg

SEQ ID NO: 1197 - DNA, 18 bp, Influenza virus
gttcttggtccgatcatg

SEQ ID NO: 1198 - DNA, 18 bp, Influenza virus
gttcttggtccgatcatg

SEQ ID NO: 1199 - DNA, 18 bp, Influenza virus
gttcttggtccgatcatg

SEQ ID NO: 1200 - DNA, 18 bp, Influenza virus
gttcttggtccgatcatg

SEQ ID NO: 1201 - DNA, 18 bp, Influenza virus
gttcttggtccgatcatg

SEQ ID NO: 1202 - DNA, 18 bp, Influenza virus
gttcttggtccgatcatg

SEQ ID NO: 1203 - DNA, 18 bp, Influenza virus
gttcttggtccgatcatg

SEQ ID NO: 1204 - DNA, 18 bp, Influenza virus
gttcttggtccgatcatg

SEQ ID NO: 1205 - DNA, 18 bp, Influenza virus
gttcttggcccaatcatg

SEQ ID NO: 1206 - DNA, 18 bp, Influenza virus
gttcttggcccaatcatg

SEQ ID NO: 1207 - DNA, 18 bp, Influenza virus
gttcttggcccaatcatg

SEQ ID NO: 1208 - DNA, 18 bp, Influenza virus
gttcatggcccaatcatg

SEQ ID NO: 1209 - DNA, 18 bp, Influenza virus
gttcatggcccaatcatg

SEQ ID NO: 1210 - DNA, 18 bp, Influenza virus
gttcatggcccaatcatg

SEQ ID NO: 1211 - DNA, 18 bp, Influenza virus
gttcatggcccaatcatg

SEQ ID NO: 1212 - DNA, 18 bp, Influenza virus
gttcatggcccaatcatg

SEQ ID NO: 1213 - DNA, 18 bp, Influenza virus
gttcatggcccaatcatg

SEQ ID NO: 1214 - DNA, 18 bp, Influenza virus
gttcatggcccaatcatg

SEQ ID NO: 1215 - DNA, 18 bp, Influenza virus
gttcatggcccaatcatg

SEQ ID NO: 1216 - DNA, 18 bp, Influenza virus
gttcatggcccaatcatg

SEQ ID NO: 1217 - DNA, 18 bp, Influenza virus
gttcatggcccaatcatg

SEQ ID NO: 1218 - DNA, 18 bp, Influenza virus
gttcatggcccaatcatg

SEQ ID NO: 1219 - DNA, 18 bp, Influenza virus
gttcatggcccaatcatg

SEQ ID NO: 1220 - DNA, 18 bp, Influenza virus
gttcatggcccaatcatg

SEQ ID NO: 1221 - DNA, 18 bp, Influenza virus
gttcatggcccaatcatg

SEQ ID NO: 1222 - DNA, 18 bp, Influenza virus
gttcatggcccaatcatg

SEQ ID NO: 1223 - DNA, 18 bp, Influenza virus
gttcatggcccaatcatg

SEQ ID NO: 1224 - DNA, 18 bp, Influenza virus
gttcatggcccaatcatg

SEQ ID NO: 1225 - DNA, 18 bp, Influenza virus
gttcatggcccaatcatg

SEQ ID NO: 1226 - DNA, 18 bp, Influenza virus
gttcatggcccaatcatg

SEQ ID NO: 1227 - DNA, 18 bp, Influenza virus
gttcatggcccaatcatg

SEQ ID NO: 1228 - DNA, 18 bp, Influenza virus
gttcatggcccaatcatg

SEQ ID NO: 1229 - DNA, 18 bp, Influenza virus
gttcatggcccaatcatg

SEQ ID NO: 1230 - DNA, 18 bp, Influenza virus
gttcatggcccaatcatg

SEQ ID NO: 1231 - DNA, 18 bp, Influenza virus
gttcatggcccaatcatg

SEQ ID NO: 1232 - DNA, 18 bp, Influenza virus
gttcatggcccaatcatg

SEQ ID NO: 1233 - DNA, 18 bp, Influenza virus
gttcatggcccaatcatg

SEQ ID NO: 1234 - DNA, 18 bp, Influenza virus
gttcatggcccaatcatg

SEQ ID NO: 1235 - DNA, 18 bp, Influenza virus
gttcatggcccaatcatg

SEQ ID NO: 1236 - DNA, 18 bp, Influenza virus
gttcatggcccaatcatg

SEQ ID NO: 1237 - DNA, 18 bp, Influenza virus
gttcatggcccaatcatg

SEQ ID NO: 1238 - DNA, 18 bp, Influenza virus
gttcatggcccaatcatg

SEQ ID NO: 1239 - DNA, 18 bp, Influenza virus
gttcatggcccaatcatg

SEQ ID NO: 1240 - DNA, 18 bp, Influenza virus
gttcatggcccaatcatg

SEQ ID NO: 1241 - DNA, 18 bp, Influenza virus
gttcatggcccaatcatg

SEQ ID NO: 1242 - DNA, 18 bp, Influenza virus
gttcatggcccaatcatg

SEQ ID NO: 1243 - DNA, 18 bp, Influenza virus
gttcatggcccaatcatg

SEQ ID NO: 1244 - DNA, 18 bp, Influenza virus
gttcatggcccaatcatg

SEQ ID NO: 1245 - DNA, 18 bp, Influenza virus
gttcatggcccaatcatg

SEQ ID NO: 1246 - DNA, 18 bp, Influenza virus
gttcatggcccaatcatg

SEQ ID NO: 1247 - DNA, 18 bp, Influenza virus
gttcatggcccaatcatg

SEQ ID NO: 1248 - DNA, 18 bp, Influenza virus
gttcatggcccaatcatg

SEQ ID NO: 1249 - DNA, 18 bp, Influenza virus
gttcatggcccaatcatg

SEQ ID NO: 1250 - DNA, 18 bp, Influenza virus
gttcatggcccaatcatg

SEQ ID NO: 1251 - DNA, 18 bp, Influenza virus
gttcatggcccaatcatg

SEQ ID NO: 1252 - DNA, 18 bp, Influenza virus
gttcatggcccaatcatg

SEQ ID NO: 1253 - DNA, 18 bp, Influenza virus
gttcatggcccaatcatg

SEQ ID NO: 1254 - DNA, 18 bp, Influenza virus
attcatggcccaatcatg

SEQ ID NO: 1255 - DNA, 18 bp, Influenza virus
attcatggcccaatcatg

SEQ ID NO: 1256 - DNA, 18 bp, Influenza virus
attcatggcccaatcatg

SEQ ID NO: 1257 - DNA, 18 bp, Influenza virus
attcatggcccaatcatg

SEQ ID NO: 1258 - DNA, 18 bp, Influenza virus
attcatggcccaatcatg

SEQ ID NO: 1259 - DNA, 18 bp, Influenza virus
attcatggcccaatcatg

SEQ ID NO: 1260 - DNA, 18 bp, Influenza virus
attcatggcccaatcatg

SEQ ID NO: 1261 - DNA, 18 bp, Influenza virus
attcatggcccaatcatg

SEQ ID NO: 1262 - DNA, 18 bp, Influenza virus
attcatggcccaatcatg

SEQ ID NO: 1263 - DNA, 18 bp, Influenza virus
attcatggcccaatcatg

SEQ ID NO: 1264 - DNA, 18 bp, Influenza virus
attcatggcccaatcatg

SEQ ID NO: 1265 - DNA, 18 bp, Influenza virus
attcatggcccaatcatg

SEQ ID NO: 1266 - DNA, 18 bp, Influenza virus
gttcgtggcccaatcatg

SEQ ID NO: 1267 - DNA, 18 bp, Influenza virus
gttcgtggcccaatcatg

SEQ ID NO: 1268 - DNA, 18 bp, Influenza virus
gttcgtggcccaatcatg

SEQ ID NO: 1269 - DNA, 18 bp, Influenza virus
gttcgtggcccaatcatg

SEQ ID NO: 1270 - DNA, 18 bp, Influenza virus
gttcgtggcccaatcatg

SEQ ID NO: 1271 - DNA, 18 bp, Influenza virus
gttcgtggcccaatcatg

SEQ ID NO: 1272 - DNA, 18 bp, Influenza virus
gttcgtggcccaatcatg

SEQ ID NO: 1273 - DNA, 18 bp, Influenza virus
gttcgtggcccaatcatg

SEQ ID NO: 1274 - DNA, 18 bp, Influenza virus
gttcgtggcccaatcatg

SEQ ID NO: 1275 - DNA, 18 bp, Influenza virus
gttcgtggcccaatcatg

SEQ ID NO: 1276 - DNA, 18 bp, Influenza virus
gttcgtggcccaatcatg

SEQ ID NO: 1277 - DNA, 18 bp, Influenza virus
gttcgtggcccaatcatg

SEQ ID NO: 1278 - DNA, 18 bp, Influenza virus
gttcgtggcccaatcatg

SEQ ID NO: 1279 - DNA, 18 bp, Influenza virus
gctcgtggcccaatcatg

SEQ ID NO: 1280 - DNA, 18 bp, Influenza virus
gctcgtggcccaatcatg

SEQ ID NO: 1281 - DNA, 18 bp, Influenza virus
gctcgtggcccaatcatg

SEQ ID NO: 1282 - DNA, 18 bp, Influenza virus
gctcgtggcccaatcatg

SEQ ID NO: 1283 - DNA, 18 bp, Influenza virus
gctcgtggcccaatcatg

SEQ ID NO: 1284 - DNA, 18 bp, Influenza virus
gctcgtggcccaatcatg

SEQ ID NO: 1285 - DNA, 18 bp, Influenza virus
gctcgtggcccaatcatg

SEQ ID NO: 1286 - DNA, 18 bp, Influenza virus
gctcgtggcccaatcatg

SEQ ID NO: 1287 - DNA, 18 bp, Influenza virus
gctcgtggcccaatcatg

SEQ ID NO: 1288 - DNA, 18 bp, Influenza virus
gctcgtggcccaatcatg

SEQ ID NO: 1289 - DNA, 208 bp, Influenza virus
aattgtttagagtagagtgtggtttatatagtttggtaaggcctcgtagcaccaattttgctatggctaccagtgacagtccggtcgccaggaaagagaagctcaggcagttattagttaaacttttcactgcacaattaaccttactgggccgacggttggacgacaactaaacgtgcagcggaggggggttaattcaaccgctcca

SEQ ID NO: 1290 - DNA, 214 bp, Influenza virus
aattgtttagagtatagtgtggcctatatagtttggtaaggcctcgtagcagcaattttgctatggctaccagtgacagtccgatcgccaagagagggaagctcaggcagttatcagttaagcttttcactgcacgattaaccttactgggcccaaggttggtatgacaacgaaacgtgcagtcagagtgtgtgagtttaattcaaccgcttca

SEQ ID NO: 1291 - DNA, 208 bp, Influenza virus
agttgtttggaatagagtgtgatctatatagtttggtaaggcttcgtagcacagattttgctatggctaccagtgacagtccgaccgacaggagaagaaagcttagacagttatcagttaagctttccactgcacgattgaccttactgggccgaaggtcggacgacaactaaacgtgcagcagaggggggttaattcaaccgctcca

SEQ ID NO: 1292 - DNA, 206 bp, Influenza virus
gttgtttggaatagagtgtgacctatatagtttggtaaggcttcgtagcagagattttgctatggctaccagtgacagtccgaccgacaggagaagaaagcttagacagttatcagttaagcttttcactgcacgatcaattccgtcaaattaggaagaagtagtgatagggtacataaaggaaaaaaaccggcatggtcatcctg

SEQ ID NO: 1293 - DNA, 214 bp, Influenza virus
gacaacccgaggccaaacaaggttcgcgagacccaacttattcctacgataacagccccatcgcaactaccagtgatagtccggtcgccaggaaagggaagctcaggcagttattagttaagcttttcactgcacaatcaaccttactgggttaggggttggtacgacaacgaaacgtgcagtcagagtgtgtaagtttaatatgaccatccca

SEQ ID NO: 1294 - DNA, 207 bp, Influenza virus
gacaacccgaggccaaacaaggttcgcgagacccaacttattcctacgataaaagccccatcgcaatgtgtggtagtgacttacccactgaagaggaagggtccgagtgaccgcttacaggatctcctgtcctgtagacgattccgtcgaattaggaagaagtagtgatagggtacatgaagggaaaaaaccggcatggtcatcttg

SEQ ID NO: 1295 - DNA, 207 bp, Influenza virus
gacaacccaaggccaggcaaggttcgcgagacccaacttagtcccacggtaacaaccccatcgcaatgtgtggtagtgacttacccactgaagaggaagagtccgagtggccgcttacaggatctcctgtcctgtagacaattccgtcgaattaggaagaagcagtgatagggtacataaagagaaaaaaccggcatgattatcctg

SEQ ID NO: 1296 - DNA, 207 bp, Influenza virus; identical to SEQ ID NO: 1295

SEQ ID NO: 1297 - DNA, 207 bp, Influenza virus
gataacccaaggccaagcaaggttcgcgagacccaacttagtcccacggtaacaaccccatcgcaatgtgtggtagtgacttacccactgaagaggaagagtccgagtgaccgcttacaggatctcctgtcctgtagacaattccgtcgaattaggaagaagtagtgatagggtacataaagggaaaaaaccggcatggtcatcctg

SEQ ID NO: 1298 - DNA, 207 bp, Influenza virus
gacaacccaaggccaagcaaagttcgcgagacccaacttagtcccacggtaacaaccccatcgcaatgtgtgacagttactcacctactgaagaggaagagtccgagtgaccgcttacaggatcacctgtcctgtagacaattccgtcaaattaggaagaagtagtgatagggtacatgaaggaaaaaaaccggcatggtcatcctg

SEQ ID NO: 1299 - DNA, 207 bp, Influenza virus; identical to SEQ ID NO: 1298

SEQ ID NO: 1300 - DNA, 207 bp, Influenza virus
gacaacccaaggccaagcaaggttcgcgagacccaacttagtcccacggtaacaaccccatcgcaatgtgtggtagtgacttacccactgaagaggaagagtccgagtgaccgcttacaggatctcctgtcctgtagacagttccgtcgaattaggaagaagtagtgatagggtacataaagagaaaaaaccggcatggtcatcctg

SEQ ID NO: 1301 - DNA, 207 bp, Influenza virus
gacaacccaaggccaagcaaggttcgcgagacccaacttagtcccacggtaacaaccccatcgcaatgtgtggtagtgactcacccactgaagaggaagagtccgagtgaccgcttacaggatctcctgtcctgtagacagttccgtcgaattaggaagaagtagtgatagggtacgtaaagggagaaaaccggcatggtcatcctg

SEQ ID NO: 1302 - DNA, 148 bp, Influenza virus
ttgcgacccatcaagacggttgagctggcatttcatatagacagcgacaggagagggcccaaactagcagatatctaagatcttaacagactgtagcgctatggtcagccgtggcatcaacccgggctcggcaaatggcgatggagat

SEQ ID NO: 1303 - DNA, 148 bp, Influenza virus
ttgcgacccaccaaggcggttgagctggcattccatatagatcgcgacaagagagggcccaaattagcagacatctaagatcttaacagaatgtagcgctatggtcagccgtagcattaacccgggctcggcaaatggcgatggagat

SEQ ID NO: 1304 - DNA, 148 bp, Influenza virus
tcgtgacccatcgaggcggttgagctggcgttccacatagatcgcgacaagagagggcccaaattagcagacactcgtagcttcgtcgacacacgatatggtgattgatcactataacagttcaatatcggcggaagaagacgttggt

SEQ ID NO: 1305 - DNA, 148 bp, Influenza virus
tcgtgacccatcgaggcggttgagcttgcgttccacatagatcgcgacaagagagggcccaaattagcagacatctaagatcttaacagaatgtagcgctatggtcagccgttgcattaaccagggcttagtagatggcgatggaggc

SEQ ID NO: 1306 - DNA, 148 bp, Influenza virus
tcgtgacccatcgaggcggttgagcttgcgttccacatagatcgcgacaagagagggcccaaattagcagacatctaagatcttaacagaatgtagcgctatggtcagccgtagcattaaccagggctcaatagatggcgatggagat

SEQ ID NO: 1307 - DNA, 95 bp, Influenza virus
tcgtgacccatcgggacggttgagcttgcgttccacatagatcgcgacaagagagggcccaaattagcagacatctaagatcttaacagaatgta

SEQ ID NO: 1308 - DNA, 148 bp, Influenza virus
tcgtgacccatcgagacggttgagctggcattccacatagatcgcgacaggagagggcccaaattagcagatatctaagatcttaacagaatgtagcgctatggtcagccgtagcattaaccagggctcagtagatggcgatggagat

SEQ ID NO: 1309 - DNA, 148 bp, Influenza virus
tcgcgacccatcaagacggttgagctggtattccacatagatcgcgtcaggagaggacccaaactagcagatatctaagatcttaacagaatgtagcgctatggtcagccgtaacgttaaccaaggctcggtaaatggcgatggagat

SEQ ID NO: 1310 - DNA, 147 bp, Influenza virus
ttgcagatctcctggatggttaagctgacatctcccacaagccgcgatagacagaagtttacaccagcaagtacctacgatcttaacagcatgtagtgcggtgatcaaccataacgtcagtcaaaggtcggtggatgacgatggtgg

SEQ ID NO: 1311 - DNA, 148 bp, Influenza virus
ctacagattacttgaatgaccaaaccgataactcctgcaaactgcagtggacggaaatctacactaaagaatgaccgcgatcccgttggaatatgatatggcagccgatcataacgtcggtccaatgttgatgggaaacagcagtagc

SEQ ID NO: 1312 - DNA, 148 bp, Influenza virus
ctacagattacttgaataaccaaaccgataactcctgcaaactgcagtggacggaaatctacactaaagaatgaccgcgatcccgttggaatatgatatggcagccgatcatagcatcggtccaatgtcgatgggaaacagcagtaat

SEQ ID NO: 1313 - DNA, 148 bp, Influenza virus
ctacagattatttgaatgaccaaaccgataactcctgcaaactgcagtggacggaaatctacactaaagaatgaccgcgatcccgttggaatatgatatggcagccgatcataacgtcggtccaatgtcgatgggaaacagcagtagt

SEQ ID NO: 1314 - DNA, 148 bp, Influenza virus
ttgcagattttttgaataaccaggtcgatatcttctacgagccataaaagacagaggtttgcgccgacgagtactcgtaacttcgtcgacaaacgatatggtgattgattactgtaacagttcaatatcggcggaagaagacggtggt

SEQ ID NO: 1315 - DNA, 148 bp, Influenza virus
ttgcagattttttgaataaccaggtcgatatcttctacgagccataaaagacagaggtttgcgccgacgagtactcgtagcttcgtcgacacacgatatggtgattgattactataacagttcaatatcggcggaagaagacgttggt

SEQ ID NO: 1316 - DNA, 148 bp, Influenza virus
ttgcagattttttgagtaaccaggtcgatatcttctacgagccataaaagacagaggtttgcgccgacgagtactcgtaatttcgttgacatacgatatggtgattgattattacaacagttcaatattggtggaagaagacggtggc

SEQ ID NO: 1317 - DNA, 183 bp, Influenza virus
aacgaggtacgaggggtccgatctgtaacaatgatagcgccccaaaaatacattgacagtactcatcaaatagcaatatcatcggaaccccgaataactggttaccatccgggagcgagctacaaaaatacggtggggcatttgaccagcttttacttacatgcacggcttacggagtcaaca

SEQ ID NO: 1318 - DNA, 183 bp, Influenza virus
aacgaggtacgaggggttcgatctgtaacaatgatagcgccccaaaaatacgttgacagtactcatcaaatagcaatatcatcggaacccagaataactggtcaccatccgggagcgaattacaaaaacacggtggggcatttgaccagcttctacttacatgcacggcctacggaatcaaca

SEQ ID NO: 1319 - DNA, 183 bp, Influenza virus
aacgaggtacgaggggtccgatctgtaacaatgatagcgccccaaaaatacattgacagtactcatcaaatagcaatatcatcggaaccccgaataactggtcattgtctaaagttgggccgtaggggtgcggcggggcattcagttgaccctcgactagatatgtaatctgcaaaactgata

SEQ ID NO: 1320 - DNA, 183 bp, Influenza virus
ggtagaacacggaaaactcaacccatggcggcagagataatttcggcgcatgtcggcgacatccgcaggacaatagcgctgcagaggtttaaagcgctgagaaattgcctagagttgggccgtaggggtgcggcgagatgctcggttgaccctcgactaggtatgtaatctgcaaaactgatg

SEQ ID NO: 1321 - DNA, 183 bp, Influenza virus
ggtagaacacggaaagctcaacccacggcggcagagataatttcggcgcatgtcggcaacatctgcaggacagtagctctgcagaggtttaaagcgctgagaaattgcctagagtcgggtcgtaggggtgcgacgagatgctcagttgacctccgactaggtaagtaatccgcaaaactgatg

SEQ ID NO: 1322 - DNA, 183 bp, Influenza virus
agcaaggtgcgagggatccgacctgcaataatagtagcactcctaaaatacgccagcaatacccgtagaatagcagaatcatcggaatccagaataactagacattatcagagagcggactataaaagcatgatggggcatttgaccagccattacttgcataaacgacctacggagtcaaca

SEQ ID NO: 1323 - DNA, 183 bp, Influenza virus
agcgaggtaaaagggatctgtattatgataatagtagcgccccgaaaatgcgccaatggcactcgtagagtgacggtatcatccaaacttagaataactgaccgctattcgggaacaaactacgaaaacatgatagagcatctgaccagtccctattcgcacacacggcccatgggatcgctg

SEQ ID NO: 1324 - DNA, 183 bp, Influenza virus
agcgaggtaaaaaggatctgtattatgataatagtagcgccccgaaaatgcgccaatggcattcatagagtgacggtatcatccaaacttagaataactgaccactattcgagaacaaactacgaaaacatgatagggcatctgaccagtccctattcgcacacacggcccatggggtcgctg

SEQ ID NO: 1325 - DNA, 183 bp, Influenza virus
agcgaggtaaaagggatctgtcttataataatagtagcgccccgaaaatgcgccaacggcattcgtagagtgacggtatcatccaaacttagaataactgaccaccattcgggaacaaactacgaaaacatgatagagcatctgaccagtccctattcgcacacacggcccatgggatcgctg

SEQ ID NO: 1326 - DNA, 183 bp, Influenza virus
agcgaggtaaaagggatctgtcttataataatagtagcgccccgaaaatgcgccaacggcactcgtagagtgacggtatcatccaaactcaggataactgaccactattcgggaacaaactacgaaaacaggatagagcatctgaccagcctctattcgcacacacggcccatgggatcgctg

SEQ ID NO: 1327 - DNA, 183 bp, Influenza virus
agcgaggtaaaagggatctgtcttataataatagtagcgccccgaaaatgcgccaacggcactcgtagagtgacggtatcatccaaactcaggataactgaccgctattcgggaacaaactacgaaaacaggatagagcatctgaccagcctctattcgcacgcacggcccatgggatcgatg

SEQ ID NO: 1328 - DNA, 183 bp, Influenza virus
agcgaggtaaaagggatctgtcttataataatagtagcgccccgaaaatgcgccaacggcgctcatagagtgacggtatcatccaaactcagaataactgaccactattcgggaacaaactacgaaaacatgatagagcatctgaccagtctctattcggacacacggcccatgggatcgctg

SEQ ID NO: 1329 - DNA, 183 bp, Influenza virus
agcgaggtaaaagggatctgtcttataataatagtagcgccccgaaaatgcgccaacggcactcgtagagtgacggtatcatccaaacttagaataactgaccactattcgggaacaaactacgaaaacatgatagagcatctgaccagtccctagtcgcacacacggcccatgggatcgctg

SEQ ID NO: 1330 - DNA, 102 bp, Influenza virus
gcagttattagttaaacttttcactgcacaattaaccttactgggccgacggttggacgacaactaaacgtgcagcggaggggggttaattcaaccgctcca

SEQ ID NO: 1331 - DNA, 108 bp, Influenza virus
gcagttatcagttaagcttttcactgcacgattaaccttactgggcccaaggttggtatgacaacgaaacgtgcagtcagagtgtgtgagtttaattcaaccgcttca

SEQ ID NO: 1332 - DNA, 102 bp, Influenza virus
acagttatcagttaagctttccactgcacgattgaccttactgggccgaaggtcggacgacaactaaacgtgcagcagaggggggttaattcaaccgctcca

SEQ ID NO: 1333 - DNA, 101 bp, Influenza virus
acagttatcagttaagcttttcactgcacgatcaattccgtcaaattaggaagaagtagtgatagggtacataaaggaaaaaaaccggcatggtcatcctg

SEQ ID NO: 1334 - DNA, 108 bp, Influenza virus
gcagttattagttaagcttttcactgcacaatcaaccttactgggttaggggttggtacgacaacgaaacgtgcagtcagagtgtgtaagtttaatatgaccatccca

SEQ ID NO: 1335 - DNA, 101 bp, Influenza virus
gtgaccgcttacaggatctcctgtcctgtagacgattccgtcgaattaggaagaagtagtgatagggtacatgaagggaaaaaaccggcatggtcatcttg

SEQ ID NO: 1336 - DNA, 101 bp, Influenza virus
gtggccgcttacaggatctcctgtcctgtagacaattccgtcgaattaggaagaagcagtgatagggtacataaagagaaaaaaccggcatgattatcctg

SEQ ID NO: 1337 - DNA, 101 bp, Influenza virus; identical to SEQ ID NO: 1336

SEQ ID NO: 1338 - DNA, 101 bp, Influenza virus
gtgaccgcttacaggatctcctgtcctgtagacaattccgtcgaattaggaagaagtagtgatagggtacataaagggaaaaaaccggcatggtcatcctg

SEQ ID NO: 1339 - DNA, 101 bp, Influenza virus
gtgaccgcttacaggatcacctgtcctgtagacaattccgtcaaattaggaagaagtagtgatagggtacatgaaggaaaaaaaccggcatggtcatcctg

SEQ ID NO: 1340 - DNA, 101 bp, Influenza virus; identical to SEQ ID NO: 1339

SEQ ID NO: 1341 - DNA, 101 bp, Influenza virus
gtgaccgcttacaggatctcctgtcctgtagacagttccgtcgaattaggaagaagtagtgatagggtacataaagagaaaaaaccggcatggtcatcctg

SEQ ID NO: 1342 - DNA, 101 bp, Influenza virus
gtgaccgcttacaggatctcctgtcctgtagacagttccgtcgaattaggaagaagtagtgatagggtacgtaaagggagaaaaccggcatggtcatcctg

SEQ ID NO: 1343 - DNA, 148 bp, Influenza virus; identical to SEQ ID NO: 1302

SEQ ID NO: 1344 - DNA, 148 bp, Influenza virus; identical to SEQ ID NO: 1303

SEQ ID NO: 1345 - DNA, 148 bp, Influenza virus; identical to SEQ ID NO: 1304

SEQ ID NO: 1346 - DNA, 148 bp, Influenza virus; identical to SEQ ID NO: 1305

SEQ ID NO: 1347 - DNA, 148 bp, Influenza virus; identical to SEQ ID NO: 1306

SEQ ID NO: 1348 - DNA, 95 bp, Influenza virus; identical to SEQ ID NO: 1307

SEQ ID NO: 1349 - DNA, 148 bp, Influenza virus; identical to SEQ ID NO: 1308

SEQ ID NO: 1350 - DNA, 148 bp, Influenza virus; identical to SEQ ID NO: 1309

SEQ ID NO: 1351 - DNA, 147 bp, Influenza virus; identical to SEQ ID NO: 1310

SEQ ID NO: 1352 - DNA, 148 bp, Influenza virus; identical to SEQ ID NO: 1311

SEQ ID NO: 1353 - DNA, 148 bp, Influenza virus; identical to SEQ ID NO: 1312

SEQ ID NO: 1354 - DNA, 148 bp, Influenza virus; identical to SEQ ID NO: 1313

SEQ ID NO: 1355 - DNA, 148 bp, Influenza virus; identical to SEQ ID NO: 1314

SEQ ID NO: 1356 - DNA, 148 bp, Influenza virus; identical to SEQ ID NO: 1315

SEQ ID NO: 1357 - DNA, 148 bp, Influenza virus; identical to SEQ ID NO: 1316

SEQ ID NO: 1358 - DNA, 148 bp, Influenza virus
ttgcgacccatcaagacggttgaactggcattccatatagaccgcgacaggagagggcccaaactagcagatatctaagatcttaacagaatgtagcgctatggtcagccgtagcattaacccgggctcggcaaatggcgatggaggt

SEQ ID NO: 1359 - DNA, 148 bp, Influenza virus
ttgcagattttttggatgaccaggctgatatctcccacaagccacgatagacggaagcttacactgacaaatacctacaatctcaacggcatatggtatgatagtcagtcataacgtcggtccagtctcagtggaggacgatggtgat

SEQ ID NO: 1360 - DNA, 237 bp, Influenza virus
gagtgacggtaggctcacgtaatgcaatctccctttaaggaaacacaccggatcggtgccatccgtcccgccgcctaatgcattgaatgatagagatggtgaatgcagcgctaaatggatcgtctgaactgcgccttgtaaagaaacacttcattcatgttaagaaaccagaccctagagcagaaactgcaaccgccttcagcacacgtgccaaattggcccgtatcacaggagcca

SEQ ID NO: 1361 - DNA, 237 bp, Influenza virus
gagtgacggtaggctcacgtaatgcaatctccctttaaggaaacacaccggatcggtgccatccgtccggccgcctaatgcattgaatgatagaaatggtgaatgcagcgctaaatcgatcgtctgaactgcgccttgtaaagaaacacttcattcatgttaagaaaccagaccctagagcaaaaactgcaaccgccttcagcacacgtgccaaattggcccgtatcacaggagcca

SEQ ID NO: 1362 - DNA, 237 bp, Influenza virus
gagtgacggtaggctcacgtaatgcaatctccctttaaggaaacacaccggatcggtgccatccgtccggccgcctaatgcattgaatgatagaaatggtggatgcagcgctaaatcgatcgtctgaactgcgccttgtaaagaaacacttcattcatgttaagaaaccagaccctagagcaaaaactgcaaccgccttcggcacacatgccaaattggcccgtatcacaggagcca

SEQ ID NO: 1363 - DNA, 53 bp, Influenza virus
aactgcgaccgccttcagcacacgtgccaaattggcccatgtcacaggagcca

SEQ ID NO: 1364 - DNA, 53 bp, Influenza virus
ggtcaaaaccgccttcagcacacgtgccaaattggcccgtatcacaggagcca

SEQ ID NO: 1365 - DNA, 237 bp, Influenza virus
agacagtcacgaaaatgtaaggcatgatctcctttaagaagggtgtgtaaacctaacattgctaactagatttttcggaattcgaggcagccagagcatcaggcagcttatctggcaatctactaacgtcaattaccacgtgagttagtccttaatgcaaaggagggttgagataacagatgggggtcaagcttatacctgctctgtatattgggcccattgacgcacagaagattg

SEQ ID NO: 1366 - DNA, 236 bp, Influenza virus
gagtgacggtaggctcacgtaatgcaatctccctttaaggaaacacaccggatcggtgccatccgtcccgccgcctaatgcattgaatgatagagatggtgaatgcagcgctaaatggatcgtctgaactgcgccttgtaaagaaacacttcattcatgttaagaaaccagaccctagagcagaactgcaaccgccttcagcacacgtgccaaattggcccgtatcacaggagcca

SEQ ID NO: 1367 - DNA, 236 bp, Influenza virus
gagtgacggtaggctcacgtaatgcaatctccctttaaggaaacacaccggatcggtgccatccgtccggccgcctaatgcattgaatgatagaaatggtgaatgcagcgctaaatcgatcgtctgaactgcgccttgtaaagaaacacttcattcatgttaagaaaccagaccctagagcaaaactgcaaccgccttcagcacacgtgccaaattggcccgtatcacaggagcca

SEQ ID NO: 1368 - DNA, 236 bp, Influenza virus
gagtgacggtaggctcacgtaatgcaatctccctttaaggaaacacaccggatcggtgccatccgtccggccgcctaatgcattgaatgatagaaatggtggatgcagcgctaaatcgatcgtctgaactgcgccttgtaaagaaacacttcattcatgttaagaaaccagaccctagagcaaaactgcaaccgccttcggcacacatgccaaattggcccgtatcacaggagcca

SEQ ID NO: 1369 - DNA, 236 bp, Influenza virus
agacagtcacgaaaatgtaaggcatgatctcctttaagaagggtgtgtaaacctaacattgctaactagatttttcggaattcgaggcagccagagcatcaggcagcttatctggcaatctactaacatcaattaccacgtgagttagtccttaatgcaaaggagggttgagataacagatggaactgcgaccgccttcagcacacgtgccaaattggcccatgtcacaggagcca

SEQ ID NO: 1370 - DNA, 236 bp, Influenza virus
agacagtcacgaaaatgtaaggcatgatctcctttaagaagggtgtgtaaacctaacattgctaactagatttttcggaattcgaggcagccagagcatcaggcagcttatctggcaatctactaacgtcaattaccacgtgagttagtccttaatgcaaaggagggttgagataacagatggggtcaaaaccgccttcagcacacgtgccaaattggcccgtatcacaggagcca

SEQ ID NO: 1371 - DNA, 236 bp, Influenza virus
agacagtcacgaaaatgtaaggcatgatctcctttaagaagggtgtgtaaacctaacattgctaactagatttttcggaattcgaggcagccagagcatcaggcagcttatctggcaatctactaacgtcaattaccacgtgagttagtccttaatgcaaaggagggttgagataacagatggggtcaagcttatacctgctctgtatattgggcccattgacgcacagaagattg

SEQ ID NO: 1372 - DNA, 236 bp, Influenza virus; identical to SEQ ID NO: 1366

SEQ ID NO: 1373 - DNA, 236 bp, Influenza virus; identical to SEQ ID NO: 1367

SEQ ID NO: 1374 - DNA, 236 bp, Influenza virus; identical to SEQ ID NO: 1368

SEQ ID NO: 1375 - DNA, 236 bp, Influenza virus
agacagtcacgaaaatgtaaggcatgatctcctttaagaagggtgtgtaaacctaacattgctaactagatttttcggaattcgaggcagccagagcatcaggcagcttatctggcaatctactaacatcaattaccacgtgagttagtccttaatgcaaaggagggttgagataacagatggaactgcaaccgccttcggcacacgtgccaaattggcccgtgtcacaggagcca

SEQ ID NO: 1376 - DNA, 236 bp, Influenza virus
agacagtcacgaaaatgtaaggcatgatctcctttaagaagggtgtgtaaacctaacattgctaactagatttttcggaattcgaggcagccagagcatcaggcagcttatctggcaatctactaacgtcaattaccacgtgagttagtccttaatgcaaaggagggttgagataacagatggggtcaagcccgccttcagcacacgtgccaaattggcccgtatcacaggagcca

SEQ ID NO: 1377 - DNA, 236 bp, Influenza virus; identical to SEQ ID NO: 1371

SEQ ID NO: 1378 - DNA, 96 bp, Influenza virus
ttgtccgattcttctgagattgcctcccaatttgtctggtattcctggtgagtcgcaccagtccatatgagactttgttacggatggatataggac

SEQ ID NO: 1379 - DNA, 96 bp, Influenza virus
aagtacgattctcctgaggccgcttcccaattcgtctgatactctcggtaagccgcatgtattaatagaagatctcgttacggacggatataggac

SEQ ID NO: 1380 - DNA, 96 bp, Influenza virus
aaacatgactccaccaaggccgcttcctaatttgtctgataccctcagcagacgagttgtgtttgtagaaagcttcgtcacagatgggcgcaagga

SEQ ID NO: 1381 - DNA, 96 bp, Influenza virus; identical to SEQ ID NO: 1380

SEQ ID NO: 1382 - DNA, 96 bp, Influenza virus
taacatgactccaccaaggccgcttcctaatttgtctgataccctcatcagaccacatgtgtttgtagaaagcttcgtcacagatgggcgcaagga

SEQ ID NO: 1383 - DNA, 96 bp, Influenza virus
aaacatgactccaccaaggccgcttcctaatttgtctgataccctcatcagaccacatgtgtttagagaaaatctcgtcacagatgggcgtaagga

SEQ ID NO: 1384 - DNA, 96 bp, Influenza virus
aagtatgattttaccaaggccgctttccaatgtatctcatactctcagtaaaccacatgtgtttatagaagacctcgttacaagcaaacgtaacga

SEQ ID NO: 1385 - DNA, 91 bp, Influenza virus
aagtatgattttaccaaggccgctttccaatgtatctcatactctcagtaaaccacatgtgtttatagaagacctcgttacagacggacgt

SEQ ID NO: 1386 - DNA, 96 bp, Influenza virus
atgtatgattctaccaaggccgcttcccaaagtgcctgataccctcagtaaaccacatgtgtttatagaagacctcgttacagacagacgcaagaa

SEQ ID NO: 1387 - DNA, 96 bp, Influenza virus
aagtacaattctaccaaagccgcttcccagcgtgctcgatcctctcagtaagccacatgtgtctatagaagaccctgatataagcaaacgcgagaa

SEQ ID NO: 1388 - DNA, 96 bp, Influenza virus; identical to SEQ ID NO: 1387

SEQ ID NO: 1389 - DNA, 96 bp, Influenza virus
aagtacaattctaccaaagccgcttcccagcgtgctcgatcctctcagtaagccacatgtgtctatagaggaccctaatgtaagcaaacacgagaa

SEQ ID NO: 1390 - DNA, 96 bp, Influenza virus
aagtacaattctaccaaagccgcttcccagcgtgctcgatcctctcagtaagccacatgtatctatagaggaccctaatgtaagcaaacacgagaa

SEQ ID NO: 1391 - DNA, 96 bp, Influenza virus
aagtccgatgcttttgagattatctcccgagtcgtctggcattactggtgagtcgcaccaatctatagaggaccctaatgtaagcaaacacgacaa

SEQ ID NO: 1392 - DNA, 96 bp, Influenza virus
aagtccgatgcttttgagattatctcccgagtcgtctggcattactggtgagtcgcaccaatctatagaggaccctaatgtaagcaaacacgagaa

SEQ ID NO: 1393 - DNA, 96 bp, Influenza virus
aagtccgatgcttttgagattatctcccgagtcgtctggcattactggtgagtcgcaccagccaacgggagaccctgatataagcaaacgcgagaa

SEQ ID NO: 1394 - DNA, 70 bp, Influenza virus
aagtccggtgcttttgagattgtcccccaagtcgtctggcattactggtgagtcgcaccagccaacgaga

SEQ ID NO: 1395 - DNA, 70 bp, Influenza virus
aagtccgatgcttttgggattgtctctcaagtcgtctggcattactggtgagtcgcaccagccaacgcga

SEQ ID NO: 1396 - DNA, 70 bp, Influenza virus
aagtccgatgcttttgagattgtctcccaagtcgtctggcattactggtgagtcgtaccagccaacggga

SEQ ID NO: 1397 - DNA, 70 bp, Influenza virus
aagtccgatgcttttgagattgtctcccaagtcgtctggcattactggtgagtcgcaccagccaacggga

SEQ ID NO: 1398 - DNA, 85 bp, Influenza virus
gagataacgagctcatcaggtcgtttaagcagtgaatacgcccaggttgcgactggttcaaggaagtacaccctgttagatagtt

SEQ ID NO: 1399 - DNA, 85 bp, Influenza virus
gagatagcgagctcatcaggtcgtttaagcagtgaatacgcccaggttgcgactggttcaaggaagtacaccctgttagatagtt

SEQ ID NO: 1400 - DNA, 85 bp, Influenza virus
gagatagcgagctcatcaggtcgtttaagcagtgaatacgcccaggttgcgactggttcaaggaagtacaccctgtcagatagtt

SEQ ID NO: 1401 - DNA, 85 bp, Influenza virus; identical to SEQ ID NO: 1398

SEQ ID NO: 1402 - DNA, 85 bp, Influenza virus; identical to SEQ ID NO: 1398

SEQ ID NO: 1403 - DNA, 85 bp, Influenza virus
gagataacgagctcatcaggtcgtttaaacagtgaatacgcccaggttgcgactggttcaaggaagtacaccctgttagatagtt

SEQ ID NO: 1404 - DNA, 85 bp, Influenza virus
gagataacgagctcatcaggtcgtttaaacagtgaatacgcccaggttgcgactggtccaaggaagtacaccctgttagatagtt

SEQ ID NO: 1405 - DNA, 85 bp, Influenza virus; identical to SEQ ID NO: 1404

SEQ ID NO: 1406 - DNA, 85 bp, Influenza virus
gagataacgaactcatcaggtcgtttaaacagtgaatacgcccaggttgcgactggttcaaggaagtacgccccgttagatagtt

SEQ ID NO: 1407 - DNA, 85 bp, Influenza virus; identical to SEQ ID NO: 1406

SEQ ID NO: 1408 - DNA, 85 bp, Influenza virus
gagatcacgagttcaccggatcgtttaggcagcgcatacgaacgaaccatagctggttcgaggaagcccactctattagatagtt

SEQ ID NO: 1409 - DNA, 85 bp, Influenza virus
gagaccaagagttaacctgatcatttaggcagcgcatacgcccagattgtgactggttcgaggaagcccaccctattagataatt

SEQ ID NO: 1410 - DNA, 85 bp, Influenza virus; identical to SEQ ID NO: 1409

SEQ ID NO: 1411 - DNA, 85 bp, Influenza virus; identical to SEQ ID NO: 1409

SEQ ID NO: 1412 - DNA, 85 bp, Influenza virus
gagaccaagagttaacctgatcatttaggcagcgcatacgaacgaaccatagctggttcgaggaagcccaccctattagataatt

SEQ ID NO: 1413 - DNA, 85 bp, Influenza virus
agagtcgaaagtccacttaactacttgagtggcgcgcgcaagagaactatagtcaacttggaaagaccaaccatatcaatcgacc

SEQ ID NO: 1414 - DNA, 85 bp, Influenza virus
agagtcgaaagtccacttaactacttgagtggcgcgcgcaagagaactatagtcaacttggaaagaccaaccatatcgatcgacc

SEQ ID NO: 1415 - DNA, 85 bp, Influenza virus
agagtcgagagtccacttaactacttgagtggcgcgtacaagcgaaccatagtcaacttggaaagaccaactctatcgatcaacc

SEQ ID NO: 1416 - DNA, 85 bp, Influenza virus
aaagtcgaaggtccgcttaactacccgagtgactcgtataaacgaaccatagtcaacttgagaggaccaattctagcgatcaatc

SEQ ID NO: 1417 - DNA, 85 bp, Influenza virus; identical to SEQ ID NO: 1416

SEQ ID NO: 1418 - DNA, 85 bp, Influenza virus; identical to SEQ ID NO: 1416

SEQ ID NO: 1419 - protein, 5 aa, Influenza virus
KTMVL

SEQ ID NO: 1420 - protein, 5 aa, Influenza virus
QIIII

SEQ ID NO: 1421 - protein, 4 aa, Influenza virus
SASK

SEQ ID NO: 1422 - protein, 4 aa, Influenza virus
NGNR